(19) United States
(12) Patent Application Publication
(10) Pub. No.: US 2026/0297613 A1
(43) Pub. Date: Oct. 1, 2026

(54) METHODS AND COMPOSITIONS FOR INSERTION OF ANTIBODY CODING SEQUENCES INTO A SAFE HARBOR LOCUS

(71) Applicant: Regeneron Pharmaceuticals, Inc., Tarrytown, NY (US)

(72) Inventors: Cheng Wang, Long Island City, NY (US); Suzanne Hartford, Putnam Valley, NY (US); Guochun Gong, Pleasantville, NY (US); Christos Kyratsous, Irvington, NY (US); Brian Zambrowicz, Sleepy Hollow, NY (US); George D. Yancopoulos, Yorktown Heights, NY (US)

(73) Assignee: Regeneron Pharmaceuticals, Inc., Tarrytown, NY (US)

(21) Appl. No.: 19/414,775

(22) Filed: Dec. 10, 2025

Related U.S. Application Data
(63) Continuation of application No. 18/671,080, filed on May 22, 2024, now abandoned.
Application No. 18/671,080 is a continuation of application No. 16/838,709, filed on Apr. 2, 2020, now abandoned.
(60) Provisional application No. 62/828,518, filed on Apr. 3, 2019.
(60) Provisional application No. 62/887,885, filed on Aug. 16, 2019.

(51) Int. Cl. C12N 15/85; A01K 67/0278; C07K 14/705; C12N 9/22

(52) U.S. Cl. CPC C12N 15/8509; A01K 67/0278; C07K 14/7051; C12N 9/22; A01K 2207/15; A01K 2217/15; C12N 2015/8527; C12N 2310/20

(57) ABSTRACT

Methods and compositions are provided for integrating coding sequences for antigen-binding proteins such as broadly neutralizing antibodies into a safe harbor locus such as an albumin locus in an animal in vivo.

### CROSS-REFERENCE TO RELATED APPLICATIONS

This application is a continuing application of U.S. application Ser. No. 18/671,080, filed May 22, 2024, which is a continuing application of U.S. application Ser. No. 16/838,709, filed Apr. 2, 2020, which claims the benefit of U.S. Application No. 62/828,518, filed Apr. 3, 2019, and U.S. Application No. 62/887,885, filed Aug. 16, 2019, each of which is herein incorporated by reference in its entirety for all purposes.

### REFERENCE TO A SEQUENCE LISTING SUBMITTED AS AN XML FILE

The Sequence Listing written in file 612520SEQLIST.xml is 227,635 bytes, was created on May 22, 2024, and is hereby incorporated by reference.

### BACKGROUND

Neutralizing antibodies play an essential part in antibacterial and antiviral immunity and are instrumental in preventing or modulating bacterial or viral diseases. Antibodies developed by the immune system upon infection or active vaccination tend to focus on easily accessible loops on the bacterial or viral surface, which often have great sequence and conformational variability. However, the bacteria or virus population can quickly evade these antibodies, and the antibodies are attacking portions of the protein that are not essential for function. Although broadly neutralizing antibodies can overcome these problems, these antibodies usually come too late to provide effective protection from the disease, and treatment with such antibodies provides only short-lived protection.

### SUMMARY

Animals comprising coding sequences for antigen-binding proteins integrated into a safe harbor locus, and methods for integrating coding sequences for antigen-binding proteins into a safe harbor locus in an animal in vivo are provided. Similarly, cells, genomes, or genes comprising coding sequences for antigen-binding proteins integrated into a safe harbor locus, and methods for integrating coding sequences for antigen-binding proteins into a safe harbor locus in a cell, genome, or gene in vitro or in vivo are provided. In one aspect, provided are methods for inserting an antigen-binding-protein coding sequence into a safe harbor locus in an animal in vivo. Some such methods comprise introducing into the animal a nuclease agent that targets a target site in the safe harbor locus and an exogenous donor nucleic acid comprising the antigen-binding-protein coding sequence, wherein the nuclease agent cleaves the target site and the antigen-binding protein coding sequence is inserted into the safe harbor locus to produce a modified safe harbor locus. Some such methods comprise introducing into the animal: (a) a nuclease agent that targets a target site in the safe harbor locus or one or more nucleic acids encoding the nuclease agent; and (b) an exogenous donor nucleic acid comprising the antigen-binding-protein coding sequence, wherein the nuclease agent cleaves the target site and the antigen-binding protein coding sequence is inserted into the safe harbor locus to produce a modified safe harbor locus. Likewise, provided are methods for inserting an antigen-binding-protein coding sequence into a safe harbor locus in a cell in vitro or in vivo. Some such methods comprise introducing into the cell a nuclease agent that targets a target site in the safe harbor locus and an exogenous donor nucleic acid comprising the antigen-binding-protein coding sequence, wherein the nuclease agent cleaves the target site and the antigen-binding protein coding sequence is inserted into the safe harbor locus to produce a modified safe harbor locus. Some such methods comprise introducing into the cell: (a) a nuclease agent that targets a target site in the safe harbor locus or one or more nucleic acids encoding the nuclease agent; and (b) an exogenous donor nucleic acid comprising the antigen-binding-protein coding sequence, wherein the nuclease agent cleaves the target site and the antigen-binding protein coding sequence is inserted into the safe harbor locus to produce a modified safe harbor locus. In another aspect, provided is a nuclease agent and an exogenous donor nucleic acid comprising an antigen-binding-protein coding sequence, for use in inserting an antigen-binding-protein coding sequence into a safe harbor locus in a subject (e.g., animal or cell in vivo), wherein the nuclease agent targets and cleaves a target site in the safe harbor locus and wherein the exogenous donor nucleic acid is inserted into the safe harbor locus. In another aspect, provided is a nuclease agent or one or more nucleic acids encoding the nuclease agent and an exogenous donor nucleic acid comprising an antigen-binding-protein coding sequence, for use in inserting an antigen-binding-protein coding sequence into a safe harbor locus in a subject (e.g., animal or cell in vivo), wherein the nuclease agent targets and cleaves a target site in the safe harbor locus and wherein the exogenous donor nucleic acid is inserted into the safe harbor locus. Some such methods can comprise introducing into the animal or the cell a nuclease agent that targets a target site in the safe harbor locus and an exogenous donor nucleic acid comprising the antigen-binding-protein coding sequence, wherein the nuclease agent cleaves the target site and the antigen-binding protein coding sequence is inserted into the safe harbor locus to produce a modified safe harbor locus. Some such methods can comprise introducing into the animal or the cell: (a) a nuclease agent that targets a target site in the safe harbor locus or one or more nucleic acids encoding the nuclease agent; and (b) an exogenous donor nucleic acid comprising the antigen-binding-protein coding sequence, wherein the nuclease agent cleaves the target site and the antigen-binding protein coding sequence is inserted into the safe harbor locus to produce a modified safe harbor locus. In another aspect, provided is a nuclease agent and an exogenous donor nucleic acid comprising an antigen-binding-protein coding sequence, for use in treating or effecting prophylaxis of (preventing) a disease in a subject (e.g., animal), wherein the nuclease agent targets and cleaves a target site in a safe harbor locus of the subject, wherein the exogenous donor nucleic acid is inserted into the safe harbor locus, and wherein the antigen-binding protein is expressed in the subject and targets an antigen associated with the disease. In another aspect, provided is a nuclease agent or one or more nucleic acids encoding the nuclease agent and an exogenous donor nucleic acid comprising an antigen-binding-protein coding sequence, for use in treating or effecting prophylaxis of (preventing) a disease in a subject (e.g., animal), wherein the nuclease agent targets and cleaves a target site in a safe harbor locus of the subject, wherein the exogenous donor nucleic acid is inserted into the safe harbor locus, and wherein the antigen-binding protein is expressed in the subject and targets an antigen associated with the disease. Some such methods can comprise introducing into the animal a nuclease agent that targets a target site in a safe harbor locus and an exogenous donor nucleic acid comprising an antigen-binding-protein coding sequence, wherein the antigen-binding protein targets an antigen associated with the disease, wherein the nuclease agent cleaves the target site and the antigen-binding protein coding sequence is inserted into the safe harbor locus to produce a modified safe harbor locus, and whereby the antigen-binding protein is expressed in the animal and binds the antigen associated with the disease. Some such methods can comprise introducing into the animal: (a) a nuclease agent that targets a target site in a safe harbor locus or one or more nucleic acids encoding the nuclease agent; and (b) an exogenous donor nucleic acid comprising an antigen-binding-protein coding sequence, wherein the antigen-binding protein targets an antigen associated with the disease, wherein the nuclease agent cleaves the target site and the antigen-binding protein coding sequence is inserted into the safe harbor locus to produce a modified safe harbor locus, and whereby the antigen-binding protein is expressed in the animal and binds the antigen associated with the disease.

In some such methods, the antigen-binding protein targets a disease-associated antigen. In some such methods, of antigen-binding protein in the animal has a prophylactic or therapeutic effect against the disease in the animal. In another aspect, provided are methods treating or effecting prophylaxis of a disease in an animal having or at risk for the disease. Some such methods can comprise introducing into the animal a nuclease agent that targets a target site in a safe harbor locus and an exogenous donor nucleic acid comprising an antigen-binding-protein coding sequence, wherein the antigen-binding protein targets an antigen associated with the disease, wherein the nuclease agent cleaves the target site and the antigen-binding protein coding sequence is inserted into the safe harbor locus to produce a modified safe harbor locus, and whereby the antigen-binding protein is expressed in the animal and binds the antigen associated with the disease. Some such methods can comprise introducing into the animal: (a) a nuclease agent that targets a target site in a safe harbor locus or one or more nucleic acids encoding the nuclease agent; and (b) an exogenous donor nucleic acid comprising an antigen-binding-protein coding sequence, wherein the antigen-binding protein targets an antigen associated with the disease, wherein the nuclease agent cleaves the target site and the antigen-binding protein coding sequence is inserted into the safe harbor locus to produce a modified safe harbor locus, and whereby the antigen-binding protein is expressed in the animal and binds the antigen associated with the disease.

In some such methods, the inserted antigen-binding-protein coding sequence is operably linked to an endogenous promoter in the safe harbor locus. In some such methods, the modified safe harbor locus encodes a chimeric protein comprising an endogenous secretion signal and the antigen-binding-protein.

In some such methods, the safe harbor locus is an albumin locus. Optionally, the antigen-binding-protein coding sequence is inserted into the first intron of the albumin locus.

In some such methods, the antigen-binding protein coding sequence is inserted into the safe harbor locus in one or more liver cells in the animal.

In some such methods, the nuclease agent is a zinc finger nuclease (ZFN), a Transcription Activator-Like Effector Nuclease (TALEN), or a Clustered Regularly Interspaced Short Palindromic Repeats (CRISPR)-associated (Cas) protein and a guide RNA (gRNA). Optionally, the nuclease agent is the Cas protein and the gRNA, wherein the Cas protein is a Cas9 protein, and wherein the gRNA comprises: (a) a CRISPR RNA (crRNA) that targets the target site, wherein the target site is immediately flanked by a Protospacer Adjacent Motif (PAM) sequence; and (b) a trans-activating CRISPR RNA (tracrRNA). Optionally, the at least one gRNA comprises 2′-O-methyl analogs and 3′ phosphorothioate internucleotide linkages at the first three 5′ and 3′ terminal RNA residues.

In some such methods, the antigen-binding-protein coding sequence is inserted via non-homologous end joining. In some such methods, the exogenous donor nucleic acid does not comprise homology arms. In some such methods, the antigen-binding-protein coding sequence is inserted via homology-directed repair. In some such methods, the exogenous donor nucleic acid is single-stranded. In some such methods, the exogenous donor nucleic acid is double-stranded.

In some such methods, the antigen-binding protein coding sequence in the exogenous donor nucleic acid is flanked on each side by the target site for the nuclease agent, wherein the nuclease agent cleaves the target sites flanking the antigen-binding protein coding sequence. Optionally, the target site in the safe harbor locus is no longer present if the antigen-binding protein coding sequence is inserted into the safe harbor locus in the correct orientation but it is reformed if the antigen-binding protein coding sequence is inserted into the safe harbor locus in the opposite orientation. Optionally, the exogenous donor nucleic acid is delivered adeno-associated virus (AAV)-mediated delivery, and cleavage of the target sites flanking the antigen-binding protein coding sequence removes the inverted terminal repeats of the AAV.

In some such methods, the antigen-binding protein is an antibody, an antigen-binding fragment of an antibody, a multispecific antibody, an scFV, a bis-scFV, a diabody, a triabody, a tetrabody, a V-NAR, a VHH, a VL, a F (ab), a F (ab) 2, a dual variable domain antigen-binding protein, a single variable domain antigen-binding protein, a bispecific T-cell engager, or a Davisbody. In some such methods, the antigen-binding protein is not a single-chain antigen-binding protein. Optionally, the antigen-binding protein comprises a heavy chain and a separate light chain, optionally wherein the heavy chain coding sequence comprises V_{H}, D_{H}, and J_{H} segments, and the light chain coding sequence comprises V_{L} and J_{L} gene segments. In some such methods, the heavy chain coding sequence is upstream of the light chain coding sequence in the antigen-binding-protein coding sequence. Optionally, the antigen-binding-protein coding sequence comprises an exogenous secretion signal sequence upstream of the light chain coding sequence. In some such methods, the light chain coding sequence is upstream of the heavy chain coding sequence in the antigen-binding-protein coding sequence. Optionally, the antigen-binding-protein coding sequence comprises an exogenous secretion signal sequence upstream of the heavy chain coding sequence. In some such methods, the exogenous secretion signal sequence is a ROR1 secretion signal sequence.

In some such methods, the antigen-binding-protein coding sequence encodes a heavy chain and a light chain linked by a 2A peptide or an internal ribosome entry site (IRES). Optionally, the heavy chain and the light chain are linked by the 2A peptide. Optionally, the 2A peptide is a T2A peptide.

In some such methods, the disease-associated antigen is a cancer-associated antigen. In some such methods, the disease-associated antigen is an infectious-disease-associated antigen, such as a bacterial antigen. Optionally, the bacterial antigen is a Pseudomonas aeruginosa PcrV antigen. In some such methods, the disease-associated antigen is a viral antigen. Optionally, the viral antigen is an influenza antigen or a Zika antigen.

In some such methods, the viral antigen is an influenza hemagglutinin antigen. Optionally, the antigen-binding protein comprises a light chain comprising three light chain CDRs and a heavy chain comprising three heavy chain CDRs, wherein: (I) the light chain comprises, consists essentially of, or consists of a sequence at least 90% identical to the sequence set forth in SEQ ID NO: 18 and the heavy chain comprises, consists essentially of, or consists of a sequence at least 90% identical to the sequence set forth in SEQ ID NO: 20, optionally wherein the three light chain CDRs comprise, consist essentially of, or consist of sequences at least 90% identical to the sequences set forth in SEQ ID NOS: 76-78, respectively, and the three heavy chain CDRs comprise, consist essentially of, or consist of sequences at least 90% identical to the sequences set forth in SEQ ID NOS: 79-81, respectively; or (II) the modified safe harbor locus comprises a coding sequence at least 90% identical to the sequence set forth in SEQ ID NO: 120; or (III) the light chain comprises, consists essentially of, or consists of a sequence at least 90% identical to the sequence set forth in SEQ ID NO: 126 and the heavy chain comprises, consists essentially of, or consists of a sequence at least 90% identical to the sequence set forth in SEQ ID NO: 128, optionally wherein the three light chain CDRs comprise, consist essentially of, or consist of sequences at least 90% identical to the sequences set forth in SEQ ID NOS: 129-131, respectively, and the three heavy chain CDRs comprise, consist essentially of, or consist of sequences at least 90% identical to the sequences set forth in SEQ ID NOS: 132-134, respectively; or (IV) the modified safe harbor locus comprises a coding sequence at least 90% identical to the sequence set forth in SEQ ID NO: 146.

In some such methods, the viral antigen is a Zika Envelope (Env) antigen. Optionally, the antigen-binding protein comprises a light chain comprising three light chain CDRs and a heavy chain comprising three heavy chain CDRs, wherein: (I) the light chain comprises, consists essentially of, or consists of a sequence at least 90% identical to the sequence set forth in SEQ ID NO: 3 and the heavy chain comprises, consists essentially of, or consists of a sequence at least 90% identical to the sequence set forth in SEQ ID NO: 5, optionally wherein the three light chain CDRs comprise, consist essentially of, or consist of sequences at least 90% identical to the sequences set forth in SEQ ID NOS: 64-66, respectively, and the three heavy chain CDRs comprise, consist essentially of, or consist of sequences at least 90% identical to the sequences set forth in SEQ ID NOS: 67-69, respectively; or (II) the modified safe harbor locus comprises a coding sequence at least 90% identical to the sequence set forth in SEQ ID NO: 115. Optionally, antigen-binding protein comprises a light chain comprising three light chain CDRs and a heavy chain comprising three heavy chain CDRs, wherein: (I) the light chain comprises, consists essentially of, or consists of a sequence at least 90% identical to the sequence set forth in SEQ ID NO: 13 and the heavy chain comprises, consists essentially of, or consists of a sequence at least 90% identical to the sequence set forth in SEQ ID NO: 15, optionally wherein the three light chain CDRs comprise, consist essentially of, or consist of sequences at least 90% identical to the sequences set forth in SEQ ID NOS: 70-72, respectively, and the three heavy chain CDRs comprise, consist essentially of, or consist of sequences at least 90% identical to the sequences set forth in SEQ ID NOS: 73-75, respectively; or (II) the modified safe harbor locus comprises a coding sequence at least 90% identical to the sequence set forth in any one of SEQ ID NOS: 116-119.

In some such methods, the disease-associated antigen is a bacterial antigen.

In some such methods, the antigen-binding protein is a neutralizing antigen-binding protein or a neutralizing antibody. Optionally, the antigen-binding protein is a broadly neutralizing antigen-binding protein or a broadly neutralizing antibody.

In some such methods, the nuclease agent and the exogenous donor nucleic acid are introduced in separate delivery vehicles. In some such methods, the nuclease agent or the one or more nucleic acids encoding the nuclease agent and the exogenous donor nucleic acid are introduced in separate delivery vehicles. In some such methods, the nuclease agent and the exogenous donor nucleic acid are introduced together in the same delivery vehicle. In some such methods, the nuclease agent or the one or more nucleic acids encoding the nuclease agent and the exogenous donor nucleic acid are introduced together in the same delivery vehicle. In some such methods, the nuclease agent and the exogenous donor nucleic acid are introduced simultaneously. In some such methods, the nuclease agent or the one or more nucleic acids encoding the nuclease agent and the exogenous donor nucleic acid are introduced simultaneously. In some such methods, the nuclease agent and the exogenous donor nucleic acid are introduced sequentially. In some such methods, the nuclease agent or the one or more nucleic acids encoding the nuclease agent and the exogenous donor nucleic acid are introduced sequentially. In some such methods, the nuclease agent and the exogenous donor nucleic acid are introduced in single doses. In some such methods, the nuclease agent or the one or more nucleic acids encoding the nuclease agent and the exogenous donor nucleic acid are introduced in single doses. In some such methods, the nuclease agent and/or the exogenous donor nucleic acid are introduced in multiple doses. In some such methods, the nuclease agent or the one or more nucleic acids encoding the nuclease agent and/or the exogenous donor nucleic acid are introduced in multiple doses. In some such methods, the nuclease agent and the exogenous donor nucleic acid are delivered via intravenous injection. In some such methods, the nuclease agent or the one or more nucleic acids encoding the nuclease agent and the exogenous donor nucleic acid are delivered via intravenous injection.

In some such methods, the nuclease agent and the exogenous donor nucleic acid are introduced via lipid-nanoparticle-mediated delivery or via adeno-associated virus (AAV)-mediated delivery. Optionally, the nuclease agent and the exogenous donor nucleic acid are both introduced by AAV-mediated delivery. Optionally, the nuclease agent and the exogenous donor nucleic acid are introduced by multiple different AAV vectors (e.g., by two different AAV vectors). Optionally, the AAV is AAV8 or AAV2/8. In some such methods, the nuclease agent or the one or more nucleic acids encoding the nuclease agent and the exogenous donor nucleic acid are introduced via lipid-nanoparticle-mediated delivery or via adeno-associated virus (AAV)-mediated delivery. Optionally, the nuclease agent or the one or more nucleic acids encoding the nuclease agent and the exogenous donor nucleic acid are both introduced by AAV-mediated delivery. Optionally, the nuclease agent or the one or more nucleic acids encoding the nuclease agent and the exogenous donor nucleic acid are introduced by multiple different AAV vectors (e.g., by two different AAV vectors). Optionally, the AAV is AAV8 or AAV2/8. In some such methods, the nuclease agent is introduced via lipid-nanoparticle-mediated delivery. Optionally, the lipid nanoparticle comprises Dlin-MC3-DMA (MC3), cholesterol, DSPC, and PEG-DMG in a 50:38.5:10:1.5 molar ratio. In some such methods, the nuclease agent in the lipid nanoparticle is a Clustered Regularly Interspaced Short Palindromic Repeats (CRISPR)-associated 9 (Cas9) protein and a guide RNA (gRNA). Optionally, the Cas9 is in the form of mRNA, and the gRNA is in the form of RNA. In some such methods, the nuclease agent or the one or more nucleic acids encoding the nuclease agent is introduced via lipid-nanoparticle-mediated delivery. Optionally, the lipid nanoparticle comprises Dlin-MC3-DMA (MC3), cholesterol, DSPC, and PEG-DMG in a 50:38.5:10:1.5 molar ratio. In some such methods, the nuclease agent is a Clustered Regularly Interspaced Short Palindromic Repeats (CRISPR)-associated 9 (Cas9) protein and a guide RNA (gRNA). Optionally, the Cas9 is in the lipid nanoparticle is in the form of mRNA, and the gRNA in the lipid nanoparticle is in the form of RNA.

In some such methods, the exogenous donor nucleic acid is introduced via AAV-mediated delivery. Optionally, the AAV is a single-stranded AAV (ssAAV). Optionally, the AAV is a self-complementary AAV (scAAV). Optionally, the AAV is AAV8 or AAV2/8.

In some such methods, the nuclease agent comprises a Clustered Regularly Interspaced Short Palindromic Repeats (CRISPR)-associated 9 (Cas9)-encoding mRNA and a guide RNA (gRNA) introduced via lipid-nanoparticle-mediated delivery, and the exogenous donor nucleic acid is introduced via AAV8-mediated or AAV2/8-mediated delivery. In some such methods, the nuclease agent comprises a Clustered Regularly Interspaced Short Palindromic Repeats (CRISPR)-associated 9 (Cas9)-encoding DNA and a guide RNA (gRNA)-encoding DNA, wherein the Cas9-encoding DNA is introduced via AAV8-mediated delivery in a first AAV8 or AAV2/8-mediated delivery in a first AAV2/8, and the gRNA-encoding DNA and exogenous donor nucleic acids are introduced via AAV8-mediated delivery in a second AAV8 or AAV2/8-mediated delivery in a second AAV2/8. In some such methods, the nuclease agent comprises a Clustered Regularly Interspaced Short Palindromic Repeats (CRISPR)-associated 9 (Cas9) and a guide RNA (gRNA), wherein the method comprises introducing the gRNA and an mRNA encoding the Cas9 via lipid-nanoparticle-mediated delivery, and the exogenous donor nucleic acid is introduced via AAV8-mediated or AAV2/8-mediated delivery. In some such methods, the nuclease agent comprises a Clustered Regularly Interspaced Short Palindromic Repeats (CRISPR)-associated 9 (Cas9) and a guide RNA (gRNA), wherein the method comprises introducing a DNA encoding the Cas9 via AAV8-mediated delivery in a first AAV8 or AAV2/8-mediated delivery in a first AAV2/8, and introducing the exogenous donor nucleic acid and a DNA encoding the gRNA via AAV8-mediated delivery in a second AAV8 or AAV2/8-mediated delivery in a second AAV2/8.

In some such methods, expression of the antigen-binding protein in the animal results in plasma levels of at least about 2.5, at least about 5, at least about 10, at least about 100, at least about 200 μg/mL, at least about 300 μg/mL, at least about 400 μg/mL or at least about 500 μg/mL about 2 weeks, about 4 weeks, or about 8 weeks after introducing the nuclease agent and the exogenous donor sequence. In some such methods, expression of the antigen-binding protein in the animal results in plasma levels of at least about 2.5 μg/mL, at least about 5 μg/mL, at least about 10 μg/mL, at least about 100 μg/mL, at least about 200 μg/mL, at least about 300 μg/mL, at least about 400 μg/mL, at least about 500 μg/mL, at least about 600 μg/mL, at least about 700 μg/mL, at least about 800 μg/mL, at least about 900 μg/mL, or at least about 1000 μg/mL about 2 weeks, about 4 weeks, about 8 weeks, about 12 weeks, or about 16 weeks after introducing the nuclease agent or the one or more nucleic acids encoding the nuclease agent and the exogenous donor sequence.

In some such methods, the animal is a non-human animal. Optionally, the animal is a non-human mammal. Optionally, the non-human mammal is a rat or a mouse. In some such methods, the animal is a human.

In some such methods, the nuclease agent is a Clustered Regularly Interspaced Short Palindromic Repeats (CRISPR)-associated 9 (Cas9) protein and a guide RNA (gRNA), wherein the nuclease agent and the exogenous donor sequence are delivered via lipid-nanoparticle-mediated delivery, adeno-associated-virus 8 (AAV8)-mediated delivery, or AAV2/8-mediated delivery, wherein the antigen-binding-protein coding sequence is inserted into the first intron of an endogenous albumin locus via non-homologous end joining in one or more liver cells in the animal, wherein the inserted antigen-binding-protein coding sequence is operably linked to the endogenous albumin promoter, wherein the modified albumin locus encodes a chimeric protein comprising an endogenous albumin secretion signal and the antigen-binding-protein, wherein the antigen-binding protein targets a viral antigen or a bacterial antigen, wherein the antigen-binding protein is a broadly neutralizing antibody, and wherein the antigen-binding-protein coding sequences encodes a heavy chain and a separate light chain linked by a 2A peptide. Optionally, the heavy chain coding sequence is upstream of the light chain coding sequence in the antigen-binding-protein coding sequence, wherein the antigen-binding-protein coding sequence comprises an exogenous secretion signal sequence upstream of the light chain coding sequence, and wherein the exogenous secretion signal sequence is an ROR1 secretion signal sequence.

In some such methods, the nuclease agent is a Clustered Regularly Interspaced Short Palindromic Repeats (CRISPR)-associated 9 (Cas9) protein and a guide RNA (gRNA), the nuclease agent or the one or more nucleic acids encoding the nuclease agent and the exogenous donor sequence are delivered via lipid-nanoparticle-mediated delivery, adeno-associated-virus 8 (AAV8)-mediated delivery, or AAV2/8-mediated delivery, the antigen-binding-protein coding sequence is inserted into the first intron of an endogenous albumin locus via non-homologous end joining in one or more liver cells in the animal, the inserted antigen-binding-protein coding sequence is operably linked to the endogenous albumin promoter, the modified albumin locus encodes a chimeric protein comprising an endogenous albumin secretion signal and the antigen-binding-protein, the antigen-binding protein targets a viral antigen or a bacterial antigen, the antigen-binding protein is a broadly neutralizing antibody, and the antigen-binding-protein coding sequences encodes a heavy chain and a separate light chain linked by a 2A peptide. Optionally, the heavy chain coding sequence is upstream of the light chain coding sequence in the antigen-binding-protein coding sequence, wherein the antigen-binding-protein coding sequence comprises an exogenous secretion signal sequence upstream of the light chain coding sequence, and wherein the exogenous secretion signal sequence is an ROR1 secretion signal sequence.

In another aspect, provided are animals produced by any of the above methods. In another aspect, provided are cells, modified genomes, or modified safe harbor genes produced by any of the above methods. In another aspect, provided are animals, cells, or genomes comprising an exogenous antigen-binding-protein coding sequence integrated into a safe harbor locus.

In some such animals, cells, or genomes, the inserted antigen-binding-protein coding sequence is operably linked to an endogenous promoter in the safe harbor locus. In some such animals, cells, or genomes, the modified safe harbor locus encodes a chimeric protein comprising an endogenous secretion signal and the antigen-binding-protein.

In some such animals, cells, or genomes, the safe harbor locus is an albumin locus. Optionally, the antigen-binding-protein coding sequence is inserted into the first intron of the albumin locus.

In some such animals, cells, or genomes, the antigen-binding protein coding sequence is inserted into the safe harbor locus in one or more liver cells in the animal.

In some such animals, cells, or genomes, the antigen-binding protein is an antibody, an antigen-binding fragment of an antibody, a multispecific antibody, an scFV, a bis-scFV, a diabody, a triabody, a tetrabody, a V-NAR, a VHH, a VL, a F (ab), a F (ab) 2, a dual variable domain antigen-binding protein, a single variable domain antigen-binding protein, a bispecific T-cell engager, or a Davisbody. Optionally, the antigen-binding protein is not a single-chain antigen-binding protein. Optionally, the antigen-binding protein comprises a heavy chain and a separate light chain, optionally wherein the heavy chain coding sequence comprises V_{H}, D_{H}, and J_{H} segments, and the light chain coding sequence comprises V_{L} and J_{L} gene segments. In some such animals, cells, or genomes, the heavy chain coding sequence is upstream of the light chain coding sequence in the antigen-binding-protein coding sequence. Optionally, the antigen-binding-protein coding sequence comprises an exogenous secretion signal sequence upstream of the light chain coding sequence. In some such animals, cells, or genomes, the light chain coding sequence is upstream of the heavy chain coding sequence in the antigen-binding-protein coding sequence. Optionally, the antigen-binding-protein coding sequence comprises an exogenous secretion signal sequence upstream of the heavy chain coding sequence. In some such animals, cells, or genomes, the exogenous secretion signal sequence is a ROR1 secretion signal sequence.

In some such animals, cells, or genomes, the antigen-binding-protein coding sequence encodes a heavy chain and a light chain linked by a 2A peptide or an internal ribosome entry site (IRES). Optionally, the heavy chain and the light chain are linked by the 2A peptide. Optionally, the 2A peptide is a T2A peptide.

In some such animals, cells, or genomes, the antigen-binding protein targets a disease-associated antigen. In some such animals, cells, or genomes, expression of antigen-binding protein in the animal has a prophylactic or therapeutic effect against the disease in the animal. In some such animals, cells, or genomes, the disease-associated antigen is a cancer-associated antigen. In some such animals, cells, or genomes, the disease-associated antigen is an infectious-disease-associated antigen. Optionally, the disease-associated antigen is a viral antigen. Optionally, the viral antigen is an influenza antigen or a Zika antigen.

In some such animals, cells, or genomes, the viral antigen is an influenza hemagglutinin antigen. Optionally, the antigen-binding protein comprises a light chain comprising three light chain CDRs and a heavy chain comprising three heavy chain CDRs, wherein: (I) the light chain comprises, consists essentially of, or consists of a sequence at least 90% identical to the sequence set forth in SEQ ID NO: 18 and the heavy chain comprises, consists essentially of, or consists of a sequence at least 90% identical to the sequence set forth in SEQ ID NO: 20, optionally wherein the three light chain CDRs comprise, consist essentially of, or consist of sequences at least 90% identical to the sequences set forth in SEQ ID NOS: 76-78, respectively, and the three heavy chain CDRs comprise, consist essentially of, or consist of sequences at least 90% identical to the sequences set forth in SEQ ID NOS: 79-81, respectively; or (II) the modified safe harbor locus comprises a coding sequence at least 90% identical to the sequence set forth in SEQ ID NO: 120; or (III) the light chain comprises, consists essentially of, or consists of a sequence at least 90% identical to the sequence set forth in SEQ ID NO: 126 and the heavy chain comprises, consists essentially of, or consists of a sequence at least 90% identical to the sequence set forth in SEQ ID NO: 128, optionally wherein the three light chain CDRs comprise, consist essentially of, or consist of sequences at least 90% identical to the sequences set forth in SEQ ID NOS: 129-131, respectively, and the three heavy chain CDRs comprise, consist essentially of, or consist of sequences at least 90% identical to the sequences set forth in SEQ ID NOS: 132-134, respectively; or (IV) the modified safe harbor locus comprises a coding sequence at least 90% identical to the sequence set forth in SEQ ID NO: 146.

In some such animals, cells, or genomes, the viral antigen is a Zika Envelope (Env) antigen. Optionally, the antigen-binding protein comprises a light chain comprising three light chain CDRs and a heavy chain comprising three heavy chain CDRs, wherein: (I) the light chain comprises, consists essentially of, or consists of a sequence at least 90% identical to the sequence set forth in SEQ ID NO: 3 and the heavy chain comprises, consists essentially of, or consists of a sequence at least 90% identical to the sequence set forth in SEQ ID NO: 5, optionally wherein the three light chain CDRs comprise, consist essentially of, or consist of sequences at least 90% identical to the sequences set forth in SEQ ID NOS: 64-66, respectively, and the three heavy chain CDRs comprise, consist essentially of, or consist of sequences at least 90% identical to the sequences set forth in SEQ ID NOS: 67-69, respectively; or (II) the modified safe harbor locus comprises a coding sequence at least 90% identical to the sequence set forth in SEQ ID NO: 115. In some such animals, cells, or genomes, the antigen-binding protein comprises a light chain comprising three light chain CDRs and a heavy chain comprising three heavy chain CDRs, wherein: (I) the light chain comprises, consists essentially of, or consists of a sequence at least 90% identical to the sequence set forth in SEQ ID NO: 13 and the heavy chain comprises, consists essentially of, or consists of a sequence at least 90% identical to the sequence set forth in SEQ ID NO: 15, optionally wherein the three light chain CDRs comprise, consist essentially of, or consist of sequences at least 90% identical to the sequences set forth in SEQ ID NOS: 70-72, respectively, and the three heavy chain CDRs comprise, consist essentially of, or consist of sequences at least 90% identical to the sequences set forth in SEQ ID NOS: 73-75, respectively; or (II) the modified safe harbor locus comprises a coding sequence at least 90% identical to the sequence set forth in any one of SEQ ID NOS: 116-119.

In some such animals, cells, or genomes, the disease-associated antigen is a bacterial antigen. Optionally, the bacterial antigen is a Pseudomonas aeruginosa PcrV antigen.

In some such animals, cells, or genomes, the antigen-binding protein is a neutralizing antigen-binding protein or a neutralizing antibody. Optionally, the antigen-binding protein is a broadly neutralizing antigen-binding protein or a broadly neutralizing antibody.

In some such animals, cells, or genomes, expression of the antigen-binding protein in the animal results in plasma levels of at least about 2.5 μg/mL, at least about 5 μg/mL, at least about 10 μg/mL, at least about 100 μg/mL, at least about 200 μg/mL, at least about 300 μg/mL, at least about 400 μg/mL or at least about 500 μg/mL about 2 weeks, about 4 weeks, or about 8 weeks after introducing the nuclease agent and the exogenous donor sequence. In some such animals, cells, or genomes, expression of the antigen-binding protein in the animal results in plasma levels of at least about 2.5 μg/mL, at least about 5 μg/mL, at least about 10 μg/mL, at least about 100 μg/mL, at least about 200 μg/mL, at least about 300 μg/mL, at least about 400 μg/mL, at least about 500 μg/mL, at least about 600 μg/mL, at least about 700 μg/mL, at least about 800 μg/mL, at least about 900 μg/mL, or at least about 1000 μg/mL about 2 weeks, about 4 weeks, about 8 weeks, about 12 weeks, or about 16 weeks after introducing the nuclease agent and the exogenous donor sequence. In some such animals, cells, or genomes, expression of the antigen-binding protein in the animal results in plasma levels of at least about 2.5 μg/mL, at least about 5 μg/mL, at least about 10 μg/mL, at least about 100 μg/mL, at least about 200 μg/mL, at least about 300 μg/mL, at least about 400 μg/mL or at least about 500 μg/mL about 2 weeks, about 4 weeks, or about 8 weeks after introducing the nuclease agent or the one or more nucleic acids encoding the nuclease agent and the exogenous donor sequence. In some such animals, cells, or genomes, expression of the antigen-binding protein in the animal results in plasma levels of at least about 2.5 μg/mL, at least about 5 μg/mL, at least about 10 μg/mL, at least about 100 μg/mL, at least about 200 μg/mL, at least about 300 μg/mL, at least about 400 μg/mL, at least about 500 μg/mL, at least about 600 μg/mL, at least about 700 μg/mL, at least about 800 μg/mL, at least about 900 μg/mL, or at least about 1000 μg/mL about 2 weeks, about 4 weeks, about 8 weeks, about 12 weeks, or about 16 weeks after introducing the nuclease agent or the one or more nucleic acids encoding the nuclease agent and the exogenous donor sequence.

In some such animals, cells, or genomes, the animal is a non-human animal. Optionally, the animal is a non-human mammal. Optionally, the non-human mammal is a rat or a mouse. In some such animals, cells, or genomes, the animal is a human.

In some such animals, cells, or genomes, the antigen-binding-protein coding sequence is inserted into the first intron of an endogenous albumin locus in one or more liver cells in the animal, wherein the inserted antigen-binding-protein coding sequence is operably linked to the endogenous albumin promoter, wherein the modified albumin locus encodes a chimeric protein comprising an endogenous albumin secretion signal and the antigen-binding-protein, wherein the antigen-binding protein targets a viral antigen or a bacterial antigen, wherein the antigen-binding protein is a broadly neutralizing antibody, and wherein the antigen-binding-protein coding sequences encodes a heavy chain and a separate light chain linked by a 2A peptide. Optionally, the heavy chain coding sequence is upstream of the light chain coding sequence in the antigen-binding-protein coding sequence, wherein the antigen-binding-protein coding sequence comprises an exogenous secretion signal sequence upstream of the light chain coding sequence, and wherein the exogenous secretion signal sequence is an ROR1 secretion signal sequence.

In another aspect, provided are exogenous donor nucleic acids comprising an antigen-binding-protein coding sequence for insertion into a safe harbor locus. In another aspect, provided is a safe harbor gene comprising a coding sequence for an antigen-binding protein integrated into the safe harbor gene. In another aspect, provided is a method for generating a modified safe harbor gene, comprising contacting the safe harbor gene with a nuclease agent that targets a target site in the safe harbor gene and an exogenous donor nucleic acid comprising an antigen-binding-protein coding sequence, wherein the nuclease agent cleaves the target site and the antigen-binding protein coding sequence is inserted into the safe harbor gene to produce the modified safe harbor gene. In another aspect, provided is a method for generating a modified safe harbor gene, comprising contacting the safe harbor gene with an exogenous donor nucleic acid comprising an antigen-binding-protein coding sequence, wherein the antigen-binding protein coding sequence is inserted into the safe harbor gene to produce the modified safe harbor gene.

### BRIEF DESCRIPTION OF THE FIGURES

FIG. 1 (not to scale) shows a generic schematic for inserting antibody genes into the first intron of an endogenous albumin locus. SD refers to splice donor site, SA refers to splice acceptor site from the first intron of the mouse albumin gene, LC refers to antibody light chain (e.g., of anti-Zika, REGN4504), HC refers to antibody heavy chain (e.g., of anti-Zika, REGN4504), mAlbss refers to albumin secretion signal peptide encoded by exon 1 of the endogenous albumin gene, ss refers to the mouse Ror1 signal peptide; sWPRE refers to the woodchuck hepatitis virus posttranscriptional regulatory element, PolyA refers to the SV40 polyA sequence, and 2A refers to the 2A self-cleaving peptide from porcine teschovirus-1 (P2A).

FIG. 2 shows an experimental design to test insertion of an anti-Zika antibody into the first intron of the mouse albumin locus following delivery of Cas9 mRNA and albumin-targeting gRNA (either guide RNA 1 version 1 (N-Cap) or version 2) to the mouse liver via lipid nanoparticle (LNP) and delivery of an AAV2/8 AlbSA 4504 anti-Zika antibody donor sequence (light chain and heavy chain linked by P2A self-cleavage peptide).

FIG. 3 shows expression of the REGN4504 anti-Zika antibody (integrated AAV) as measured by ELISA in plasma samples from mice at 7 days (Week 1), 14 days (Week 2), and 28 days (Week 4) following co-injection of the LNP comprising of Cas9 mRNA and albumin-targeting gRNA (either guide RNA 1 version 1 (N-Cap) or version 2) and the AAV2/8 AlbSA 4504 anti-Zika antibody donor sequence. The y-axis shows hIgG concentration.

FIG. 4 shows Zika neutralization assay results in plasma samples drawn four weeks after injection of the Cas9-gRNA LNP and the AAV2/8 AlbSA 4504 anti-Zika antibody donor sequence. Results with a positive control antibody (REGN4504 anti-Zika antibody) are also shown.

FIG. 5 shows western blot analysis of antibodies produced by integrated AAV. #15 is one of the mice injected with LNP with Cas9 mRNA and guide RNA 1 v1. #17 is one of the mice injected with LNP with Cas9 mRNA and guide RNA 1 v2.

FIG. 6 shows a schematic for homology-independent-targeted-insertion-mediated unidirectional AAV-REGN4446 targeted insertion into intron 1 of the mouse albumin locus. hU6 gRNA1 is the expression cassette of guide RNA 1 v1 driven by the human U6 promoter. SA refers to the splicing acceptor from the first intron of mouse albumin gene, HC refers to the heavy chain of anti-Zika REGN4446, furin refers to the furin cleavage site, 2A refers to a 2A self-cleaving peptide (2A from Foot and mouth disease virus 18 (F2A), porcine teschovirus-1 (P2A), and thosea asigna virus (T2A) were tested), Ss refers to signal sequence (mouse albumin signal sequence and mouse Ror1 signal sequence were tested in this example), LC refers to light chain of anti-Zika REGN4446, WPRE refers to woodchuck hepatitis virus posttranscriptional regulatory element, and PolyA refers to the bovine growth hormone polyA sequence. The AAVs were injected into Cas9-ready mice.

FIG. 7 shows an experimental design to test insertion of an anti-Zika antibody (REGN4446) into the first intron of the mouse albumin locus following delivery of albumin-targeting gRNA (gRNA 1 v1) anti-Zika (REGN4446) antibody donor sequences to the Cas9-ready mouse via AAV2/8 as shown in FIG. 6. Viruses were injected into Cas9-ready mice intravenously. Serum was collected at Day 10, Day 28 and Day 56 for antibody titer, binding, and functional assays. Mice were taken down at Day 70 for insertion rate and mRNA level measurement.

FIG. 8 shows expression of the 4446 anti-Zika antibody (integrated AAV) in plasma samples from Cas9-ready mice at Day 10, Day 28, and Day 56 following injection of AAVs encoding albumin-targeting gRNA (gRNA 1 v1) and the various anti-Zika (REGN4446) antibody donor sequences. Results for episomal AAV (CMV and CASI) and integrated AAV (F2A/Albss, P2A/Albss, T2A/Albss, and T2A/RORss) are shown.

FIG. 9 shows western blot analysis of the antibodies expressed from episomal AAV (CMV LC T2A RORss HC; CASI HC T2A RORss LC) or integrated AAV (gRNA1v1 HC T2A RORss LC).

FIG. 10 shows the binding ability (binding to Zika envelope protein) of antibodies expressed from episomal AAV (CMV LC T2A RORss HC; CASI HC T2A RORss LC) or integrated AAV (gRNA1v1 HC F2A Albss LC; gRNA1 HC P2A Albss LC; gRNA1 HC T2A Albss LC; gRNA1 HC T2A RORss LC; and gRNA1 HC T2A LC). Results with a positive control antibody (REGN4446 anti-Zika antibody) are also shown.

FIG. 11 shows neutralization assay results (Zika infection) of antibodies expressed from episomal AAV (CMV LC T2A RORss HC; CASI HC T2A RORss LC) or integrated AAV (gRNA1v1 HC F2A Albss LC; gRNA1 HC P2A Albss LC; gRNA1 HC T2A Albss LC; gRNA1 HC T2A RORss LC; and gRNA1 HC T2A LC). Results with a positive control antibody (REGN4446 anti-Zika antibody) are also shown.

FIG. 12A shows indel rates in the livers of Cas9-ready mice following injection of episomal AAV (CMV LC T2A RORss HC; CASI HC T2A RORss LC) or integrated AAV (F2A/Albss; P2A/Albss; T2A/Albss; and T2A/RORss).

FIG. 12B shows mRNA levels of antibody (mAlb-REGN4446) expressed from episomal AAV (CMV LC T2A RORss HC; CASI HC T2A RORss LC) or integrated AAV (F2A/Albss; P2A/Albss; T2A/Albss; and T2A/RORss) in the livers of Cas9-ready mice as measured by TAQMAN qPCR.

FIG. 13 shows the genome structure of AAVs carrying both Cas9 and gRNA expression cassettes.

FIG. 14 shows serum Target Protein 1 levels before and after injection (35-days post-injection) of AAV2/8 viruses carrying tRNAGIn gRNA (targeting Target Gene 1) and Cas9 driven by four different promoters.

FIG. 15 shows antibody levels in mice injected with two AAVs, one carrying Cas9 and one carrying gRNA and insertion template. The figure shows expression of the 4446 anti-Zika antibody (integrated AAV) in serum samples from C57BL/6 mice at Day 11 and Day 28 following injection of two AAVs, one encoding albumin-targeting gRNA (gRNA1 v1) and the anti-Zika (REGN4446) antibody donor sequences (T2A/RORss) and one carrying the Cas9 sequence driven by the SerpinAP promoter. Results for episomal AAV (CASI HC T2A RORss LC) and integrated AAV at two different levels of viral genomes per mouse (Dual-Low and Dual-High) are shown. In the guide-only group, no AAV carrying the Cas9 sequence was delivered so integration did not occur.

FIG. 16 shows neutralization assay results (Zika infection) expressed from episomal AAV or integrated AAV (dual AAV experiments).

FIG. 17 shows an experimental design to test insertion of an anti-HA (influenza hemagglutinin) antibody into the first intron of the mouse albumin locus following delivery of Cas9 mRNA and albumin-targeting gRNA (gRNA 1 v1) to the mouse liver via lipid nanoparticle (LNP) and delivery of an AAV2/8 AlbSA 3263 anti-HA antibody donor sequence (light chain and heavy chain linked by P2A self-cleavage peptide).

FIG. 18 shows circulating antibody levels in mouse serum in mice injected with two AAVs, one carrying Cas9 and one carrying gRNA and insertion template, at Days 11, 28, 42, 56, and 118 post-injection. Comparison of episomal expression and Cas9-mediated integration is shown. Results from experiments in C57BL/6 mice are shown in the left panel, and results from experiments in BALB/c mice are shown in the right panel.

FIG. 19 shows the binding ability (binding to Zika envelope protein) of antibody expressed from episomal AAV or integrated AAV (dual AAV experiments). Closed circles and diamonds represent experiments in C57BL/6 mice, and open circles and diamonds represent experiments in BALB/c mice. Results with a positive control antibody (REGN4446 anti-Zika antibody) spiked into naïve mouse serum are also shown.

FIG. 20 shows an experimental design to test insertion of an anti-Zika antibody into the first intron of the mouse albumin locus, including assays for titer, binding, antibody quality, and neutralization. It also shows the genome structure of the two AAVs co-delivered in this experiment.

FIG. 21 shows neutralization assay results (Zika infection) of antibody expressed from episomal AAV or integrated AAV (dual AAV experiments) in C57BL/6 mice and in BALB/c mice. Results with a positive control antibody (REGN4446 anti-Zika antibody) spiked into naïve mouse serum are also shown.

FIG. 22 shows an in vivo Zika challenge experimental design for antibody expressed from episomal AAV or integrated AAV (dual AAV experiments).

FIG. 23 shows hIgG serum levels one day pre-challenge with Zika virus in mice treated with: (1) PBS (saline); (2) AAV2/8 to episomally express an off-target control antibody (CAG HC T2A RORss LC) (non-Zika mAB); a (3) low dose (1.0E+11 VG/Mouse) or (4) high dose (5.0E+11 VG/mouse) of AAV2/8 to episomally express the REGN4446 anti-Zika antibody (CASI HC_T2A_RORss_LC) (Episomal-Low Dose and Episomal-High Dose, respectively); a (5) low dose (5E+11 VG/mouse/vector) or (6) high dose (1E+12 VG/mouse/vector) of two AAVs, one carrying gRNA1 and the REGN4446 mAb expression cassette (HC_T2A_RORss_LC) and the second carrying the Cas9 cassette driven by the serpinAP promoter (Inserted-Low and Inserted-High, respectively); or (7) 200 μg of CHO-purified REGN4446 anti-Zika mAB (CHO Purified).

FIG. 24A shows the results of the Zika challenge experiment (percent survival) with the same groups as in FIG. 23 but also including an uninfected control.

FIG. 24B shows the same data as in FIG. 24A, but rearranged by titer. The values in the table on the top of the figure are the levels of monoclonal antibodies measured one day prior to challenge with Zika virus in μg/mL, and the coding is the type of AAV that delivered the mAB template (either single AAV for episomal expression or dual AAV for Cas9-mediated integration and a low or high dose for either).

FIG. 25 shows hIgG serum levels in mice treated with: (1) PBS (Saline); (2) REGN4446 anti-Zika (CASI HC_T2A_RORss_LC) (Episomal-Day 5-Anti-Zika); (3) H1H29339P anti-PcrV (CAG HC_T2A_RORss_LC) (Episomal-Day 5-Anti-PcrV); (4) H1H11829N₂ anti-HA (CAG LC_T2A_RORss_HC) (Episomal-Day 5-Anti-HA); (5) H1H29339P anti-PcrV (HC_T2A_RORss_LC) (Inserted-Day 12-Anti-PcrV); or (6) H1H11829N₂ anti-HA (LC_T2A_RORss_HC) (Inserted-Day 12-Anti-HA). Episomal AAV experiments performed in C57BL/6 mice and inserted experiments were performed in Cas9-ready mice.

FIG. 26 shows the binding ability (binding to PcrV protein) of anti-PcrV antibodies expressed from episomal AAV (CAG HC_T2A_RORss_LC) or integrated AAV (HC_T2A_RORss_LC). Results with a purified positive control antibody (H1H29339P anti-PcrV antibody) are also shown. Episomal anti-Zika antibody was used as a negative control.

FIG. 27 shows cytotoxicity assay results. P. aeruginosa strain 6077 PcrV-mediated cytotoxicity effects are neutralized by anti-PcrV antibodies expressed from episomal AAV (CAG HC_T2A_RORss_LC) or integrated AAV (HC_T2A_RORss_LC). Results with CHO-purified anti-PcrV antibody diluted in either PBS or in naïve mouse serum are shown for comparison. Anti-Zika antibody expressed from episomal AAV (CASI HC_T2A_RORss_LC) was used as a negative control.

FIG. 28 shows the binding ability (binding to HA protein) of antibodies expressed from episomal AAV (CAG LC_T2A_RORss_HC) or integrated AAV (LC_T2A_RORss_HC). Results with a purified positive control antibody (H1H11829N₂ anti-HA antibody) are also shown. Episomal anti-Zika antibody was used as a negative control.

FIG. 29 shows neutralization assay results. Influenza strain H1N1 A/PR/8/1934 is neutralized by anti-HA antibodies expressed from episomal AAV (CAG LC_T2A_RORss_HC) or integrated AAV (LC_T2A_RORss_HC). Results with a purified positive control antibody (H1H11829N₂ anti-HA antibody) are also shown. Purified anti-Feld1 antibody and serum alone were used as negative controls.

FIG. 30 shows in vivo Pseudomonas challenge experimental design for antibody expressed from episomal AAV or integrated AAV (dual AAV experiments).

FIG. 31 shows hIgG titers of C57BL/6 and BALB/c mice injected with AAV nine days prior (this is 7 days prior to challenge with Pseudomonas) in mice treated with: (1) PBS; (2) AAV2/8 to episomally express an isotype control antibody H1H11829N₂ anti-HA (CAG LC_T2A_RORss_HC) (anti-HA); a (3) low dose (1.0E+10 VG/Mouse) or (4) high dose (1.0E+11 VG/mouse) of AAV2/8 to episomally express the H1H29339P anti-PcrV antibody (CAG HC_T2A_RORss_LC) (Episomal-Low and Episomal-High, respectively), a (5) low dose (1E+11 VG/mouse/vector) or (6) high dose (1E+12 VG/mouse/vector) of two AAVs, one carrying gRNA1 and the H1H29339P anti-PcrV mAb expression cassette (HC_T2A_RORss_LC) and the second carrying the Cas9 cassette driven by the serpinAP promoter (Inserted-Low and Inserted-High, respectively), or a (7) low dose (0.2 mg/kg) or (8) high dose (1.0 mg/kg) of CHO-purified H1H29339P anti-PcrV mAB (0.2 mpk CHO and 1.0 mpk CHO, respectively).

FIG. 32A shows the results of the Pseudomonas challenge experiment (percent survival) in C57BL/6 mice with the Episomal-Low (CAG Low), Episomal-High (CAG High), Inserted-Low (KI Low), and Inserted-High (KI High) groups in FIG. 31 and also including an uninfected control, a non-protected bacteria-only control, and a non-protected isotype control.

FIG. 32B shows the results of the Pseudomonas challenge experiment (percent survival) in BALB/c mice with the Episomal-Low (CAG Low), Episomal-High (CAG High), Inserted-Low (KI Low), and Inserted-High (KI High) groups in FIG. 31 and also including an uninfected control, a non-protected bacteria-only control, and a non-protected isotype control.

### DEFINITIONS

The terms “protein,” “polypeptide,” and “peptide,” used interchangeably herein, include polymeric forms of amino acids of any length, including coded and non-coded amino acids and chemically or biochemically modified or derivatized amino acids. The terms also include polymers that have been modified, such as polypeptides having modified peptide backbones. The term “domain” refers to any part of a protein or polypeptide having a particular function or structure.

The terms “nucleic acid” and “polynucleotide,” used interchangeably herein, include polymeric forms of nucleotides of any length, including ribonucleotides, deoxyribonucleotides, or analogs or modified versions thereof. They include single-, double-, and multi-stranded DNA or RNA, genomic DNA, cDNA, DNA-RNA hybrids, and polymers comprising purine bases, pyrimidine bases, or other natural, chemically modified, biochemically modified, non-natural, or derivatized nucleotide bases.

The term “genomically integrated” refers to a nucleic acid that has been introduced into a cell such that the nucleotide sequence integrates into the genome of the cell. Any protocol may be used for the stable incorporation of a nucleic acid into the genome of a cell.

The term “expression vector” or “expression construct” or “expression cassette” refers to a recombinant nucleic acid containing a desired coding sequence operably linked to appropriate nucleic acid sequences necessary for the expression of the operably linked coding sequence in a particular host cell or organism. Nucleic acid sequences necessary for expression in prokaryotes usually include a promoter, an operator (optional), and a ribosome binding site, as well as other sequences. Eukaryotic cells are generally known to utilize promoters, enhancers, and termination and polyadenylation signals, although some elements may be deleted and other elements added without sacrificing the necessary expression.

The term “targeting vector” refers to a recombinant nucleic acid that can be introduced by homologous recombination, non-homologous-end-joining-mediated ligation, or any other means of recombination to a target position in the genome of a cell.

The term “viral vector” refers to a recombinant nucleic acid that includes at least one element of viral origin and includes elements sufficient for or permissive of packaging into a viral vector particle. The vector and/or particle can be utilized for the purpose of transferring DNA, RNA, or other nucleic acids into cells either in vitro, ex vivo, or in vivo. Numerous forms of viral vectors are known.

The term “isolated” with respect to cells, tissues (e.g., liver samples), proteins, and nucleic acids includes cells, tissues (e.g., liver samples), proteins, and nucleic acids that are relatively purified with respect to other bacterial, viral, cellular, or other components that may normally be present in situ, up to and including a substantially pure preparation of the cells, tissues (e.g., liver samples), proteins, and nucleic acids. The term “isolated” also includes cells, tissues (e.g., liver samples), proteins, and nucleic acids that have no naturally occurring counterpart, have been chemically synthesized and are thus substantially uncontaminated by other cells, tissues (e.g., liver samples), proteins, and nucleic acids, or has been separated or purified from most other components (e.g., cellular components) with which they are naturally accompanied (e.g., other cellular proteins, polynucleotides, or cellular components).

The term “wild type” includes entities having a structure and/or activity as found in a normal (as contrasted with mutant, diseased, altered, or so forth) state or context. Wild type genes and polypeptides often exist in multiple different forms (e.g., alleles).

The term “endogenous sequence” refers to a nucleic acid sequence that occurs naturally within a cell or animal. For example, an endogenous albumin sequence of an animal refers to a native albumin sequence that naturally occurs at the albumin locus in the animal.

“Exogenous” molecules or sequences include molecules or sequences that are not normally present in a cell in that form. Normal presence includes presence with respect to the particular developmental stage and environmental conditions of the cell. An exogenous molecule or sequence, for example, can include a mutated version of a corresponding endogenous sequence within the cell, such as a humanized version of the endogenous sequence, or can include a sequence corresponding to an endogenous sequence within the cell but in a different form (i.e., not within a chromosome). In contrast, endogenous molecules or sequences include molecules or sequences that are normally present in that form in a particular cell at a particular developmental stage under particular environmental conditions.

The term “heterologous” when used in the context of a nucleic acid or a protein indicates that the nucleic acid or protein comprises at least two segments that do not naturally occur together in the same molecule. For example, the term “heterologous,” when used with reference to segments of a nucleic acid or segments of a protein, indicates that the nucleic acid or protein comprises two or more sub-sequences that are not found in the same relationship to each other (e.g., joined together) in nature. As one example, a “heterologous” region of a nucleic acid vector is a segment of nucleic acid within or attached to another nucleic acid molecule that is not found in association with the other molecule in nature. For example, a heterologous region of a nucleic acid vector could include a coding sequence flanked by sequences not found in association with the coding sequence in nature. Likewise, a “heterologous” region of a protein is a segment of amino acids within or attached to another peptide molecule that is not found in association with the other peptide molecule in nature (e.g., a fusion protein, or a protein with a tag). Similarly, a nucleic acid or protein can comprise a heterologous label or a heterologous secretion or localization sequence.

“Codon optimization” takes advantage of the degeneracy of codons, as exhibited by the multiplicity of three-base pair codon combinations that specify an amino acid, and generally includes a process of modifying a nucleic acid sequence for enhanced expression in particular host cells by replacing at least one codon of the native sequence with a codon that is more frequently or most frequently used in the genes of the host cell while maintaining the native amino acid sequence. For example, a nucleic acid encoding a Cas9 protein can be modified to substitute codons having a higher frequency of usage in a given prokaryotic or eukaryotic cell, including a bacterial cell, a yeast cell, a human cell, a non-human cell, a mammalian cell, a rodent cell, a mouse cell, a rat cell, a hamster cell, or any other host cell, as compared to the naturally occurring nucleic acid sequence. Codon usage tables are readily available, for example, at the “Codon Usage Database.” These tables can be adapted in a number of ways. See Nakamura et al. (2000) Nucleic Acids Research 28:292, herein incorporated by reference in its entirety for all purposes. Computer algorithms for codon optimization of a particular sequence for expression in a particular host are also available (see, e.g., Gene Forge).

The term “locus” refers to a specific location of a gene (or significant sequence), DNA sequence, polypeptide-encoding sequence, or position on a chromosome of the genome of an organism. For example, an “albumin locus” may refer to the specific location of an albumin gene, albumin DNA sequence, albumin-encoding sequence, or albumin position on a chromosome of the genome of an organism that has been identified as to where such a sequence resides. An “albumin locus” may comprise a regulatory element of an albumin gene, including, for example, an enhancer, a promoter, 5′ and/or 3′ untranslated region (UTR), or a combination thereof.

The term “gene” refers to DNA sequences in a chromosome that may contain, if naturally present, at least one coding and at least one non-coding region. The DNA sequence in a chromosome that codes for a product (e.g., but not limited to, an RNA product and/or a polypeptide product) can include the coding region interrupted with non-coding introns and sequence located adjacent to the coding region on both the 5′ and 3′ ends such that the gene corresponds to the full-length mRNA (including the 5′ and 3′ untranslated sequences). Additionally, other non-coding sequences including regulatory sequences (e.g., but not limited to, promoters, enhancers, and transcription factor binding sites), polyadenylation signals, internal ribosome entry sites, silencers, insulating sequence, and matrix attachment regions may be present in a gene. These sequences may be close to the coding region of the gene (e.g., but not limited to, within 10 kb) or at distant sites, and they influence the level or rate of transcription and translation of the gene.

The term “allele” refers to a variant form of a gene. Some genes have a variety of different forms, which are located at the same position, or genetic locus, on a chromosome. A diploid organism has two alleles at each genetic locus. Each pair of alleles represents the genotype of a specific genetic locus. Genotypes are described as homozygous if there are two identical alleles at a particular locus and as heterozygous if the two alleles differ.

A “promoter” is a regulatory region of DNA usually comprising a TATA box capable of directing RNA polymerase II to initiate RNA synthesis at the appropriate transcription initiation site for a particular polynucleotide sequence. A promoter may additionally comprise other regions which influence the transcription initiation rate. The promoter sequences disclosed herein modulate transcription of an operably linked polynucleotide. A promoter can be active in one or more of the cell types disclosed herein (e.g., a eukaryotic cell, a non-human mammalian cell, a human cell, a rodent cell, a pluripotent cell, a one-cell stage embryo, a differentiated cell, or a combination thereof). A promoter can be, for example, a constitutively active promoter, a conditional promoter, an inducible promoter, a temporally restricted promoter (e.g., a developmentally regulated promoter), or a spatially restricted promoter (e.g., a cell-specific or tissue-specific promoter). Examples of promoters can be found, for example, in WO 2013/176772, herein incorporated by reference in its entirety for all purposes.

A constitutive promoter is one that is active in all tissues or particular tissues at all developing stages. Examples of constitutive promoters include the human cytomegalovirus immediate early (hCMV), mouse cytomegalovirus immediate early (mCMV), human elongation factor 1 alpha (hEF1a), mouse elongation factor 1 alpha (mEF1α), mouse phosphoglycerate kinase (PGK), chicken beta actin hybrid (CAG or CBh), SV40 early, and beta 2 tubulin promoters.

Examples of inducible promoters include, for example, chemically regulated promoters and physically-regulated promoters. Chemically regulated promoters include, for example, alcohol-regulated promoters (e.g., an alcohol dehydrogenase (alcA) gene promoter), tetracycline-regulated promoters (e.g., a tetracycline-responsive promoter, a tetracycline operator sequence (tetO), a tet-On promoter, or a tet-Off promoter), steroid regulated promoters (e.g., a rat glucocorticoid receptor, a promoter of an estrogen receptor, or a promoter of an ecdysone receptor), or metal-regulated promoters (e.g., a metalloprotein promoter). Physically regulated promoters include, for example temperature-regulated promoters (e.g., a heat shock promoter) and light-regulated promoters (e.g., a light-inducible promoter or a light-repressible promoter).

Tissue-specific promoters can be, for example, neuron-specific promoters, glia-specific promoters, muscle cell-specific promoters, heart cell-specific promoters, kidney cell-specific promoters, bone cell-specific promoters, endothelial cell-specific promoters, or immune cell-specific promoters (e.g., a B cell promoter or a T cell promoter).

Developmentally regulated promoters include, for example, promoters active only during an embryonic stage of development, or only in an adult cell.

“Operable linkage” or being “operably linked” includes juxtaposition of two or more components (e.g., a promoter and another sequence element) such that both components function normally and allow the possibility that at least one of the components can mediate a function that is exerted upon at least one of the other components. For example, a promoter can be operably linked to a coding sequence if the promoter controls the level of transcription of the coding sequence in response to the presence or absence of one or more transcriptional regulatory factors. Operable linkage can include such sequences being contiguous with each other or acting in trans (e.g., a regulatory sequence can act at a distance to control transcription of the coding sequence).

“Complementarity” of nucleic acids means that a nucleotide sequence in one strand of nucleic acid, due to orientation of its nucleobase groups, forms hydrogen bonds with another sequence on an opposing nucleic acid strand. The complementary bases in DNA are typically A with T and C with G. In RNA, they are typically C with G and U with A. Complementarity can be perfect or substantial/sufficient. Perfect complementarity between two nucleic acids means that the two nucleic acids can form a duplex in which every base in the duplex is bonded to a complementary base by Watson-Crick pairing. “Substantial” or “sufficient” complementary means that a sequence in one strand is not completely and/or perfectly complementary to a sequence in an opposing strand, but that sufficient bonding occurs between bases on the two strands to form a stable hybrid complex in set of hybridization conditions (e.g., salt concentration and temperature). Such conditions can be predicted by using the sequences and standard mathematical calculations to predict the Tm (melting temperature) of hybridized strands, or by empirical determination of Tm by using routine methods. Tm includes the temperature at which a population of hybridization complexes formed between two nucleic acid strands are 50% denatured (i.e., a population of double-stranded nucleic acid molecules becomes half dissociated into single strands). At a temperature below the Tm, formation of a hybridization complex is favored, whereas at a temperature above the Tm, melting or separation of the strands in the hybridization complex is favored. Tm may be estimated for a nucleic acid having a known G+C content in an aqueous 1 M NaCl solution by using, e.g., Tm=81.5+0.41 (% G+C), although other known Tm computations consider nucleic acid structural characteristics.

Hybridization requires that the two nucleic acids contain complementary sequences, although mismatches between bases are possible. The conditions appropriate for hybridization between two nucleic acids depend on the length of the nucleic acids and the degree of complementation, variables which are well known. The greater the degree of complementation between two nucleotide sequences, the greater the value of the melting temperature (Tm) for hybrids of nucleic acids having those sequences. For hybridizations between nucleic acids with short stretches of complementarity (e.g. complementarity over 35 or fewer, 30 or fewer, 25 or fewer, 22 or fewer, 20 or fewer, or 18 or fewer nucleotides) the position of mismatches becomes important (see Sambrook et al., supra, 11.7-11.8). Typically, the length for a hybridizable nucleic acid is at least about 10 nucleotides. Illustrative minimum lengths for a hybridizable nucleic acid include at least about 15 nucleotides, at least about 20 nucleotides, at least about 22 nucleotides, at least about 25 nucleotides, and at least about 30 nucleotides. Furthermore, the temperature and wash solution salt concentration may be adjusted as necessary according to factors such as length of the region of complementation and the degree of complementation.

The sequence of polynucleotide need not be 100% complementary to that of its target nucleic acid to be specifically hybridizable. Moreover, a polynucleotide may hybridize over one or more segments such that intervening or adjacent segments are not involved in the hybridization event (e.g., a loop structure or hairpin structure). A polynucleotide (e.g., gRNA) can comprise at least 70%, at least 80%, at least 90%, at least 95%, at least 99%, or 100% sequence complementarity to a target region within the target nucleic acid sequence to which they are targeted. For example, a gRNA in which 18 of 20 nucleotides are complementary to a target region, and would therefore specifically hybridize, would represent 90% complementarity. In this example, the remaining noncomplementary nucleotides may be clustered or interspersed with complementary nucleotides and need not be contiguous to each other or to complementary nucleotides.

Percent complementarity between particular stretches of nucleic acid sequences within nucleic acids can be determined routinely using BLAST programs (basic local alignment search tools) and PowerBLAST programs (Altschul et al. (1990) J. Mol. Biol. 215:403-410; Zhang and Madden (1997) Genome Res. 7:649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489).

The methods and compositions provided herein employ a variety of different components. Some components throughout the description can have active variants and fragments. Such components include, for example, Cas proteins, CRISPR RNAs, tracrRNAs, and guide RNAs. Biological activity for each of these components is described elsewhere herein. The term “functional” refers to the innate ability of a protein or nucleic acid (or a fragment or variant thereof) to exhibit a biological activity or function. Such biological activities or functions can include, for example, the ability of a Cas protein to bind to a guide RNA and to a target DNA sequence. The biological functions of functional fragments or variants may be the same or may in fact be changed (e.g., with respect to their specificity or selectivity or efficacy) in comparison to the original molecule, but with retention of the molecule's basic biological function.

The term “variant” refers to a nucleotide sequence differing from the sequence most prevalent in a population (e.g., by one nucleotide) or a protein sequence different from the sequence most prevalent in a population (e.g., by one amino acid).

The term “fragment,” when referring to a protein, means a protein that is shorter or has fewer amino acids than the full-length protein. The term “fragment,” when referring to a nucleic acid, means a nucleic acid that is shorter or has fewer nucleotides than the full-length nucleic acid. A fragment can be, for example, when referring to a protein fragment, an N-terminal fragment (i.e., removal of a portion of the C-terminal end of the protein), a C-terminal fragment (i.e., removal of a portion of the N-terminal end of the protein), or an internal fragment (i.e., removal of a portion of each of the N-terminal and C-terminal ends of the protein). A fragment can be, for example, when referring to a nucleic acid fragment, a 5′ fragment (i.e., removal of a portion of the 3′ end of the nucleic acid), a 3′ fragment (i.e., removal of a portion of the 5′ end of the nucleic acid), or an internal fragment (i.e., removal of a portion each of the 5′ and 3′ ends of the nucleic acid).

“Sequence identity” or “identity” in the context of two polynucleotides or polypeptide sequences refers to the residues in the two sequences that are the same when aligned for maximum correspondence over a specified comparison window. When percentage of sequence identity is used in reference to proteins, residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, California).

“Percentage of sequence identity” includes the value determined by comparing two optimally aligned sequences (greatest number of perfectly matched residues) over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. Unless otherwise specified (e.g., the shorter sequence includes a linked heterologous sequence), the comparison window is the full length of the shorter of the two sequences being compared.

Unless otherwise stated, sequence identity/similarity values include the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. “Equivalent program” includes any sequence comparison program that, for any two sequences in question, generates an alignment having identical nucleotide or amino acid residue matches and an identical percent sequence identity when compared to the corresponding alignment generated by GAP Version 10.

The term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine, or leucine for another non-polar residue. Likewise, examples of conservative substitutions include the substitution of one polar (hydrophilic) residue for another such as between arginine and lysine, between glutamine and asparagine, or between glycine and serine. Additionally, the substitution of a basic residue such as lysine, arginine, or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, or methionine for a polar (hydrophilic) residue such as cysteine, glutamine, glutamic acid or lysine and/or a polar residue for a non-polar residue. Typical amino acid categorizations are summarized in Table 1 below.

TABLE 1
Amino Acid Categorizations.

| | | | | | |
|---|---|---|---|---|---|
| Alanine | Ala | A | Nonpolar | Neutral | 1.8 |
| Arginine | Arg | R | Polar | Positive | −4.5 |
| Asparagine | Asn | N | Polar | Neutral | −3.5 |
| Aspartic acid | Asp | D | Polar | Negative | −3.5 |
| Cysteine | Cys | C | Nonpolar | Neutral | 2.5 |
| Glutamic acid | Glu | E | Polar | Negative | −3.5 |
| Glutamine | Gln | Q | Polar | Neutral | −3.5 |
| Glycine | Gly | G | Nonpolar | Neutral | −0.4 |
| Histidine | His | H | Polar | Positive | −3.2 |
| Isoleucine | Ile | I | Nonpolar | Neutral | 4.5 |
| Leucine | Leu | L | Nonpolar | Neutral | 3.8 |
| Lysine | Lys | K | Polar | Positive | −3.9 |
| Methionine | Met | M | Nonpolar | Neutral | 1.9 |
| Phenylalanine | Phe | F | Nonpolar | Neutral | 2.8 |
| Proline | Pro | P | Nonpolar | Neutral | −1.6 |
| Serine | Ser | S | Polar | Neutral | −0.8 |
| Threonine | Thr | T | Polar | Neutral | −0.7 |
| Tryptophan | Trp | W | Nonpolar | Neutral | −0.9 |
| Tyrosine | Tyr | Y | Polar | Neutral | −1.3 |
| Valine | Val | V | Nonpolar | Neutral | 4.2 |

A “homologous” sequence (e.g., nucleic acid sequence) includes a sequence that is either identical or substantially similar to a known reference sequence, such that it is, for example, at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical to the known reference sequence. Homologous sequences can include, for example, orthologous sequence and paralogous sequences. Homologous genes, for example, typically descend from a common ancestral DNA sequence, either through a speciation event (orthologous genes) or a genetic duplication event (paralogous genes). “Orthologous” genes include genes in different species that evolved from a common ancestral gene by speciation. Orthologs typically retain the same function in the course of evolution. “Paralogous” genes include genes related by duplication within a genome. Paralogs can evolve new functions in the course of evolution.

The term “in vitro” includes artificial environments and to processes or reactions that occur within an artificial environment (e.g., a test tube or an isolated cell or cell line). The term “in vivo” includes natural environments (e.g., a cell or organism or body) and to processes or reactions that occur within a natural environment. The term “ex vivo” includes cells that have been removed from the body of an individual and processes or reactions that occur within such cells.

The term “reporter gene” refers to a nucleic acid having a sequence encoding a gene product (typically an enzyme) that is easily and quantifiably assayed when a construct comprising the reporter gene sequence operably linked to an endogenous or heterologous promoter and/or enhancer element is introduced into cells containing (or which can be made to contain) the factors necessary for the activation of the promoter and/or enhancer elements. Examples of reporter genes include, but are not limited, to genes encoding beta-galactosidase (lacZ), the bacterial chloramphenicol acetyltransferase (cat) genes, firefly luciferase genes, genes encoding beta-glucuronidase (GUS), and genes encoding fluorescent proteins. A “reporter protein” refers to a protein encoded by a reporter gene.

The term “fluorescent reporter protein” as used herein means a reporter protein that is detectable based on fluorescence wherein the fluorescence may be either from the reporter protein directly, activity of the reporter protein on a fluorogenic substrate, or a protein with affinity for binding to a fluorescent tagged compound. Examples of fluorescent proteins include green fluorescent proteins (e.g., GFP, GFP-2, tagGFP, turboGFP, eGFP, Emerald, Azami Green, Monomeric Azami Green, CopGFP, AceGFP, and ZsGreenl), yellow fluorescent proteins (e.g., YFP, eYFP, Citrine, Venus, YPet, PhiYFP, and ZsYellowl), blue fluorescent proteins (e.g., BFP, eBFP, eBFP2, Azurite, mKalamal, GFPuv, Sapphire, and T-sapphire), cyan fluorescent proteins (e.g., CFP, eCFP, Cerulean, CyPet, AmCyanl, and Midoriishi-Cyan), red fluorescent proteins (e.g., RFP, mKate, mKate2, mPlum, DsRed monomer, mCherry, mRFP1, DsRed-Express, DsRed2, DsRed-Monomer, HcRed-Tandem, HcRed1, AsRed2, eqFP611, mRaspberry, mStrawberry, and Jred), orange fluorescent proteins (e.g., mOrange, mKO, Kusabira-Orange, Monomeric Kusabira-Orange, mTangerine, and tdTomato), and any other suitable fluorescent protein whose presence in cells can be detected by flow cytometry methods.

Repair in response to double-strand breaks (DSBs) occurs principally through two conserved DNA repair pathways: homologous recombination (HR) and non-homologous end joining (NHEJ). See Kasparek & Humphrey (2011) Semin. Cell Dev. Biol. 22 (8): 886-897, herein incorporated by reference in its entirety for all purposes. Likewise, repair of a target nucleic acid mediated by an exogenous donor nucleic acid can include any process of exchange of genetic information between the two polynucleotides.

The term “recombination” includes any process of exchange of genetic information between two polynucleotides and can occur by any mechanism. Recombination can occur via homology directed repair (HDR) or homologous recombination (HR). HDR or HR includes a form of nucleic acid repair that can require nucleotide sequence homology, uses a “donor” molecule as a template for repair of a “target” molecule (i.e., the one that experienced the double-strand break), and leads to transfer of genetic information from the donor to target. Without wishing to be bound by any particular theory, such transfer can involve mismatch correction of heteroduplex DNA that forms between the broken target and the donor, and/or synthesis-dependent strand annealing, in which the donor is used to resynthesize genetic information that will become part of the target, and/or related processes. In some cases, the donor polynucleotide, a portion of the donor polynucleotide, a copy of the donor polynucleotide, or a portion of a copy of the donor polynucleotide integrates into the target DNA. See Wang et al. (2013) Cell 153:910-918; Mandalos et al. (2012) PLoS ONE 7: e45768: 1-9; and Wang et al. (2013) Nat Biotechnol. 31:530-532, each of which is herein incorporated by reference in its entirety for all purposes.

Non-homologous end joining (NHEJ) includes the repair of double-strand breaks in a nucleic acid by direct ligation of the break ends to one another or to an exogenous sequence without the need for a homologous template. Ligation of non-contiguous sequences by NHEJ can often result in deletions, insertions, or translocations near the site of the double-strand break. For example, NHEJ can also result in the targeted integration of an exogenous donor nucleic acid through direct ligation of the break ends with the ends of the exogenous donor nucleic acid (i.e., NHEJ-based capture). Such NHEJ-mediated targeted integration can be preferred for insertion of an exogenous donor nucleic acid when homology directed repair (HDR) pathways are not readily usable (e.g., in non-dividing cells, primary cells, and cells which perform homology-based DNA repair poorly). In addition, in contrast to homology-directed repair, knowledge concerning large regions of sequence identity flanking the cleavage site is not needed, which can be beneficial when attempting targeted insertion into organisms that have genomes for which there is limited knowledge of the genomic sequence. The integration can proceed via ligation of blunt ends between the exogenous donor nucleic acid and the cleaved genomic sequence, or via ligation of sticky ends (i.e., having 5′ or 3′ overhangs) using an exogenous donor nucleic acid that is flanked by overhangs that are compatible with those generated by a nuclease agent in the cleaved genomic sequence. See, e.g., US 2011/020722, WO 2014/033644, WO 2014/089290, and Maresca et al. (2013) Genome Res. 23 (3): 539-546, each of which is herein incorporated by reference in its entirety for all purposes. If blunt ends are ligated, target and/or donor resection may be needed to generation regions of microhomology needed for fragment joining, which may create unwanted alterations in the target sequence.

Compositions or methods “comprising” or “including” one or more recited elements may include other elements not specifically recited. For example, a composition that “comprises” or “includes” a protein may contain the protein alone or in combination with other ingredients. The transitional phrase “consisting essentially of” means that the scope of a claim is to be interpreted to encompass the specified elements recited in the claim and those that do not materially affect the basic and novel characteristic(s) of the claimed invention. Thus, the term “consisting essentially of” when used in a claim of this invention is not intended to be interpreted to be equivalent to “comprising.”

“Optional” or “optionally” means that the subsequently described event or circumstance may or may not occur and that the description includes instances in which the event or circumstance occurs and instances in which the event or circumstance does not.

Designation of a range of values includes all integers within or defining the range, and all subranges defined by integers within the range.

Unless otherwise apparent from the context, the term “about” encompasses values±5 of a stated value.

The term “and/or” refers to and encompasses any and all possible combinations of one or more of the associated listed items, as well as the lack of combinations when interpreted in the alternative (“or”).

The term “or” refers to any one member of a particular list and also includes any combination of members of that list.

The singular forms of the articles “a,” “an,” and “the” include plural references unless the context clearly dictates otherwise. For example, the term “a protein” or “at least one protein” can include a plurality of proteins, including mixtures thereof.

Statistically significant means p≤0.05.

### DETAILED DESCRIPTION

### I. Overview

Neutralizing antibodies play an essential part in antibacterial and antiviral immunity and are instrumental in preventing or modulating bacterial or viral diseases. Such antibodies defend a cell from an antigen or infectious body by neutralizing any effect it has biologically.

Active vaccination is generally considered the best approach to combat viral diseases, and it can similarly be used to combat bacterial diseases. Active immunity refers to the process of exposing the body to an antigen to generate an adaptive immune response. The response takes days/weeks to develop but may last for years. Passive immunity refers to the process of providing pre-formed specific antibodies from an exogenous source to protect against infection. However, because the individual's own immune system has not been stimulated, no immunological memory is generated. Consequently, passive immunization gives immediate, but short-lived protection. Protection lasts days to months rather than years. Passive immunization can have some advantages over vaccination. In particular, passive immunization has become an attractive approach because of the emergence of new and drug-resistant microorganisms, diseases that are unresponsive to drug therapy, and individuals with an impaired immune system who are unable to respond to conventional vaccines.

Antibodies developed by the immune system upon infection or active vaccination tend to focus on easily accessible loops on the bacterial or viral surface, which often have great sequence and conformational variability. This is a problem for two reasons: the bacteria or virus population can quickly evade these antibodies, and the antibodies are attacking portions of the protein that are not essential for function. For example, a roadblock to the development of an effective vaccine against some viruses like HIV is the extraordinary ability of such viruses to mutate and evolve into numerous quasi-species. Broadly neutralizing antibodies-termed “broadly” because they attack many strains or quasi-species of the bacteria or virus, and “neutralizing” because they attack key functional sites in the bacteria or virus and block infection—can overcome these problems. However, these antibodies usually come too late to provide effective protection from the disease, and treatment with such antibodies provides only short-lived protection.

Methods and compositions are provided herein for integrating coding sequences for antigen-binding proteins such as broadly neutralizing antibodies into a safe harbor locus such as an albumin locus in an animal in vivo. The antigen-binding protein coding sequence can comprise a heavy chain coding sequence and a separate light chain coding sequence integrated into the same safe harbor locus to generate an antigen-binding protein that is not a single-chain antigen-binding protein. Likewise, methods and compositions are provided herein for integrating coding sequences for antigen-binding proteins such as broadly neutralizing antibodies into any genomic locus in an animal in vivo. The antigen-binding protein coding sequence can comprise a heavy chain coding sequence and a separate light chain coding sequence integrated into the same genomic locus to generate an antigen-binding protein that is not a single-chain antigen-binding protein. Such methods lead to high levels of antibody expression that reach the therapeutic window for many diseases, including infectious diseases, and are comparable to expression levels achieved by episomal vectors that typically persist in multiple copies per cell. Integration of the coding sequence as in the methods disclosed herein is advantageous over non-integrating episomal vectors because transgene retention can be problematic with non-replicating episomal vectors due to the non-replicating episomes being progressively and rapidly diluted out through cell division. In dividing cells, the AAV DNA is diluted out through cell division making it necessary to administer more virus for continued therapeutic response. These subsequent exposures may result in rapid neutralization of the virus and, therefore, a decreased host response. However, these problems do not occur when the integration methods disclosed herein are used. The levels of antibody expression achieved by the methods disclosed herein could protect the animals from infection with infectious agents such as viruses and bacteria or treat infection with such infectious agents. However, the methods and compositions are not limited to therapeutic antibodies targeting viral or bacterial antigens and encompass other therapeutic antibodies as well.

II. Methods for Inserting Antigen-Binding Protein Coding Sequences into Safe Harbor Loci

Provided herein are methods for inserting an antigen-binding-protein coding sequence into a safe harbor locus in a cell or an animal in vivo. Also provided are methods for inserting an antigen-binding-protein coding sequence into a safe harbor locus in a cell in vitro or ex vivo. Likewise, provided herein are methods for inserting an antigen-binding-protein coding sequence into a genomic locus in a cell or an animal in vivo. Also provided are methods for inserting an antigen-binding-protein coding sequence into a genomic locus in a cell in vitro or ex vivo. Also provided is a nuclease agent (or a nucleic acid encoding the nuclease agent or one or more nucleic acids encoding the nuclease agent) and an exogenous donor nucleic acid comprising an antigen-binding-protein coding sequence, for use in inserting an antigen-binding-protein coding sequence into a genomic locus or a safe harbor locus in a subject (e.g., animal or cell in vivo), wherein the nuclease agent targets and cleaves a target site in the genomic locus or safe harbor locus and wherein the exogenous donor nucleic acid is inserted into the genomic locus or safe harbor locus. Also provided is an exogenous donor nucleic acid comprising an antigen-binding-protein coding sequence, for use in inserting an antigen-binding-protein coding sequence into a genomic locus or safe harbor locus in a subject (e.g., animal or cell in vivo), wherein the exogenous donor nucleic acid is inserted into the genomic locus or safe harbor locus. Also provided is a nuclease agent (or a nucleic acid encoding the nuclease agent or one or more nucleic acids encoding the nuclease agent) and an exogenous donor nucleic acid comprising an antigen-binding-protein coding sequence, for use in treating or effecting prophylaxis of (preventing) a disease in a subject (e.g., animal), wherein the nuclease agent targets and cleaves a target site in a genomic locus or safe harbor locus of the subject, wherein the exogenous donor nucleic acid is inserted into the genomic locus or safe harbor locus, and wherein the antigen-binding protein is expressed in the subject and targets an antigen associated with the disease. Also provided is an exogenous donor nucleic acid comprising an antigen-binding-protein coding sequence, for use in treating or effecting prophylaxis of (preventing) a disease in a subject (e.g., animal), wherein the exogenous donor nucleic acid is inserted into the genomic locus or safe harbor locus, and wherein the antigen-binding protein is expressed in the subject and targets an antigen associated with the disease. Such methods can comprise, for example, introducing into the animal or cell a nuclease agent that targets a target site in the genomic locus or safe harbor locus (or a nucleic acid encoding the nuclease agent or one or more nucleic acids encoding the nuclease agent) and an exogenous donor nucleic acid comprising the antigen-binding-protein coding sequence. The nuclease agent can cleave the target site and the antigen-binding protein coding sequence is inserted into the genomic locus or safe harbor locus to produce a modified genomic locus or safe harbor locus. Alternatively, such methods can comprise introducing into the animal or cell an exogenous donor nucleic acid comprising the antigen-binding-protein coding sequence. The antigen-binding protein coding sequence is inserted into the genomic locus or safe harbor locus (e.g., through homologous recombination or any other mechanism for recombination or insertion) to produce a modified genomic locus or safe harbor locus. Also provided are methods for inserting an antigen-binding-protein coding sequence into a genomic locus or safe harbor gene or for inserting an antigen-binding-protein coding sequence into a genomic locus or safe harbor locus in a genome. Such methods can comprise, for example, contacting the genomic gene or safe harbor gene or genomic locus or safe harbor locus with a nuclease agent that targets a target site in the genomic gene/locus or safe harbor gene/locus (or a nucleic acid encoding the nuclease agent or one or more nucleic acids encoding the nuclease agent) and an exogenous donor nucleic acid comprising an antigen-binding-protein coding sequence, wherein the nuclease agent cleaves the target site and the antigen-binding protein coding sequence is inserted into the genomic gene/locus or safe harbor gene/locus to produce the modified genomic gene/locus or safe harbor gene/locus. Alternatively, such methods can comprise contacting the genomic gene/locus or safe harbor gene/locus with an exogenous donor nucleic acid comprising an antigen-binding-protein coding sequence, wherein the antigen-binding protein coding sequence is inserted into the genomic gene/locus or safe harbor gene/locus to produce the modified genomic gene/locus or safe harbor gene/locus. Optionally two or more nuclease agents targeting different target sites in the genomic gene/locus or safe harbor gene/locus can be used. The modified genomic gene/locus or safe harbor gene/locus can be heterozygous or homozygous for the antigen-binding-protein coding sequence.

Optionally, such methods can further comprise assessing expression and/or activity of the antigen-binding-protein in the animal. Examples of such methods are disclosed elsewhere herein, as are examples of antigen-binding proteins (and coding sequences), types of nuclease agents, types of exogenous donor nucleic acids, types of genomic loci or safe harbor loci, and types of animals that can be used in such methods. In some methods, expression of the antigen-binding protein in serum or plasma samples from the animal is at least about 500, at least about 1000, at least about 1500, at least about 2000, at least about 2500, at least about 3000, at least about 3500, at least about 4000, at least about 4500, at least about 5000, at least about 5500, at least about 6000, at least about 6500, at least about 7000, at least about 7500, at least about 8000, at least about 8500, at least about 9000, at least about 9500, at least about 10000, at least about 20000, at least about 30000, at least about 40000, at least about 50000, at least about 60000, at least about 70000, at least about 80000, at least about 90000, at least about 100000, at least about 110000, at least about 120000, at least about 130000, at least about 140000, at least about 150000, at least about 200000, at least about 250000, at least about 300000, at least about 350000, at least about 400000, at least about 500000, at least about 600000, at least about 700000, at least about 800000, at least about 900000, or at least about 1000000 ng/ml (i.e., at least about 0.5, at least about 1, at least about 1.5, at least about 2, at least about 2.5, at least about 3, at least about 3.5, at least about 4, at least about 4.5, at least about 5, at least about 5.5, at least about 6, at least about 6.5, at least about 7, at least about 7.5, at least about 8, at least about 8.5, at least about 9, at least about 9.5, at least about 10, at least about 20, at least about 30, at least about 40, at least about 50, at least about 60, at least about 70, at least about 80, at least about 90, at least about 100, at least about 110, at least about 120, at least about 130, at least about 140, at least about 150, at least about 200, at least about 250, at least about 300, at least about 350, at least about 400, at least about 500, at least about 600, at least about 700, at least about 800, at least about 900, or at least about 1000 μg/mL) at a time point of about 1 week, about 2 weeks, about 3 week, about 4 weeks, about 5 weeks, about 6 weeks, about 7 weeks, about 8 weeks, about 9 weeks, about 10 weeks, about 1 month, about 2 months, about 3 months, about 4 months, about 5 months, or about 6 months after injection of the nuclease agent (or the nucleic acid encoding the nuclease agent or one or more nucleic acids encoding the nuclease agent) and the exogenous donor sequence. For example, expression can be at least about 2500, at least about 5000, at least about 10000, at least about 100000, at least about 400000, at least about 500000, at least about 600000, at least about 700000, at least about 800000, at least about 900000, or at least about 1000000 ng/ml (i.e., at least about 2.5, at least about 5, at least about 10, at least about 100, at least about 400, at least about 500, at least about 600, at least about 700, at least about 800, at least about 900, at least about 1000, at least about 1100, at least about 1200, at least about 1300, at least about 1400, or at least about 1500 μg/mL) at about 2 weeks, about 4 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 1 month, about 2 months, about 3 months, about 4 months, about 5 months, or about 6 months after injection. In some methods in which the antigen-binding protein or antibody targets a bacterial or viral antigen, percent infectivity is reduced to less than about 95%, less than about 90%, less than about 85%, less than about 80%, less than about 75%, less than about 70%, less than about 65%, less than about 55%, less than about 50%, less than about 45%, less than about 40%, less than about 35%, less than about 30%, less than about 25% (e.g., as determined in a neutralization assay) compared infectivity in a negative control sample at a time point of about 1 week, about 2 weeks, about 3 week, about 4 weeks, about 5 weeks, about 6 weeks, about 7 weeks, about 8 weeks, about 9 weeks, about 10 weeks, about 1 month, about 2 months, about 3 months, about 4 months, about 5 months, or about 6 months after injection of the nuclease agent (or the nucleic acid encoding the nuclease agent or one or more nucleic acids encoding the nuclease agent) and the exogenous donor sequence. For example, infectivity can be reduced to less than about 65%, less than about 60%, or less than about 55% at about 2 weeks after injection.

The nuclease agent (or the nucleic acid encoding the nuclease agent or one or more nucleic acids encoding the nuclease agent) and the exogenous donor sequence can be introduced in any form (e.g., DNA or RNA for guide RNAs; DNA, RNA, or protein for Cas proteins) via any delivery method (e.g., AAV, LNP, or HDD) and any route of administration as disclosed elsewhere herein. In one specific example, the nuclease agent (or the nucleic acid encoding the nuclease agent or one or more nucleic acids encoding the nuclease agent) is delivered by lipid nanoparticle (LNP)-mediated delivery, and the exogenous donor nucleic acid is delivered via adeno-associated virus (AAV)-mediated delivery (e.g., AAV8-mediated delivery or AAV2/8-mediated delivery). For example, the nuclease agent can be CRISPR/Cas9, and a Cas9 mRNA and a gRNA targeting the genomic locus or safe harbor locus (e.g., intron 1 of albumin) can be delivered via LNP-mediated delivery, and the exogenous donor nucleic acid can be delivered via AAV8-mediated delivery or AAV2/8-mediated delivery. In another specific example, both the nuclease agent (or the nucleic acid encoding the nuclease agent or one or more nucleic acids encoding the nuclease agent) and the exogenous donor nucleic acid are delivered via AAV-mediated delivery (e.g., via two separate AAVs, such as two separate AAV8s or AAV2/8s). For example, a first AAV (e.g., AAV8 or AAV2/8) can carry a Cas9 expression cassette, and a second AAV (e.g., AAV8 or AAV2/8) can carry a gRNA expression cassette and the exogenous donor nucleic acid. Alternatively, a first AAV (e.g., AAV8 or AAV2/8) can carry a Cas9 expression cassette and a gRNA expression cassette, and the second AAV (e.g., AAV8 or AAV2/8) can carry the exogenous donor nucleic acid. Different promoters can be used to drive expression of the gRNA, such as a U6 promoter or the small tRNA Gln. Likewise, different promoters can be used to drive Cas9 expression. In some methods, small promoters are used so that the Cas9 coding sequence can fit into an AAV construct. Examples of such promoters include Efs, SV40, or a synthetic promoter comprising a liver-specific enhancer (e.g., E2 from HBV virus or SerpinA from the SerpinA gene) and a core promoter (e.g., the E2P synthetic promoter or the SerpinAP synthetic promoter disclosed herein).

The antigen-binding-protein coding sequence can be inserted in particular types of cells in the animal. The method and vehicle for introducing the nuclease agent (or the nucleic acid encoding the nuclease agent or one or more nucleic acids encoding the nuclease agent) and the exogenous donor sequence into the animal can affect which types of cells in the animal are targeted. In some methods, for example, the antigen-binding-protein coding sequence is inserted into the genomic locus or safe harbor locus in liver cells. Methods and vehicles for introducing the nuclease agent (or the nucleic acid encoding the nuclease agent or one or more nucleic acids encoding the nuclease agent) and the exogenous donor sequence into the animal (including methods and vehicles that target the liver, such as lipid nanoparticle-mediated delivery and AAV8-mediated delivery or AAV2/8-mediated delivery), are disclosed in more detail elsewhere herein.

Targeted insertion of the antigen-binding-protein coding sequence into a genomic locus or safe harbor locus, and particularly the albumin safe harbor locus, offers multiple advantages. Such methods result in stable modification to allow for stable, long-term expression of the antigen-binding-protein coding sequence. With respect to the albumin safe harbor locus, such methods are able to utilize the high transcriptional activity of the native albumin enhancer/promoter. With in vivo gene targeting, it may not be possible to positively select corrected cells, and targeting a limited number of cells often may not result in enough secreted protein to correct a disease phenotype. Liver-directed gene transfer is attractive because of the liver's ability to secrete large amounts of protein into the blood, even if only a small percentage of liver cells is targeted.

The antigen-binding-protein coding sequence can be operably linked to an exogenous promoter in the exogenous donor nucleic acid. Examples of types of promoters that can be used are disclosed elsewhere herein. Alternatively, the antigen-binding-protein sequence can comprise a promoterless gene, and the inserted antigen-binding-protein coding sequence can be operably linked to an endogenous promoter in the genomic locus or safe harbor locus. Use of an endogenous promoter is advantageous because it obviates the need for inclusion of a promoter in the exogenous donor sequence, allowing packaging of larger transgenes that may not normally package efficiently, for example, in AAV. For example, the inserted antigen-binding-protein coding sequence can be inserted into an endogenous albumin locus and operably linked to the endogenous albumin promoter to produce high expression levels primarily in hepatic tissue.

Optionally, some or all of the endogenous gene at the genomic locus or safe harbor locus can be expressed upon insertion of the antigen-binding-protein coding sequence. Alternatively, none of the endogenous genomic gene or safe harbor gene can be expressed in some embodiments. As one example, the modified genomic locus or safe harbor locus can encode a chimeric protein comprising an endogenous secretion signal and the antigen-binding-protein. For example, the first intron of an albumin locus can be targeted, because the first exon of the albumin gene encodes a secretory peptide that is cleaved from the final protein product. In such a scenario, a promoterless antigen-binding-protein cassette bearing a splice acceptor and the antigen-binding-protein coding sequence will support expression and secretion of the antigen-binding protein. Splicing between albumin exon 1 and the integrated antigen-binding-protein coding sequence creates a chimeric mRNA and protein including the endogenous secretory peptide operably linked to the antigen-binding protein sequence.

The antigen-binding-protein coding sequence in the exogenous donor sequence can be inserted into the genomic locus or safe harbor locus by any means. Repair in response to double-strand breaks (DSBs) occurs principally through two conserved DNA repair pathways: homologous recombination (HR) and non-homologous end joining (NHEJ). See Kasparek & Humphrey (2011) Seminars in Cell & Dev. Biol. 22:886-897, herein incorporated by reference in its entirety for all purposes. Likewise, repair of a target nucleic acid mediated by an exogenous donor nucleic acid can include any process of exchange of genetic information between the two polynucleotides.

The term “recombination” includes any process of exchange of genetic information between two polynucleotides and can occur by any mechanism. Recombination can occur via homology directed repair (HDR) or homologous recombination (HR). HDR or HR includes a form of nucleic acid repair that can require nucleotide sequence homology, uses a “donor” molecule as a template for repair of a “target” molecule (i.e., the one that experienced the double-strand break), and leads to transfer of genetic information from the donor to target. Without wishing to be bound by any particular theory, such transfer can involve mismatch correction of heteroduplex DNA that forms between the broken target and the donor, and/or synthesis-dependent strand annealing, in which the donor is used to resynthesize genetic information that will become part of the target, and/or related processes. In some cases, the donor polynucleotide, a portion of the donor polynucleotide, a copy of the donor polynucleotide, or a portion of a copy of the donor polynucleotide integrates into the target DNA. See Wang et al. (2013) Cell 153:910-918; Mandalos et al. (2012) PLoS ONE 7: e45768: 1-9; and Wang et al. (2013) Nat Biotechnol. 31:530-532, each of which is herein incorporated by reference in its entirety for all purposes.

NHEJ includes the repair of double-strand breaks in a nucleic acid by direct ligation of the break ends to one another or to an exogenous sequence without the need for a homologous template. Ligation of non-contiguous sequences by NHEJ can often result in deletions, insertions, or translocations near the site of the double-strand break. For example, NHEJ can also result in the targeted integration of an exogenous donor nucleic acid through direct ligation of the break ends with the ends of the exogenous donor nucleic acid (i.e., NHEJ-based capture). Such NHEJ-mediated targeted integration can be preferred for insertion of an exogenous donor nucleic acid when homology directed repair (HDR) pathways are not readily usable (e.g., in non-dividing cells, primary cells, and cells which perform homology-based DNA repair poorly). In addition, in contrast to homology-directed repair, knowledge concerning large regions of sequence identity flanking the cleavage site is not needed, which can be beneficial when attempting targeted insertion into organisms that have genomes for which there is limited knowledge of the genomic sequence. The integration can proceed via ligation of blunt ends between the exogenous donor nucleic acid and the cleaved genomic sequence, or via ligation of sticky ends (i.e., having 5′ or 3′ overhangs) using an exogenous donor nucleic acid that is flanked by overhangs that are compatible with those generated by a nuclease agent in the cleaved genomic sequence. See, e.g., US 2011/020722, WO 2014/033644, WO 2014/089290, and Maresca et al. (2013) Genome Res. 23 (3): 539-546, each of which is herein incorporated by reference in its entirety for all purposes. If blunt ends are ligated, target and/or donor resection may be needed to generation regions of microhomology needed for fragment joining, which may create unwanted alterations in the target sequence.

In a specific example, the exogenous donor nucleic acid can be inserted via homology-independent targeted integration (e.g., directional homology-independent targeted integration). For example, the antigen-binding protein coding sequence in the exogenous donor nucleic acid is flanked on each side by a target site for a nuclease agent (e.g., the same target site as in the genomic locus or safe harbor locus, and the same nuclease agent being used to cleave the target site in the genomic locus or safe harbor locus). The nuclease agent can then cleave the target sites flanking the antigen-binding protein coding sequence. In a specific example, the exogenous donor nucleic acid is delivered AAV-mediated delivery, and cleavage of the target sites flanking the antigen-binding protein coding sequence can remove the inverted terminal repeats (ITRs) of the AAV. Removal of the ITRs can make it easier to assess successful targeting, because presence of the ITRs can hamper sequencing efforts due to the repeated sequences. In some methods, the target site in the genomic locus or safe harbor locus (e.g., a gRNA target sequence including the flanking protospacer adjacent motif) is no longer present if the antigen-binding protein coding sequence is inserted into the genomic locus or safe harbor locus in the correct orientation but it is reformed if the antigen-binding protein coding sequence is inserted into the genomic locus or safe harbor locus in the opposite orientation. This can help ensure that the antigen-binding protein coding sequence is inserted in the correct orientation for expression.

### A. CRISPR/Cas Nucleases and Other Nuclease Agents

### 1. CRISPR/Cas Systems

The methods and compositions disclosed herein can utilize Clustered Regularly Interspersed Short Palindromic Repeats (CRISPR)/CRISPR-associated (Cas) systems or components of such systems to modify a genome within a cell (e.g., a genomic locus or safe harbor locus in the genome, such as the albumin locus). CRISPR/Cas systems include transcripts and other elements involved in the expression of, or directing the activity of, Cas genes. A CRISPR/Cas system can be, for example, a type I, a type II, a type III system, or a type V system (e.g., subtype V-A or subtype V-B). The methods and compositions disclosed herein can employ CRISPR/Cas systems by utilizing CRISPR complexes (comprising a guide RNA (gRNA) complexed with a Cas protein) for site-directed binding or cleavage of nucleic acids.

CRISPR/Cas systems used in the compositions and methods disclosed herein can be non-naturally occurring. A “non-naturally occurring” system includes anything indicating the involvement of the hand of man, such as one or more components of the system being altered or mutated from their naturally occurring state, being at least substantially free from at least one other component with which they are naturally associated in nature, or being associated with at least one other component with which they are not naturally associated. For example, some CRISPR/Cas systems employ non-naturally occurring CRISPR complexes comprising a gRNA and a Cas protein that do not naturally occur together, employ a Cas protein that does not occur naturally, or employ a gRNA that does not occur naturally.

a. Cas Proteins

Cas proteins generally comprise at least one RNA recognition or binding domain that can interact with guide RNAs. Cas proteins can also comprise nuclease domains (e.g., DNase domains or RNase domains), DNA-binding domains, helicase domains, protein-protein interaction domains, dimerization domains, and other domains. Some such domains (e.g., DNase domains) can be from a native Cas protein. Other such domains can be added to make a modified Cas protein. A nuclease domain possesses catalytic activity for nucleic acid cleavage, which includes the breakage of the covalent bonds of a nucleic acid molecule. Cleavage can produce blunt ends or staggered ends, and it can be single-stranded or double-stranded. For example, a wild type Cas9 protein will typically create a blunt cleavage product. Alternatively, a wild type Cpf1 protein (e.g., FnCpf1) can result in a cleavage product with a 5-nucleotide 5′ overhang, with the cleavage occurring after the 18th base pair from the PAM sequence on the non-targeted strand and after the 23rd base on the targeted strand. A Cas protein can have full cleavage activity to create a double-strand break at a target genomic locus (e.g., a double-strand break with blunt ends), or it can be a nickase that creates a single-strand break at a target genomic locus.

Examples of Cas proteins include Cas1, Cas1B, Cas2, Cas3, Cas4, Cas5, Cas5e (CasD), Cas6, Cas6e, Casof, Cas7, Cas8a1, Cas8α2, Cas8b, Cas8c, Cas9 (Csn1 or Csx12), Cas10, Cas10d, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof.

An exemplary Cas protein is a Cas9 protein or a protein derived from a Cas9 protein. Cas9 proteins are from a type II CRISPR/Cas system and typically share four key motifs with a conserved architecture. Motifs 1, 2, and 4 are RuvC-like motifs, and motif 3 is an HNH motif. Exemplary Cas9 proteins are from Streptococcus pyogenes, Streptococcus thermophilus, Streptococcus sp., Staphylococcus aureus, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, Neisseria meningitidis, or Campylobacter jejuni. Additional examples of the Cas9 family members are described in WO 2014/131833, herein incorporated by reference in its entirety for all purposes. Cas9 from S. pyogenes (SpCas9) (assigned SwissProt accession number Q99ZW2) is an exemplary Cas9 protein. An exemplary SpCas9 protein sequence is set forth in SEQ ID NO: 62 (encoded by the DNA sequence set forth in SEQ ID NO: 61). An exemplary SpCas9 mRNA sequence is set forth in SEQ ID NO: 63. Cas9 from S. aureus (SaCas9) (assigned UniProt accession number J7RUA5) is another exemplary Cas9 protein. Cas9 from Campylobacter jejuni (CjCas9) (assigned UniProt accession number QOP897) is another exemplary Cas9 protein. See, e.g., Kim et al. (2017) Nat. Comm. 8:14500, herein incorporated by reference in its entirety for all purposes. SaCas9 is smaller than SpCas9, and CjCas9 is smaller than both SaCas9 and SpCas9. Cas9 from Neisseria meningitidis (Nme2Cas9) is another exemplary Cas9 protein. See, e.g., Edraki et al. (2019) Mol. Cell 73 (4): 714-726, herein incorporated by reference in its entirety for all purposes. Cas9 proteins from Streptococcus thermophilus (e.g., Streptococcus thermophilus LMD-9 Cas9 encoded by the CRISPR1 locus (St1Cas9) or Streptococcus thermophilus Cas9 from the CRISPR3 locus (St3Cas9)) are other exemplary Cas9 proteins. Cas9 from Francisella novicida (FnCas9) or the RHA Francisella novicida Cas9 variant that recognizes an alternative PAM (E1369R/E1449H/R1556A substitutions) are other exemplary Cas9 proteins. These and other exemplary Cas9 proteins are reviewed, e.g., in Cebrian-Serrano and Davies (2017) Mamm. Genome 28 (7): 247-261, herein incorporated by reference in its entirety for all purposes.

Another example of a Cas protein is a Cpf1 (CRISPR from Prevotella and Francisella 1) protein. Cpf1 is a large protein (about 1300 amino acids) that contains a RuvC-like nuclease domain homologous to the corresponding domain of Cas9 along with a counterpart to the characteristic arginine-rich cluster of Cas9. However, Cpf1 lacks the HNH nuclease domain that is present in Cas9 proteins, and the RuvC-like domain is contiguous in the Cpf1 sequence, in contrast to Cas9 where it contains long inserts including the HNH domain. See, e.g., Zetsche et al. (2015) Cell 163 (3): 759-771, herein incorporated by reference in its entirety for all purposes. Exemplary Cpf1 proteins are from Francisella tularensis 1, Francisella tularensis subsp. novicida, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2-44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens, and Porphyromonas macacae. Cpf1 from Francisella novicida U112 (FnCpf1; assigned UniProt accession number AOQ7Q2) is an exemplary Cpf1 protein.

Cas proteins can be wild type proteins (i.e., those that occur in nature), modified Cas proteins (i.e., Cas protein variants), or fragments of wild type or modified Cas proteins. Cas proteins can also be active variants or fragments with respect to catalytic activity of wild type or modified Cas proteins. Active variants or fragments with respect to catalytic activity can comprise at least 80%, 85%, 90%, 91%, 92%, 93%, 94%, 95%, 96%, 97%, 98%, 99% or more sequence identity to the wild type or modified Cas protein or a portion thereof, wherein the active variants retain the ability to cut at a desired cleavage site and hence retain nick-inducing or double-strand-break-inducing activity. Assays for nick-inducing or double-strand-break-inducing activity are known and generally measure the overall activity and specificity of the Cas protein on DNA substrates containing the cleavage site.

One example of a modified Cas protein is the modified SpCas9-HF1 protein, which is a high-fidelity variant of Streptococcus pyogenes Cas9 harboring alterations (N₄₉₇A/R661A/Q695A/Q926A) designed to reduce non-specific DNA contacts. See, e.g., Kleinstiver et al. (2016) Nature 529 (7587): 490-495, herein incorporated by reference in its entirety for all purposes. Another example of a modified Cas protein is the modified eSpCas9 variant (K848A/K1003A/R1060A) designed to reduce off-target effects. See, e.g., Slaymaker et al. (2016) Science 351 (6268): 84-88, herein incorporated by reference in its entirety for all purposes. Other SpCas9 variants include K855A and K810A/K1003A/R1060A. These and other modified Cas proteins are reviewed, e.g., in Cebrian-Serrano and Davies (2017) Mamm. Genome 28 (7): 247-261, herein incorporated by reference in its entirety for all purposes. Another example of a modified Cas9 protein is xCas9, which is a SpCas9 variant that can recognize an expanded range of PAM sequences. See, e.g., Hu et al. (2018) Nature 556:57-63, herein incorporated by reference in its entirety for all purposes.

Cas proteins can be modified to increase or decrease one or more of nucleic acid binding affinity, nucleic acid binding specificity, and enzymatic activity. Cas proteins can also be modified to change any other activity or property of the protein, such as stability. For example, one or more nuclease domains of the Cas protein can be modified, deleted, or inactivated, or a Cas protein can be truncated to remove domains that are not essential for the function of the protein or to optimize (e.g., enhance or reduce) the activity of or a property of the Cas protein.

Cas proteins can comprise at least one nuclease domain, such as a DNase domain. For example, a wild type Cpf1 protein generally comprises a RuvC-like domain that cleaves both strands of target DNA, perhaps in a dimeric configuration. Cas proteins can also comprise at least two nuclease domains, such as DNase domains. For example, a wild type Cas9 protein generally comprises a RuvC-like nuclease domain and an HNH-like nuclease domain. The RuvC and HNH domains can each cut a different strand of double-stranded DNA to make a double-stranded break in the DNA. See, e.g., Jinek et al. (2012) Science 337 (6096): 816-821, herein incorporated by reference in its entirety for all purposes.

One or more or all of the nuclease domains can be deleted or mutated so that they are no longer functional or have reduced nuclease activity. For example, if one of the nuclease domains is deleted or mutated in a Cas9 protein, the resulting Cas9 protein can be referred to as a nickase and can generate a single-strand break within a double-stranded target DNA but not a double-strand break (i.e., it can cleave the complementary strand or the non-complementary strand, but not both). If both of the nuclease domains are deleted or mutated, the resulting Cas protein (e.g., Cas9) will have a reduced ability to cleave both strands of a double-stranded DNA (e.g., a nuclease-null or nuclease-inactive Cas protein, or a catalytically dead Cas protein (dCas)). An example of a mutation that converts Cas9 into a nickase is a D10A (aspartate to alanine at position 10 of Cas9) mutation in the RuvC domain of Cas9 from S. pyogenes. Likewise, H939A (histidine to alanine at amino acid position 839), H840A (histidine to alanine at amino acid position 840), or N₈₆₃A (asparagine to alanine at amino acid position N₈₆₃) in the HNH domain of Cas9 from S. pyogenes can convert the Cas9 into a nickase. Other examples of mutations that convert Cas9 into a nickase include the corresponding mutations to Cas9 from S. thermophilus. See, e.g., Sapranauskas et al. (2011) Nucleic Acids Res. 39 (21): 9275-9282 and WO 2013/141680, each of which is herein incorporated by reference in its entirety for all purposes. Such mutations can be generated using methods such as site-directed mutagenesis, PCR-mediated mutagenesis, or total gene synthesis. Examples of other mutations creating nickases can be found, for example, in WO 2013/176772 and WO 2013/142578, each of which is herein incorporated by reference in its entirety for all purposes. If all of the nuclease domains are deleted or mutated in a Cas protein (e.g., both of the nuclease domains are deleted or mutated in a Cas9 protein), the resulting Cas protein (e.g., Cas9) will have a reduced ability to cleave both strands of a double-stranded DNA (e.g., a nuclease-null or nuclease-inactive Cas protein). One specific example is a D10A/H840A S. pyogenes Cas9 double mutant or a corresponding double mutant in a Cas9 from another species when optimally aligned with S. pyogenes Cas9. Another specific example is a D10A/N₈₆₃A S. pyogenes Cas9 double mutant or a corresponding double mutant in a Cas9 from another species when optimally aligned with S. pyogenes Cas9.

Examples of inactivating mutations in the catalytic domains of xCas9 are the same as those described above for SpCas9. Examples of inactivating mutations in the catalytic domains of Staphylococcus aureus Cas9 proteins are also known. For example, the Staphylococcus aureus Cas9 enzyme (SaCas9) may comprise a substitution at position N₅₈₀ (e.g., N₅₈₀A substitution) and a substitution at position D10 (e.g., D10A substitution) to generate a nuclease-inactive Cas protein. See, e.g., WO 2016/106236, herein incorporated by reference in its entirety for all purposes. Examples of inactivating mutations in the catalytic domains of Nme2Cas9 are also known (e.g., combination of D16A and H588A). Examples of inactivating mutations in the catalytic domains of St1Cas9 are also known (e.g., combination of D9A, D598A, H599A, and N₆₂₂A). Examples of inactivating mutations in the catalytic domains of St3Cas9 are also known (e.g., combination of D10A and N₈₇₀A). Examples of inactivating mutations in the catalytic domains of CjCas9 are also known (e.g., combination of D8A and H559A). Examples of inactivating mutations in the catalytic domains of FnCas9 and RHA FnCas9 are also known (e.g., N₉₉₅A).

Examples of inactivating mutations in the catalytic domains of Cpf1 proteins are also known. With reference to Cpf1 proteins from Francisella novicida U112 (FnCpf1), Acidaminococcus sp. BV3L6 (AsCpf1), Lachnospiraceae bacterium ND2006 (LbCpf1), and Moraxella bovoculi 237 (MbCpf1 Cpf1), such mutations can include mutations at positions 908, 993, or 1263 of AsCpf1 or corresponding positions in Cpf1 orthologs, or positions 832, 925, 947, or 1180 of LbCpf1 or corresponding positions in Cpf1 orthologs. Such mutations can include, for example one or more of mutations D908A, E993A, and D1263A of AsCpf1 or corresponding mutations in Cpf1 orthologs, or D832A, E925A, D947A, and D1180A of LbCpf1 or corresponding mutations in Cpf1 orthologs. See, e.g., US 2016/0208243, herein incorporated by reference in its entirety for all purposes.

Cas proteins can also be operably linked to heterologous polypeptides as fusion proteins. For example, a Cas protein can be fused to a cleavage domain or an epigenetic modification domain. See WO 2014/089290, herein incorporated by reference in its entirety for all purposes. Cas proteins can also be fused to a heterologous polypeptide providing increased or decreased stability. The fused domain or heterologous polypeptide can be located at the N-terminus, the C-terminus, or internally within the Cas protein.

As one example, a Cas protein can be fused to one or more heterologous polypeptides that provide for subcellular localization. Such heterologous polypeptides can include, for example, one or more nuclear localization signals (NLS) such as the monopartite SV40 NLS and/or a bipartite alpha-importin NLS for targeting to the nucleus, a mitochondrial localization signal for targeting to the mitochondria, an ER retention signal, and the like. See, e.g., Lange et al. (2007) J. Biol. Chem. 282 (8): 5101-5105, herein incorporated by reference in its entirety for all purposes. Such subcellular localization signals can be located at the N-terminus, the C-terminus, or anywhere within the Cas protein. An NLS can comprise a stretch of basic amino acids, and can be a monopartite sequence or a bipartite sequence. Optionally, a Cas protein can comprise two or more NLSs, including an NLS (e.g., an alpha-importin NLS or a monopartite NLS) at the N-terminus and an NLS (e.g., an SV40 NLS or a bipartite NLS) at the C-terminus. A Cas protein can also comprise two or more NLSs at the N-terminus and/or two or more NLSs at the C-terminus.

Cas proteins can also be operably linked to a cell-penetrating domain or protein transduction domain. For example, the cell-penetrating domain can be derived from the HIV-1 TAT protein, the TLM cell-penetrating motif from human hepatitis B virus, MPG, Pep-1, VP22, a cell penetrating peptide from Herpes simplex virus, or a polyarginine peptide sequence. See, e.g., WO 2014/089290 and WO 2013/176772, each of which is herein incorporated by reference in its entirety for all purposes. The cell-penetrating domain can be located at the N-terminus, the C-terminus, or anywhere within the Cas protein.

Cas proteins can also be operably linked to a heterologous polypeptide for ease of tracking or purification, such as a fluorescent protein, a purification tag, or an epitope tag. Examples of fluorescent proteins include green fluorescent proteins (e.g., GFP, GFP-2, tagGFP, turboGFP, eGFP, Emerald, Azami Green, Monomeric Azami Green, CopGFP, AceGFP, ZsGreenl), yellow fluorescent proteins (e.g., YFP, eYFP, Citrine, Venus, YPet, PhiYFP, ZsYellowl), blue fluorescent proteins (e.g., eBFP, eBFP2, Azurite, mKalamal, GFPuv, Sapphire, T-sapphire), cyan fluorescent proteins (e.g., eCFP, Cerulean, CyPet, AmCyanl, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRed1, AsRed2, eqFP611, mRaspberry, mStrawberry, Jred), orange fluorescent proteins (e.g., mOrange, mKO, Kusabira-Orange, Monomeric Kusabira-Orange, mTangerine, tdTomato), and any other suitable fluorescent protein. Examples of tags include glutathione-S-transferase (GST), chitin binding protein (CBP), maltose binding protein, thioredoxin (TRX), poly(NANP), tandem affinity purification (TAP) tag, myc, AcV5, AU1, AU5, E, ECS, E2, FLAG, hemagglutinin (HA), nus, Softag 1, Softag 3, Strep, SBP, Glu-Glu, HSV, KT3, S, S1, T7, V5, VSV-G, histidine (His), biotin carboxyl carrier protein (BCCP), and calmodulin.

Cas proteins can also be tethered to labeled nucleic acids or donor sequences. Such tethering (i.e., physical linking) can be achieved through covalent interactions or noncovalent interactions, and the tethering can be direct (e.g., through direct fusion or chemical conjugation, which can be achieved by modification of cysteine or lysine residues on the protein or intein modification), or can be achieved through one or more intervening linkers or adapter molecules such as streptavidin or aptamers. See, e.g., Pierce et al. (2005) Mini Rev. Med. Chem. 5 (1): 41-55; Duckworth et al. (2007) Angew. Chem. Int. Ed. Engl. 46 (46): 8819-8822; Schaeffer and Dixon (2009) Australian J. Chem. 62 (10): 1328-1332; Goodman et al. (2009) Chembiochem. 10 (9): 1551-1557; and Khatwani et al. (2012) Bioorg. Med. Chem. 20 (14): 4532-4539, each of which is herein incorporated by reference in its entirety for all purposes. Noncovalent strategies for synthesizing protein-nucleic acid conjugates include biotin-streptavidin and nickel-histidine methods. Covalent protein-nucleic acid conjugates can be synthesized by connecting appropriately functionalized nucleic acids and proteins using a wide variety of chemistries. Some of these chemistries involve direct attachment of the oligonucleotide to an amino acid residue on the protein surface (e.g., a lysine amine or a cysteine thiol), while other more complex schemes require post-translational modification of the protein or the involvement of a catalytic or reactive protein domain. Methods for covalent attachment of proteins to nucleic acids can include, for example, chemical cross-linking of oligonucleotides to protein lysine or cysteine residues, expressed protein-ligation, chemoenzymatic methods, and the use of photoaptamers. The labeled nucleic acid or donor sequence can be tethered to the C-terminus, the N-terminus, or to an internal region within the Cas protein. In one example, the labeled nucleic acid or donor sequence is tethered to the C-terminus or the N-terminus of the Cas protein. Likewise, the Cas protein can be tethered to the 5′ end, the 3′ end, or to an internal region within the labeled nucleic acid or donor sequence. That is, the labeled nucleic acid or donor sequence can be tethered in any orientation and polarity. For example, the Cas protein can be tethered to the 5′ end or the 3′ end of the labeled nucleic acid or donor sequence.

Cas proteins can be provided in any form. For example, a Cas protein can be provided in the form of a protein, such as a Cas protein complexed with a gRNA. Alternatively, a Cas protein can be provided in the form of a nucleic acid encoding the Cas protein, such as an RNA (e.g., messenger RNA (mRNA)) or DNA. Optionally, the nucleic acid encoding the Cas protein can be codon optimized for efficient translation into protein in a particular cell or organism. For example, the nucleic acid encoding the Cas protein can be modified to substitute codons having a higher frequency of usage in a bacterial cell, a yeast cell, a human cell, a non-human cell, a mammalian cell, a rodent cell, a mouse cell, a rat cell, or any other host cell of interest, as compared to the naturally occurring polynucleotide sequence. When a nucleic acid encoding the Cas protein is introduced into the cell, the Cas protein can be transiently, conditionally, or constitutively expressed in the cell.

Cas proteins provided as mRNAs can be modified for improved stability and/or immunogenicity properties. The modifications may be made to one or more nucleosides within the mRNA. Examples of chemical modifications to mRNA nucleobases include pseudouridine, 1-methyl-pseudouridine, and 5-methyl-cytidine. For example, capped and polyadenylated Cas mRNA containing N₁-methyl pseudouridine can be used. Likewise, Cas mRNAs can be modified by depletion of uridine using synonymous codons.

Nucleic acids encoding Cas proteins can be stably integrated in the genome of a cell and operably linked to a promoter active in the cell. Alternatively, nucleic acids encoding Cas proteins can be operably linked to a promoter in an expression construct. Expression constructs include any nucleic acid constructs capable of directing expression of a gene or other nucleic acid sequence of interest (e.g., a Cas gene) and which can transfer such a nucleic acid sequence of interest to a target cell. For example, the nucleic acid encoding the Cas protein can be in a vector comprising a DNA encoding a gRNA. Alternatively, it can be in a vector or plasmid that is separate from the vector comprising the DNA encoding the gRNA. Promoters that can be used in an expression construct include promoters active, for example, in one or more of a eukaryotic cell, a human cell, a non-human cell, a mammalian cell, a non-human mammalian cell, a rodent cell, a mouse cell, a rat cell, a pluripotent cell, an embryonic stem (ES) cell, an adult stem cell, a developmentally restricted progenitor cell, an induced pluripotent stem (iPS) cell, or a one-cell stage embryo. Such promoters can be, for example, conditional promoters, inducible promoters, constitutive promoters, or tissue-specific promoters. Optionally, the promoter can be a bidirectional promoter driving expression of both a Cas protein in one direction and a guide RNA in the other direction. Such bidirectional promoters can consist of (1) a complete, conventional, unidirectional Pol III promoter that contains 3 external control elements: a distal sequence element (DSE), a proximal sequence element (PSE), and a TATA box; and (2) a second basic Pol III promoter that includes a PSE and a TATA box fused to the 5′ terminus of the DSE in reverse orientation. For example, in the H1 promoter, the DSE is adjacent to the PSE and the TATA box, and the promoter can be rendered bidirectional by creating a hybrid promoter in which transcription in the reverse direction is controlled by appending a PSE and TATA box derived from the U6 promoter. See, e.g., US 2016/0074535, herein incorporated by references in its entirety for all purposes. Use of a bidirectional promoter to express genes encoding a Cas protein and a guide RNA simultaneously allow for the generation of compact expression cassettes to facilitate delivery.

Different promoters can be used to drive Cas expression or Cas9 expression. In some methods, small promoters are used so that the Cas or Cas9 coding sequence can fit into an AAV construct. Examples of such promoters include Efs, SV40, or a synthetic promoter comprising a liver-specific enhancer (e.g., E2 from HBV virus or SerpinA from the SerpinA gene) and a core promoter (e.g., the E2P synthetic promoter or the SerpinAP synthetic promoter).

b. Guide RNAs

A “guide RNA” or “gRNA” is an RNA molecule that binds to a Cas protein (e.g., Cas9 protein) and targets the Cas protein to a specific location within a target DNA. Guide RNAs can comprise two segments: a “DNA-targeting segment” and a “protein-binding segment.” “Segment” includes a section or region of a molecule, such as a contiguous stretch of nucleotides in an RNA. Some gRNAs, such as those for Cas9, can comprise two separate RNA molecules: an “activator-RNA” (e.g., tracrRNA) and a “targeter-RNA” (e.g., CRISPR RNA or crRNA). Other gRNAs are a single RNA molecule (single RNA polynucleotide), which can also be called a “single-molecule gRNA,” a “single-guide RNA,” or an “sgRNA.” See, e.g., WO 2013/176772, WO 2014/065596, WO 2014/089290, WO 2014/093622, WO 2014/099750, WO 2013/142578, and WO 2014/131833, each of which is herein incorporated by reference in its entirety for all purposes. For Cas9, for example, a single-guide RNA can comprise a crRNA fused to a tracrRNA (e.g., via a linker). For Cpf1, for example, only a crRNA is needed to achieve binding to a target sequence. The terms “guide RNA” and “gRNA” include both double-molecule (i.e., modular) gRNAs and single-molecule gRNAs.

An exemplary two-molecule gRNA comprises a crRNA-like (“CRISPR RNA” or “targeter-RNA” or “crRNA” or “crRNA repeat”) molecule and a corresponding tracrRNA-like (“trans-acting CRISPR RNA” or “activator-RNA” or “tracrRNA”) molecule. A crRNA comprises both the DNA-targeting segment (single-stranded) of the gRNA and a stretch of nucleotides that forms one half of the dsRNA duplex of the protein-binding segment of the gRNA. An example of a crRNA tail, located downstream (3′) of the DNA-targeting segment, comprises, consists essentially of, or consists of GUUUUAGAGCUAUGCU (SEQ ID NO: 51). Any of the DNA-targeting segments disclosed herein can be joined to the 5′ end of SEQ ID NO: 51 to form a crRNA.

A corresponding tracrRNA (activator-RNA) comprises a stretch of nucleotides that forms the other half of the dsRNA duplex of the protein-binding segment of the gRNA. A stretch of nucleotides of a crRNA are complementary to and hybridize with a stretch of nucleotides of a tracrRNA to form the dsRNA duplex of the protein-binding domain of the gRNA. As such, each crRNA can be said to have a corresponding tracrRNA. Exemplary tracrRNA sequences comprise, consist essentially of, or consist of AGCAUAGCAAGUUAAAAUAAGGCUAGUCCGUUAUCAACUUGAAAAAGUGGCACC GAGUCGGUGCUUU (SEQ ID NO: 52), AAACAGCAUAGCAAGUUAAAAUAAGGCUAGUCCGUUAUCAACUUGAAAAAGUGG CACCGAGUCGGUGCUUUU (SEQ ID NO: 121), or GUUGGAACCAUUCAAAACAGCAUAGCAAGUUAAAAUAAGGCUAGUCCGUUAUCA ACUUGAAAAAGUGGCACCGAGUCGGUGC (SEQ ID NO: 122).

In systems in which both a crRNA and a tracrRNA are needed, the crRNA and the corresponding tracrRNA hybridize to form a gRNA. In systems in which only a crRNA is needed, the crRNA can be the gRNA. The crRNA additionally provides the single-stranded DNA-targeting segment that hybridizes to the complementary strand of a target DNA. If used for modification within a cell, the exact sequence of a given crRNA or tracrRNA molecule can be designed to be specific to the species in which the RNA molecules will be used. See, e.g., Mali et al. (2013) Science 339 (6121): 823-826; Jinek et al. (2012) Science 337 (6096): 816-821; Hwang et al. (2013) Nat. Biotechnol. 31 (3): 227-229; Jiang et al. (2013) Nat. Biotechnol. 31 (3): 233-239; and Cong et al. (2013) Science 339 (6121): 819-823, each of which is herein incorporated by reference in its entirety for all purposes.

The DNA-targeting segment (crRNA) of a given gRNA comprises a nucleotide sequence that is complementary to a sequence on the complementary strand of the target DNA, as described in more detail below. The DNA-targeting segment of a gRNA interacts with the target DNA in a sequence-specific manner via hybridization (i.e., base pairing). As such, the nucleotide sequence of the DNA-targeting segment may vary and determines the location within the target DNA with which the gRNA and the target DNA will interact. The DNA-targeting segment of a subject gRNA can be modified to hybridize to any desired sequence within a target DNA. Naturally occurring crRNAs differ depending on the CRISPR/Cas system and organism but often contain a targeting segment of between 21 to 72 nucleotides length, flanked by two direct repeats (DR) of a length of between 21 to 46 nucleotides (see, e.g., WO 2014/131833, herein incorporated by reference in its entirety for all purposes). In the case of S. pyogenes, the DRs are 36 nucleotides long and the targeting segment is 30 nucleotides long. The 3′ located DR is complementary to and hybridizes with the corresponding tracrRNA, which in turn binds to the Cas protein.

The DNA-targeting segment can have, for example, a length of at least about 12, 15, 17, 18, 19, 20, 25, 30, 35, or 40 nucleotides. Such DNA-targeting segments can have, for example, a length from about 12 to about 100, from about 12 to about 80, from about 12 to about 50, from about 12 to about 40, from about 12 to about 30, from about 12 to about 25, or from about 12 to about 20 nucleotides. For example, the DNA targeting segment can be from about 15 to about 25 nucleotides (e.g., from about 17 to about 20 nucleotides, or about 17, 18, 19, or 20 nucleotides). See, e.g., US 2016/0024523, herein incorporated by reference in its entirety for all purposes. For Cas9 from S. pyogenes, a typical DNA-targeting segment is between 16 and 20 nucleotides in length or between 17 and 20 nucleotides in length. For Cas9 from S. aureus, a typical DNA-targeting segment is between 21 and 23 nucleotides in length. For Cpf1, a typical DNA-targeting segment is at least 16 nucleotides in length or at least 18 nucleotides in length.

TracrRNAs can be in any form (e.g., full-length tracrRNAs or active partial tracrRNAs) and of varying lengths. They can include primary transcripts or processed forms. For example, tracrRNAs (as part of a single-guide RNA or as a separate molecule as part of a two-molecule gRNA) may comprise, consist essentially of, or consist of all or a portion of a wild type tracrRNA sequence (e.g., about or more than about 20, 26, 32, 45, 48, 54, 63, 67, 85, or more nucleotides of a wild type tracrRNA sequence). Examples of wild type tracrRNA sequences from S. pyogenes include 171-nucleotide, 89-nucleotide, 75-nucleotide, and 65-nucleotide versions. See, e.g., Deltcheva et al. (2011) Nature 471 (7340): 602-607; WO 2014/093661, each of which is herein incorporated by reference in its entirety for all purposes. Examples of tracrRNAs within single-guide RNAs (sgRNAs) include the tracrRNA segments found within +48, +54, +67, and +85 versions of sgRNAs, where “+n” indicates that up to the +n nucleotide of wild type tracrRNA is included in the sgRNA. See U.S. Pat. No. 8,697,359, herein incorporated by reference in its entirety for all purposes.

The percent complementarity between the DNA-targeting segment of the guide RNA and the complementary strand of the target DNA can be at least 60% (e.g., at least 65%, at least 70%, at least 75%, at least 80%, at least 85%, at least 90%, at least 95%, at least 97%, at least 98%, at least 99%, or 100%). The percent complementarity between the DNA-targeting segment and the complementary strand of the target DNA can be at least 60% over about 20 contiguous nucleotides. As an example, the percent complementarity between the DNA-targeting segment and the complementary strand of the target DNA can be 100% over the 14 contiguous nucleotides at the 5′ end of the complementary strand of the target DNA and as low as 0% over the remainder. In such a case, the DNA-targeting segment can be considered to be 14 nucleotides in length. As another example, the percent complementarity between the DNA-targeting segment and the complementary strand of the target DNA can be 100% over the seven contiguous nucleotides at the 5′ end of the complementary strand of the target DNA and as low as 0% over the remainder. In such a case, the DNA-targeting segment can be considered to be 7 nucleotides in length. In some guide RNAs, at least 17 nucleotides within the DNA-targeting segment are complementary to the complementary strand of the target DNA. For example, the DNA-targeting segment can be 20 nucleotides in length and can comprise 1, 2, or 3 mismatches with the complementary strand of the target DNA. In one example, the mismatches are not adjacent to the region of the complementary strand corresponding to the protospacer adjacent motif (PAM) sequence (i.e., the reverse complement of the PAM sequence) (e.g., the mismatches are in the 5′ end of the DNA-targeting segment of the guide RNA, or the mismatches are at least 2, 3, 4, 5, 6, 7, 8, 9, 10, 11, 12, 13, 14, 15, 16, 17, 18, or 19 base pairs away from the region of the complementary strand corresponding to the PAM sequence).

The protein-binding segment of a gRNA can comprise two stretches of nucleotides that are complementary to one another. The complementary nucleotides of the protein-binding segment hybridize to form a double-stranded RNA duplex (dsRNA). The protein-binding segment of a subject gRNA interacts with a Cas protein, and the gRNA directs the bound Cas protein to a specific nucleotide sequence within target DNA via the DNA-targeting segment.

Single-guide RNAs can comprise a DNA-targeting segment and a scaffold sequence (i.e., the protein-binding or Cas-binding sequence of the guide RNA). For example, such guide RNAs can have a 5′ DNA-targeting segment joined to a 3′ scaffold sequence. Exemplary scaffold sequences comprise, consist essentially of, or consist of: GUUUUAGAGCUAGAAAUAGCAAGUUAAAAUAAGGCUAGUCCGUUAUCAACUUGA AAAAGUGGCACCGAGUCGGUGCU (version 1; SEQ ID NO: 53); GUUGGAACCAUUCAAAACAGCAUAGCAAGUUAAAAUAAGGCUAGUCCGUUAUCA ACUUGAAAAAGUGGCACCGAGUCGGUGC (version 2; SEQ ID NO: 54); GUUUUAGAGCUAGAAAUAGCAAGUUAAAAUAAGGCUAGUCCGUUAUCAACUUGA AAAAGUGGCACCGAGUCGGUGC (version 3; SEQ ID NO: 55); GUUUAAGAGCUAUGCUGGAAACAGCAUAGCAAGUUUAAAUAAGGCUAGUCCGUU AUCAACUUGAAAAAGUGGCACCGAGUCGGUGC (version 4; SEQ ID NO: 56); and GUUUUAGAGCUAGAAAUAGCAAGUUAAAAUAAGGCUAGUCCGUUAUCAACUUGA AAAAGUGGCACCGAGUCGGUGCUUUUUUU (version 5; SEQ ID NO: 57); GUUUUAGAGCUAGAAAUAGCAAGUUAAAAUAAGGCUAGUCCGUUAUCAACUUGA AAAAGUGGCACCGAGUCGGUGCUUUU (version 6; SEQ ID NO: 123); or GUUUAAGAGCUAUGCUGGAAACAGCAUAGCAAGUUUAAAUAAGGCUAGUCCGUU AUCAACUUGAAAAAGUGGCACCGAGUCGGUGCUUUUUU (version 7; SEQ ID NO: 124). Guide RNAs targeting any of the guide RNA target sequences disclosed herein can include, for example, a DNA-targeting segment on the 5′ end of the guide RNA fused to any of the exemplary guide RNA scaffold sequences on the 3′ end of the guide RNA. That is, any of the DNA-targeting segments disclosed herein can be joined to the 5′ end of any one of the above scaffold sequences to form a single guide RNA (chimeric guide RNA).

Guide RNAs can include modifications or sequences that provide for additional desirable features (e.g., modified or regulated stability; subcellular targeting; tracking with a fluorescent label; a binding site for a protein or protein complex; and the like). Examples of such modifications include, for example, a 5′ cap (e.g., a 7-methylguanylate cap (m7G)); a 3′ polyadenylated tail (i.e., a 3′ poly(A) tail); a riboswitch sequence (e.g., to allow for regulated stability and/or regulated accessibility by proteins and/or protein complexes); a stability control sequence; a sequence that forms a dsRNA duplex (i.e., a hairpin); a modification or sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, and so forth); a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like); and combinations thereof. Other examples of modifications include engineered stem loop duplex structures, engineered bulge regions, engineered hairpins 3′ of the stem loop duplex structure, or any combination thereof. See, e.g., US 2015/0376586, herein incorporated by reference in its entirety for all purposes. A bulge can be an unpaired region of nucleotides within the duplex made up of the crRNA-like region and the minimum tracrRNA-like region. A bulge can comprise, on one side of the duplex, an unpaired 5′-XXXY-3′ where X is any purine and Y can be a nucleotide that can form a wobble pair with a nucleotide on the opposite strand, and an unpaired nucleotide region on the other side of the duplex.

Unmodified nucleic acids can be prone to degradation. Exogenous nucleic acids can also induce an innate immune response. Modifications can help introduce stability and reduce immunogenicity. Guide RNAs can comprise modified nucleosides and modified nucleotides including, for example, one or more of the following: (1) alteration or replacement of one or both of the non-linking phosphate oxygens and/or of one or more of the linking phosphate oxygens in the phosphodiester backbone linkage; (2) alteration or replacement of a constituent of the ribose sugar such as alteration or replacement of the 2′ hydroxyl on the ribose sugar; (3) replacement of the phosphate moiety with dephospho linkers; (4) modification or replacement of a naturally occurring nucleobase; (5) replacement or modification of the ribose-phosphate backbone; (6) modification of the 3′ end or 5′ end of the oligonucleotide (e.g., removal, modification or replacement of a terminal phosphate group or conjugation of a moiety); and (7) modification of the sugar. Other possible guide RNA modifications include modifications of or replacement of uracils or poly-uracil tracts. See, e.g., WO 2015/048577 and US 2016/0237455, each of which is herein incorporated by reference in its entirety for all purposes. Similar modifications can be made to Cas-encoding nucleic acids, such as Cas mRNAs. For example, Cas mRNAs can be modified by depletion of uridine using synonymous codons.

As one example, nucleotides at the 5′ or 3′ end of a guide RNA can include phosphorothioate linkages (e.g., the bases can have a modified phosphate group that is a phosphorothioate group). For example, a guide RNA can include phosphorothioate linkages between the 2, 3, or 4 terminal nucleotides at the 5′ or 3′ end of the guide RNA. As another example, nucleotides at the 5′ and/or 3′ end of a guide RNA can have 2′-O-methyl modifications. For example, a guide RNA can include 2′-O-methyl modifications at the 2, 3, or 4 terminal nucleotides at the 5′ and/or 3′ end of the guide RNA (e.g., the 5′ end). See, e.g., WO 2017/173054 A1 and Finn et al. (2018) Cell Rep. 22 (9): 2227-2235, each of which is herein incorporated by reference in its entirety for all purposes. In one specific example, the guide RNA comprises 2′-O-methyl analogs and 3′ phosphorothioate internucleotide linkages at the first three 5′ and 3′ terminal RNA residues. In another specific example, the guide RNA is modified such that all 2′OH groups that do not interact with the Cas9 protein are replaced with 2′-O-methyl analogs, and the tail region of the guide RNA, which has minimal interaction with Cas9, is modified with 5′ and 3′ phosphorothioate internucleotide linkages. Additionally, the DNA-targeting segment also has 2′-fluoro modifications on some bases. See, e.g., Yin et al. (2017) Nat. Biotech. 35 (12): 1179-1187, herein incorporated by reference in its entirety for all purposes. Other examples of modified guide RNAs are provided, e.g., in WO 2018/107028 A1, herein incorporated by reference in its entirety for all purposes. Such chemical modifications can, for example, provide greater stability and protection from exonucleases to guide RNAs, allowing them to persist within cells for longer than unmodified guide RNAs. Such chemical modifications can also, for example, protect against innate intracellular immune responses that can actively degrade RNA or trigger immune cascades that lead to cell death.

Guide RNAs can be provided in any form. For example, the gRNA can be provided in the form of RNA, either as two molecules (separate crRNA and tracrRNA) or as one molecule (sgRNA), and optionally in the form of a complex with a Cas protein. The gRNA can also be provided in the form of DNA encoding the gRNA. The DNA encoding the gRNA can encode a single RNA molecule (sgRNA) or separate RNA molecules (e.g., separate crRNA and tracrRNA). In the latter case, the DNA encoding the gRNA can be provided as one DNA molecule or as separate DNA molecules encoding the crRNA and tracrRNA, respectively.

When a gRNA is provided in the form of DNA, the gRNA can be transiently, conditionally, or constitutively expressed in the cell. DNAs encoding gRNAs can be stably integrated into the genome of the cell and operably linked to a promoter active in the cell. Alternatively, DNAs encoding gRNAs can be operably linked to a promoter in an expression construct. For example, the DNA encoding the gRNA can be in a vector comprising a heterologous nucleic acid, such as a nucleic acid encoding a Cas protein. Alternatively, it can be in a vector or a plasmid that is separate from the vector comprising the nucleic acid encoding the Cas protein. Promoters that can be used in such expression constructs include promoters active, for example, in one or more of a eukaryotic cell, a human cell, a non-human cell, a mammalian cell, a non-human mammalian cell, a rodent cell, a mouse cell, a rat cell, a pluripotent cell, an embryonic stem (ES) cell, an adult stem cell, a developmentally restricted progenitor cell, an induced pluripotent stem (iPS) cell, or a one-cell stage embryo. Such promoters can be, for example, conditional promoters, inducible promoters, constitutive promoters, or tissue-specific promoters. Such promoters can also be, for example, bidirectional promoters. Specific examples of suitable promoters include an RNA polymerase III promoter, such as a human U6 promoter, a rat U6 polymerase III promoter, or a mouse U6 polymerase III promoter. In another example, the small tRNA Gln can be used to drive expression of a guide RNA.

Alternatively, gRNAs can be prepared by various other methods. For example, gRNAs can be prepared by in vitro transcription using, for example, T7 RNA polymerase (see, e.g., WO 2014/089290 and WO 2014/065596, each of which is herein incorporated by reference in its entirety for all purposes). Guide RNAs can also be a synthetically produced molecule prepared by chemical synthesis. For example, a guide RNA can be chemically synthesized to include 2′-O-methyl analogs and 3′ phosphorothioate internucleotide linkages at the first three 5′ and 3′ terminal RNA residues.

Guide RNAs (or nucleic acids encoding guide RNAs) can be in compositions comprising one or more guide RNAs (e.g., 1, 2, 3, 4, or more guide RNAs) and a carrier increasing the stability of the guide RNA (e.g., prolonging the period under given conditions of storage (e.g., −20° C., 4° C., or ambient temperature) for which degradation products remain below a threshold, such below 0.5% by weight of the starting nucleic acid or protein; or increasing the stability in vivo). Non-limiting examples of such carriers include poly(lactic acid) (PLA) microspheres, poly(D,L-lactic-coglycolic-acid) (PLGA) microspheres, liposomes, micelles, inverse micelles, lipid cochleates, and lipid microtubules. Such compositions can further comprise a Cas protein, such as a Cas9 protein, or a nucleic acid encoding a Cas protein.

c. Guide RNA Target Sequences

Target DNAs for guide RNAs include nucleic acid sequences present in a DNA to which a DNA-targeting segment of a gRNA will bind, provided sufficient conditions for binding exist. Suitable DNA/RNA binding conditions include physiological conditions normally present in a cell. Other suitable DNA/RNA binding conditions (e.g., conditions in a cell-free system) are known in the art (see, e.g., Molecular Cloning: A Laboratory Manual, 3rd Ed. (Sambrook et al., Harbor Laboratory Press 2001), herein incorporated by reference in its entirety for all purposes). The strand of the target DNA that is complementary to and hybridizes with the gRNA can be called the “complementary strand,” and the strand of the target DNA that is complementary to the “complementary strand” (and is therefore not complementary to the Cas protein or gRNA) can be called “noncomplementary strand” or “template strand.”

The target DNA includes both the sequence on the complementary strand to which the guide RNA hybridizes and the corresponding sequence on the non-complementary strand (e.g., adjacent to the protospacer adjacent motif (PAM)). Unless otherwise specified, the term “guide RNA target sequence” as used herein refers specifically to the sequence on the non-complementary strand corresponding to (i.e., the reverse complement of) the sequence to which the guide RNA hybridizes on the complementary strand. That is, the guide RNA target sequence refers to the sequence on the non-complementary strand adjacent to the PAM (e.g., upstream or 5′ of the PAM in the case of Cas9). A guide RNA target sequence is equivalent to the DNA-targeting segment of a guide RNA, but with thymines instead of uracils. As one example, a guide RNA target sequence for an SpCas9 enzyme can refer to the sequence upstream of the 5′-NGG-3′ PAM on the non-complementary strand. A guide RNA is designed to have complementarity to the complementary strand of a target DNA, where hybridization between the DNA-targeting segment of the guide RNA and the complementary strand of the target DNA promotes the formation of a CRISPR complex. Full complementarity is not necessarily required, provided that there is sufficient complementarity to cause hybridization and promote formation of a CRISPR complex. If a guide RNA is referred to herein as targeting a guide RNA target sequence, what is meant is that the guide RNA hybridizes to the complementary strand sequence of the target DNA that is the reverse complement of the guide RNA target sequence on the non-complementary strand.

A target DNA or guide RNA target sequence can comprise any polynucleotide, and can be located, for example, in the nucleus or cytoplasm of a cell or within an organelle of a cell, such as a mitochondrion or chloroplast. A target DNA or guide RNA target sequence can be any nucleic acid sequence endogenous or exogenous to a cell. The guide RNA target sequence can be a sequence coding a gene product (e.g., a protein) or a non-coding sequence (e.g., a regulatory sequence) or can include both.

Site-specific binding and cleavage of a target DNA by a Cas protein can occur at locations determined by both (i) base-pairing complementarity between the guide RNA and the complementary strand of the target DNA and (ii) a short motif, called the protospacer adjacent motif (PAM), in the non-complementary strand of the target DNA. The PAM can flank the guide RNA target sequence. Optionally, the guide RNA target sequence can be flanked on the 3′ end by the PAM (e.g., for Cas9). Alternatively, the guide RNA target sequence can be flanked on the 5′ end by the PAM (e.g., for Cpf1). For example, the cleavage site of Cas proteins can be about 1 to about 10 or about 2 to about 5 base pairs (e.g., 3 base pairs) upstream or downstream of the PAM sequence (e.g., within the guide RNA target sequence). In the case of SpCas9, the PAM sequence (i.e., on the non-complementary strand) can be 5′-N₁GG-3′, where N₁ is any DNA nucleotide, and where the PAM is immediately 3′ of the guide RNA target sequence on the non-complementary strand of the target DNA. As such, the sequence corresponding to the PAM on the complementary strand (i.e., the reverse complement) would be 5′-CCN₂-3′, where N₂ is any DNA nucleotide and is immediately 5′ of the sequence to which the DNA-targeting segment of the guide RNA hybridizes on the complementary strand of the target DNA. In some such cases, N₁ and N₂ can be complementary and the N₁-N₂ base pair can be any base pair (e.g., N₁═C and N₂=G; N₁=G and N₂=C; N₁=A and N₂-T; or N₁=T, and N₂=A). In the case of Cas9 from S. aureus, the PAM can be NNGRRT or NNGRR, where N can A, G, C, or T, and R can be G or A. In the case of Cas9 from C. jejuni, the PAM can be, for example, NNNNACAC or NNNNRYAC, where N can be A, G, C, or T, and R can be Gor A. In some cases (e.g., for FnCpf1), the PAM sequence can be upstream of the 5′ end and have the sequence 5′-TTN-3′.

An example of a guide RNA target sequence is a 20-nucleotide DNA sequence immediately preceding an NGG motif recognized by an SpCas9 protein. For example, two examples of guide RNA target sequences plus PAMs are GN₁₉NGG (SEQ ID NO: 58) or N₂₀NGG (SEQ ID NO: 59). See, e.g., WO 2014/165825, herein incorporated by reference in its entirety for all purposes. The guanine at the 5′ end can facilitate transcription by RNA polymerase in cells. Other examples of guide RNA target sequences plus PAMs can include two guanine nucleotides at the 5′ end (e.g., GGN₂₀NGG; SEQ ID NO: 60) to facilitate efficient transcription by T7 polymerase in vitro. See, e.g., WO 2014/065596, herein incorporated by reference in its entirety for all purposes. Other guide RNA target sequences plus PAMs can have between 4-22 nucleotides in length of SEQ ID NOS: 58-60, including the 5′ G or GG and the 3′ GG or NGG. Yet other guide RNA target sequences plus PAMs can have between 14 and 20 nucleotides in length of SEQ ID NOS: 58-60.

Guide RNAs targeting an albumin gene can target, for example, the first intron of the albumin gene, or a sequence adjacent to the first intron of the albumin gene (e.g., in the first exon or the second exon of the albumin gene.

Formation of a CRISPR complex hybridized to a target DNA can result in cleavage of one or both strands of the target DNA within or near the region corresponding to the guide RNA target sequence (i.e., the guide RNA target sequence on the non-complementary strand of the target DNA and the reverse complement on the complementary strand to which the guide RNA hybridizes). For example, the cleavage site can be within the guide RNA target sequence (e.g., at a defined location relative to the PAM sequence). The “cleavage site” includes the position of a target DNA at which a Cas protein produces a single-strand break or a double-strand break. The cleavage site can be on only one strand (e.g., when a nickase is used) or on both strands of a double-stranded DNA. Cleavage sites can be at the same position on both strands (producing blunt ends; e.g. Cas9)) or can be at different sites on each strand (producing staggered ends (i.e., overhangs); e.g., Cpf1). Staggered ends can be produced, for example, by using two Cas proteins, each of which produces a single-strand break at a different cleavage site on a different strand, thereby producing a double-strand break. For example, a first nickase can create a single-strand break on the first strand of double-stranded DNA (dsDNA), and a second nickase can create a single-strand break on the second strand of dsDNA such that overhanging sequences are created. In some cases, the guide RNA target sequence or cleavage site of the nickase on the first strand is separated from the guide RNA target sequence or cleavage site of the nickase on the second strand by at least 2, 3, 4, 5, 6, 7, 8, 9, 10, 15, 20, 25, 30, 40, 50, 75, 100, 250, 500, or 1,000 base pairs.

### 2. Other Nuclease Agents and Target Sequences for Nuclease Agents

Any nuclease agent that induces a nick or double-strand break in a desired target sequence can be used in the methods and compositions disclosed herein. A naturally occurring or native nuclease agent can be employed so long as the nuclease agent induces a nick or double-strand break at a desired target sequence. Alternatively, a modified or engineered nuclease agent can be employed. An “engineered nuclease agent” includes a nuclease that is engineered (modified or derived) from its native form to specifically recognize and induce a nick or double-strand break in the desired target sequence. Thus, an engineered nuclease agent can be derived from a native, naturally occurring nuclease agent or it can be artificially created or synthesized. The engineered nuclease can induce a nick or double-strand break in a target sequence, for example, wherein the target sequence is not a sequence that would have been recognized by a native (non-engineered or non-modified) nuclease agent. The modification of the nuclease agent can be as little as one amino acid in a protein cleavage agent or one nucleotide in a nucleic acid cleavage agent. Producing a nick or double-strand break at a target sequence or other DNA can be referred to herein as “cutting” or “cleaving” the target sequence or other DNA.

Active variants and fragments of the exemplified target sequences are also provided. Such active variants can comprise at least 65%, 70%, 75%, 80%, 85%, 90%, 91%, 92%, 93%, 94%, 95%, 96%, 97%, 98%, 99% or more sequence identity to the given target sequence, wherein the active variants retain biological activity and hence are capable of being recognized and cleaved by a nuclease agent in a sequence-specific manner. Assays to measure the double-strand break of a target sequence by a nuclease agent are known in the art (e.g., TAQMAN® qPCR assay, Frendewey et al. (2010) Methods in Enzymology 476:295-307, which is herein incorporated by reference herein in its entirety for all purposes).

The target sequence of the nuclease agent can be positioned anywhere in or near the target locus. The target sequence can be located within a coding region of a gene, or within regulatory regions that influence the expression of the gene. A target sequence of the nuclease agent can be located in an intron, an exon, a promoter, an enhancer, a regulatory region, or any non-protein coding region. Alternatively, the target sequence can be positioned within the polynucleotide encoding the selection marker. Such a position can be located within the coding region of the selection marker or within the regulatory regions, which influence the expression of the selection marker. Thus, a target sequence of the nuclease agent can be located in an intron of the selection marker, a promoter, an enhancer, a regulatory region, or any non-protein-coding region of the polynucleotide encoding the selection marker. A nick or double-strand break at the target sequence can disrupt the activity of the selection marker, and methods to assay for the presence or absence of a functional selection marker are known.

One type of nuclease agent is a Transcription Activator-Like Effector Nuclease (TALEN). TAL effector nucleases are a class of sequence-specific nucleases that can be used to make double-strand breaks at specific target sequences in the genome of a prokaryotic or eukaryotic organism. TAL effector nucleases are created by fusing a native or engineered transcription activator-like (TAL) effector, or functional part thereof, to the catalytic domain of an endonuclease, such as, for example, FokI. The unique, modular TAL effector DNA binding domain allows for the design of proteins with potentially any given DNA recognition specificity. Thus, the DNA binding domains of the TAL effector nucleases can be engineered to recognize specific DNA target sites and thus, used to make double-strand breaks at desired target sequences. See WO 2010/079430; Morbitzer et al. (2010) Proc. Natl. Acad. Sci. U.S.A. 107 (50): 21617-21622; Scholze & Boch (2010) Virulence 1:428-432; Christian et al. Genetics (2010) 186:757-761; Li et al. (2010) Nucleic Acids Res. (2010) doi: 10.1093/nar/gkq704; and Miller et al. (2011) Nat. Biotechnol. 29:143-148, each of which is herein incorporated by reference in its entirety for all purposes.

Examples of suitable TAL nucleases, and methods for preparing suitable TAL nucleases, are disclosed, e.g., in US 2011/0239315 A1, US 2011/0269234 A1, US 2011/0145940 A1, US 2003/0232410 A1, US 2005/0208489 A1, US 2005/0026157 A1, US 2005/0064474 A1, US 2006/0188987 A1, and US 2006/0063231 A1, each of which is herein incorporated by reference in its entirety for all purposes. In various embodiments, TAL effector nucleases are engineered that cut in or near a target nucleic acid sequence in, e.g., a locus of interest or a genomic locus of interest, wherein the target nucleic acid sequence is at or near a sequence to be modified by a targeting vector. The TAL nucleases suitable for use with the various methods and compositions provided herein include those that are specifically designed to bind at or near target nucleic acid sequences to be modified by targeting vectors as described herein.

In some TALENs, each monomer of the TALEN comprises 33-35 TAL repeats that recognize a single base pair via two hypervariable residues. In some TALENs, the nuclease agent is a chimeric protein comprising a TAL-repeat-based DNA binding domain operably linked to an independent nuclease such as a FokI endonuclease. For example, the nuclease agent can comprise a first TAL-repeat-based DNA binding domain and a second TAL-repeat-based DNA binding domain, wherein each of the first and the second TAL-repeat-based DNA binding domains is operably linked to a FokI nuclease, wherein the first and the second TAL-repeat-based DNA binding domain recognize two contiguous target DNA sequences in each strand of the target DNA sequence separated by a spacer sequence of varying length (12-20 bp), and wherein the FokI nuclease subunits dimerize to create an active nuclease that makes a double strand break at a target sequence.

The nuclease agent employed in the various methods and compositions disclosed herein can further comprise a zinc-finger nuclease (ZFN). In some ZFNs, each monomer of the ZFN comprises 3 or more zinc finger-based DNA binding domains, wherein each zinc finger-based DNA binding domain binds to a 3 bp subsite. In other ZFNs, the ZFN is a chimeric protein comprising a zinc finger-based DNA binding domain operably linked to an independent nuclease such as a FokI endonuclease. For example, the nuclease agent can comprise a first ZFN and a second ZFN, wherein each of the first ZFN and the second ZFN is operably linked to a FokI nuclease subunit, wherein the first and the second ZFN recognize two contiguous target DNA sequences in each strand of the target DNA sequence separated by about 5-7 bp spacer, and wherein the FokI nuclease subunits dimerize to create an active nuclease that makes a double strand break. See, e.g., US20060246567; US20080182332; US20020081614; US20030021776; WO/2002/057308A2; US20130123484; US20100291048; WO/2011/017293A2; and Gaj et al. (2013) Trends Biotechnol., 31 (7): 397-405, each of which is herein incorporated by reference in its entirety for all purposes.

Active variants and fragments of nuclease agents (i.e., an engineered nuclease agent) are also provided. Such active variants can comprise at least 65%, 70%, 75%, 80%, 85%, 90%, 91%, 92%, 93%, 94%, 95%, 96%, 97%, 98%, 99% or more sequence identity to the native nuclease agent, wherein the active variants retain the ability to cut at a desired target sequence and hence retain nick or double-strand-break-inducing activity. For example, any of the nuclease agents described herein can be modified from a native endonuclease sequence and designed to recognize and induce a nick or double-strand break at a target sequence that was not recognized by the native nuclease agent. Thus, some engineered nucleases have a specificity to induce a nick or double-strand break at a target sequence that is different from the corresponding native nuclease agent target sequence. Assays for nick or double-strand-break-inducing activity are known and generally measure the overall activity and specificity of the endonuclease on DNA substrates containing the target sequence.

The nuclease agent may be introduced into the cell by any means known in the art. The polypeptide encoding the nuclease agent may be directly introduced into the cell. Alternatively, a polynucleotide encoding the nuclease agent can be introduced into the cell. When a polynucleotide encoding the nuclease agent is introduced into the cell, the nuclease agent can be transiently, conditionally, or constitutively expressed within the cell. Thus, the polynucleotide encoding the nuclease agent can be contained in an expression cassette and be operably linked to a conditional promoter, an inducible promoter, a constitutive promoter, or a tissue-specific promoter. Such promoters of interest are discussed in further detail elsewhere herein. Alternatively, the nuclease agent is introduced into the cell as an mRNA encoding a nuclease agent.

A polynucleotide encoding a nuclease agent can be stably integrated in the genome of the cell and operably linked to a promoter active in the cell. Alternatively, a polynucleotide encoding a nuclease agent can be in a targeting vector (e.g., a targeting vector comprising an insert polynucleotide, or in a vector or a plasmid that is separate from the targeting vector comprising the insert polynucleotide).

When the nuclease agent is provided to the cell through the introduction of a polynucleotide encoding the nuclease agent, such a polynucleotide encoding a nuclease agent can be modified to substitute codons having a higher frequency of usage in the cell of interest, as compared to the naturally occurring polynucleotide sequence encoding the nuclease agent. For example, the polynucleotide encoding the nuclease agent can be modified to substitute codons having a higher frequency of usage in a given prokaryotic or eukaryotic cell of interest, including a bacterial cell, a yeast cell, a human cell, a non-human cell, a mammalian cell, a rodent cell, a mouse cell, a rat cell or any other host cell of interest, as compared to the naturally occurring polynucleotide sequence.

The term “target sequence for a nuclease agent” includes a DNA sequence at which a nick or double-strand break is induced by a nuclease agent. The target sequence for a nuclease agent can be endogenous (or native) to the cell or the target sequence can be exogenous to the cell. A target sequence that is exogenous to the cell is not naturally occurring in the genome of the cell. The target sequence can also exogenous to the polynucleotides of interest that one desires to be positioned at the target locus. In some cases, the target sequence is present only once in the genome of the host cell.

The length of the target sequence can vary, and includes, for example, target sequences that are about 30-36 bp for a zinc finger nuclease (ZFN) pair (i.e., about 15-18 bp for each ZFN), about 36 bp for a Transcription Activator-Like Effector Nuclease (TALEN), or about 20 bp for a CRISPR/Cas9 guide RNA.

### B. Exogenous Donor Nucleic Acids and Antigen-Binding Protein Coding Sequences

### 1. Exogenous Donor Nucleic Acids

The methods and compositions disclosed herein utilize exogenous donor nucleic acids to modify a target genomic locus (e.g., a genomic locus or safe harbor locus) following cleavage of the target genomic locus with a nuclease agent such as a Cas protein.

In such methods, the Cas protein cleaves the target genomic locus to create a single-strand break (nick) or double-strand break, and the cleaved or nicked locus is repaired by the exogenous donor nucleic acid via non-homologous end joining (NHEJ)-mediated ligation or homology-directed repair. Optionally, repair with the exogenous donor nucleic acid removes or disrupts the nuclease target sequence so that alleles that have been targeted cannot be re-targeted by the nuclease agent.

The exogenous donor nucleic acid can target any sequence in a genomic locus or safe harbor locus such as the albumin locus. Some exogenous donor nucleic acids comprise homology arms. Other exogenous donor nucleic acids do not comprise homology arms. The exogenous donor nucleic acids can be capable of insertion into a genomic locus or safe harbor locus by homology-directed repair, and/or they can be capable of insertion into a genomic locus or safe harbor locus by non-homologous end joining. In one example, the exogenous donor nucleic acid (e.g., a targeting vector) can target intron 1, intron 12, or intron 13 of an albumin locus. For example, the exogenous donor nucleic acid can target intron 1 of an albumin gene.

Exogenous donor nucleic acids can comprise deoxyribonucleic acid (DNA) or ribonucleic acid (RNA), they can be single-stranded or double-stranded, and they can be in linear or circular form. For example, an exogenous donor nucleic acid can be a single-stranded oligodeoxynucleotide (ssODN). See, e.g., Yoshimi et al. (2016) Nat. Commun. 7:10431, herein incorporated by reference in its entirety for all purposes. Exogenous donor nucleic acids can be naked nucleic acids or can be delivered by viruses, such as AAV. In a specific example, the exogenous donor nucleic acid can be delivered via AAV and can be capable of insertion into a genomic locus or safe harbor locus by non-homologous end joining (e.g., the exogenous donor nucleic acid can be one that does not comprise homology arms).

An exemplary exogenous donor nucleic acid is between about 50 nucleotides to about 5 kb in length or between about 50 nucleotides to about 3 kb in length. Alternatively, an exogenous donor nucleic acid can be between about 1 kb to about 1.5 kb, about 1.5 kb to about 2 kb, about 2 kb to about 2.5 kb, about 2.5 kb to about 3 kb, about 3 kb to about 3.5 kb, about 3.5 kb to about 4 kb, about 4 kb to about 4.5 kb, or about 4.5 kb to about 5 kb in length. Alternatively, an exogenous donor nucleic acid can be, for example, no more than 5 kb, 4.5 kb, 4 kb, 3.5 kb, 3 kb, or 2.5 kb in length.

In one example, an exogenous donor nucleic acid is an ssODN that is between about 80 nucleotides and about 3 kb in length. Such an ssODN can have homology arms or short single-stranded regions at the 5′ end and/or the 3′ end that are complementary to one or more overhangs created by nuclease-agent-mediated cleavage at the target genomic locus, for example, that are each between about 40 nucleotides and about 60 nucleotides in length. Such an ssODN can also have homology arms or complementary regions, for example, that are each between about 30 nucleotides and 100 nucleotides in length. The homology arms or complementary regions can be symmetrical (e.g., each 40 nucleotides or each 60 nucleotides in length), or they can be asymmetrical (e.g., one homology arm or complementary region that is 36 nucleotides in length, and one homology arm or complementary region that is 91 nucleotides in length).

Exogenous donor nucleic acids can include modifications or sequences that provide for additional desirable features (e.g., modified or regulated stability; tracking or detecting with a fluorescent label; a binding site for a protein or protein complex; and so forth). Exogenous donor nucleic acids can comprise one or more fluorescent labels, purification tags, epitope tags, or a combination thereof. For example, an exogenous donor nucleic acid can comprise one or more fluorescent labels (e.g., fluorescent proteins or other fluorophores or dyes), such as at least 1, at least 2, at least 3, at least 4, or at least 5 fluorescent labels. Exemplary fluorescent labels include fluorophores such as fluorescein (e.g., 6-carboxyfluorescein (6-FAM)), Texas Red, HEX, Cy3, Cy5, Cy5.5, Pacific Blue, 5-(and-6)-carboxytetramethylrhodamine (TAMRA), and Cy7. A wide range of fluorescent dyes are available commercially for labeling oligonucleotides (e.g., from Integrated DNA Technologies). Such fluorescent labels (e.g., internal fluorescent labels) can be used, for example, to detect an exogenous donor nucleic acid that has been directly integrated into a cleaved target nucleic acid having protruding ends compatible with the ends of the exogenous donor nucleic acid. The label or tag can be at the 5′ end, the 3′ end, or internally within the exogenous donor nucleic acid. For example, an exogenous donor nucleic acid can be conjugated at 5′ end with the IR700 fluorophore from Integrated DNA Technologies (5′IRDYE®700).

The exogenous donor nucleic acids disclosed herein also comprise nucleic acid inserts including segments of DNA to be integrated at target genomic loci (i.e., coding sequences for antigen-binding proteins). Integration of a nucleic acid insert at a target genomic locus can result in addition of a nucleic acid sequence of interest to the target genomic locus or replacement of a nucleic acid sequence of interest at the target genomic locus (i.e., deletion and insertion). Some exogenous donor nucleic acids are designed for insertion of a nucleic acid insert at a target genomic locus without any corresponding deletion at the target genomic locus. Other exogenous donor nucleic acids are designed to delete a nucleic acid sequence of interest at a target genomic locus and replace it with a nucleic acid insert.

The nucleic acid insert or the corresponding nucleic acid at the target genomic locus being deleted and/or replaced can be various lengths. An exemplary nucleic acid insert or corresponding nucleic acid at the target genomic locus being deleted and/or replaced is between about 1 nucleotide to about 5 kb in length or is between about 1 nucleotide to about 3 kb nucleotides in length. For example, a nucleic acid insert or a corresponding nucleic acid at the target genomic locus being deleted and/or replaced can be between about 1 to about 100, about 100 to about 200, about 200 to about 300, about 300 to about 400, about 400 to about 500, about 500 to about 600, about 600 to about 700, about 700 to about 800, about 800 to about 900, or about 900 to about 1,000 nucleotides in length. Likewise, a nucleic acid insert or a corresponding nucleic acid at the target genomic locus being deleted and/or replaced can be between about 1 kb to about 1.5 kb, about 1.5 kb to about 2 kb, about 2 kb to about 2.5 kb, about 2.5 kb to about 3 kb, about 3 kb to about 3.5 kb, about 3.5 kb to about 4 kb, about 4 kb to about 4.5 kb, about 4.5 kb to about 5 kb in length, or longer.

The nucleic acid insert or the corresponding nucleic acid at the target genomic locus being deleted and/or replaced can be a coding region such as an exon; a non-coding region such as an intron, an untranslated region, or a regulatory region (e.g., a promoter, an enhancer, or a transcriptional repressor-binding element); or any combination thereof.

The nucleic acid insert can also comprise a conditional allele. The conditional allele can be a multifunctional allele, as described in US 2011/0104799, herein incorporated by reference in its entirety for all purposes. For example, the conditional allele can comprise: (a) an actuating sequence in sense orientation with respect to transcription of a target gene; (b) a drug selection cassette (DSC) in sense or antisense orientation; (c) a nucleotide sequence of interest (NSI) in antisense orientation; and (d) a conditional by inversion module (COIN, which utilizes an exon-splitting intron and an invertible gene-trap-like module) in reverse orientation. See, e.g., US 2011/0104799. The conditional allele can further comprise recombinable units that recombine upon exposure to a first recombinase to form a conditional allele that (i) lacks the actuating sequence and the DSC; and (ii) contains the NSI in sense orientation and the COIN in antisense orientation. See, e.g., US 2011/0104799.

Nucleic acid inserts can also comprise a polynucleotide encoding a selection marker. Alternatively, the nucleic acid inserts can lack a polynucleotide encoding a selection marker. The selection marker can be contained in a selection cassette. Optionally, the selection cassette can be a self-deleting cassette. See, e.g., U.S. Pat. No. 8,697,851 and US 2013/0312129, each of which is herein incorporated by reference in its entirety for all purposes. As an example, the self-deleting cassette can comprise a Crei gene (comprises two exons encoding a Cre recombinase, which are separated by an intron) operably linked to a mouse Prm1 promoter and a neomycin resistance gene operably linked to a human ubiquitin promoter. By employing the Prm1 promoter, the self-deleting cassette can be deleted specifically in male germ cells of F0 animals. Exemplary selection markers include neomycin phosphotransferase (neo′), hygromycin B phosphotransferase (hyg′), puromycin-N-acetyltransferase (puro′), blasticidin S deaminase (bsr′), xanthine/guanine phosphoribosyl transferase (gpt), or herpes simplex virus thymidine kinase (HSV-k), or a combination thereof. The polynucleotide encoding the selection marker can be operably linked to a promoter active in a cell being targeted. Examples of promoters are described elsewhere herein.

The nucleic acid insert can also comprise a reporter gene. Exemplary reporter genes include those encoding luciferase, β-galactosidase, green fluorescent protein (GFP), enhanced green fluorescent protein (eGFP), cyan fluorescent protein (CFP), yellow fluorescent protein (YFP), enhanced yellow fluorescent protein (eYFP), blue fluorescent protein (BFP), enhanced blue fluorescent protein (eBFP), DsRed, ZsGreen, MmGFP, mPlum, mCherry, tdTomato, mStrawberry, J-Red, mOrange, mKO, mCitrine, Venus, YPet, Emerald, CyPet, Cerulean, T-Sapphire, and alkaline phosphatase. Such reporter genes can be operably linked to a promoter active in a cell being targeted. Examples of promoters are described elsewhere herein.

The nucleic acid insert can also comprise one or more expression cassettes or deletion cassettes. A given cassette can comprise one or more of a nucleotide sequence of interest, a polynucleotide encoding a selection marker, and a reporter gene, along with various regulatory components that influence expression. Examples of selectable markers and reporter genes that can be included are discussed in detail elsewhere herein.

The nucleic acid insert can comprise a nucleic acid flanked with site-specific recombination target sequences. Alternatively, the nucleic acid insert can comprise one or more site-specific recombination target sequences. Although the entire nucleic acid insert can be flanked by such site-specific recombination target sequences, any region or individual polynucleotide of interest within the nucleic acid insert can also be flanked by such sites. Site-specific recombination target sequences, which can flank the nucleic acid insert or any polynucleotide of interest in the nucleic acid insert can include, for example, loxP, lox511, lox2272, lox66, lox71, loxM2, lox5171, FRT, FRT11, FRT71, attp, att, FRT, rox, or a combination thereof. In one example, the site-specific recombination sites flank a polynucleotide encoding a selection marker and/or a reporter gene contained within the nucleic acid insert. Following integration of the nucleic acid insert at a targeted locus, the sequences between the site-specific recombination sites can be removed. Optionally, two exogenous donor nucleic acids can be used, each with a nucleic acid insert comprising a site-specific recombination site. The exogenous donor nucleic acids can be targeted to 5′ and 3′ regions flanking a nucleic acid of interest. Following integration of the two nucleic acid inserts into the target genomic locus, the nucleic acid of interest between the two inserted site-specific recombination sites can be removed.

Nucleic acid inserts can also comprise one or more restriction sites for restriction endonucleases (i.e., restriction enzymes), which include Type I, Type II, Type III, and Type IV endonucleases. Type I and Type III restriction endonucleases recognize specific recognition sites, but typically cleave at a variable position from the nuclease binding site, which can be hundreds of base pairs away from the cleavage site (recognition site). In Type II systems the restriction activity is independent of any methylase activity, and cleavage typically occurs at specific sites within or near to the binding site. Most Type II enzymes cut palindromic sequences, however Type Ila enzymes recognize non-palindromic recognition sites and cleave outside of the recognition site, Type IIb enzymes cut sequences twice with both sites outside of the recognition site, and Type IIs enzymes recognize an asymmetric recognition site and cleave on one side and at a defined distance of about 1-20 nucleotides from the recognition site. Type IV restriction enzymes target methylated DNA. Restriction enzymes are further described and classified, for example in the REBASE database (webpage at rebase.neb.com; Roberts et al., (2003) Nucleic Acids Res. 31:418-420; Roberts et al., (2003) Nucleic Acids Res. 31:1805-1812; and Belfort et al. (2002) in Mobile DNA II, pp. 761-783, Eds. Craigie et al., (ASM Press, Washington, DC)).

a. Donor Nucleic Acids for Non-Homologous-End-Joining-Mediated Insertion

Some exogenous donor nucleic acids are capable of insertion into a genomic locus or safe harbor locus by non-homologous end joining. In some cases, such exogenous donor nucleic acids do not comprise homology arms. For example, such exogenous donor nucleic acids can be inserted into a blunt end double-strand break following cleavage with a nuclease agent. In a specific example, the exogenous donor nucleic acid can be delivered via AAV and can be capable of insertion into a genomic locus or safe harbor locus by non-homologous end joining (e.g., the exogenous donor nucleic acid can be one that does not comprise homology arms).

In a specific example, the exogenous donor nucleic acid can be inserted via homology-independent targeted integration. For example, the antigen-binding protein coding sequence in the exogenous donor nucleic acid is flanked on each side by a target site for a nuclease agent (e.g., the same target site as in the genomic locus or safe harbor locus, and the same nuclease agent being used to cleave the target site in the genomic locus or safe harbor locus). The nuclease agent can then cleave the target sites flanking the antigen-binding protein coding sequence. In a specific example, the exogenous donor nucleic acid is delivered AAV-mediated delivery, and cleavage of the target sites flanking the antigen-binding protein coding sequence can remove the inverted terminal repeats (ITRs) of the AAV. In some methods, the target site in the genomic locus or safe harbor locus (e.g., a gRNA target sequence including the flanking protospacer adjacent motif) is no longer present if the antigen-binding protein coding sequence is inserted into the genomic locus or safe harbor locus in the correct orientation but it is reformed if the antigen-binding protein coding sequence is inserted into the genomic locus or safe harbor locus in the opposite orientation. This can help ensure that the antigen-binding protein coding sequence is inserted in the correct orientation for expression.

Other exogenous donor nucleic acids have short single-stranded regions at the 5′ end and/or the 3′ end that are complementary to one or more overhangs created by nuclease-agent-mediated cleavage at the target genomic locus. For example, some exogenous donor nucleic acids have short single-stranded regions at the 5′ end and/or the 3′ end that are complementary to one or more overhangs created by nuclease-mediated cleavage at 5′ and/or 3′ target sequences at the target genomic locus. Some such exogenous donor nucleic acids have a complementary region only at the 5′ end or only at the 3′ end. For example, some such exogenous donor nucleic acids have a complementary region only at the 5′ end complementary to an overhang created at a 5′ target sequence at the target genomic locus or only at the 3′ end complementary to an overhang created at a 3′ target sequence at the target genomic locus. Other such exogenous donor nucleic acids have complementary regions at both the 5′ and 3′ ends. For example, other such exogenous donor nucleic acids have complementary regions at both the 5′ and 3′ ends (e.g., complementary to first and second overhangs, respectively) generated by nuclease-mediated cleavage at the target genomic locus. For example, if the exogenous donor nucleic acid is double-stranded, the single-stranded complementary regions can extend from the 5′ end of the top strand of the donor nucleic acid and the 5′ end of the bottom strand of the donor nucleic acid, creating 5′ overhangs on each end. Alternatively, the single-stranded complementary region can extend from the 3′ end of the top strand of the donor nucleic acid and from the 3′ end of the bottom strand of the template, creating 3′ overhangs.

The complementary regions can be of any length sufficient to promote ligation between the exogenous donor nucleic acid and the target nucleic acid. Exemplary complementary regions are between about 1 to about 5 nucleotides in length, between about 1 to about 25 nucleotides in length, or between about 5 to about 150 nucleotides in length. For example, a complementary region can be at least about 1, 2, 3, 4, 5, 6, 7, 8, 9, 10, 11, 12, 13, 14, 15, 16, 17, 18, 19, 20, 21, 22, 23, 24, or 25 nucleotides in length. Alternatively, the complementary region can be about 5 to about 10, about 10 to about 20, about 20 to about 30, about 30 to about 40, about 40 to about 50, about 50 to about 60, about 60 to about 70, about 70 to about 80, about 80 to about 90, about 90 to about 100, about 100 to about 110, about 110 to about 120, about 120 to about 130, about 130 to about 140, about 140 to about 150 nucleotides in length, or longer.

Such complementary regions can be complementary to overhangs created by two pairs of nickases. Two double-strand breaks with staggered ends can be created by using first and second nickases that cleave opposite strands of DNA to create a first double-strand break, and third and fourth nickases that cleave opposite strands of DNA to create a second double-strand break. For example, a Cas protein can be used to nick first, second, third, and fourth guide RNA target sequences corresponding with first, second, third, and fourth guide RNAs. The first and second guide RNA target sequences can be positioned to create a first cleavage site such that the nicks created by the first and second nickases on the first and second strands of DNA create a double-strand break (i.e., the first cleavage site comprises the nicks within the first and second guide RNA target sequences). Likewise, the third and fourth guide RNA target sequences can be positioned to create a second cleavage site such that the nicks created by the third and fourth nickases on the first and second strands of DNA create a double-strand break (i.e., the second cleavage site comprises the nicks within the third and fourth guide RNA target sequences). The nicks within the first and second guide RNA target sequences and/or the third and fourth guide RNA target sequences can be off-set nicks that create overhangs. The offset window can be, for example, at least about 5 bp, 10 bp, 20 bp, 30 bp, 40 bp, 50 bp, 60 bp, 70 bp, 80 bp, 90 bp, 100 bp or more. See Ran et al. (2013) Cell 154:1380-1389; Mali et al. (2013) Nat. Biotechnol. 31:833-838; and Shen et al. (2014) Nat. Methods 11:399-404, each of which is herein incorporated by reference in its entirety for all purposes. In such cases, a double-stranded exogenous donor nucleic acid can be designed with single-stranded complementary regions that are complementary to the overhangs created by the nicks within the first and second guide RNA target sequences and by the nicks within the third and fourth guide RNA target sequences. Such an exogenous donor nucleic acid can then be inserted by non-homologous-end-joining-mediated ligation.

b. Donor Nucleic Acids for Insertion by Homology-Directed Repair

Some exogenous donor nucleic acids comprise homology arms. If the exogenous donor nucleic acid also comprises a nucleic acid insert, the homology arms can flank the nucleic acid insert. For ease of reference, the homology arms are referred to herein as 5′ and 3′ (i.e., upstream and downstream) homology arms. This terminology relates to the relative position of the homology arms to the nucleic acid insert within the exogenous donor nucleic acid. The 5′ and 3′ homology arms correspond to regions within the target genomic locus, which are referred to herein as “5′ target sequence” and “3′ target sequence,” respectively.

A homology arm and a target sequence “correspond” or are “corresponding” to one another when the two regions share a sufficient level of sequence identity to one another to act as substrates for a homologous recombination reaction. The term “homology” includes DNA sequences that are either identical or share sequence identity to a corresponding sequence. The sequence identity between a given target sequence and the corresponding homology arm found in the exogenous donor nucleic acid can be any degree of sequence identity that allows for homologous recombination to occur. For example, the amount of sequence identity shared by the homology arm of the exogenous donor nucleic acid (or a fragment thereof) and the target sequence (or a fragment thereof) can be at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity, such that the sequences undergo homologous recombination. Moreover, a corresponding region of homology between the homology arm and the corresponding target sequence can be of any length that is sufficient to promote homologous recombination. Exemplary homology arms are between about 25 nucleotides to about 2.5 kb in length, are between about 25 nucleotides to about 1.5 kb in length, or are between about 25 to about 500 nucleotides in length. For example, a given homology arm (or each of the homology arms) and/or corresponding target sequence can comprise corresponding regions of homology that are between about 25 to about 30, about 30 to about 40, about 40 to about 50, about 50 to about 60, about 60 to about 70, about 70 to about 80, about 80 to about 90, about 90 to about 100, about 100 to about 150, about 150 to about 200, about 200 to about 250, about 250 to about 300, about 300 to about 350, about 350 to about 400, about 400 to about 450, or about 450 to about 500 nucleotides in length, such that the homology arms have sufficient homology to undergo homologous recombination with the corresponding target sequences within the target nucleic acid. Alternatively, a given homology arm (or each homology arm) and/or corresponding target sequence can comprise corresponding regions of homology that are between about 0.5 kb to about 1 kb, about 1 kb to about 1.5 kb, about 1.5 kb to about 2 kb, or about 2 kb to about 2.5 kb in length. For example, the homology arms can each be about 750 nucleotides in length. The homology arms can be symmetrical (each about the same size in length), or they can be asymmetrical (one longer than the other).

When a CRISPR/Cas system or other nuclease agent is used in combination with an exogenous donor nucleic acid, the 5′ and 3′ target sequences can be located in sufficient proximity to the nuclease cleavage site (e.g., within sufficient proximity to a guide RNA target sequence) so as to promote the occurrence of a homologous recombination event between the target sequences and the homology arms upon a single-strand break (nick) or double-strand break at the nuclease cleavage site or nuclease cleavage site. The term “nuclease cleavage site” includes a DNA sequence at which a nick or double-strand break is created by a nuclease agent (e.g., a Cas9 protein complexed with a guide RNA). The target sequences within the targeted locus that correspond to the 5′ and 3′ homology arms of the exogenous donor nucleic acid are “located in sufficient proximity” to a nuclease cleavage site if the distance is such as to promote the occurrence of a homologous recombination event between the 5′ and 3′ target sequences and the homology arms upon a single-strand break or double-strand break at the nuclease cleavage site. Thus, the target sequences corresponding to the 5′ and/or 3′ homology arms of the exogenous donor nucleic acid can be, for example, within at least 1 nucleotide of a given nuclease cleavage site or within at least 10 nucleotides to about 1,000 nucleotides of a given nuclease cleavage site. As an example, the nuclease cleavage site can be immediately adjacent to at least one or both of the target sequences.

The spatial relationship of the target sequences that correspond to the homology arms of the exogenous donor nucleic acid and the nuclease cleavage site can vary. For example, target sequences can be located 5′ to the nuclease cleavage site, target sequences can be located 3′ to the nuclease cleavage site, or the target sequences can flank the nuclease cleavage site.

### 2. Antigen-Binding Proteins

The exogenous donor nucleic acids disclosed herein comprise coding sequences for antigen-binding proteins. An “antigen-binding protein” as disclosed herein includes any protein that binds to an antigen. Examples of antigen-binding proteins include an antibody, an antigen-binding fragment of an antibody, a multi-specific antibody (e.g., a bi-specific antibody), an scFV, a bis-scFV, a diabody, a triabody, a tetrabody, a V-NAR, a VHH, a VL, a F (ab), a F (ab) 2, a DVD (dual variable domain antigen-binding protein), an SVD (single variable domain antigen-binding protein), a bispecific T-cell engager (BiTE), or a Davisbody (U.S. Pat. No. 8,586,713, herein incorporated by reference herein in its entirety for all purposes).

The term “antibody” includes immunoglobulin molecules comprising four polypeptide chains, two heavy (H) chains and two light (L) chains inter-connected by disulfide bonds. Each heavy chain comprises a heavy chain variable domain and a heavy chain constant region (CH). The heavy chain constant region comprises three domains: C_{H}1, C_{H}2 and C_{H}3. Each light chain comprises a light chain variable domain and a light chain constant region (CL). The heavy chain and light chain variable domains can be further subdivided into regions of hypervariability, termed complementarity determining regions (CDR), interspersed with regions that are more conserved, termed framework regions (FR). Each heavy and light chain variable domain comprises three CDRs and four FRs, arranged from amino-terminus to carboxy-terminus in the following order: FR1, CDR1, FR2, CDR2, FR3, CDR3, FR4 (heavy chain CDRs may be abbreviated as HCDR1, HCDR2 and HCDR3; light chain CDRs may be abbreviated as LCDR1, LCDR2 and LCDR3). The term “high affinity” antibody refers to an antibody that has a K_{D} with respect to its target epitope about of 10⁻⁹M or lower (e.g., about 1×10⁻⁹M, 1×10⁻¹⁰M, 1×10⁻¹¹ M, or about 1×10⁻¹²M). In one embodiment, K_{D} is measured by surface plasmon resonance, e.g., BIACORE™; in another embodiment, K_{D} is measured by ELISA.

An antigen-binding protein or antibody can be, for example, a neutralizing antigen-binding protein or antibody or a broadly neutralizing antigen-binding protein or antibody. A neutralizing antibody is an antibody that defends a cell from an antigen or infectious body by neutralizing any effect it has biologically. Broadly-neutralizing antibodies (bNAbs) affect multiple strains of a particular bacteria or virus. For example, broadly neutralizing antibodies can focus on conserved functional targets, attacking a vulnerable site on conserved bacterial or viral proteins (e.g., a vulnerable site on the influenza viral protein hemagglutinin). Antibodies developed by the immune system upon infection or vaccination tend to focus on easily accessible loops on the bacterial or viral surface, which often have great sequence and conformational variability. This is a problem for two reasons: the bacteria or virus population can quickly evade these antibodies, and the antibodies are attacking portions of the protein that are not essential for function. Broadly neutralizing antibodies-termed “broadly” because they attack many strains of the bacteria or virus, and “neutralizing” because they attack key functional sites in the bacteria or virus and block infection—can overcome these problems. Unfortunately, however, these antibodies usually come too late and do not provide effective protection from the disease.

The antigen-binding proteins disclosed herein can target any antigen. The term “antigen” refers to a substance, whether an entire molecule or a domain within a molecule, which is capable of eliciting production of antibodies with binding specificity to that substance. The term antigen also includes substances, which in wild type host organisms would not elicit antibody production by virtue of self-recognition, but can elicit such a response in a host animal with appropriate genetic engineering to break immunological tolerance.

As one example, the targeted antigen can be a disease-associated antigen. The term “disease-associated antigen” refers to an antigen whose presence is correlated with the occurrence or progression of a particular disease. For example, the antigen can be in a disease-associated protein (i.e., a protein whose expression is correlated with the occurrence or progression of the disease). Optionally, a disease-associated protein can be a protein that is expressed in a particular type of disease but is not normally expressed in healthy adult tissue (i.e., a protein with disease-specific expression or disease-restricted expression). However, a disease-associated protein does not have to have disease-specific or disease-restricted expression.

As one example, a disease-associated antigen can be a cancer-associated antigen. The term “cancer-associated antigen” refers to an antigen whose presence is correlated with the occurrence or progression of one or more types of cancer. For example, the antigen can be in a cancer-associated protein (i.e., a protein whose expression is correlated with the occurrence or progression of one or more types of cancer). For example, a cancer-associated protein can be an oncogenic protein (i.e., a protein with activity that can contribute to cancer progression, such as proteins that regulate cell growth), or it can be a tumor-suppressor protein (i.e., a protein that typically acts to alleviate the potential for cancer formation, such as through negative regulation of the cell cycle or by promoting apoptosis). Optionally, a cancer-associated protein can be a protein that is expressed in a particular type of cancer but is not normally expressed in healthy adult tissue (i.e., a protein with cancer-specific expression, cancer-restricted expression, tumor-specific expression, or tumor-restricted expression). However, a cancer-associated protein does not have to have cancer-specific, cancer-restricted, tumor-specific, or tumor-restricted expression. Examples of proteins that are considered cancer-specific or cancer-restricted are cancer testis antigens or oncofetal antigens. Cancer testis antigens (CTAs) are a large family of tumor-associated antigens expressed in human tumors of different histological origin but not in normal tissue, except for male germ cells. In cancer, these developmental antigens can be re-expressed and can serve as a locus of immune activation. Oncofetal antigens (OFAs) are proteins that are typically present only during fetal development but are found in adults with certain kinds of cancer.

As another example, a disease-associated antigen can be an infectious-disease-associated antigen. The term “infectious-disease-associated antigen” refers to an antigen whose presence is correlated with the occurrence or progression of a particular infectious disease. For example, the antigen can be in an infectious-disease-associated protein (i.e., a protein whose expression is correlated with the occurrence or progression of the infectious disease). Optionally, an infectious-disease-associated protein can be a protein that is expressed in a particular type of infectious disease but is not normally expressed in healthy adult tissue (i.e., a protein with infectious-disease-specific expression or infectious-disease-restricted expression). However, an infectious-disease-associated protein does not have to have infectious-disease-specific or infectious-disease-restricted expression. For example, the antigen can be a viral antigen or a bacterial antigen. Such antigens include, for example, molecular structures on the surface of viruses or bacteria (e.g., viral proteins or bacterial proteins) that are recognized by the immune system and are capable of triggering an immune response.

Examples of viral antigens include antigens within proteins expressed by the Zika virus or influenza (flu) viruses. Zika is a virus spread to people primarily through the bite of an infected Aedes species mosquito (Ae. aegypti and Ae. Albopictus). Zika virus infection during pregnancy can cause microcephaly and other severe brain defects. For example, a Zika antigen can be, but is not limited to, an antigen within a Zika virus envelope (Env) protein. Influenza virus is a virus that causes an infectious disease called influenza (commonly known as “the flu”). Three types of influenza viruses affect people, called Type A, Type B, and Type C. An influenza antigen can be, but is not limited to, an antigen within the hemagglutinin protein. Viral antigens and bacterial antigens also include antigens on other viruses and other bacteria. Examples of antibodies targeting influenza hemagglutinin are provided, e.g., in WO 2016/100807, herein incorporated by reference in its entirety for all purposes.

Examples of bacterial antigens include antigens within proteins expressed by Pseudomonas aeruginosa (e.g., an antigen within PcrV, which is a type III virulence system translocating protein). Pseudomonas aeruginosa is an opportunistic bacterial pathogen that causes fatal acute lung infections in critically ill individuals. Its pathogenesis is associated with bacterial virulence conferred by the type III secretion system (TTSS), through which P. aeruginosa causes necrosis of the lung epithelium and disseminates into the circulation, resulting in bacteremia, sepsis, and mortality. TTSS allows P. aeruginosa to directly translocate cytotoxins into eukaryotic cells, inducing cell death. The P. aeruginosa V-antigen PcrV, a homolog of the Yersinia V-antigen LcrV, is an indispensable contributor to TTS toxin translocation.

The term “epitope” refers to a site on an antigen to which an antigen-binding protein (e.g., antibody) binds. An epitope can be formed from contiguous amino acids or noncontiguous amino acids juxtaposed by tertiary folding of one or more proteins. Epitopes formed from contiguous amino acids (also known as linear epitopes) are typically retained on exposure to denaturing solvents whereas epitopes formed by tertiary folding (also known as conformational epitopes) are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols, in Methods in Molecular Biology, Vol. 66, Glenn E. Morris, Ed. (1996), herein incorporated by reference in its entirety for all purposes.

The term “heavy chain,” or “immunoglobulin heavy chain” includes an immunoglobulin heavy chain sequence, including immunoglobulin heavy chain constant region sequence, from any organism. Heavy chain variable domains include three heavy chain CDRs and four FR regions, unless otherwise specified. Fragments of heavy chains include CDRs, CDRs and FRs, and combinations thereof. A typical heavy chain has, following the variable domain (from N-terminal to C-terminal), a C_{H}1 domain, a hinge, a C_{H}2 domain, and a C_{H}3 domain. A functional fragment of a heavy chain includes a fragment that is capable of specifically recognizing an epitope (e.g., recognizing the epitope with a K_{D} in the micromolar, nanomolar, or picomolar range), that is capable of expressing and secreting from a cell, and that comprises at least one CDR. Heavy chain variable domains are encoded by variable region nucleotide sequence, which generally comprises V_{H}, D_{H}, and J_{H} segments derived from a repertoire of V_{H}, D_{H}, and J_{H} segments present in the germline. Sequences, locations and nomenclature for V, D, and J heavy chain segments for various organisms can be found in IMGT database, which is accessible via the internet on the world wide web (www) at the URL “imgt.org.”

The term “light chain” includes an immunoglobulin light chain sequence from any organism, and unless otherwise specified includes human kappa (κ) and lambda (2) light chains and a VpreB, as well as surrogate light chains. Light chain variable domains typically include three light chain CDRs and four framework (FR) regions, unless otherwise specified. Generally, a full-length light chain includes, from amino terminus to carboxyl terminus, a variable domain that includes FR1-CDR1-FR2-CDR2-FR3-CDR3-FR4, and a light chain constant region amino acid sequence. Light chain variable domains are encoded by the light chain variable region nucleotide sequence, which generally comprises light chain V_{L} and light chain J_{L} gene segments, derived from a repertoire of light chain V and J gene segments present in the germline. Sequences, locations and nomenclature for light chain V and J gene segments for various organisms can be found in IMGT database, which is accessible via the internet on the world wide web (www) at the URL “imgt.org.” Light chains include those, e.g., that do not selectively bind either a first or a second epitope selectively bound by the epitope-binding protein in which they appear. Light chains also include those that bind and recognize, or assist the heavy chain with binding and recognizing, one or more epitopes selectively bound by the epitope-binding protein in which they appear.

The term “complementary determining region” or “CDR,” as used herein, includes an amino acid sequence encoded by a nucleic acid sequence of an organism's immunoglobulin genes that normally (i.e., in a wild type animal) appears between two framework regions in a variable region of a light or a heavy chain of an immunoglobulin molecule (e.g., an antibody or a T cell receptor). A CDR can be encoded by, for example, a germline sequence or a rearranged sequence, and, for example, by a naïve or a mature B cell or a T cell. A CDR can be somatically mutated (e.g., vary from a sequence encoded in an animal's germline), humanized, and/or modified with amino acid substitutions, additions, or deletions. In some circumstances (e.g., for a CDR3), CDRs can be encoded by two or more sequences (e.g., germline sequences) that are not contiguous (e.g., in an unrearranged nucleic acid sequence) but are contiguous in a B cell nucleic acid sequence, e.g., as a result of splicing or connecting the sequences (e.g., V-D-J recombination to form a heavy chain CDR3.

The term “unrearranged” includes the state of an immunoglobulin locus wherein V gene segments and J gene segments (for heavy chains, D gene segments as well) are maintained separately but are capable of being joined to form a rearranged V (D) J gene that comprises a single V, (D), J of the V (D) J repertoire. The term “rearranged” includes a configuration of a heavy chain or light chain immunoglobulin locus wherein a V segment is positioned immediately adjacent to a D-J or J segment in a conformation encoding essentially a complete V_{H} or V_{L} domain, respectively.

The nucleic acids encoding the antigen-binding proteins in the exogenous donor nucleic acids can be RNA or DNA, can be single-stranded or double-stranded, and can be linear or circular. They can be part of a vector, such as an expression vector or a targeting vector. The vector can also be a viral vector such as adenoviral, adeno-associated viral (AAV), lentiviral, and retroviral vectors. For example, the exogenous donor nucleic acid can be part of an AAV, such as AAV8 or AAV2/8.

Optionally, the nucleic acids can be codon optimized for efficient translation into protein in a particular cell or organism. For example, the nucleic acid can be modified to substitute codons having a higher frequency of usage in a human cell, a non-human cell, a mammalian cell, a rodent cell, a mouse cell, a rat cell, or any other host cell of interest.

The antigen-binding-protein coding sequence in the exogenous donor nucleic acid can optionally be operably linked to any suitable promoter for expression in vivo within an animal or ex vivo within a cell. Alternatively, the exogenous donor nucleic acid can be designed such that the antigen-binding-protein coding sequence will be operably linked to an endogenous promoter at the genomic locus or safe harbor locus once it is genomically integrated. The animal can be any suitable animal as described elsewhere herein. The promoter can be a constitutively active promoter (e.g., a CAG promoter or a U6 promoter), a conditional promoter, an inducible promoter, a temporally restricted promoter (e.g., a developmentally regulated promoter), or a spatially restricted promoter (e.g., a cell-specific or tissue-specific promoter). Such promoters are well-known and are discussed elsewhere herein. Promoters that can be used in an expression construct include promoters active, for example, in one or more of a eukaryotic cell, a human cell, a non-human cell, a mammalian cell, a non-human mammalian cell, a rodent cell, a mouse cell, a rat cell, a hamster cell, a rabbit cell, a pluripotent cell, an embryonic stem (ES) cell, or a zygote. Such promoters can be, for example, conditional promoters, inducible promoters, constitutive promoters, or tissue-specific promoters.

Optionally, the promoter can be a bidirectional promoter driving expression of one gene (e.g., a gene encoding a light chain) and a second gene (e.g., a gene encoding a heavy chain) in the other direction. Such bidirectional promoters can consist of (1) a complete, conventional, unidirectional Pol III promoter that contains 3 external control elements: a distal sequence element (DSE), a proximal sequence element (PSE), and a TATA box; and (2) a second basic Pol III promoter that includes a PSE and a TATA box fused to the 5′ terminus of the DSE in reverse orientation. For example, in the H1 promoter, the DSE is adjacent to the PSE and the TATA box, and the promoter can be rendered bidirectional by creating a hybrid promoter in which transcription in the reverse direction is controlled by appending a PSE and TATA box derived from the U6 promoter. See, e.g., US 2016/0074535, herein incorporated by references in its entirety for all purposes. Use of a bidirectional promoter to express two genes simultaneously allows for the generation of compact expression cassettes to facilitate delivery.

The antigen-binding protein can be a single-chain antigen-binding protein such as an scFv. Alternatively, the antigen-binding protein is not a single-chain antigen-binding protein. For example, the antigen-binding protein can include separate light and heavy chains. The heavy chain coding sequence can be upstream of the light chain coding sequence, or the light chain coding sequence can be upstream of the heavy chain coding sequence. In one specific example, the heavy chain coding sequence is upstream of the light chain coding sequence. For example, the heavy chain coding sequence can comprise V_{H}, D_{H}, and J_{H} segments, and the light chain coding sequence can comprise light chain V_{L} and light chain J_{L} gene segments. The antigen-binding protein coding sequence can be operably linked to an exogenous promoter in the exogenous donor nucleic acid, or the exogenous donor nucleic acid can be designed such that the antigen-binding protein coding sequence will be operably linked to an endogenous promoter at the genomic locus or safe harbor locus once it is genomically integrated. In one specific example, the exogenous donor nucleic acid can be designed such that the antigen-binding protein coding sequence will be operably linked to an endogenous promoter at the genomic locus or safe harbor locus once it is genomically integrated. Likewise, the antigen-binding protein coding sequence in the exogenous donor nucleic acid can include an exogenous signal sequence for secretion and/or the exogenous donor nucleic acid can be designed so that the antigen-binding protein coding sequence will be operably linked to an endogenous signal sequence at the genomic locus or safe harbor locus once it is genomically integrated. In one example, the exogenous donor nucleic acid can be designed so that the antigen-binding protein coding sequence will be operably linked to an endogenous signal sequence at the genomic locus or safe harbor locus once it is genomically integrated. In a specific example, the antigen-binding protein comprises separate light and heavy chains, and the exogenous donor nucleic acid is designed such that the coding sequence for one chain will be operably linked to an endogenous signal sequence at the genomic locus or safe harbor locus once it is genomically integrated and the coding sequence for the other chain is operably linked to a separate exogenous signal sequence. In a specific example, the antigen-binding protein comprises separate light and heavy chains, and the exogenous donor nucleic acid is designed such that the whichever chain coding sequence is upstream in the exogenous donor nucleic acid will be operably linked to an endogenous signal sequence at the genomic locus or safe harbor locus once it is genomically integrated, and an exogenous signal sequence is operably linked to the whichever chain coding sequence is downstream in the exogenous donor nucleic acid. Alternatively, the exogenous donor nucleic acid can be designed such that the coding sequences for both chains will be operably linked to an endogenous signal sequence at the genomic locus or safe harbor locus once it is genomically integrated, or the coding sequence for both chains can be operably linked to the same exogenous signal sequence or the coding sequence for each chain can be operably linked to separate exogenous signal sequences.

Signal sequences (i.e., N-terminal signal sequences) mediate targeting of nascent secretory and membrane proteins to the endoplasmic reticulum (ER) in a signal recognition particle (SRP)-dependent manner. Usually, signal sequences are cleaved off co-translationally so that signal peptides and mature proteins are generated. Examples of exogenous signal sequences or signal peptides that can be used include, for example, the signal sequence/peptide from mouse albumin, human albumin, mouse ROR1, human ROR1, human azurocidin, Cricetulus griseus Ig kappa chain V III region MOPC 63 like, and human Ig kappa chain V III region VG. Any other known signal sequence/peptide can also be used. In a specific example, an ROR1 signal sequence is used. An example of such a signal sequence is set forth in SEQ ID NO: 33 (encoded by SEQ ID NO: 31 or 32).

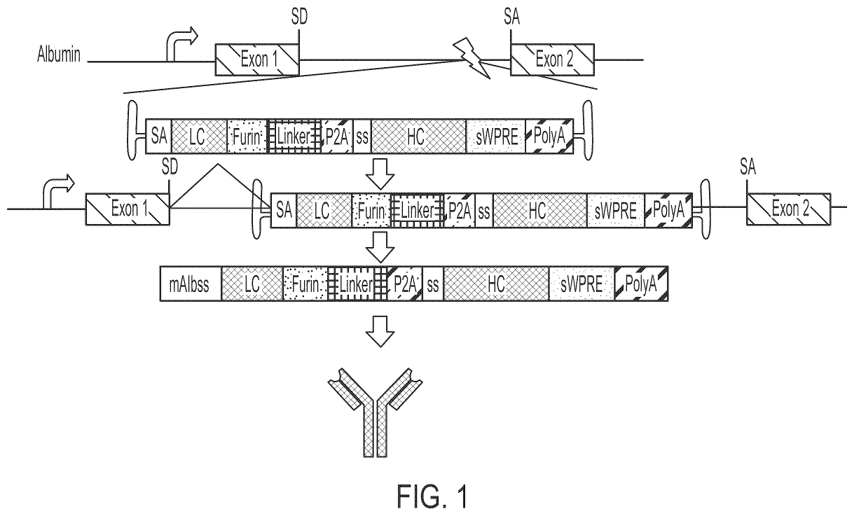

One or more of the nucleic acids in the antigen-binding-protein coding sequence (e.g., a heavy chain coding sequence and a light chain coding sequence) can be together in a multicistronic expression construct. For example, a nucleic acid encoding a heavy chain and a light chain can be together in a bicistronic expression construct. See, e.g., FIG. 1. Multicistronic expression vectors simultaneously express two or more separate proteins from the same mRNA (i.e., a transcript produced from the same promoter). Suitable strategies for multicistronic expression of proteins include, for example, the use of a 2A peptide and the use of an internal ribosome entry site (IRES). As one example, such multicistronic vectors can use one or more internal ribosome entry sites (IRES) to allow for initiation of translation from an internal region of an mRNA. As another example, such multicistronic vectors can use one or more 2A peptides. These peptides are small “self-cleaving” peptides, generally having a length of 18-22 amino acids and produce equimolar levels of multiple genes from the same mRNA. Ribosomes skip the synthesis of a glycyl-prolyl peptide bond at the C-terminus of a 2A peptide, leading to the “cleavage” between a 2A peptide and its immediate downstream peptide. See, e.g., Kim et al. (2011) PLoS One 6 (4): e18556, herein incorporated by reference in its entirety for all purposes. The “cleavage” occurs between the glycine and proline residues found on the C-terminus, meaning the upstream cistron will have a few additional residues added to the end, while the downstream cistron will start with the proline. As a result, the “cleaved-off” downstream peptide has proline at its N-terminus. 2A-mediated cleavage is a universal phenomenon in all eukaryotic cells. 2A peptides have been identified from picornaviruses, insect viruses and type C rotaviruses. See, e.g., Szymczak et al. (2005) Expert Opin Biol Ther 5:627-638, herein incorporated by reference in its entirety for all purposes. Examples of 2A peptides that can be used include Thosea asigna virus 2A (T2A); porcine teschovirus-1 2A (P2A); equine rhinitis A virus (ERAV) 2A (E2A); and FMDV 2A (F2A). Exemplary T2A, P2A, E2A, and F2A sequences include the following: T2A (EGRGSLLTCGDVEENPGP; SEQ ID NO: 29); P2A (ATNFSLLKQAGDVEENPGP; SEQ ID NO: 25); E2A (QCTNYALLKLAGDVESNPGP; SEQ ID NO: 30); and F2A (VKQTLNFDLLKLAGDVESNPGP; SEQ ID NO: 27). GSG residues can be added to the 5′ end of any of these peptides to improve cleavage efficiency.

In some exogenous donor nucleic acids, a nucleic acid encoding a furin cleavage site is included between the light chain coding sequence and the heavy chain coding sequence. In some exogenous donor nucleic acids, a nucleic acid encoding a linker (e.g., GSG) is included between the light chain coding sequence and the heavy chain coding sequence (e.g., directly upstream of the 2A peptide coding sequence). For example, a furin cleavage site can be included upstream of a 2A peptide, with both the furin cleavage site and the 2A peptide being located between the light chain and the heavy chain (i.e., upstream chain-furin cleavage site-2A peptide-downstream chain). During translation, a first cleavage event will occur at the 2A peptide sequence. However, most of the 2A peptide will remain attached as a remnant to the C-terminus of the upstream chain (e.g., light chain if the light chain is upstream of the heavy chain, or heavy chain if the heavy chain is upstream of the light chain), with one amino acid added to the N-terminus of the downstream chain (or the N-terminus of a signal sequence, if a signal sequence is included upstream of the downstream chain). A second cleavage event, initiated at the furin cleavage site, yields the upstream chain without the 2A remnants in order to obtain a more native heavy chain or light chain by post-translational processing.

The exogenous donor nucleic acids can also comprise a polyadenylation signal or transcription terminator downstream of the antigen-binding-protein coding sequence. The exogenous donor nucleic acids can also comprise a polyadenylation signal or transcription terminator upstream of the antigen-binding-protein coding sequence. The polyadenylation signal or transcription terminator upstream of the antigen-binding-protein coding sequence can be flanked by recombinase recognition sites recognized by a site-specific recombinase. Optionally, the recombinase recognition sites also flank a selection cassette comprising, for example, the coding sequence for a drug resistance protein. Optionally the recombinase recognition sites do not flank a selection cassette. The polyadenylation signal or transcription terminator prevents transcription and expression of the protein or RNA encoded by the coding sequence (e.g., chimeric Cas protein, chimeric adaptor protein, guide RNA, or recombinase). However, upon exposure to the site-specific recombinase, the polyadenylation signal or transcription terminator will be excised, and the protein or RNA can be expressed.

Such a configuration can enable tissue-specific expression or developmental-stage-specific expression in animals comprising the antigen-binding-protein coding sequence if the polyadenylation signal or transcription terminator is excised in a tissue-specific or developmental-stage-specific manner. Excision of the polyadenylation signal or transcription terminator in a tissue-specific or developmental-stage-specific manner can be achieved if an animal comprising the antigen-binding-protein expression cassette further comprises a coding sequence for the site-specific recombinase operably linked to a tissue-specific or developmental-stage-specific promoter. The polyadenylation signal or transcription terminator will then be excised only in those tissues or at those developmental stages, enabling tissue-specific expression or developmental-stage-specific expression. In one example, an antigen-binding-protein can be expressed in a liver-specific manner. Examples of such promoters are well-known.

Any transcription terminator or polyadenylation signal can be used. A “transcription terminator” as used herein refers to a DNA sequence that causes termination of transcription. In eukaryotes, transcription terminators are recognized by protein factors, and termination is followed by polyadenylation, a process of adding a poly(A) tail to the mRNA transcripts in presence of the poly(A) sipolymerase. The mammalian poly(A) signal typically consists of a core sequence, about 45 nucleotides long, that may be flanked by diverse auxiliary sequences that serve to enhance cleavage and polyadenylation efficiency. The core sequence consists of a highly conserved upstream element (AATAAA or AAUAAA) in the mRNA, referred to as a poly A recognition motif or poly A recognition sequence), recognized by cleavage and polyadenylation-specificity factor (CPSF), and a poorly defined downstream region (rich in Us or Gs and Us), bound by cleavage stimulation factor (CstF). Examples of transcription terminators that can be used include, for example, the human growth hormone (HGH) polyadenylation signal, the simian virus 40 (SV40) late polyadenylation signal, the rabbit beta-globin polyadenylation signal, the bovine growth hormone (BGH) polyadenylation signal, the phosphoglycerate kinase (PGK) polyadenylation signal, an AOX1 transcription termination sequence, a CYC1 transcription termination sequence, or any transcription termination sequence known to be suitable for regulating gene expression in eukaryotic cells.

Site-specific recombinases include enzymes that can facilitate recombination between recombinase recognition sites, where the two recombination sites are physically separated within a single nucleic acid or on separate nucleic acids. Examples of recombinases include Cre, Flp, and Dre recombinases. One example of a Cre recombinase gene is Crei, in which two exons encoding the Cre recombinase are separated by an intron to prevent its expression in a prokaryotic cell. Such recombinases can further comprise a nuclear localization signal to facilitate localization to the nucleus (e.g., NLS-Crei). Recombinase recognition sites include nucleotide sequences that are recognized by a site-specific recombinase and can serve as a substrate for a recombination event. Examples of recombinase recognition sites include FRT, FRT11, FRT71, attp, att, rox, and lox sites such as loxP, lox511, lox2272, lox66, lox71, loxM2, and lox5171.

The exogenous donor nucleic acids disclosed herein can comprise other components as well. Such exogenous donor nucleic acids can further comprise a 3′ splicing sequence (splice acceptor site) at the 5′ end of the antigen-binding-protein coding sequence. The term 3′ splicing sequence refers to a nucleic acid sequence at a 3′ intron/exon boundary that can be recognized and bound by splicing machinery. The exogenous donor nucleic acids can also comprise post-transcriptional regulatory elements, such as the woodchuck hepatitis virus post-transcriptional regulatory element.

A specific example of a donor nucleic acid encoding an antigen-binding protein targeting Zika virus envelope (Env) proteins comprises SA-LC-P2A-HC-pA, where SA refers to splice acceptor site, LC refers to antibody light chain, P2A refers to the P2A peptide, HC refers to antibody heavy chain, and pA refers to a polyadenylation signal. An example of such a donor is set forth in SEQ ID NO: 1. The light chain nucleotide sequence is set forth in SEQ ID NO: 2 and encodes the protein sequence set forth in SEQ ID NO: 3. The heavy chain nucleotide sequence is set forth in SEQ ID NO: 4 and encodes the protein sequence set forth in SEQ ID NO: 5. The light chain variable region nucleotide sequence is set forth in SEQ ID NO: 103 and encodes the protein set forth in SEQ ID NO: 104. The heavy chain variable region nucleotide sequence is set forth in SEQ ID NO: 105 and encodes the protein set forth in SEQ ID NO: 106. The three light chain CDRs are set forth in SEQ ID NOS: 64-66, respectively, and are encoded by SEQ ID NOS: 85-87, respectively. The three heavy chain CDRs are set forth in SEQ ID NOS: 67-69, respectively, and are encoded by SEQ ID NOS: 88-90, respectively. An example of an anti-Zika antibody comprises a light chain that is at least 90%, 95%, 96%, 97%, 98%, 99%, or 100% identical to SEQ ID NO: 3 (optionally comprising CDRs at least 90%, 95%, 96%, 97%, 98%, 99%, or 100% identical to those set forth in SEQ ID NOS: 64-66) and a heavy chain that is at least 90%, 95%, 96%, 97%, 98%, 99%, or 100% identical to SEQ ID NO: 5 (optionally comprising CDRs at least 90%, 95%, 96%, 97%, 98%, 99%, or 100% identical to those set forth in SEQ ID NOS: 67-69). An example of an anti-Zika antibody comprises a light chain variable region that is at least 90%, 95%, 96%, 97%, 98%, 99%, or 100% identical to SEQ ID NO: 104 (optionally comprising CDRs at least 90%, 95%, 96%, 97%, 98%, 99%, or 100% identical to those set forth in SEQ ID NOS: 64-66) and a heavy chain variable region that is at least 90%, 95%, 96%, 97%, 98%, 99%, or 100% identical to SEQ ID NO: 106 (optionally comprising CDRs at least 90%, 95%, 96%, 97%, 98%, 99%, or 100% identical to those set forth in SEQ ID NOS: 67-69). In a specific example, a modified albumin locus (comprising endogenous mouse albumin exon 1 and the integrated antibody coding sequence) can comprise a coding sequence at least 90%, 95%, 96%, 97%, 98%, 99%, or 100% identical to the sequence set forth in SEQ ID NO: 115.

Other specific examples of donor nucleic acids encoding an antigen-binding protein targeting Zika virus envelope (Env) proteins comprise SA-HC-F2A-Albss-LC-pA, SA-HC-P2A-Albss-LC-pA, Sa-HC-T2A-Albss-LC-pA, or HC-T2A-RORss-LC-pA, where SA refers to splice acceptor site, LC refers to antibody light chain, P2A refers to the P2A peptide, HC refers to antibody heavy chain, Albss refers to an albumin signal sequence (e.g., from mouse albumin), and pA refers to a polyadenylation signal. Example of such donors are set forth in SEQ ID NOS: 6-9. The light chain nucleotide sequence is set forth in SEQ ID NO: 12 and encodes the protein sequence set forth in SEQ ID NO: 13. The heavy chain nucleotide sequence is set forth in SEQ ID NO: 14 and encodes the protein sequence set forth in SEQ ID NO: 15. The light chain variable region nucleotide sequence is set forth in SEQ ID NO: 107 and encodes the protein sequence set forth in SEQ ID NO: 108. The heavy chain variable region nucleotide sequence is set forth in SEQ ID NO: 109 and encodes the protein sequence set forth in SEQ ID NO: 110. The three light chain CDRs are set forth in SEQ ID NOS: 70-72, respectively, and are encoded by SEQ ID NOS: 91-93, respectively. The three heavy chain CDRs are set forth in SEQ ID NOS: 73-75, respectively, and are encoded by SEQ ID NOS: 94-96, respectively. An example of an anti-Zika antibody comprises a light chain that is at least 90%, 95%, 96%, 97%, 98%, 99%, or 100% identical to SEQ ID NO: 13 (optionally comprising CDRs at least 90%, 95%, 96%, 97%, 98%, 99%, or 100% identical to those set forth in SEQ ID NOS: 70-72) and a heavy chain that is at least 90%, 95%, 96%, 97%, 98%, 99%, or 100% identical to SEQ ID NO: 15 (optionally comprising CDRs at least 90%, 95%, 96%, 97%, 98%, 99%, or 100% identical to those set forth in SEQ ID NOS: 73-75). An example of an anti-Zika antibody comprises a light chain variable region that is at least 90%, 95%, 96%, 97%, 98%, 99%, or 100% identical to SEQ ID NO: 108 (optionally comprising CDRs at least 90%, 95%, 96%, 97%, 98%, 99%, or 100% identical to those set forth in SEQ ID NOS: 70-72) and a heavy chain variable region that is at least 90%, 95%, 96%, 97%, 98%, 99%, or 100% identical to SEQ ID NO: 110 (optionally comprising CDRs at least 90%, 95%, 96%, 97%, 98%, 99%, or 100% identical to those set forth in SEQ ID NOS: 73-75). In a specific example, a modified albumin locus (comprising endogenous mouse albumin exon 1 and the integrated antibody coding sequence) can comprise a coding sequence at least 90%, 95%, 96%, 97%, 98%, 99%, or 100% identical to the sequence set forth in any one of SEQ ID NOS: 116-119.

A specific example of a donor nucleic acid encoding an antigen-binding protein targeting influenza virus hemagglutinin (HA) protein comprises SA-LC-P2A-HC-pA, where SA refers to splice acceptor site, LC refers to antibody light chain, P2A refers to the P2A peptide, HC refers to antibody heavy chain, and pA refers to a polyadenylation signal. Another specific example of a donor nucleic acid encoding an antigen-binding protein targeting influenza virus hemagglutinin (HA) protein comprises SA-LC-T2A-HC-pA, where SA refers to splice acceptor site, LC refers to antibody light chain, T2A refers to the T2A peptide, HC refers to antibody heavy chain, and pA refers to a polyadenylation signal. An example of such a donor is set forth in SEQ ID NO: 16. The light chain nucleotide sequence is set forth in SEQ ID NO: 17 and encodes the protein sequence set forth in SEQ ID NO: 18. The heavy chain nucleotide sequence is set forth in SEQ ID NO: 19 and encodes the protein sequence set forth in SEQ ID NO: 20. The light chain variable region nucleotide sequence is set forth in SEQ ID NO: 111 and encodes the protein sequence set forth in SEQ ID NO: 112. The heavy chain variable region nucleotide sequence is set forth in SEQ ID NO: 113 and encodes the protein sequence set forth in SEQ ID NO: 114. The three light chain CDRs are set forth in SEQ ID NOS: 76-78, respectively, and are encoded by SEQ ID NOS: 97-99, respectively. The three heavy chain CDRs are set forth in SEQ ID NOS: 79-81, respectively, and are encoded by SEQ ID NOS: 100-102, respectively. An example of an anti-HA antibody comprises a light chain that is at least 90%, 95%, 96%, 97%, 98%, 99%, or 100% identical to SEQ ID NO: 18 (optionally comprising CDRs at least 90%, 95%, 96%, 97%, 98%, 99%, or 100% identical to those set forth in SEQ ID NOS: 76-78) and a heavy chain that is at least 90%, 95%, 96%, 97%, 98%, 99%, or 100% identical to SEQ ID NO: 20 (optionally comprising CDRs at least 90%, 95%, 96%, 97%, 98%, 99%, or 100% identical to those set forth in SEQ ID NOS: 79-81). An example of an anti-HA antibody comprises a light chain variable region that is at least 90%, 95%, 96%, 97%, 98%, 99%, or 100% identical to SEQ ID NO: 112 (optionally comprising CDRs at least 90%, 95%, 96%, 97%, 98%, 99%, or 100% identical to those set forth in SEQ ID NOS: 76-78) and a heavy chain variable region that is at least 90%, 95%, 96%, 97%, 98%, 99%, or 100% identical to SEQ ID NO: 114 (optionally comprising CDRs at least 90%, 95%, 96%, 97%, 98%, 99%, or 100% identical to those set forth in SEQ ID NOS: 79-81). In a specific example, a modified albumin locus (comprising endogenous mouse albumin exon 1 and the integrated antibody coding sequence) can comprise a coding sequence at least 90%, 95%, 96%, 97%, 98%, 99%, or 100% identical to the sequence set forth in SEQ ID NO: 120.

Another specific example of a donor nucleic acid encoding an antigen-binding protein targeting influenza virus hemagglutinin (HA) protein comprises SA-LC-T2A-RoRss-HC-pA, where SA refers to splice acceptor site, LC refers to antibody light chain, T2A refers to the T2A peptide, RORss refers to an ROR signal sequence, HC refers to antibody heavy chain, and pA refers to a polyadenylation signal. An example of such a donor is set forth in SEQ ID NO: 145. The light chain nucleotide sequence is set forth in SEQ ID NO: 125 and encodes the protein sequence set forth in SEQ ID NO: 126. The heavy chain nucleotide sequence is set forth in SEQ ID NO: 127 and encodes the protein sequence set forth in SEQ ID NO: 128. The light chain variable region nucleotide sequence is set forth in SEQ ID NO: 141 and encodes the protein sequence set forth in SEQ ID NO: 142. The heavy chain variable region nucleotide sequence is set forth in SEQ ID NO: 143 and encodes the protein sequence set forth in SEQ ID NO: 144. The three light chain CDRs are set forth in SEQ ID NOS: 129-131, respectively, and are encoded by SEQ ID NOS: 135-137, respectively. The three heavy chain CDRs are set forth in SEQ ID NOS: 132-134, respectively, and are encoded by SEQ ID NOS: 138-140, respectively. An example of an anti-HA antibody comprises a light chain that is at least 90%, 95%, 96%, 97%, 98%, 99%, or 100% identical to SEQ ID NO: 126 (optionally comprising CDRs at least 90%, 95%, 96%, 97%, 98%, 99%, or 100% identical to those set forth in SEQ ID NOS: 129-131) and a heavy chain that is at least 90%, 95%, 96%, 97%, 98%, 99%, or 100% identical to SEQ ID NO: 128 (optionally comprising CDRs at least 90%, 95%, 96%, 97%, 98%, 99%, or 100% identical to those set forth in SEQ ID NOS: 132-134). An example of an anti-HA antibody comprises a light chain variable region that is at least 90%, 95%, 96%, 97%, 98%, 99%, or 100% identical to SEQ ID NO: 142 (optionally comprising CDRs at least 90%, 95%, 96%, 97%, 98%, 99%, or 100% identical to those set forth in SEQ ID NOS: 129-131) and a heavy chain variable region that is at least 90%, 95%, 96%, 97%, 98%, 99%, or 100% identical to SEQ ID NO: 144 (optionally comprising CDRs at least 90%, 95%, 96%, 97%, 98%, 99%, or 100% identical to those set forth in SEQ ID NOS: 132-134). In a specific example, a modified albumin locus (comprising the integrated antibody coding sequence) can comprise a coding sequence at least 90%, 95%, 96%, 97%, 98%, 99%, or 100% identical to the sequence set forth in SEQ ID NO: 146.

A specific example of a donor nucleic acid encoding an antigen-binding protein targeting Pseudomonas aeruginosa PcrV protein comprises SA-HC-T2A-LC-pA, where SA refers to splice acceptor site, LC refers to antibody light chain, T2A refers to the T2A peptide, HC refers to antibody heavy chain, and pA refers to a polyadenylation signal.

### C. Safe Harbor Loci and the Albumin Locus

The antigen-binding protein coding sequences described elsewhere herein can be genomically integrated at a target genomic locus in a cell or an animal. Any target genomic locus capable of expressing a gene can be used, such as a safe harbor locus (safe harbor gene). Interactions between integrated exogenous DNA and a host genome can limit the reliability and safety of integration and can lead to overt phenotypic effects that are not due to the targeted genetic modification but are instead due to unintended effects of the integration on surrounding endogenous genes. For example, randomly inserted transgenes can be subject to position effects and silencing, making their expression unreliable and unpredictable. Likewise, integration of exogenous DNA into a chromosomal locus can affect surrounding endogenous genes and chromatin, thereby altering cell behavior and phenotypes. Safe harbor loci include chromosomal loci where transgenes or other exogenous nucleic acid inserts can be stably and reliably expressed in all tissues of interest without overtly altering cell behavior or phenotype (i.e., without any deleterious effects on the host cell). See, e.g., Sadelain et al. (2012) Nat. Rev. Cancer 12:51-58, herein incorporated by reference in its entirety for all purposes. For example, the safe harbor locus can be one in which expression of the inserted gene sequence is not perturbed by any read-through expression from neighboring genes. For example, safe harbor loci can include chromosomal loci where exogenous DNA can integrate and function in a predictable manner without adversely affecting endogenous gene structure or expression. Safe harbor loci can include extragenic regions or intragenic regions such as, for example, loci within genes that are non-essential, dispensable, or able to be disrupted without overt phenotypic consequences.

Such safe harbor loci can offer an open chromatin configuration in all tissues and can be ubiquitously expressed during embryonic development and in adults. See, e.g., Zambrowicz et al. (1997) Proc. Natl. Acad. Sci. U.S.A. 94:3789-3794, herein incorporated by reference in its entirety for all purposes. In addition, the safe harbor loci can be targeted with high efficiency, and safe harbor loci can be disrupted with no overt phenotype. Examples of safe harbor loci include albumin, CCR5, HPRT, AAVS1, and Rosa26. See, e.g., U.S. Pat. Nos. 7,888,121; 7,972,854; 7,914,796; 7,951,925; 8,110,379; 8,409,861; 8,586,526; and US Patent Publication Nos. 2003/0232410; 2005/0208489; 2005/0026157; 2006/0063231; 2008/0159996; 2010/00218264; 2012/0017290; 2011/0265198; 2013/0137104; 2013/0122591; 2013/0177983; 2013/0177960; and 2013/0122591, each of which is herein incorporated by reference in its entirety for all purposes. Another example of a suitable safe harbor locus is TTR.

The antigen-binding protein coding sequence can be integrated into any part of the genomic locus or safe harbor locus. For example, they can be inserted into an intron or an exon of a safe harbor locus, or can replace one or more introns and/or exons of a genomic locus or safe harbor locus. Expression cassettes integrated into a target genomic locus can be operably linked to an endogenous promoter at the target genomic locus (e.g., the endogenous albumin promoter) or can be operably linked to an exogenous promoter that is heterologous to the target genomic locus. In one example, an antigen-binding protein coding sequence is integrated into a target genomic locus (e.g., the albumin locus) and is operably linked to the endogenous promoter at the target genomic locus (e.g., the albumin promoter). In another example, an antigen-binding protein coding sequence is integrated into a target genomic locus (e.g., the albumin locus) and is operably linked to a heterologous promoters (e.g., a CMV promoter).

In one example, the safe harbor locus is an albumin locus. Albumin is a protein that is produced in the liver and secreted into the blood. Serum albumin the majority of the protein found in blood in humans. The albumin locus is highly expressed, resulting in the production of approximately 15 g of albumin protein in humans each day. Albumin has no autocrine function, and there does not appear to be any phenotype associated with monoallelic knockouts and only mild phenotypic observations are found for biallelic knockouts. See, e.g., Watkins et al (1994) Proc. Natl. Acad. Sci. U.S.A. 91:9417-9421, herein incorporated by reference in its entirety for all purposes. The albumin gene locus is a safe and effective site for therapeutic gene insertion and expression. Insertion into the albumin locus in the liver for long-term expression is an attractive therapeutic modality. In one example, the antigen-binding protein sequence is integrated into an intron of the albumin locus, such as the first intron of the albumin locus. See, e.g., FIG. 1. The albumin gene structure is suited for transgene targeting into intronic sequences because its first exon encodes a secretory peptide (signal peptide or signal sequence) that is cleaved from the final protein product. For example, integration of a promoterless cassette bearing a splice acceptor and a therapeutic transgene would support expression and secretion of many different proteins.

Human ALB maps to human 4q13.3 on chromosome 4 (NCBI RefSeq Gene ID 213; Assembly GRCh38.p12 (GCF_000001405.38); location NC_000004.12 (73404239 . . . 73421484 (+))). The gene has been reported to have 15 exons. The wild type human albumin protein has been assigned UniProt accession number P02768. At least three isoforms are known (P02768-1 through P02768-3). Mouse Alb maps to mouse 5 E1; 5 44.7 cM on chromosome 5 (NCBI RefSeq Gene ID 11657; Assembly GRCm38.p4 (GCF_000001635.24); location NC_000071.6 (90,460,870 . . . 90,476,602 (+))). The gene has been reported to have 15 exons. The wild type mouse albumin protein has been assigned UniProt accession number P07724. Albumin sequences for many other non-human animals are also known. These include, for example, bovine (UniProt accession number P02769; NCBI RefSeq Gene ID 280717), rat (UniProt accession number P02770; NCBI RefSeq Gene ID 24186), chicken (UniProt accession number P19121), Sumatran orangutan (UniProt accession number Q5NVH5; NCBI RefSeq Gene ID 100174145), horse (UniProt accession number P35747; NCBI RefSeq Gene ID 100034206), cat (UniProt accession number P49064; NCBI RefSeq Gene ID 448843), rabbit (UniProt accession number P49065; NCBI RefSeq Gene ID 100009195), dog (UniProt accession number P49822; NCBI RefSeq Gene ID 403550), pig (UniProt accession number P08835; NCBI RefSeq Gene ID 396960), Mongolian gerbil (UniProt accession number 035090), rhesus macaque (UniProt accession number Q28522; NCBI RefSeq Gene ID 704892), donkey (UniProt accession number Q5XLE4; NCBI RefSeq Gene ID 106835108), sheep (UniProt accession number P14639; NCBI RefSeq Gene ID 443393), American bullfrog (UniProt accession number P21847), golden hamster (UniProt accession number A6YF56; NCBI RefSeq Gene ID 101837229), and goat (UniProt accession number P85295).

D. Introducing Nuclease Agents and Donor Nucleic Acids into Cells and Animals

The methods disclosed herein comprise introducing into a cell or animal nuclease agents (or nucleic acids encoding nuclease agents) and exogenous donor nucleic acids. “Introducing” includes presenting to the cell or animal the nucleic acid or protein in such a manner that the nucleic acid or protein gains access to the interior of the cell or to the interior of cells within the animal. The introducing can be accomplished by any means, and two or more of the components (e.g., two of the components, or all of the components) can be introduced into the cell or animal simultaneously or sequentially in any combination. For example, a nuclease agent (or nucleic acid encoding the nuclease agent or one or more nucleic acids encoding the nuclease agent) can be introduced into a cell or animal before introduction of an exogenous donor nucleic acid. In addition, two or more of the components can be introduced into the cell or animal by the same delivery method or different delivery methods. Similarly, two or more of the components can be introduced into an animal by the same route of administration or different routes of administration.

A guide RNA can be introduced into the cell in the form of an RNA (e.g., in vitro transcribed RNA) or in the form of a DNA encoding the guide RNA. Likewise, protein components such as Cas9 proteins, ZFNs, or TALENs can be introduced into the cell in the form of DNA, RNA, or protein. For example, a guide RNA and a Cas9 protein can both be introduced in the form of RNA. When introduced in the form of a DNA, the DNA encoding a guide RNA can be operably linked to a promoter active in the cell. For example, a guide RNA may be delivered via AAV and expressed in vivo under a U6 promoter. Such DNAs can be in one or more expression constructs. For example, such expression constructs can be components of a single nucleic acid molecule. Alternatively, they can be separated in any combination among two or more nucleic acid molecules (i.e., DNAs encoding one or more CRISPR RNAs and DNAs encoding one or more tracrRNAs can be components of a separate nucleic acid molecules).

Nucleic acids encoding guide RNAs or nuclease agents can be operably linked to a promoter in an expression construct. Expression constructs include any nucleic acid constructs capable of directing expression of a gene or other nucleic acid sequence of interest and which can transfer such a nucleic acid sequence of interest to a target cell. Suitable promoters that can be used in an expression construct include promoters active, for example, in one or more of a eukaryotic cell, a human cell, a non-human cell, a mammalian cell, a non-human mammalian cell, a rodent cell, a mouse cell, a rat cell, a hamster cell, a rabbit cell, a pluripotent cell, an embryonic stem (ES) cell, an adult stem cell, a developmentally restricted progenitor cell, an induced pluripotent stem (iPS) cell, or a one-cell stage embryo. Such promoters can be, for example, conditional promoters, inducible promoters, constitutive promoters, or tissue-specific promoters. Optionally, the promoter can be a bidirectional promoter driving expression of both a guide RNA in one direction and another component in the other direction. Such bidirectional promoters can consist of (1) a complete, conventional, unidirectional Pol III promoter that contains 3 external control elements: a distal sequence element (DSE), a proximal sequence element (PSE), and a TATA box; and (2) a second basic Pol III promoter that includes a PSE and a TATA box fused to the 5′ terminus of the DSE in reverse orientation. For example, in the H1 promoter, the DSE is adjacent to the PSE and the TATA box, and the promoter can be rendered bidirectional by creating a hybrid promoter in which transcription in the reverse direction is controlled by appending a PSE and TATA box derived from the U6 promoter. See, e.g., US 2016/0074535, herein incorporated by references in its entirety for all purposes. Use of a bidirectional promoter to express genes encoding a guide RNA and another component simultaneously allows for the generation of compact expression cassettes to facilitate delivery.

Guide RNAs or nucleic acids encoding guide RNAs (or other components) can be provided in compositions comprising a carrier increasing the stability of the guide RNA (e.g., prolonging the period under given conditions of storage (e.g., −20° C., 4° C., or ambient temperature) for which degradation products remain below a threshold, such below 0.5% by weight of the starting nucleic acid or protein; or increasing the stability in vivo). Non-limiting examples of such carriers include poly(lactic acid) (PLA) microspheres, poly(D,L-lactic-coglycolic-acid) (PLGA) microspheres, liposomes, micelles, inverse micelles, lipid cochleates, and lipid microtubules.

Various methods and compositions are provided herein to allow for introduction of a nucleic acid or protein into a cell or animal. Such methods for introducing nucleic acid or proteins into a cell or animal can include, for example, vector delivery, particle-mediated delivery, exosome-mediated delivery, lipid-nanoparticle (LNP)-mediated delivery, cell-penetrating-peptide-mediated delivery, or implantable-device-mediated delivery. As specific examples, a nucleic acid or protein can be introduced into a cell or animal in a carrier such as a poly(lactic acid) (PLA) microsphere, a poly(D,L-lactic-coglycolic-acid) (PLGA) microsphere, a liposome, a micelle, an inverse micelle, a lipid cochleate, or a lipid microtubule. Some specific examples of delivery to an animal include hydrodynamic delivery, virus-mediated delivery (e.g., adeno-associated virus (AAV)-mediated delivery, or by adenovirus, by lentivirus, or by retrovirus), and lipid-nanoparticle-mediated delivery. In one specific example, both the nuclease agent (or nucleic acid encoding the nuclease agent or one or more nucleic acids encoding the nuclease agent) and exogenous donor sequence can be delivered via LNP-mediated delivery. In another specific example, both the nuclease agent (or nucleic acid encoding the nuclease agent or one or more nucleic acids encoding the nuclease agent) and exogenous donor sequence can be delivered via AAV-mediated delivery. For example, the nuclease agent (or nucleic acid encoding the nuclease agent or one or more nucleic acids encoding the nuclease agent) and the exogenous donor sequence can be delivered via multiple different AAV vectors (e.g., two different AAV vectors). In a specific example in which the nuclease agent is CRISPR/Cas (e.g., CRISPR/Cas9), a first AAV vector can deliver the Cas (e.g., Cas9) or a nucleic acid encoding the Cas, and a second AAV vector can deliver the gRNA (or a nucleic acid encoding the gRNA) and the exogenous donor sequence. For example, small promoters can be used so that the Cas9 coding sequence can fit into an AAV construct. Examples of such promoters include Efs, SV40, or a synthetic promoter comprising a liver-specific enhancer (e.g., E2 from HBV virus or SerpinA from the SerpinA gene) and a core promoter (e.g., the E2P synthetic promoter or the SerpinAP synthetic promoter disclosed herein). Exemplary promoters include: (1) elongation factor 1 alpha short (EFs) (SEQ ID NO: 40); (2) simian virus 40 (SV40) (SEQ ID NO: 41); and two synthetic promoters ((3) early region 2 promoter (E2P) (SEQ ID NO: 42) and (4) SerpinAP (SEQ ID NO: 43)). However, other promoters can also be used.

When the Cas9 (nucleic acid encoding Cas9) is delivered in a first AAV and the gRNA (nucleic acid encoding gRNA) and exogenous donor sequence are delivered in a second AAV, the first and second AAVs can be delivered in any suitable ratio (e.g., the ratio of viral genomes delivered). For example, the ratio of the first AAV to the second AAV can be from about 25:1 to about 1:25, from about 10:1 to about 1:10, from about 5:1 to about 1:5, from about 4:1 to about 1:4, from about 4:1 to about 1:1, from about 1:1 to about 1:4, from about 3:1 to about 1:3, from about 3:1 to about 1:1, from about 1:1 to about 1:3, from about 2:1 to about 1:2, from about 2:1 to about 1:1, from about 1:1 to about 1:2, or about 1:1. In a specific example, the ratio of the first AAV to the second AAV is about 1:2. In another specific example, the ratio of the first AAV to the second AAV is about 2:1. In another specific example, the ratio of the first AAV to the second AAV is about 1:1. In another specific example, the ratio of the first AAV to the second AAV is about 5:1. In another specific example, the ratio of the first AAV to the second AAV is about 10:1. In another specific example, the ratio of the first AAV to the second AAV is about 1:5. In another specific example, the ratio of the first AAV to the second AAV is about 1:10.

In another specific example, the nuclease agent (or nucleic acid encoding the nuclease agent or one or more nucleic acids encoding the nuclease agent) can be delivered via LNP-mediated delivery and exogenous donor sequence can be delivered via AAV-mediated delivery. In another specific example, the nuclease agent (or nucleic acid encoding the nuclease agent or one or more nucleic acids encoding the nuclease agent) can be delivered via AAV-mediated delivery and exogenous donor sequence can be delivered via LNP-mediated delivery.

Introduction of nucleic acids and proteins into cells or animals can be accomplished by hydrodynamic delivery (HDD). Hydrodynamic delivery has emerged as a method for intracellular DNA delivery in vivo. For gene delivery to parenchymal cells, only essential DNA sequences need to be injected via a selected blood vessel, eliminating safety concerns associated with current viral and synthetic vectors. When injected into the bloodstream, DNA is capable of reaching cells in the different tissues accessible to the blood. Hydrodynamic delivery employs the force generated by the rapid injection of a large volume of solution into the incompressible blood in the circulation to overcome the physical barriers of endothelium and cell membranes that prevent large and membrane-impermeable compounds from entering parenchymal cells. In addition to the delivery of DNA, this method is useful for the efficient intracellular delivery of RNA, proteins, and other small compounds in vivo. See, e.g., Bonamassa et al. (2011) Pharm. Res. 28 (4): 694-701, herein incorporated by reference in its entirety for all purposes.

Introduction of nucleic acids can also be accomplished by virus-mediated delivery, such as AAV-mediated delivery or lentivirus-mediated delivery. Other exemplary viruses/viral vectors include retroviruses, adenoviruses, vaccinia viruses, poxviruses, and herpes simplex viruses. The viruses can infect dividing cells, non-dividing cells, or both dividing and non-dividing cells. The viruses can integrate into the host genome or alternatively do not integrate into the host genome. Such viruses can also be engineered to have reduced immunity. The viruses can be replication-competent or can be replication-defective (e.g., defective in one or more genes necessary for additional rounds of virion replication and/or packaging). Viruses can cause transient expression, long-lasting expression (e.g., at least 1 week, 2 weeks, 1 month, 2 months, or 3 months), or permanent expression (e.g., of Cas9 and/or gRNA). Exemplary viral titers (e.g., AAV titers) include 10¹², 10¹³, 10¹⁴, 10¹⁵, and 10¹⁶ vector genomes/mL.

The ssDNA AAV genome consists of two open reading frames, Rep and Cap, flanked by two inverted terminal repeats that allow for synthesis of the complementary DNA strand. When constructing an AAV transfer plasmid, the transgene is placed between the two ITRs, and Rep and Cap can be supplied in trans. In addition to Rep and Cap, AAV can require a helper plasmid containing genes from adenovirus. These genes (E4, E2a, and VA) mediate AAV replication. For example, the transfer plasmid, Rep/Cap, and the helper plasmid can be transfected into HEK293 cells containing the adenovirus gene E1+ to produce infectious AAV particles. Alternatively, the Rep, Cap, and adenovirus helper genes may be combined into a single plasmid. Similar packaging cells and methods can be used for other viruses, such as retroviruses.

Multiple serotypes of AAV have been identified. These serotypes differ in the types of cells they infect (i.e., their tropism), allowing preferential transduction of specific cell types. Serotypes for CNS tissue include AAV1, AAV2, AAV4, AAV5, AAV8, and AAV9. Serotypes for heart tissue include AAV1, AAV8, and AAV9. Serotypes for kidney tissue include AAV2. Serotypes for lung tissue include AAV4, AAV5, AAV6, and AAV9. Serotypes for pancreas tissue include AAV8. Serotypes for photoreceptor cells include AAV2, AAV5, and AAV8. Serotypes for retinal pigment epithelium tissue include AAV1, AAV2, AAV4, AAV5, and AAV8. Serotypes for skeletal muscle tissue include AAV1, AAV6, AAV7, AAV8, and AAV9. Serotypes for liver tissue include AAV7, AAV8, and AAV9, and particularly AAV8.

Tropism can be further refined through pseudotyping, which is the mixing of a capsid and a genome from different viral serotypes. For example AAV2/5 indicates a virus containing the genome of serotype 2 packaged in the capsid from serotype 5. Use of pseudotyped viruses can improve transduction efficiency, as well as alter tropism. Hybrid capsids derived from different serotypes can also be used to alter viral tropism. For example, AAV-DJ contains a hybrid capsid from eight serotypes and displays high infectivity across a broad range of cell types in vivo. AAV-DJ8 is another example that displays the properties of AAV-DJ but with enhanced brain uptake. AAV serotypes can also be modified through mutations. Examples of mutational modifications of AAV2 include Y444F, Y500F, Y73OF, and S662V. Examples of mutational modifications of AAV3 include Y705F, Y731F, and T492V. Examples of mutational modifications of AAV6 include S663V and T492V. Other pseudotyped/modified AAV variants include AAV2/1, AAV2/6, AAV2/7, AAV2/8, AAV2/9, AAV2.5, AAV8.2, and AAV/SASTG. In a specific example, the AAV is AAV2/8 (AAV2 genome and rep proteins with AAV8 capsid proteins).

To accelerate transgene expression, self-complementary AAV (scAAV) variants can be used. Because AAV depends on the cell's DNA replication machinery to synthesize the complementary strand of the AAV's single-stranded DNA genome, transgene expression may be delayed. To address this delay, scAAV containing complementary sequences that are capable of spontaneously annealing upon infection can be used, eliminating the requirement for host cell DNA synthesis. However, single-stranded AAV (ssAAV) vectors can also be used.

To increase packaging capacity, longer transgenes may be split between two AAV transfer plasmids, the first with a 3′ splice donor and the second with a 5′ splice acceptor. Upon co-infection of a cell, these viruses form concatemers, are spliced together, and the full-length transgene can be expressed. Although this allows for longer transgene expression, expression is less efficient. Similar methods for increasing capacity utilize homologous recombination. For example, a transgene can be divided between two transfer plasmids but with substantial sequence overlap such that co-expression induces homologous recombination and expression of the full-length transgene.

In certain AAVs, the cargo can include a nuclease agent (or a nucleic acid encoding the nuclease agent or one or more nucleic acids encoding the nuclease agent). In certain AAVs, the cargo can include a guide RNA or a nucleic acid encoding a guide RNA. In certain AAVs, the cargo can include an mRNA encoding a Cas nuclease, such as Cas9, and a guide RNA or a nucleic acid encoding a guide RNA. In certain AAVs, the cargo can include an exogenous donor sequence. In certain AAVs, the cargo can include a nuclease agent (or a nucleic acid encoding the nuclease agent or one or more nucleic acids encoding the nuclease agent) and an exogenous donor sequence. In certain AAVs, the cargo can include an mRNA encoding a Cas nuclease, such as Cas9, a guide RNA or a nucleic acid encoding a guide RNA, and an exogenous donor sequence.

Introduction of nucleic acids and proteins can also be accomplished by lipid nanoparticle (LNP)-mediated delivery. For example, LNP-mediated delivery can be used to deliver a guide RNA in the form of RNA. In a specific example, the guide RNA and the Cas protein are each introduced in the form of RNA via LNP-mediated delivery in the same LNP. As discussed in more detail elsewhere herein, one or more of the RNAs can be modified to comprise one or more stabilizing end modifications at the 5′ end and/or the 3′ end. Such modifications can include, for example, one or more phosphorothioate linkages at the 5′ end and/or the 3′ end or one or more 2′-O-methyl modifications at the 5′ end and/or the 3′ end. Delivery through such methods results in transient presence of the guide RNA, and the biodegradable lipids improve clearance, improve tolerability, and decrease immunogenicity. Lipid formulations can protect biological molecules from degradation while improving their cellular uptake. Lipid nanoparticles are particles comprising a plurality of lipid molecules physically associated with each other by intermolecular forces. These include microspheres (including unilamellar and multilamellar vesicles, e.g., liposomes), a dispersed phase in an emulsion, micelles, or an internal phase in a suspension. Such lipid nanoparticles can be used to encapsulate one or more nucleic acids or proteins for delivery. Formulations which contain cationic lipids are useful for delivering polyanions such as nucleic acids. Other lipids that can be included are neutral lipids (i.e., uncharged or zwitterionic lipids), anionic lipids, helper lipids that enhance transfection, and stealth lipids that increase the length of time for which nanoparticles can exist in vivo. Examples of suitable cationic lipids, neutral lipids, anionic lipids, helper lipids, and stealth lipids can be found in WO 2016/010840 A1 and WO 2017/173054 A1, each of which is herein incorporated by reference in its entirety for all purposes. An exemplary lipid nanoparticle can comprise a cationic lipid and one or more other components. In one example, the other component can comprise a helper lipid such as cholesterol. In another example, the other components can comprise a helper lipid such as cholesterol and a neutral lipid such as DSPC. In another example, the other components can comprise a helper lipid such as cholesterol, an optional neutral lipid such as DSPC, and a stealth lipid such as S010, S024, S027, S031, or S033.

The LNP may contain one or more or all of the following: (i) a lipid for encapsulation and for endosomal escape; (ii) a neutral lipid for stabilization; (iii) a helper lipid for stabilization; and (iv) a stealth lipid. See, e.g., Finn et al. (2018) Cell Rep. 22 (9): 2227-2235 and WO 2017/173054 A1, each of which is herein incorporated by reference in its entirety for all purposes. In certain LNPs, the cargo can include a nuclease agent (or a nucleic acid encoding the nuclease agent or one or more nucleic acids encoding the nuclease agent). In certain LNPs, the cargo can include a guide RNA or a nucleic acid encoding a guide RNA. In certain LNPs, the cargo can include an mRNA encoding a Cas nuclease, such as Cas9, and a guide RNA or a nucleic acid encoding a guide RNA. In certain LNPs, the cargo can include an exogenous donor sequence. In certain LNPs, the cargo can include a nuclease agent (or a nucleic acid encoding the nuclease agent or one or more nucleic acids encoding the nuclease agent) and an exogenous donor sequence. In certain LNPs, the cargo can include an mRNA encoding a Cas nuclease, such as Cas9, a guide RNA or a nucleic acid encoding a guide RNA, and an exogenous donor sequence.

The lipid for encapsulation and endosomal escape can be a cationic lipid. The lipid can also be a biodegradable lipid, such as a biodegradable ionizable lipid. One example of a suitable lipid is Lipid A or LP01, which is (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl(9Z,12Z)-octadeca-9,12-dienoate. See, e.g., Finn et al. (2018) Cell Rep. 22 (9): 2227-2235 and WO 2017/173054 A1, each of which is herein incorporated by reference in its entirety for all purposes. Another example of a suitable lipid is Lipid B, which is ((5-((dimethylamino)methyl)-1,3-phenylene)bis(oxy))bis(octane-8,1-diyl)bis(decanoate), also called ((5-((dimethylamino)methyl)-1,3-phenylene)bis(oxy))bis(octane-8,1-diyl)bis(decanoate). Another example of a suitable lipid is Lipid C, which is 2-((4-(((3-(dimethylamino)propoxy)carbonyl)oxy) hexadecanoyl)oxy)propane-1,3-diyl(9Z,9′Z,12Z,12′Z)-bis(octadeca-9,12-dienoate). Another example of a suitable lipid is Lipid D, which is 3-(((3-(dimethylamino)propoxy)carbonyl)oxy)-13-(octanoyloxy)tridecyl 3-octylundecanoate. Other suitable lipids include heptatriaconta-6,9,28,31-tetraen-19-yl 4-(dimethylamino)butanoate (also known as [(6Z,9Z,28Z,31Z)-heptatriaconta-6,9,28,31-tetraen-19-yl] 4-(dimethylamino)butanoate or Dlin-MC3-DMA (MC3))).

Some such lipids suitable for use in the LNPs described herein are biodegradable in vivo. For example, LNPs comprising such a lipid include those where at least 75% of the lipid is cleared from the plasma within 8, 10, 12, 24, or 48 hours, or 3, 4, 5, 6, 7, or 10 days. As another example, at least 50% of the LNP is cleared from the plasma within 8, 10, 12, 24, or 48 hours, or 3, 4, 5, 6, 7, or 10 days.

Such lipids may be ionizable depending upon the pH of the medium they are in. For example, in a slightly acidic medium, the lipids may be protonated and thus bear a positive charge. Conversely, in a slightly basic medium, such as, for example, blood where pH is approximately 7.35, the lipids may not be protonated and thus bear no charge. In some embodiments, the lipids may be protonated at a pH of at least about 9, 9.5, or 10. The ability of such a lipid to bear a charge is related to its intrinsic pKa. For example, the lipid may, independently, have a pKa in the range of from about 5.8 to about 6.2.

Neutral lipids function to stabilize and improve processing of the LNPs. Examples of suitable neutral lipids include a variety of neutral, uncharged or zwitterionic lipids. Examples of neutral phospholipids suitable for use in the present disclosure include, but are not limited to, 5-heptadecylbenzene-1,3-diol (resorcinol), dipalmitoylphosphatidylcholine (DPPC), distearoylphosphatidylcholine or 1,2-distearoyl-sn-glycero-3-phosphocholine (DSPC), phosphocholine (DOPC), dimyristoylphosphatidylcholine (DMPC), phosphatidylcholine (PLPC), 1,2-diarachidonoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, dioleoyl phosphatidylethanolamine (DOPE), dilinoleoylphosphatidylcholine distearoylphosphatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine, 1-stearoyl-2-oleoyl-sn-glycero-3-phosphocholine (SOPC), and combinations thereof. For example, the neutral phospholipid may be selected from the group consisting of distearoylphosphatidylcholine (DSPC) and dimyristoyl phosphatidyl ethanolamine (DMPE).

Helper lipids include lipids that enhance transfection. The mechanism by which the helper lipid enhances transfection can include enhancing particle stability. In certain cases, the helper lipid can enhance membrane fusogenicity. Helper lipids include steroids, sterols, and alkyl resorcinols. Examples of suitable helper lipids suitable include cholesterol, 5-heptadecylresorcinol, and cholesterol hemisuccinate. In one example, the helper lipid may be cholesterol or cholesterol hemisuccinate.

Stealth lipids include lipids that alter the length of time the nanoparticles can exist in vivo. Stealth lipids may assist in the formulation process by, for example, reducing particle aggregation and controlling particle size. Stealth lipids may modulate pharmacokinetic properties of the LNP. Suitable stealth lipids include lipids having a hydrophilic head group linked to a lipid moiety.

The hydrophilic head group of stealth lipid can comprise, for example, a polymer moiety selected from polymers based on PEG (sometimes referred to as poly(ethylene oxide)), poly(oxazoline), poly(vinyl alcohol), poly(glycerol), poly(N-vinylpyrrolidone), polyaminoacids, and poly N-(2-hydroxypropyl)methacrylamide. The term PEG means any polyethylene glycol or other polyalkylene ether polymer. In certain LNP formulations, the PEG, is a PEG-2K, also termed PEG 2000, which has an average molecular weight of about 2,000 daltons. See, e.g., WO 2017/173054 A1, herein incorporated by reference in its entirety for all purposes.

The lipid moiety of the stealth lipid may be derived, for example, from diacylglycerol or diacylglycamide, including those comprising a dialkylglycerol or dialkylglycamide group having alkyl chain length independently comprising from about C4 to about C40 saturated or unsaturated carbon atoms, wherein the chain may comprise one or more functional groups such as, for example, an amide or ester. The dialkylglycerol or dialkylglycamide group can further comprise one or more substituted alkyl groups.

As one example, the stealth lipid may be selected from PEG-dilauroylglycerol, PEG-dimyristoylglycerol (PEG-DMG), PEG-dipalmitoylglycerol, PEG-distearoylglycerol (PEG-DSPE), PEG-dilaurylglycamide, PEG-dimyristylglycamide, PEG-dipalmitoylglycamide, and PEG-distearoylglycamide, PEG-cholesterol (1-[8′-(Cholest-5-en-3 [beta]-oxy)carboxamido-3′,6′-dioxaoctanyl]carbamoyl-[omega]-methyl-poly(ethylene glycol), PEG-DMB (3,4-ditetradecoxylbenzyl-[omega]-methyl-poly(ethylene glycol) ether), 1,2-dimyristoyl-sn-glycero-3-phosphoethanolamine-N-[methoxy (polyethylene glycol)-2000] (PEG2k-DMG), 1,2-distearoyl-sn-glycero-3-phosphoethanolamine-N-[methoxy (polyethylene glycol)-2000] (PEG2k-DSPE), 1,2-distearoyl-sn-glycerol, methoxypoly ethylene glycol (PEG2k-DSG), poly(ethylene glycol)-2000-dimethacrylate (PEG2k-DMA), and 1,2-distearyloxypropyl-3-amine-N-[methoxy (polyethylene glycol)-2000] (PEG2k-DSA). In one particular example, the stealth lipid may be PEG2k-DMG.

The LNPs can comprise different respective molar ratios of the component lipids in the formulation. The mol-% of the CCD lipid may be, for example, from about 30 mol-% to about 60 mol-%, from about 35 mol-% to about 55 mol-%, from about 40 mol-% to about 50 mol-%, from about 42 mol-% to about 47 mol-%, or about 45%. The mol-% of the helper lipid may be, for example, from about 30 mol-% to about 60 mol-%, from about 35 mol-% to about 55 mol-%, from about 40 mol-% to about 50 mol-%, from about 41 mol-% to about 46 mol-%, or about 44 mol-%. The mol-% of the neutral lipid may be, for example, from about 1 mol-% to about 20 mol-%, from about 5 mol-% to about 15 mol-%, from about 7 mol-% to about 12 mol-%, or about 9 mol-%. The mol-% of the stealth lipid may be, for example, from about 1 mol-% to about 10 mol-%, from about 1 mol-% to about 5 mol-%, from about 1 mol-% to about 3 mol-%, about 2 mol-%, or about 1 mol-%.

The LNPs can have different ratios between the positively charged amine groups of the biodegradable lipid (N) and the negatively charged phosphate groups (P) of the nucleic acid to be encapsulated. This may be mathematically represented by the equation N/P. For example, the N/P ratio may be from about 0.5 to about 100, from about 1 to about 50, from about 1 to about 25, from about 1 to about 10, from about 1 to about 7, from about 3 to about 5, from about 4 to about 5, about 4, about 4.5, or about 5.

In some LNPs, the cargo can comprise Cas mRNA (e.g., Cas9 mRNA) and gRNA. The Cas mRNA (e.g., Cas9 mRNA) and gRNAs can be in different ratios. For example, the LNP formulation can include a ratio of Cas mRNA (e.g., Cas9 mRNA) to gRNA nucleic acid ranging from about 25:1 to about 1:25, ranging from about 10:1 to about 1:10, ranging from about 5:1 to about 1:5, or about 1:1. Alternatively, the LNP formulation can include a ratio of Cas mRNA (e.g., Cas9 mRNA) to gRNA nucleic acid from about 1:1 to about 1:5, or about 10:1. Alternatively, the LNP formulation can include a ratio of Cas mRNA (e.g., Cas9 mRNA) to gRNA nucleic acid of about 1:10, 25:1, 10:1, 5:1, 3:1, 1:1, 1:3, 1:5, 1:10, or 1:25. Alternatively, the LNP formulation can include a ratio of Cas mRNA (e.g., Cas9 mRNA) to gRNA nucleic acid of from about 1:1 to about 1:2. In specific examples, the ratio of Cas mRNA (e.g., Cas9 mRNA) to gRNA can be about 1:1 or about 1:2.

In some LNPs, the cargo can comprise exogenous donor nucleic acid and gRNA. The exogenous donor nucleic acid and gRNAs can be in different ratios. For example, the LNP formulation can include a ratio of exogenous donor nucleic acid to gRNA nucleic acid ranging from about 25:1 to about 1:25, ranging from about 10:1 to about 1:10, ranging from about 5:1 to about 1:5, or about 1:1. Alternatively, the LNP formulation can include a ratio of exogenous donor nucleic acid to gRNA nucleic acid from about 1:1 to about 1:5, about 5:1 to about 1:1, about 10:1, or about 1:10. Alternatively, the LNP formulation can include a ratio of exogenous donor nucleic acid to gRNA nucleic acid of about 1:10, 25:1, 10:1, 5:1, 3:1, 1:1, 1:3, 1:5, 1:10, or 1:25.

A specific example of a suitable LNP has a nitrogen-to-phosphate (N/P) ratio of 4.5 and contains biodegradable cationic lipid, cholesterol, DSPC, and PEG2k-DMG in a 45:44:9:2 molar ratio. The biodegradable cationic lipid can be (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl(9Z,12Z)-octadeca-9,12-dienoate. See, e.g., Finn et al. (2018) Cell Rep. 22 (9): 2227-2235, herein incorporated by reference in its entirety for all purposes. The Cas9 mRNA can be in a 1:1 ratio by weight to the guide RNA. Another specific example of a suitable LNP contains Dlin-MC3-DMA (MC3), cholesterol, DSPC, and PEG-DMG in a 50:38.5:10:1.5 molar ratio.

Another specific example of a suitable LNP has a nitrogen-to-phosphate (N/P) ratio of 6 and contains biodegradable cationic lipid, cholesterol, DSPC, and PEG2k-DMG in a 50:38:9:3 molar ratio. The biodegradable cationic lipid can be (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl(9Z,12Z)-octadeca-9,12-dienoate. The Cas9 mRNA can be in a 1:2 ratio by weight to the guide RNA.

The mode of delivery can be selected to decrease immunogenicity. For example, a different components may be delivered by different modes (e.g., bi-modal delivery). These different modes may confer different pharmacodynamics or pharmacokinetic properties on the subject delivered molecule. For example, the different modes can result in different tissue distribution, different half-life, or different temporal distribution. Some modes of delivery (e.g., delivery of a nucleic acid vector that persists in a cell by autonomous replication or genomic integration) result in more persistent expression and presence of the molecule, whereas other modes of delivery are transient and less persistent (e.g., delivery of an RNA or a protein). Delivery of components in a more transient manner, for example as RNA, can ensure that the Cas/gRNA complex is only present and active for a short period of time and can reduce immunogenicity. Such transient delivery can also reduce the possibility of off-target modifications.

Administration in vivo can be by any suitable route including, for example, parenteral, intravenous, oral, subcutaneous, intra-arterial, intracranial, intrathecal, intraperitoneal, topical, intranasal, or intramuscular. Systemic modes of administration include, for example, oral and parenteral routes. Examples of parenteral routes include intravenous, intraarterial, intraosseous, intramuscular, intradermal, subcutaneous, intranasal, and intraperitoneal routes. A specific example is intravenous infusion. Local modes of administration include, for example, intrathecal, intracerebroventricular, intraparenchymal (e.g., localized intraparenchymal delivery to the striatum (e.g., into the caudate or into the putamen), cerebral cortex, precentral gyrus, hippocampus (e.g., into the dentate gyrus or CA3 region), temporal cortex, amygdala, frontal cortex, thalamus, cerebellum, medulla, hypothalamus, tectum, tegmentum, or substantia nigra), intraocular, intraorbital, subconjuctival, intravitreal, subretinal, and transscleral routes. Significantly smaller amounts of the components (compared with systemic approaches) may exert an effect when administered locally (for example, intraparenchymal or intravitreal) compared to when administered systemically (for example, intravenously). Local modes of administration may also reduce or eliminate the incidence of potentially toxic side effects that may occur when therapeutically effective amounts of a component are administered systemically.

A specific example is intravenous injection or infusion. Compositions comprising the nuclease agents or nucleic acids encoding the nuclease agents (e.g., Cas9 mRNAs and guide RNAs or nucleic acids encoding the guide RNAs) and/or exogenous donor nucleic acids can be formulated using one or more physiologically and pharmaceutically acceptable carriers, diluents, excipients or auxiliaries. The formulation can depend on the route of administration chosen. The term “pharmaceutically acceptable” means that the carrier, diluent, excipient, or auxiliary is compatible with the other ingredients of the formulation and not substantially deleterious to the recipient thereof.

The frequency of administration and the number of dosages can depend on the half-life of the exogenous donor nucleic acids or guide RNAs (or nucleic acids encoding the guide RNAs) and the route of administration among other factors. The introduction of nucleic acids or proteins into the cell or animal can be performed one time or multiple times over a period of time. For example, the introduction can be performed only once over a period of time, at least two times over a period of time, at least three times over a period of time, at least four times over a period of time, at least five times over a period of time, at least six times over a period of time, at least seven times over a period of time, at least eight times over a period of time, at least nine times over a period of times, at least ten times over a period of time, at least eleven times, at least twelve times over a period of time, at least thirteen times over a period of time, at least fourteen times over a period of time, at least fifteen times over a period of time, at least sixteen times over a period of time, at least seventeen times over a period of time, at least eighteen times over a period of time, at least nineteen times over a period of time, or at least twenty times over a period of time.

### E. Measuring Expression and Activity of Integrated Antigen-Binding Protein Coding Sequences In Vivo

The methods disclosed herein can further comprise assessing expression and/or activity of the inserted antigen-binding protein coding sequence. Various methods can be used to identify cells having a targeted genetic modification. The screening can comprise a quantitative assay for assessing modification of allele (MOA) of a parental chromosome. For example, the quantitative assay can be carried out via a quantitative PCR, such as a real-time PCR (qPCR). The real-time PCR can utilize a first primer set that recognizes the target locus and a second primer set that recognizes a non-targeted reference locus. The primer set can comprise a fluorescent probe that recognizes the amplified sequence. Other examples of suitable quantitative assays include fluorescence-mediated in situ hybridization (FISH), comparative genomic hybridization, isothermic DNA amplification, quantitative hybridization to an immobilized probe(s), INVADER® Probes, TAQMAN® Molecular Beacon probes, or ECLIPSE™ probe technology (see, e.g., US 2005/0144655, herein incorporated by reference in its entirety for all purposes).

Next-generation sequencing (NGS) can also be used for screening. Next-generation sequencing can also be referred to as “NGS” or “massively parallel sequencing” or “high throughput sequencing.” NGS can be used as a screening tool in addition to the MOA assays to define the exact nature of the targeted genetic modification and whether it is consistent across cell types or tissue types or organ types.

Assessing modification of the genomic locus or safe harbor locus in a non-human animal can be in any cell type from any tissue or organ. For example, the assessment can be in multiple cell types from the same tissue or organ or in cells from multiple locations within the tissue or organ. This can provide information about which cell types within a target tissue or organ are being targeted or which sections of a tissue or organ are being reached by the human-albumin-targeting reagent. As another example, the assessment can be in multiple types of tissue or in multiple organs. In methods in which a particular tissue, organ, or cell type is being targeted, this can provide information about how effectively that tissue or organ is being targeted and whether there are off-target effects in other tissues or organs.

Methods for measuring expression of antigen-binding proteins can include, for example, measuring antibody levels in plasma or serum from the animal. Such methods are well-known. Such methods can also comprise assessing expression of the antibody mRNA encoded by the exogenous donor nucleic acid or assessing expression of the antibody. This measuring can be within the liver or particular cell types or regions within the liver, or it can involve measuring serum levels of secreted antibody. Assays that can be done include, for example, ELISA for titer (hIgG), ELISA for binding to the target antigen, and western blot for antibody quality as described in Example 1 below.

One example of an assay that can be used are the RNASCOPE™ and BASESCOPE™ RNA in situ hybridization (ISH) assays, which are methods that can quantify cell-specific edited transcripts, including single nucleotide changes, in the context of intact fixed tissue. The BASESCOPE™ RNA ISH assay can complement NGS and qPCR in characterization of gene editing. Whereas NGS/qPCR can provide quantitative average values of wild type and edited sequences, they provide no information on heterogeneity or percentage of edited cells within a tissue. The BASESCOPE™ ISH assay can provide a landscape view of an entire tissue and quantification of wild type versus edited transcripts with single-cell resolution, where the actual number of cells within the target tissue containing the edited mRNA transcript can be quantified. The BASESCOPE™ assay achieves single-molecule RNA detection using paired oligo (“ZZ”) probes to amplify signal without non-specific background. However, the BASESCOPE™ probe design and signal amplification system enables single-molecule RNA detection with a ZZ probe, and it can differentially detect single nucleotide edits and mutations in intact fixed tissue.

Assays for measuring activity of an antigen-binding protein can include virus or bacteria neutralization assays if the antigen-binding protein is a neutralizing antigen-binding protein targeting a viral or bacterial antigen. Examples include plaque reduction neutralization tests (viral plaque assays) or focus-forming assays that employ immunostaining techniques using fluorescently labeled antibodies specific for a viral or bacterial antigen to detect infected host cells and infectious virus particles. Similar assays are well known. See, e.g., Shan et al. (2017) EBioMedicine 17:157-162 and Wilson et al. (2017) J. Clin. Microbiol. 55 (10): 3104-3112, each of which is herein incorporated by reference in its entirety for all purposes.

The activity of the antigen-binding protein can also be tested by exposing the animal to the virus or bacteria targeted by the antigen-binding protein and assessing whether the antigen-binding protein protects against infection. Similar tumor assay models could be used for antigen-binding proteins targeting cancer-associated antigens. Similar assays exist or could be developed for antigen-binding proteins targeting other disease-associated antigens.

### III. Prophylactic or Therapeutic Applications

The methods disclosed herein can be used for treating or effecting prophylaxis of a disease in an animal (human or non-human) having or at risk for the disease. An individual is at increased risk of a disease if the subject has at least one known risk-factor (e.g., genetic, biochemical, family history, situational exposure) placing individuals with that risk factor at a statistically significant greater risk of developing the disease than individuals without the risk factor.

For example, such methods can comprise introducing into the animal a nuclease agent (or a nucleic acid encoding the nuclease agent or one or more nucleic acids encoding the nuclease agent) that targets a target site in a genomic locus or safe harbor locus and an exogenous donor nucleic acid comprising an antigen-binding-protein coding sequence, wherein the antigen-binding protein targets an antigen associated with the disease. The nuclease agent can cleave the target site, and the antigen-binding protein coding sequence can be inserted into the genomic locus or safe harbor locus to produce a modified genomic locus or safe harbor locus. The antigen-binding protein can then be expressed in the animal and bind the antigen associated with the disease. Methods for inserting an antigen-binding-protein coding sequence into a genomic locus or safe harbor locus in an animal in vivo are discussed in more detail elsewhere herein.

An antigen-binding protein or antibody can be, for example, a therapeutic antigen-binding protein or antibody. Such antigen-binding proteins or antibodies can be used for neutralization or clearance of target proteins that cause disease or to selectively kill or clear disease-associated cells (e.g., cancer cells). Such antibodies can act via several different mechanisms of action, including, for example, neutralization, antibody-dependent cell-mediated cytotoxic (ADCC) activity, or complement-dependent cytotoxic (CDC) activity.

An antigen-binding protein or antibody can be, for example, a neutralizing antigen-binding protein or antibody or a broadly neutralizing antigen-binding protein or antibody. A neutralizing antibody is an antibody that defends a cell from an antigen or infectious body by neutralizing any effect it has biologically. Broadly-neutralizing antibodies (bNAbs) affect multiple strains of a particular bacteria or virus.

Disease-associated antigens are explained in more detail elsewhere herein. As a few examples, such antigens can be cancer-associated antigens, infectious-disease-associated antigens, bacterial antigens, or viral antigens. Examples of each are disclosed elsewhere herein.

IV. Cells or Animals or Genomes Comprising An Antigen-Binding-Protein Coding Sequence Inserted into a Safe Harbor Locus

Genomes, cells, and animals produced by the methods disclosed herein or comprising the antigen-binding-protein coding sequences in a genomic locus or safe harbor locus as described herein are also provided. Antigen-binding proteins and coding sequences that can be inserted are described in more detail elsewhere herein. Likewise, examples of genomic loci or safe harbor loci, such as the albumin locus, are described in more detail elsewhere herein. The genomic locus or safe harbor locus at which the antigen-binding-protein coding sequence is stably integrated can be heterozygous for the antigen-binding-protein coding sequence or homozygous for the antigen-binding-protein coding sequence. A diploid organism has two alleles at each genetic locus. Each pair of alleles represents the genotype of a specific genetic locus. Genotypes are described as homozygous if there are two identical alleles at a particular locus and as heterozygous if the two alleles differ. An animal comprising an antigen-binding-protein coding sequences in a genomic locus or safe harbor locus as described herein can comprise the antigen-binding-protein coding sequences in a genomic locus or safe harbor locus in its germline.

The genomes, cells, or animals provided herein can be, for example, eukaryotic, including, for example, animal, mammalian, non-human mammalian, and human. The term “animal” includes mammals, fishes, and birds. A mammal can be, for example, a non-human mammal, a human, a rodent, a rat, a mouse, or a hamster. Other non-human mammals include, for example, non-human primates, monkeys, apes, cats, dogs, rabbits, horses, bulls, deer, bison, livestock (e.g., bovine species such as cows, steer, and so forth; ovine species such as sheep, goats, and so forth; and porcine species such as pigs and boars). Birds include, for example, chickens, turkeys, ostrich, geese, ducks, and so forth. Domesticated animals and agricultural animals are also included. The term “non-human” excludes humans.

Cells can also be any type of undifferentiated or differentiated state. For example, a cell can be a totipotent cell, a pluripotent cell (e.g., a human pluripotent cell or a non-human pluripotent cell such as a mouse embryonic stem (ES) cell or a rat ES cell), or a non-pluripotent cell. Totipotent cells include undifferentiated cells that can give rise to any cell type, and pluripotent cells include undifferentiated cells that possess the ability to develop into more than one differentiated cell types.

The cells provided herein can also be germ cells (e.g., sperm or oocytes). The cells can be mitotically competent cells or mitotically-inactive cells, meiotically competent cells or meiotically-inactive cells. Similarly, the cells can also be primary somatic cells or cells that are not a primary somatic cell. Somatic cells include any cell that is not a gamete, germ cell, gametocyte, or undifferentiated stem cell. For example, the cells can be liver cells, kidney cells, hematopoietic cells, endothelial cells, epithelial cells, fibroblasts, mesenchymal cells, keratinocytes, blood cells, melanocytes, monocytes, mononuclear cells, monocytic precursors, B cells, erythroid-megakaryocytic cells, eosinophils, macrophages, T cells, islet beta cells, exocrine cells, pancreatic progenitors, endocrine progenitors, adipocytes, preadipocytes, neurons, glial cells, neural stem cells, neurons, hepatoblasts, hepatocytes, cardiomyocytes, skeletal myoblasts, smooth muscle cells, ductal cells, acinar cells, alpha cells, beta cells, delta cells, PP cells, cholangiocytes, white or brown adipocytes, or ocular cells (e.g., trabecular meshwork cells, retinal pigment epithelial cells, retinal microvascular endothelial cells, retinal pericyte cells, conjunctival epithelial cells, conjunctival fibroblasts, iris pigment epithelial cells, keratocytes, lens epithelial cells, non-pigment ciliary epithelial cells, ocular choroid fibroblasts, photoreceptor cells, ganglion cells, bipolar cells, horizontal cells, or amacrine cells). For example, the cells can be liver cells, such as hepatoblasts or hepatocytes.

The cells provided herein can be normal, healthy cells, or can be diseased or mutant-bearing cells.

The animals provided herein can be humans or they can be non-human animals. Non-human animals comprising a nucleic acid or expression cassette as described herein can be made by the methods described elsewhere herein. The term “animal” includes mammals, fishes, and birds. Mammals include, for example, humans, non-human primates, monkeys, apes, cats, dogs, horses, bulls, deer, bison, sheep, rabbits, rodents (e.g., mice, rats, hamsters, and guinea pigs), and livestock (e.g., bovine species such as cows and steer; ovine species such as sheep and goats; and porcine species such as pigs and boars). Birds include, for example, chickens, turkeys, ostrich, geese, and ducks. Domesticated animals and agricultural animals are also included. The term “non-human animal” excludes humans. Particular examples of non-human animals include rodents, such as mice and rats.

Non-human animals can be from any genetic background. For example, suitable mice can be from a 129 strain, a C57BL/6 strain, a mix of 129 and C57BL/6, a BALB/c strain, or a Swiss Webster strain. Examples of 129 strains include 129P1, 129P2, 129P3, 129X1, 129S1 (e.g., 129S1/SV, 129S1/Svlm), 129S2, 129S4, 129S5, 129S9/SvEvH, 129S6 (129/SvEvTac), 129S7, 129S8, 129T1, and 129T2. See, e.g., Festing et al. (1999) Mamm. Genome 10 (8): 836, herein incorporated by reference in its entirety for all purposes. Examples of C57BL strains include C57BL/A, C57BL/An, C57BL/GrFa, C57BL/Kal_wN, C57BL/6, C57BL/6J, C57BL/6ByJ, C57BL/6NJ, C57BL/10, C57BL/10ScSn, C57BL/10Cr, and C57BL/Ola. Suitable mice can also be from a mix of an aforementioned 129 strain and an aforementioned C57BL/6 strain (e.g., 50% 129 and 50% C57BL/6). Likewise, suitable mice can be from a mix of aforementioned 129 strains or a mix of aforementioned BL/6 strains (e.g., the 129S6 (129/SvEvTac) strain).

Similarly, rats can be from any rat strain, including, for example, an ACI rat strain, a Dark Agouti (DA) rat strain, a Wistar rat strain, a LEA rat strain, a Sprague Dawley (SD) rat strain, or a Fischer rat strain such as Fisher F344 or Fisher F6. Rats can also be obtained from a strain derived from a mix of two or more strains recited above. For example, a suitable rat can be from a DA strain or an ACI strain. The ACI rat strain is characterized as having black agouti, with white belly and feet and an RTI^{av1} haplotype. Such strains are available from a variety of sources including Harlan Laboratories. The Dark Agouti (DA) rat strain is characterized as having an agouti coat and an RTI^{av1} haplotype. Such rats are available from a variety of sources including Charles River and Harlan Laboratories. In some cases, suitable rats can be from an inbred rat strain. See, e.g., US 2014/0235933, herein incorporated by reference in its entirety for all purposes.

In some animals, expression of the antigen-binding protein in serum or plasma is at least about 500, 1000, 1500, 2000, 2500, 3000, 3500, 4000, 4500, 5000, 5500, 6000, 6500, 7000, 7500, 8000, 8500, 9000, 9500, 10000, 20000, 30000, 40000, 50000, 60000, 70000, 80000, 90000, 100000, 110000, 120000, 130000, or 140000, 150000, 200000, 250000, 300000, 350000, or 400000 ng/ml (i.e., at least about 0.5, 1, 1.5, 2, 2.5, 3, 3.5, 4, 4.5, 5, 5.5, 6, 6.5, 7, 7.5, 8, 8.5, 9, 9.5, 10, 20, 30, 40, 50, 60, 70, 80, 90, 100, 110, 120, 130, or 140, 150, 200, 250, 300, 350, or 400 μg/mL). For example, expression can be at least about 2500, 5000, 10000, 100000, or 400000 ng/ml (i.e., at least about 2.5, 5, 10, 100, or 400 μg/mL).

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the invention can be used in combination with any other unless specifically indicated otherwise. Although the present invention has been described in some detail by way of illustration and example for purposes of clarity and understanding, it will be apparent that certain changes and modifications may be practiced within the scope of the appended claims.

### BRIEF DESCRIPTION OF THE SEQUENCES

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. When a nucleotide sequence encoding an amino acid sequence is provided, it is understood that codon degenerate variants thereof that encode the same amino acid sequence are also provided. The amino acid sequences follow the standard convention of beginning at the amino terminus of the sequence and proceeding forward (i.e., from left to right in each line) to the carboxy terminus.

TABLE 2
Description of Sequences.

| SEQ | | |
|---|---|---|
| ID | | |
| NO | Type | Description |
| 1 | DNA | REGN4504 anti-Env (Zika) SA-LC-P2A-HC-pA |
| | | Donor Nucleic Acid (pAAV AlbSA REGN4504) |
| 2 | DNA | REGN4504 anti-Zika LC Nucleotide |
| 3 | Protein | REGN4504 anti-Zika LC Protein |
| 4 | DNA | REGN4504 anti-Zika HC Nucleotide |
| 5 | Protein | REGN4504 anti-Zika HC Protein |
| 6 | DNA | hU6 gRNA1 REGN4446 HC F2A Albss LC |
| 7 | DNA | hU6 gRNA1 REGN4446 HC P2A Albss LC |
| 8 | DNA | hU6 gRNA1 REGN4446 HC T2A Albss LC |
| 9 | DNA | hU6 gRNA1 REGN4446 HC T2A RORss LC |
| 10 | DNA | AAV CAST REGN4446 HC T2A LC |
| 11 | DNA | AAV CMV REGN4446 LC T2A HC |
| 12 | DNA | REGN4446 anti-Zika LC Nucleotide |
| 13 | Protein | REGN4446 anti-Zika LC Protein |
| 14 | DNA | REGN4446 anti-Zika HC Nucleotide |
| 15 | Protein | REGN4446 anti-Zika HC Protein |
| 16 | DNA | REGN3263 anti-HA SA-LC-P2A-HC-pA Donor |
| | | Nucleic Acid |
| 17 | DNA | REGN3263 anti-HA LC Nucleotide |
| 18 | Protein | REGN3263 anti-HA LC Protein |
| 19 | DNA | REGN3263 anti-HA HC Nucleotide |
| 20 | Protein | REGN3263 anti-HA HC Protein |
| 21 | DNA | AlbSA |
| 22 | DNA | Furin Cleavage Site (Nucleic Acid) |
| 23 | Protein | Furin Cleavage Site (Protein) |
| 24 | DNA | P2A Nucleic Acid |
| 25 | Protein | P2A Protein |
| 26 | DNA | F2A Nucleic Acid |
| 27 | Protein | F2A Protein |
| 28 | DNA | T2A Nucleic Acid |
| 29 | Protein | T2A Protein |
| 30 | Protein | E2A Protein |
| 31 | DNA | mRORss Nucleic Acid vi |
| 32 | DNA | mRORss Nucleic Acid v2 |
| 33 | Protein | mRORss Protein |
| 34 | DNA | mAlbss Nucleic Acid |
| 35 | Protein | mAlbss Protein |
| 36 | DNA | sWPRE |
| 37 | DNA | SV40 PolyA |
| 38 | DNA | tRNAGln |
| 39 | DNA | SerpinAP.Cas9 |
| 40 | DNA | EFs |
| 41 | DNA | SV40p |
| 42 | DNA | E2P |
| 43 | DNA | SerpinAP |
| 44 | DNA | E2 Enhancer |
| 45 | DNA | SerpinA Enhancer |
| 46 | DNA | P Core Promoter |
| 47 | DNA | tGln gRNA EFs Cas9 |
| 48 | DNA | tGln gRNA SV40 Cas9 |
| 49 | DNA | tGln gRNA E2P Cas9 |
| 50 | DNA | tGln gRNA SerpinAP Cas9 |
| 51 | RNA | crRNA Tail |
| 52 | RNA | tracrRNA |
| 53 | RNA | gRNA Scaffold v1 |
| 54 | RNA | gRNA Scaffold v2 |
| 55 | RNA | gRNA Scaffold v3 |
| 56 | RNA | gRNA Scaffold v4 |
| 57 | RNA | gRNA Scaffold v5 |
| 58 | DNA | Guide RNA Target Sequence Plus PAM v1 |
| 59 | DNA | Guide RNA Target Sequence Plus PAM v2 |
| 60 | DNA | Guide RNA Target Sequence Plus PAM v3 |
| 61 | DNA | Cas9 DNA |
| 62 | Protein | Cas9 Protein |
| 63 | DNA | Cas9 mRNA |
| 64 | Protein | REGN4504 anti-Zika LC CDR1 |
| 65 | Protein | REGN4504 anti-Zika LC CDR2 |
| 66 | Protein | REGN4504 anti-Zika LC CDR3 |
| 67 | Protein | REGN4504 anti-Zika HC CDR1 |
| 68 | Protein | REGN4504 anti-Zika HC CDR2 |
| 69 | Protein | REGN4504 anti-Zika HC CDR3 |
| 70 | Protein | REGN4446 anti-Zika LC CDR1 |
| 71 | Protein | REGN4446 anti-Zika LC CDR2 |
| 72 | Protein | REGN4446 anti-Zika LC CDR3 |
| 73 | Protein | REGN4446 anti-Zika HC CDR1 |
| 74 | Protein | REGN4446 anti-Zika HC CDR2 |
| 75 | Protein | REGN4446 anti-Zika HC CDR3 |
| 76 | Protein | REGN3263 anti-HA LC CDR1 |
| 77 | Protein | REGN3263 anti-HA LC CDR2 |
| 78 | Protein | REGN3263 anti-HA LC CDR3 |
| 79 | Protein | REGN3263 anti-HA HC CDR1 |
| 80 | Protein | REGN3263 anti-HA HC CDR2 |
| 81 | Protein | REGN3263 anti-HA HC CDR3 |
| 82 | DNA | AAV ITR Fwd Primer |
| 83 | DNA | AAV ITR Ref Primer |
| 84 | DNA | AAV ITR Probe |
| 85 | DNA | REGN4504 anti-Zika LC CDR1 |
| 86 | DNA | REGN4504 anti-Zika LC CDR2 |
| 87 | DNA | REGN4504 anti-Zika LC CDR3 |
| 88 | DNA | REGN4504 anti-Zika HC CDR1 |
| 89 | DNA | REGN4504 anti-Zika HC CDR2 |
| 90 | DNA | REGN4504 anti-Zika HC CDR3 |
| 91 | DNA | REGN4446 anti-Zika LC CDR1 |
| 92 | DNA | REGN4446 anti-Zika LC CDR2 |
| 93 | DNA | REGN4446 anti-Zika LC CDR3 |
| 94 | DNA | REGN4446 anti-Zika HC CDR1 |
| 95 | DNA | REGN4446 anti-Zika HC CDR2 |
| 96 | DNA | REGN4446 anti-Zika HC CDR3 |
| 97 | DNA | REGN3263 anti-HA LC CDR1 |
| 98 | DNA | REGN3263 anti-HA LC CDR2 |
| 99 | DNA | REGN3263 anti-HA LC CDR3 |
| 100 | DNA | REGN3263 anti-HA HC CDR1 |
| 101 | DNA | REGN3263 anti-HA HC CDR2 |
| 102 | DNA | REGN3263 anti-HA HC CDR3 |
| 103 | DNA | REGN4504 anti-Zika VL Nucleotide |
| 104 | Protein | REGN4504 anti-Zika VL Protein |
| 105 | DNA | REGN4504 anti-Zika VH Nucleotide |
| 106 | Protein | REGN4504 anti-Zika VH Protein |
| 107 | DNA | REGN4446 anti-Zika VL Nucleotide |
| 108 | Protein | REGN4446 anti-Zika VL Protein |
| 109 | DNA | REGN4446 anti-Zika VH Nucleotide |
| 110 | Protein | REGN4446 anti-Zika VH Protein |
| 111 | DNA | REGN3263 anti-HA VL Nucleotide |
| 112 | Protein | REGN3263 anti-HA VL Protein |
| 113 | DNA | REGN3263 anti-HA VH Nucleotide |
| 114 | Protein | REGN3263 anti-HA VH Protein |
| 115 | DNA | Coding Sequence for Integrated mAlbss-LC-P2A- |
| | | mRORss-HC REGN4504(including endogenous |
| | | mouse albumin exon 1) |
| 116 | DNA | Coding Sequence for Integrated mAlbss-HC-F2A- |
| | | Albss-LC REGN4446(including endogenous mouse |
| | | albumin exon 1) |
| 117 | DNA | Coding Sequence for Integrated mAlbss-HC-P2A- |
| | | Albss-LC REGN4446(including endogenous mouse |
| | | albumin exon 1) |
| 118 | DNA | Coding Sequence for Integrated mAlbss-HC-T2A- |
| | | Albss-LC REGN4446(including endogenous mouse |
| | | albumin exon 1) |
| 119 | DNA | Coding Sequence for Integrated mAlbss-HC-T2A- |
| | | RORss-LC REGN4446(including endogenous mouse |
| | | albumin exon 1) |
| 120 | DNA | Coding Sequence for Integrated mAlbss-LC-P2A-HC |
| | | REGN3263(including endogenous mouse albumin |
| | | exon 1) |
| 121 | RNA | tracrRNA v2 |
| 122 | RNA | tracrRNA v3 |
| 123 | RNA | gRNA Scaffold v6 |
| 124 | RNA | gRNA Scaffold v7 |
| 125 | DNA | H1H11829N2 anti-HA LC Nucleotide |
| 126 | Protein | H1H11829N2 anti-HA LC Protein |
| 127 | DNA | H1H11829N2 anti-HA HC Nucleotide |
| 128 | Protein | H1H11829N2 anti-HA HC Protein |
| 129 | Protein | H1H11829N2 anti-HA LC CDR1 |
| 130 | Protein | H1H11829N2 anti-HA LC CDR2 |
| 131 | Protein | H1H11829N2 anti-HA LC CDR3 |
| 132 | Protein | H1H11829N2 anti-HA HC CDR1 |
| 133 | Protein | H1H11829N2 anti-HA HC CDR2 |
| 134 | Protein | H1H11829N2 anti-HA HC CDR3 |
| 135 | DNA | H1H11829N2 anti-HA LC CDR1 |
| 136 | DNA | H1H11829N2 anti-HA LC CDR2 |
| 137 | DNA | H1H11829N2 anti-HA LC CDR3 |
| 138 | DNA | H1H11829N2 anti-HA HC CDR1 |
| 139 | DNA | H1H11829N2 anti-HA HC CDR2 |
| 140 | DNA | H1H11829N2 anti-HA HC CDR3 |
| 141 | DNA | H1H11829N2 anti-HA VL Nucleotide |
| 142 | Protein | H1H11829N2 anti-HA VL Protein |
| 143 | DNA | H1H11829N2 anti-HA VH Nucleotide |
| 144 | Protein | H1H11829N2 anti-HA VH Protein |
| 145 | DNA | H1H11829N2 anti-HA (LC_T2A_RORss_HC) |
| 146 | DNA | Coding Sequence for Integrated H1H11829N2 |
| | | anti-HA (LC_T2A_RORss_HC) |

### Examples

### Example 1. Insertion of Anti-Zika Antibody Genes into Mouse Albumin Locus

Lipid Nanoparticle and AAV-Mediated Antibody Insertion into Mouse Albumin Locus

The albumin gene locus is a safe and effective site for therapeutic gene insertion and expression. Combining the CRIPSR/Cas9 technology and safe AAV vector to knock in a prophylactic or therapeutic antibody gene into the albumin locus in the liver for long-term expression is an attractive therapeutic modality.

To knock in a prophylactic or therapeutic antibody gene into the albumin locus in the liver, we used lipid nanoparticles (LNPs) carrying Cas9 mRNA and gRNA targeting the first intron of the mouse albumin gene and AAV2/8 encoding antibody light chain and heavy chain joined by a self-cleavage peptide to insert antibody genes into the mouse albumin locus for antibody expression as shown in FIG. 1 and described in more detail below. AAV2/8 has the AAV2 genome and rep proteins combined with AAV8 capsid proteins. The heavy chain coding sequence comprised V_{H}, D_{H}, and J_{H} segments, and the light chain coding sequence comprised light chain V_{L} and light chain J_{L} gene segments.

The insertion strategy involved using lipid nanoparticles to deliver Cas9 mRNA and gRNA to the mouse liver to induce a double-strand break in the first intron of the mouse albumin gene. The albumin gene structure is suited for transgene targeting into intronic sequences because its first exon encodes a secretory peptide (signal peptide or signal sequence) that is cleaved from the final protein product. Thus, integration of a promoterless cassette bearing a splice acceptor and a therapeutic antibody transgene supported expression and secretion of the therapeutic antibody transgene. AAV2/8 encoding antibody light chain and heavy chain was then able to integrate into the double-strand break site through the non-homologous end joining (NHEJ) pathway, and the antibody gene were transcribed by the endogenous albumin promoter as shown in FIG. 1.

The AAV genome (pAAV-AlbSA-REGN4504; SEQ ID NO: 1) used in the experiment was flanked by two inverted terminal repeats (ITRs). The AAV included a splicing acceptor for the first intron of mouse albumin gene (AlbSA; SEQ ID NO: 21), a REGN4504 antibody light chain cDNA (4504LC; SEQ ID NO: 2 (nucleic acid) and SEQ ID NO: 3 (protein)) with two additional C bases to keep the sequence in the correct open reading frame, a furin cleavage site (SEQ ID NO: 22 (nucleic acid) and SEQ ID NO: 23 (protein)), a linker composed of GSG amino acids, a mouse Ror1 signal sequence (mRORss; SEQ ID NO: 31 or 32 (nucleic acid) and SEQ ID NO: 33 (protein)), a REGN4504 antibody heavy chain coding sequence (4504HC; SEQ ID NO: 4 (nucleic acid) and SEQ ID NO: 5 (protein)), a short form of woodchuck hepatitis virus posttranscriptional regulatory element (sWPRE; SEQ ID NO: 36), and SV40polyA (SV40polyA; SEQ ID NO: 37). The coding sequence for the donor construct integrated at the mouse albumin locus (including endogenous mouse albumin exon 1: mAlbss-LC-P2A-mRORss-HC REGN4504) is set forth in SEQ ID NO: 115.

In a first experiment, the AAV donor sequence was the AAV2/8 AlbSA 4504 anti-Env (Zika) antibody donor sequence set forth in SEQ ID NO: 1. The donor comprised an antibody light chain upstream of an antibody heavy chain linked by a P2A self-cleavage peptide. The sequence identifiers for the sequences are provided in Table 3 below.

TABLE 3
Anti-Zika Antibody Sequences (REGN 4504).

| | Protein | DNA |
|---|---|---|
| Sequence | SEQ ID NO | SEQ ID NO |
| Light Chain | 3 | 2 |
| Light Chain Variable Region | 104 | 103 |
| Light Chain CDR1 | 64 | 85 |
| Light Chain CDR2 | 65 | 86 |
| Light Chain CDR3 | 66 | 87 |
| Heavy Chain | 5 | 4 |
| Heavy Chain Variable Region | 106 | 105 |
| Heavy Chain CDR1 | 67 | 88 |
| Heavy Chain CDR2 | 68 | 89 |
| Heavy Chain CDR3 | 69 | 90 |

The lipid nanoparticles were designed to deliver two different versions of guide RNAs targeting intron 1 of the mouse albumin locus. The first version (gRNA 1 v1) was N-cap modified and comprised 2′-O-methyl analogs and 3′ phosphorothioate internucleotide linkages at the first three 5′ and 3′ terminal RNA residues. The second version (gRNA 1 v2) was modified such that all 2′OH groups that do not interact with the Cas9 protein are replaced with 2′-O-methyl analogs, and the tail region of the guide RNA, which has minimal interaction with Cas9, is modified with 5′ and 3′ phosphorothioate internucleotide linkages. Additionally, the DNA-targeting segment also has 2′-fluoro modifications on some bases.

The formulations of the lipid nanoparticles are provided in Table 4. The Cas9 mRNA (capped and including modified uridine) and gRNA (were included at a ratio of 1:1 by weight. The LNPs were formulated on NANOASSEMBLER™ Benchtop. The nanoparticles self-assembled in microfluidics chips.

TABLE 4
LNP Formulation.

| | Molar Ratio | Molecular Weight |
|---|---|---|
| Lipid | in Mixture | (g/mol) |
| Dlin-MC3-DMA | 50 | 642.09 |
| (MC3) | | |
| DSPC | 10 | 790.14 |
| Cholesterol | 38.5 | 386.65 |
| PEG-DMG | 1.5 | 2000 |

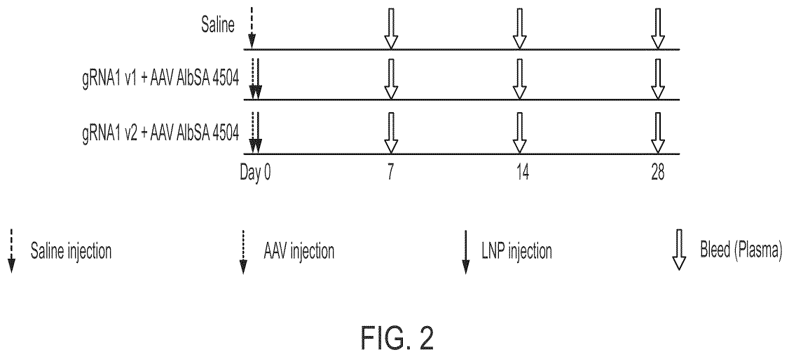

The experimental design is set forth in FIG. 2. Three C57BL/6 mice were used per group. Lipid nanoparticles (LNPs) were injected intravenously at a concentration of 1 mg/kg, and AAV AlbSA 4504 (3E11 vg/mouse) was co-injected on Day 0. Three groups were included in the experiment: (1) LNP delivering Cas9 mRNA and the first version of the guide RNA 1 v1 plus AAV2/8 AlbSA 4504; (2) LNP delivering Cas9 mRNA and the second version of the guide RNA 1 described above plus AAV2/8 AlbSA 4504; and (3) a saline negative control. As shown in FIG. 2, the LNP and AAV2/8 injections were on Day 0. Plasma bleeds were obtained at Days 7, 14, and 28 (i.e., Weeks 1, 2, and 4).

Adeno-associated virus production was performed using a triple transfection method with HEK293 cells. See, e.g., Arden and Metzger (2016) J. Biol. Methods 3 (2): e38, herein incorporated by reference in its entirety for all purposes. Cells were plated one day prior to PEFpro (Polyplus transfection, New York, NY)-mediated transfection with appropriate vectors, one helper plasmid, pHelper (Agilent, Cat #240074), one plasmid containing AAV rep/cap gene (pAAV RC2 (Cell biolabs, Cat #VPK-422), pAAV RC2/8 (Cell Biolabs, Cat #VPK-426), and one plasmid providing AAV ITR and transgene (pAAV-AlbSA-REGN4504; SEQ ID NO: 1). Seventy-two hours after transfection, media were collected and cells were lysed in buffer [50 mM Tris-HCl, 150 mM NaCl and 0.5% Sodium Deoxycholate (Sigma, Cat #D6750-100G)]. Next, benzonase (Sigma, St. Louis, MO) was added to both medium and cell lysate to a final concentration of 0.5 U/μL before incubation at 37° C. for 60 minutes. Cell lysate was spun down at 4000 rpm for 30 minutes. Cell lysate and medium were combined together and precipitated with PEG 8000 (Teknova Cat #P4340) at a final concentration of 8%. The pellet was resuspended in 400 mM NaCl and centrifuged at 10000 g for 10 minutes. Viruses in the supernatant were pelleted by ultracentrifugation at 149,000 g for 3 hours and titered by qPCR.

For qPCR to titrate AAV genomes, AAV samples were treated with DNaseI (Thermofisher Scientific, Cat #EN0525) at 37° C. for one hour and lysed using DNA extract All Reagents (Thermofisher Scientific Cat #4403319). Encapsidated viral genomes were quantified using a QuantStudio 3 Real-Time PCR System (Thermofisher Scientific) using primers directed to the AAV2 ITRs. The sequences of the AAV2 ITR primers were 5′-GGAACCCCTAGTGATGGAGTT-3′ (fwd ITR; SEQ ID NO: 82) and 5′-CGGCCTCAGTGAGCGA-3′ (rev ITR; SEQ ID NO: 83), derived the left internal inverted repeat (ITR) sequence from of the AAV and the right internal inverted repeat (ITR) sequence from of the AAV, respectively. The sequence of the AAV2 ITR probe was 5′-6-FAM-CACTCCCTCTCTGCGCGCTCG-TAMRA-3′ (SEQ ID NO: 84). See, e.g., Aurnhammer et al. (2012) Hum. Gene Ther. Methods 23 (1): 18-28, herein incorporated by reference in its entirety for all purposes. After a 95° C. activation step for 10 minutes, a two-step PCR cycle was performed at 95° C. for 15 seconds and 60° C. for 30 seconds for 40 cycles. The TAQMAN Universal PCR Master Mix (Thermofisher Scientific, Cat #4304437) was used in the qPCR. DNA plasmid (Agilent, Cat #240074) was used as standard to determine absolute titers.

An ELISA assay was performed to quantify the antibody titer in the sera. Black 96-well Maxisorp plates (ThermoFisher #437111) were coated with 1 μg/mL of AffiniPure Goat Anti-Human IgG Fc gamma fragment specific antibody (Jackson ImmunoResearch #109-005-098) overnight at 4° C. The plate was washed with KPL wash buffer (VWR #5151-0011) and then blocked with 3%-BSA blocking buffer (SeraCare #5140-0008) for 1 hour at room temperature. Plates were washed 4 times and then incubated with either purified REGN4504 (anti-Zika Ab) antibody as a standard or mouse sera at 1:3 serial dilutions after an initial dilution of 1:100 in 0.5%-BSA, 0.05% Tween-20 ADB solution (SeraCare #5140-0000, ThermoFisher #85114) for 1 hour at room temperature. Following incubation with standard antibody and sera, plates were washed 4 times and incubated with goat anti-Human IgG HRP antibody (ThermoFisher #31412) at 1:10,000 in ADB solution for 1 hour at room temperature. Finally, plates were washed 8 times and then developed using SuperSignal ELISA Pico Chemiluminescent Substrate (ThermoFisher #37070) followed by read out on a PerkinElmer 2030 Victor X3 Multilabel reader.

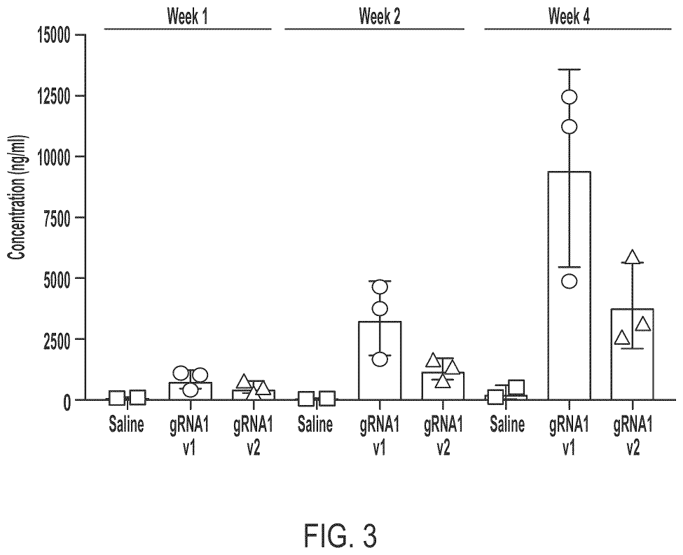

Co-injection of LNP and AAV resulted in around 1 μg/mL of antibody expression in mice inject with gRNA 1 v1 and 0.5 μg/mL of antibody expression in mice injected with gRNA1 v2 (FIG. 3). The antibody expression continued to increase to week 4. Co-injection of LNP with gRNA 1 v1 and AAV2/8-AlbSA-REGN4504 resulted in around 10 μg/mL antibody expression in week 4 and 5 μg/mL of antibody in mice injected with gRNA 1 v2 (FIG. 3). LNPs with the first guide RNA version (N-cap gRNAs) worked better than the second guide RNA version. Ten μg/mL of antibody in the serum reaches the therapeutic window for many diseases, such as infectious diseases. Antibody expressed from integrated AAV could protect mice from lethal infection by Zika, influenza, or other infectious disease agents.

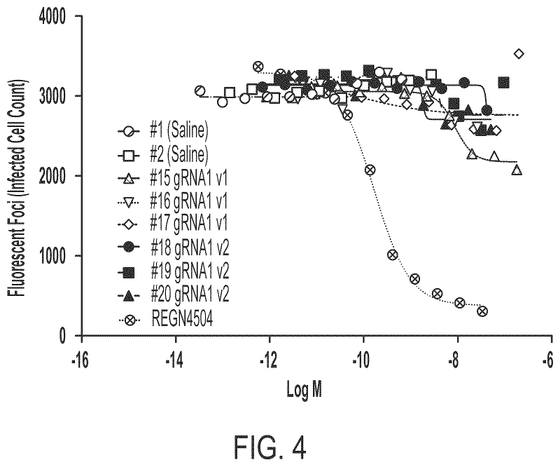

To determine if the antibody produced from integrated AAV was functional and had neutralizing activity against the Zika virus, a Zika neutralization assay was performed using plasma samples drawn four weeks after injection of the Cas9-gRNA LNP and the AAV2/8 AlbSA 4504 anti-Zika antibody donor sequence. Ten thousand Vero cells (Cat #CCL-81, ATCC, Manassas, VA) were plated per well in DMEM complete media (10% FBS, PSG) (Cat #10313-021, Life Technologies, Carlsbad, CA) in black, clear bottom 96-well cell culture treated plates (Cat #3904, Corning, Teterboro, NJ) and incubated at 37° C., 5% CO₂ one day before infection. Then 12 μL of serum was used as the starting point. Plasma was then diluted with DMEM at a 1:3 dilution factor, keeping the total volume 12 μL. Twelve μL of 2.0E+04 ffu/mL MR 766 virus (obtained from the UTMB Arbovirus Reference Collection) was incubated with plasma and added to the cells after 30 minutes of incubation. One day after infection, the cells were fixed with an ice cold 1:1 mix of methanol and acetone for 30 minutes at 4° C., permeabilized with PBS containing 5% FBS and 0.1% Triton-X for 15 minutes at room temperature, blocked with PBS+5% FBS for 30 minutes at room temperature, stained with primary antibody (Zika mouse immunized ascites fluid obtained from University of Texas Medical Branch at a 1:10,000 dilution in PBS+5% FBS) for 1 hour at room temperature, and incubated with secondary antibody (Alexa Fluor 488 Goat Anti-Mouse 1 μg/mL in PBS+5% FBS, Cat #A11001, ThermoFisher, Waltham, MA) for 1 hour at room temperature. The plates were then read on the Spectramax i3 (Cat #353701346, Molecular Devices) plate reader with MiniMax module. The antibodies in the mouse serum did not have neutralizing activity (FIG. 4).

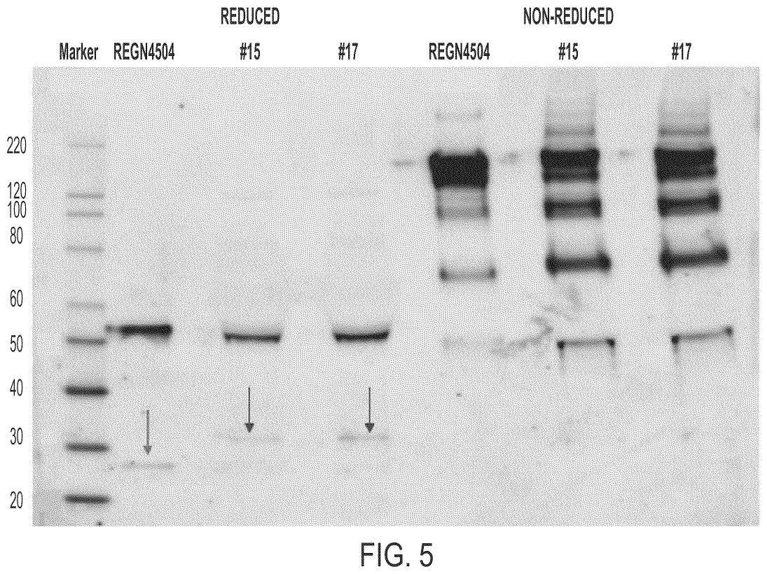

Western blots were used to assess the quality of the antibodies in the sera from the termination drawing. Briefly, 15 μg of sera was diluted in NuPAGE LDS Sample Buffer (ThermoFisher #NP0007) with and without NuPAGE Sample Reducing Agent (ThermoFisher #NP0009) and incubated at 70° C. for 10 minutes. Samples were then loaded onto NuPAGE 4-12% Bis-Tris Protein Gels (ThermoFisher #NP0321BOX) and run for roughly 35 minutes at 200V in NuPAGE MOPS SDS Run Buffer (ThermoFisher #NP0001). MagicMark Western Standard was used (ThermoFisher #LC5602) as a ladder, and REGN4504 (anti-Zika Ab) was used as a positive control for the gel. The gel was transferred to iBlot2 PVDF MiniStacks (ThermoFisher #IB24002) via the iBlot2 Dry Blotting System (ThermoFisher #IB21001). The membrane was blocked in 5% milk (VWR #M203-10G-10PK) in TBST (ThermoFisher #28360) for 1 hour at room temperature and then probed with goat anti-human IgG HRP antibody (ThermoFisher #31412) at 1:5,000 in PBS for 1 hour at room temperature. The blot was then developed using SuperSignal West Femto Maximum Sensitivity Substrate (ThermoFisher #34095) and then imaged on a BioRad ChemiDoc MP Imaging System. Western blotting showed that the light chain expression is abnormal and suggested that the light chain was improperly cleaved (FIG. 5).

Antibody Insertion into Mouse Albumin Locus in Cas9-Ready Mice

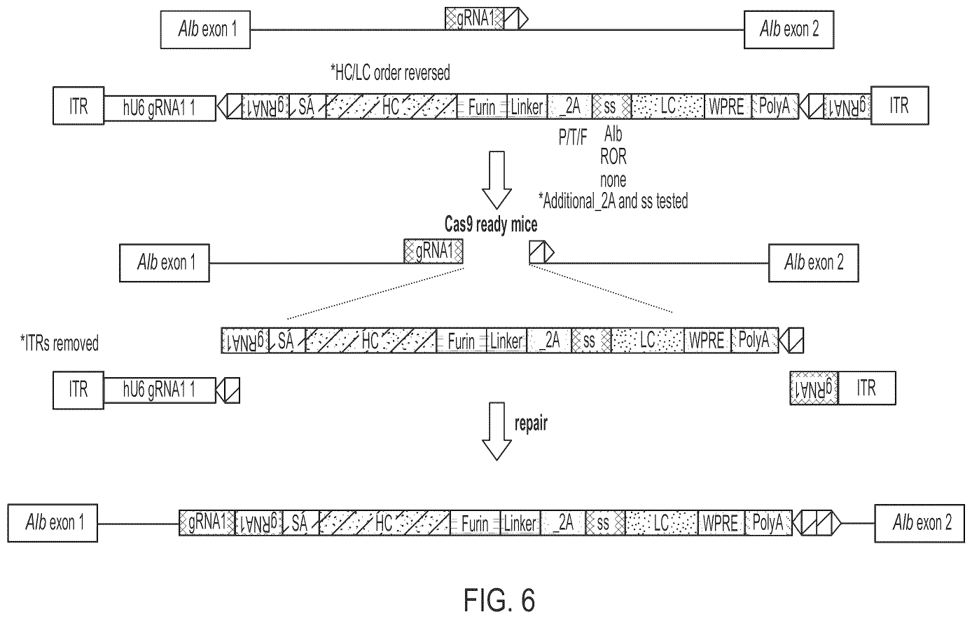

After the initial proof-of-concept experiment, a transgene was designed for homology-independent-targeted-insertion-mediated unidirectional targeted insertion of AAV-REGN4446 into the first intron of the mouse albumin gene in Cas9-ready mice (FIG. 6). The Cas9-ready mice, which have a Cas9-coding sequence integrated into the first intron of the Rosa26 locus of the mouse genome, are described in US 2019/0032155 and WO 2019/028032, each of which is herein incorporated by reference in its entirety.

In this strategy, the heavy-chain-encoding segment was upstream of the light-chain-encoding segment (FIG. 6), so the secretion of heavy chain was driven by endogenous albumin secretion signal. Different 2A peptides, F2A (SEQ ID NOS: 26 (nucleic acid) and 27 (protein)), P2A (SEQ ID NOS: 24 (nucleic acid) and 25 (protein)), and T2A (SEQ ID NOS: 28 (nucleic acid) and 29 (protein)), and both albumin (SEQ ID NOS: 34 (nucleic acid) and 35 (protein)) and mouse Ror1 signal sequence (SEQ ID NOS: 31 or 32 (nucleic acid) and 33 (protein)) were tested for driving light chain expression (FIG. 6). In addition, in contrast to the above experiment with REGN4504, the ITRs were removed. Four different insertion constructs ((1) AAV2/8. hU6 gRNA1. REGN4446 HC F2A Albss LC (SEQ ID NO: 6); (2) AAV2/8. hU6 gRNA1. REGN4446 HC P2A Albss LC (SEQ ID NO: 7); (3) AAV2/8. hU6 gRNA1. REGN4446 HC T2A Albss LC (SEQ ID NO: 8); and (4) AAV2/8. hU6 gRNA1. REGN4446 HC T2A RORss LC (SEQ ID NO: 9)) and two episomal antibody expression constructs ((5) AAV2/8. CMV. REGN4446 LC T2A HC (SEQ ID NO: 11) and (6) AAV2/8.CASI.REGN4446 HC T2A LC (SEQ ID NO: 10)) were injected into Cas9-ready mice (Table 5). The sequence identifiers for the sequences are provided in Table 6 below. The coding sequences for the donor constructs integrated at the mouse albumin locus (including endogenous mouse albumin exon 1: (1) mAlbss-HC-F2A-Albss-LC REGN4446; (2) mAlbss-HC-P2A-Albss-LC REGN4446; (3) mAlbss-HC-T2A-Albss-LC REGN4446; and (4) mAlbss-HC-T2A-RORss-LC REGN4446) are set forth in SEQ ID NOS: 116-119, respectively.

TABLE 5
Study Design for Comparison of Various REGN4446
Transgene Formats in Cas9-Ready Mice.

| Group | Virus | Vg/Mouse |
|---|---|---|
| 1 | Saline | — |
| 2 | AAV2/8.CMV.REGN4446 | 5.00E+11 |
| | RORss LC T2A RORss HC | |
| 3 | AAV2/8.CASI.REGN4446 | 5.00E+11 |
| | Albss HC T2A RORss LC | |
| 4 | AAV2/8.hU6 gRNA1v1 REGN4446 | 1.00E+12 |
| | HC F2A Albss LC | |
| 5 | AAV2/8.hU6 gRNA1v1 REGN4446 | 1.00E+12 |
| | HC P2A Albss LC | |
| 6 | AAV2/8.hU6 gRNA1v1 REGN4446 | 1.00E+12 |
| | HC T2A Albss LC | |
| 7 | AAV2/8.hU6 gRNA1v1 REGN4446 | 1.00E+12 |
| | HC T2A RORss LC | |

TABLE 6
REGN4446 Anti-Zika Antibody Sequences.

| | Protein | DNA |
|---|---|---|
| Sequence | SEQ ID NO | SEQ ID NO |
| Light Chain | 13 | 12 |
| Light Chain Variable Region | 108 | 107 |
| Light Chain CDR1 | 70 | 91 |
| Light Chain CDR2 | 71 | 92 |
| Light Chain CDR3 | 72 | 93 |
| Heavy Chain | 15 | 14 |
| Heavy Chain Variable Region | 110 | 109 |
| Heavy Chain CDR1 | 73 | 94 |
| Heavy Chain CDR2 | 74 | 95 |
| Heavy Chain CDR3 | 75 | 96 |

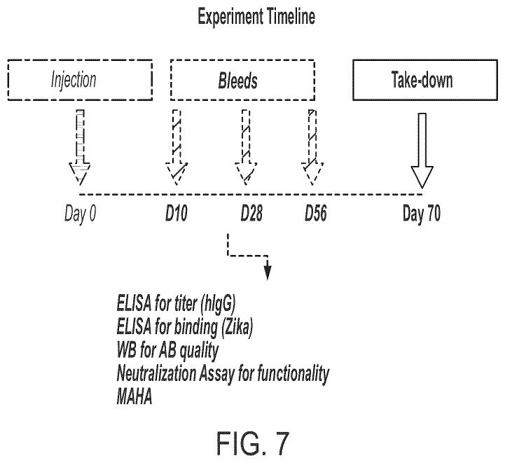

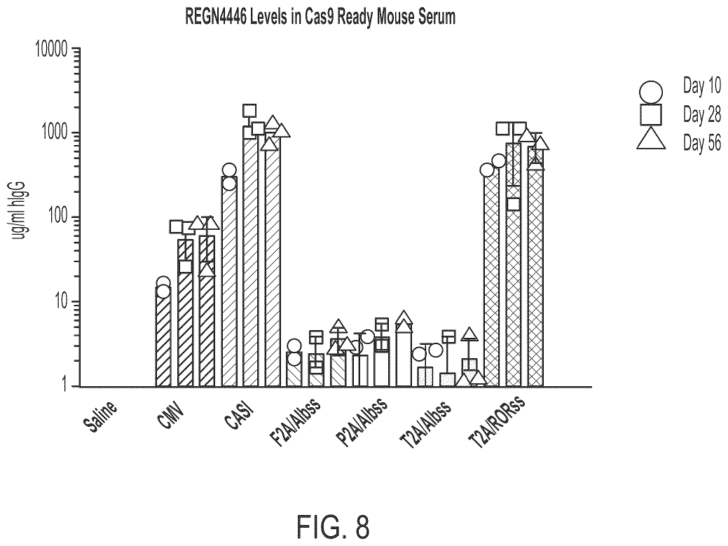

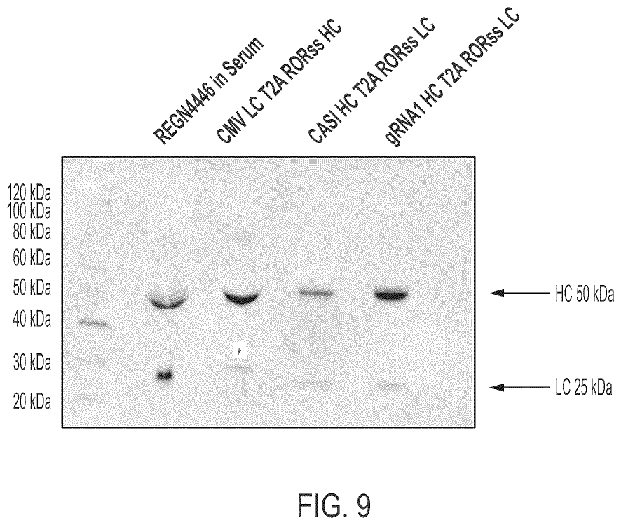

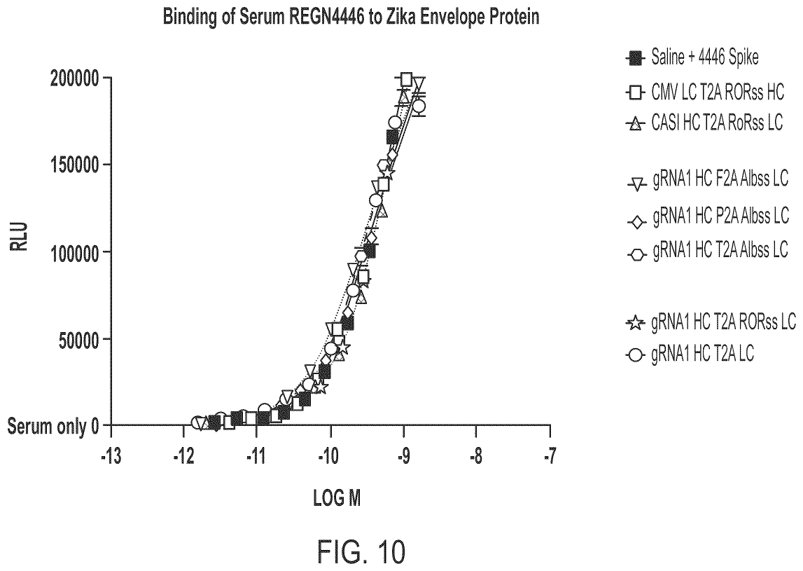

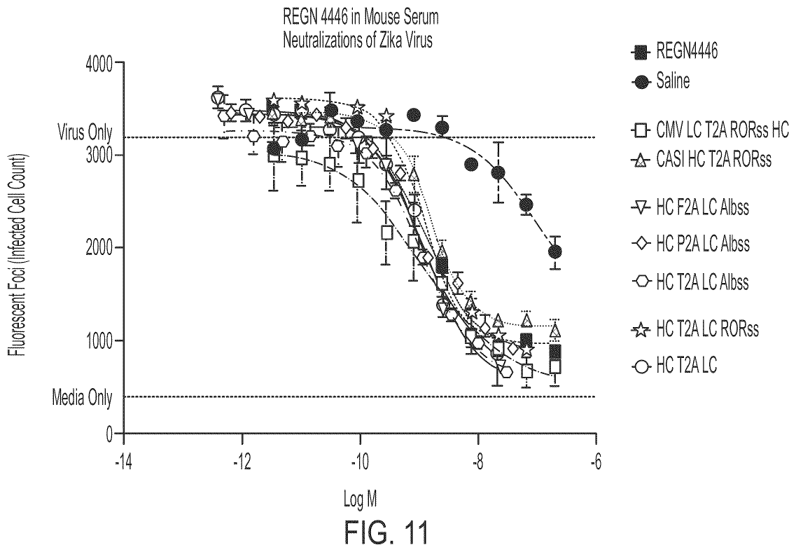

The experimental design is set forth in FIG. 7. Three male pRosa26@XbaI-loxP-Cas9-2A-eGFP (2600KO/3040WT) mice aged 7-11 weeks were used per group. AAV2/8 was injected on Day 0 (200 μL IV injection). As shown in FIG. 7, the AAV2/8 injections were on Day 0, and serum bleeds were obtained at Day 10, Day 28, or Day 56. Mice were taken down at Day 70 after injection for further analysis. Tests done following the serum bleeds included ELISA for titer (hIgG; FIG. 8), ELISA for binding (Zika; FIG. 10), western blot for antibody quality (FIG. 9), and neutralization assays for functionality (FIG. 11). Mouse anti-human antibody (MAHA) assays were also done (data not shown).

The episomal antibody expression constructs resulted in about 100 g/mL to 1000 μg/mL of antibody titers in mouse serum after Day 28. The inserted AAVs with albumin signal sequence before light chain resulted in around 5 μg/mL of antibody expression. Surprisingly, the integrated AAV with the mRor1 signal sequence before the light chain expressed around 1000 μg/mL antibody in mouse serum (FIG. 8). The titers using the ROR signal sequence upstream of the light chain were significantly higher than the titers using the albumin signal sequence upstream of the light chain. Western blotting showed the molecular weight of the heavy chain and the light chain of the antibody expressed from integrated AAV was similar to purified antibody (FIG. 9).

ELISA was used to measure the binding affinity of antibodies expressed from episomal AAV and integrated AAV. Zika (prM80E)-mmh (Lot #REGN4233-L4 May 12, 2016 PBSG 0.279 mg/mL) was incubated in Black 96-well Maxisorp plates (ThermoFisher #437111) overnight at 4° C. The plate was then washed with KPL wash buffer (VWR #5151-0011) and then blocked with 3%-BSA blocking buffer (SeraCare #5140-0008) for 1 hour at room temperature. Plates were washed 4 times and then incubated with either purified REGN4446 (anti-Zika Ab) antibody as a standard or mouse sera (from terminal blood draws) at 1:3 serial dilutions after an initial dilution of 1:100 in 0.5%-BSA, 0.05% Tween-20 ADB solution (SeraCare #5140-0000, ThermoFisher #85114) for 1 hour at room temperature. Following incubation with standard antibody and sera, plates were washed 4 times and incubated with goat anti-Human IgG HRP antibody (ThermoFisher #31412) at 1:10,000 in ADB solution for 1 hour at room temperature. Finally, plates were washed 8 times and then developed using SuperSignal ELISA Pico Chemiluminescent Substrate (ThermoFisher #37070) followed by read out on a PerkinElmer 2030 Victor X3 Multilabel reader. ELISA showed that the binding ability of the antibodies expressed from both episomal AAVs and integrated AAVs is comparable to purified REGN4446 (FIG. 10).

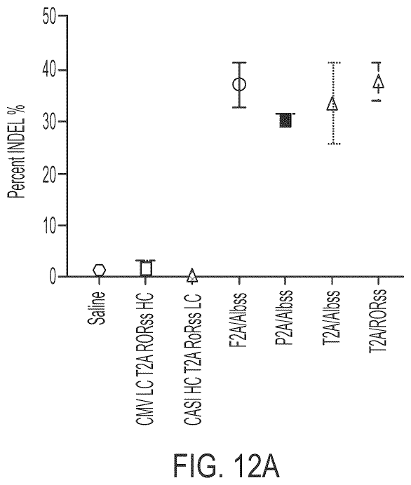

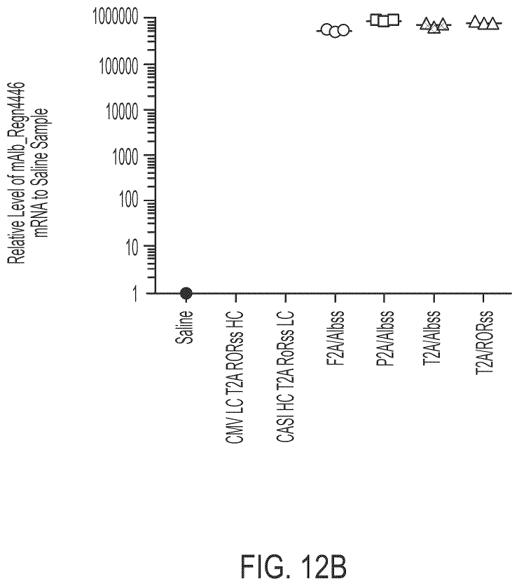

To determine if the antibodies produced by the mice were functional, a Zika neutralization assay was performed with sera from the terminal blood draws. The Zika neutralization assay (performed as described for FIG. 4) showed that the neutralizing activities of the antibodies expressed from both episomal AAVs and integrated AAVs was similar to purified REGN4446 (FIG. 11). NGS analysis of indels in mice sacrificed for tissue collection showed that the indel rates (caused by the Cas9/gRNA1 cutting in the first intron of the albumin gene) are similar among the mice injected with insertion constructs while mice injected with saline and episomal AAV had background levels of indel rates (FIG. 12A). TAQMAN qPCR with one primer binding to albumin exon 1 and one binding to antibody heavy chain showed that the mRNA levels of antibodies were similar, which indicated that the mRor1 signal sequence before the light chain promotes the antibody production more than 2 logs in mouse liver (FIG. 12B). Comparing the T2A/Albss and T2A/RORss, in which the only difference between the two constructs is the signal sequence upstream of the light chain coding sequence, it appears that the RORss dramatically promotes antibody secretion compared to the albumin signal sequence. Compare FIG. 8 with FIG. 12B.

Two-AAV-Mediated Antibody Insertion into Albumin Gene

As demonstrated above, insertion of antibody genes into intron 1 of the mouse albumin locus in Cas9-ready mice resulted in high level of antibody expression. In order to perform the insertion in non-Cas9-ready organisms, another AAV carrying a Cas9 expression cassette could be used. Because the cDNA of Cas9 (4.1 kb) is close to the packaging capacity of AAV), we first screened some small promoters that could fit into an AAV/Cas9 construct and drive Cas9 expression in the liver.

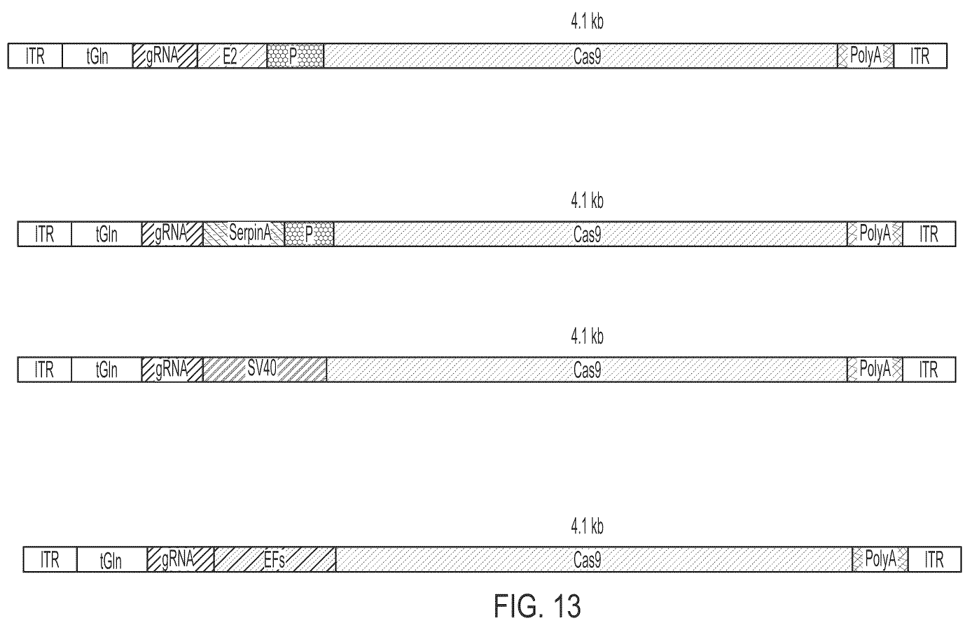

The small tRNAGIn promoter (SEQ ID NO: 38) was used to drive the expression of a guide RNA targeting Target Gene 1. Four promoters were tested for driving Cas9 expression: (1) elongation factor 1 alpha short (EFs) (SEQ ID NO: 40); (2) simian virus 40 (SV40) (SEQ ID NO: 41); and two synthetic promoters ((3) early region 2 promoter (E2P) (SEQ ID NO: 42) and (4) SerpinAP (SEQ ID NO: 43)). The synthetic promoters were composed of a liver-specific enhancer-E2, from HBV virus (SEQ ID NO: 44) or the SerpinA enhancer from the SerpinA gene (SEQ ID NO: 45)—and a core promoter (SEQ ID NO: 46) (FIG. 13).

1E12 VG of AAV2/8 viruses carrying tRNAGIn gRNA and Cas9 driven by four different promoters (tGln gRNA EFs Cas9 (SEQ ID NO: 47), tGln gRNA SV40 Cas9 (SEQ ID NO: 48), tGln gRNA E2P Cas9 (SEQ ID NO: 49), and tGln gRNA SerpinAP Cas9 (SEQ ID NO: 50)) were injected into mice. Five groups were tested: (1) saline control; (2) AAV2/8.tGln gRNA e2P Cas9; (3) AAV2/8.tGln gRNA SerpinAP Cas9; (4) AAV2/8.tGln gRNA Efs Cas9; and (5) AAV2/8.tGln gRNA SV40p Cas9.

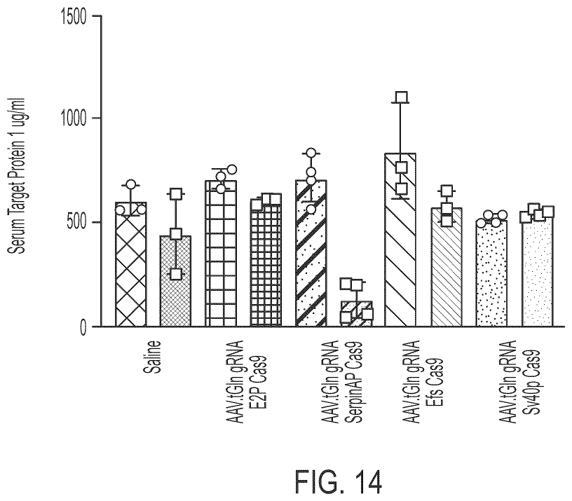

Five weeks later, serum was taken, and Target Protein 1 levels were analyzed by ELISA according to the manufacture's protocol (FIG. 14). The Target Protein 1 levels were knocked down in the mice injected with synthetic promoters, with the SerpinA promoter appearing to work best (FIG. 14).

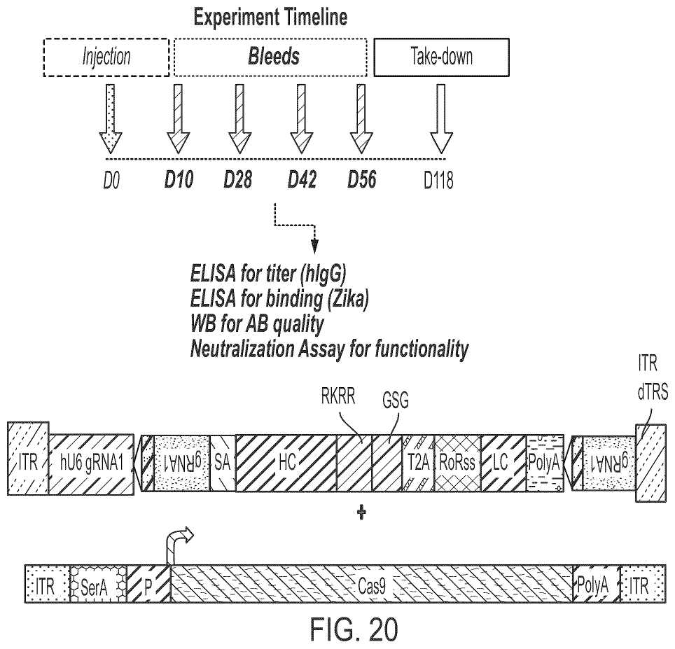

We next injected two AAVs, either 5E11 VG or 1E12 VG/mouse of AAV2/8.SerpinAP.Cas9 (SEQ ID NO: 39) and 1E12 VG/mouse of AAV2/8.hU6gRNA1.REGN4446 HC T2A mRORss LC (SEQ ID NO: 9) into 5-week-old female C57BL/6 mice or 8-week-old female BALB/c mice. Three mice were used per group. The experimental design is set forth in FIG. 20 and Table 7.

TABLE 7
Study Design.

| | | | | gRNA & |
|---|---|---|---|---|
| | | Episomal | Cas9 | REGN4446 |
| Group | Virus | VG/Mouse | VG/Mouse | VG/Mouse |
| 1 | Saline | — | — | — |
| 2 | AAV2/8.hU6.gRNA1. | — | — | 1.00E+12 |
| | REGN4446.HC.T2A. | | | |
| | RoRss.L | | | |
| 3 | AAV2/8.CASI.REGN4446. | 5.00E+11 | — | — |
| | HC.T2A.RoRss.LC_LOW | | | |
| 4 | AAV2/8.SerpinAP. | — | 5.00E+11 | 1.00E+12 |
| | mspCas9.SV40pA_LOW | | | |
| 5 | AAV2/8.SerpinAP. | — | 1.00E+12 | 1.00E+12 |
| | mspCas9.SV40pA_HIGH | | | |

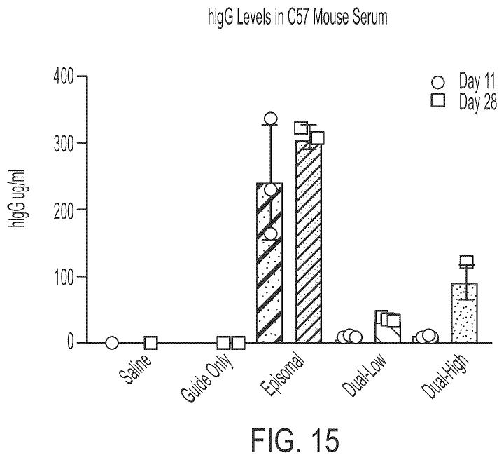

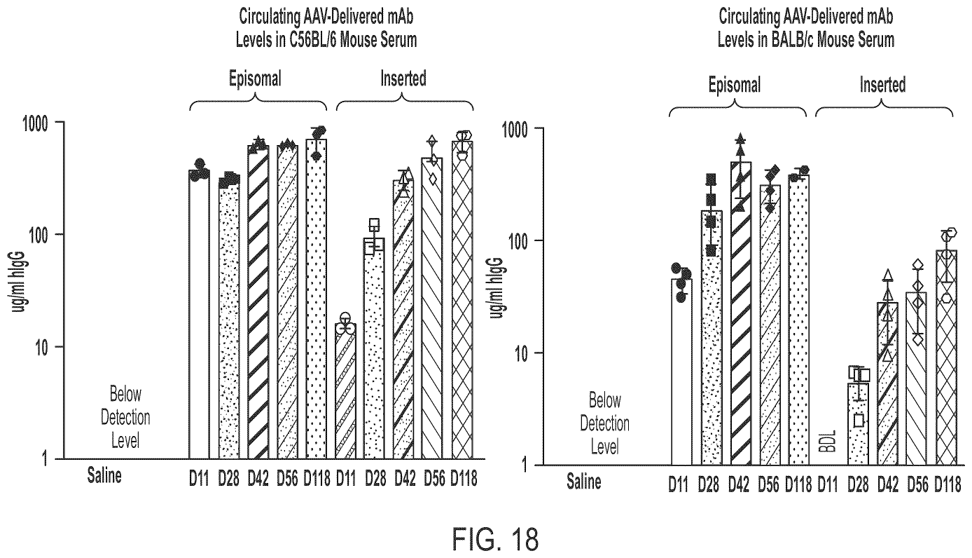

The gRNA1 coding sequence was included in the REGN4446 HC T2A mRORss LC AAV instead of the Cas9 AAV so that only cells infected by both AAVs would have indels and antibody gene insertion. Episomal AAV2/8.CASI.REGN4446 HC T2A LC (SEQ ID NO: 10) was used as a positive control. Four weeks after injection, the antibody expression level in groups with high titer of AAV2/8.SerpinAP.Cas9 was around 100 μg/mL, while the low titer group was around 50 g/mL in C57BL/6 mice (FIG. 15), while AAV2/8.hU6gRNA1v1.REGN4446 HC T2A mRORss LC injected mice (no Cas9 AAV injected) had no antibody expression. A time course with the high titer group was then extended out to 118 days for mice injected with AAV2/8.SerpinAP.Cas9 (SEQ ID NO: 39; 1E12 VG/mouse) and AAV2/8.hU6gRNA1.REGN4446 HC T2A mRORss LC (SEQ ID NO: 9; 1E12 VG/mouse) and for mice injected with episomal AAV2/8.CASI.REGN4446 (5E11 VG/mouse). Both C57BL/6 mice and BALB/c mice were used. At 118 days after injection, the antibody expression level in mice injected with AAV2/8.SerpinAP.Cas9 (SEQ ID NO: 39) and AAV2/8.hU6gRNA1.REGN4446 HC T2A mRORss LC (SEQ ID NO: 9) for integration was approaching 1000 μg/mL and was equivalent to the antibody expression level in the episomal AAV2/8.CASI.REGN4446 HC T2A LC (SEQ ID NO: 10) control group in C57BL/6 mice (FIG. 18, left panel). The same trend was also observed in BALB/c mice—a persistent increase in antibody (human IgG) levels was observed over the time course, approaching levels of expression in the episomal control group (FIG. 18, right panel)—showing that these results were not strain-specific.

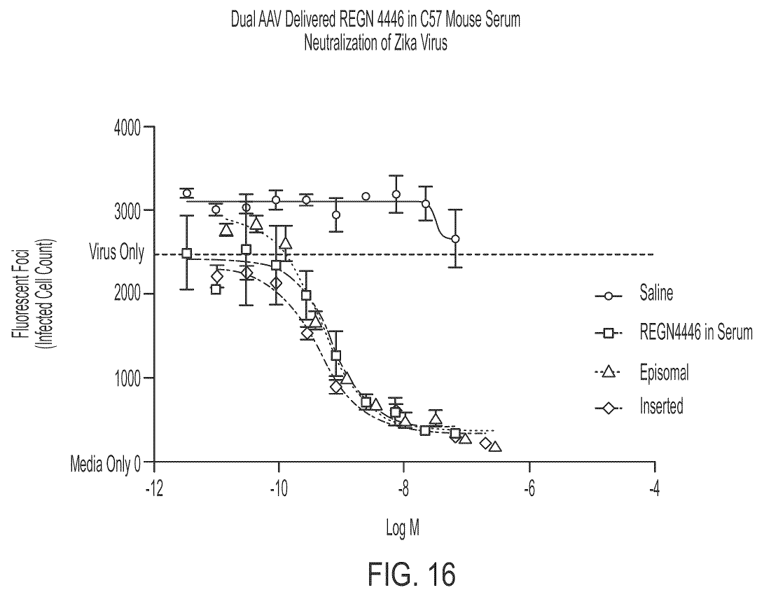

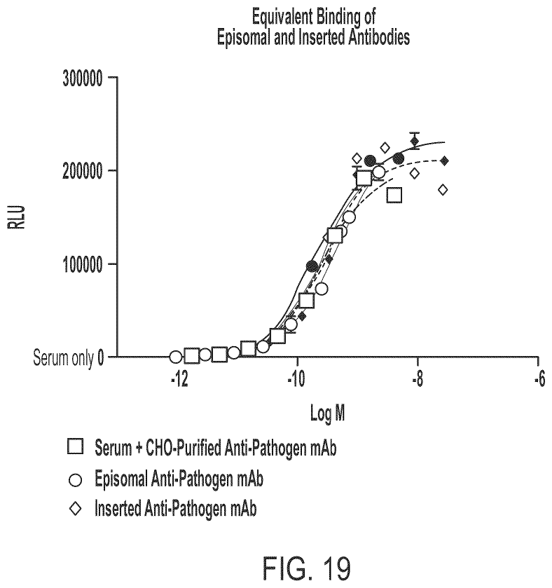

To determine if the antibodies produced by the mice were functional, a Zika neutralization assay was performed using serum from Day 28 from the high titer group in FIG. 15. The Zika neutralization assay (performed as described for FIG. 4) showed that the antibodies produced by this method neutralized Zika virus equally to purified REGN4446 (FIG. 16). In addition, the binding ability (binding to Zika envelope protein) was assessed as described above to compare binding of purified REGN4446 to antibody expressed from episomal AAV or following Cas9-mediated AAV integration. ELISA showed that the binding ability of the antibodies expressed from both episomal AAVs and integrated AAVs is comparable to purified REGN4446. See FIG. 19. Thus, monoclonal antibodies expressed via episome and insertion strategies were functionally equivalent to CHO-produced purified antibody as assessed both by binding assays and neutralization assays. A quantification of the binding and neutralization results is provided in Table 8 below.

TABLE 8
Episomal and Liver-Inserted Anti-Zika Monoclonal
Antibodies are Equivalent to CHO-Produced Purified
Antibody In Vitro and in Wild Type Mice.

| Transgene Format - Strain | Binding EC50 | Neutralization EC50 |
|---|---|---|
| Saline Serum + Purified | 2.53E−10 | 6.87E−10 |
| REGN4446 | | |
| Episomal - C57BL/6 | 2.96E−10 | 4.69E−10 |
| Episomal - BALB/c | 5.21E−10 | 6.05E−10 |
| Inserted - C57BL/6 | 3.10E−10 | 4.32E−10 |
| Inserted - BALB/c | 1.62E−10 | 8.49E−10 |

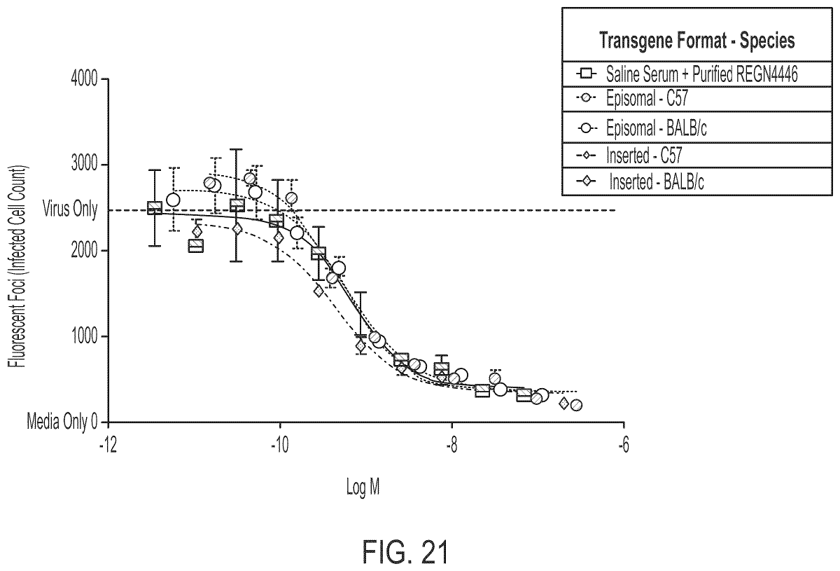

For the neutralization, Vero cells were seeded 1 day prior to infection at 10,000 cells/well in DMEM complete media (10% FBS, PSG) in black, clear-bottom, 96-well cell culture treated plates and incubated at 37° C., 5% CO₂ until the time of infection. On the day of infection, mouse serum samples were diluted in DMEM infection media (2% FBS, PSG) to two times their final neutralization reaction concentration. The serum was added to the media for a starting concentration of 12 μL serum per neutralization well (24 μL serum per dilution, which will yield 12 μL/serum in the final neutralization well when combined 1:1 with virus). The samples were then serially diluted 3-fold across a 96-well V-bottom microtiter plate for a total of 11 serum concentrations, ending with 0.0002 μL serum per neutralization well. The control antibody REGN4446 (Lot H4yH25703N) was also diluted in DMEM infection media to two times its final neutralization reaction concentration along with serum from a vehicle injected mouse, for a starting concentration of 5 μg/mL (3.33E-08 M, or 33.33 nM) in the neutralization reaction, and serially diluted 3-fold across a 96-well microtiter plate for a total of 11 dilutions ending with 0.00008 μg/mL (5.65E-13 M or 565 fM). Control wells were also prepared containing DMEM infection media or DMEM infection media mixed with the maximum volume of serum used in the assay, in order to allow for serum/media uninfected and infected controls. Virus was prepared by diluting MR766 virus (obtained from the UTMB Arbovirus Reference Collection and propagated in Vero cells to passage 3) from its stock concentration of 2.0E+06 ffu/mL in DMEM infection media to give a multiplicity of infection of 2 ffu/cell, or 20,000 ffu/neutralization well. Antibody and serum dilutions were combined 1:1 with the diluted virus in a V-bottom 96-well microtiter plate and incubated at 37° C., 5% CO₂ for 30 minutes. The virus/antibody/serum dilutions were then added to the cells. After the 1 hour incubation, the inoculum was removed, and the cells were overlaid with 100 μL DMEM+1% FBS, PSG, 1% methyl cellulose and incubated overnight (16-20 hours) at 37° C., 5% CO₂. The methyl cellulose overlay was aspirated off the cells and they were washed twice with PBS. The cells were then fixed, stained, and quantified following the protocol outlined for FIG. 4. The results are shown in FIG. 21, which shows equivalent neutralization by episomal and liver-inserted anti-Zika antibodies in serum from AAV-injected mice. The episomal and liver-inserted anti-Zika monoclonal antibodies in serum of both C57BL/6 and BALB/c mice were functionally equivalent to CHO-purified antibody spiked into naïve mouse serum.

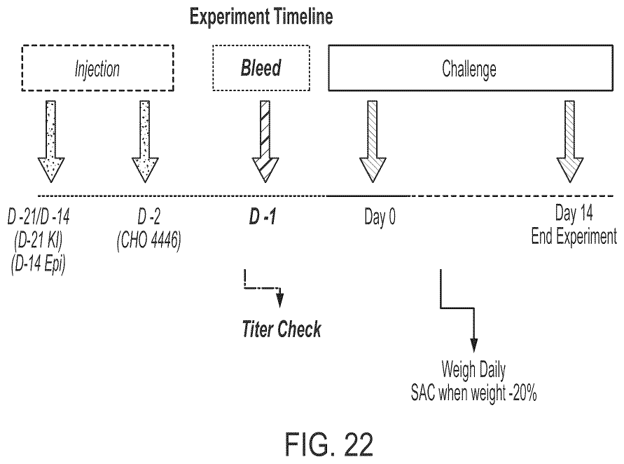

To test the functionality of the monoclonal antibodies produced from either the episomal or dual AAV insertion strategies, an in vivo Zika challenge model was employed. See FIG. 22. Female interferon alpha and beta receptor 1 knockout mice (IFNAR) between 10 and 11 weeks old were divided into 7 groups of N=4 mice. The groups received either an injection of (1) PBS; (2) AAV2/8 to episomally express an off-target control antibody driven by a CAG promoter; a (3) low dose (1.0E+11 VG/mouse) or a (4) high dose (5.0E+11 VG/mouse) of AAV2/8.CASI.REGN4446 HC T2A LC (SEQ ID NO: 10) to episomally express the REGN4446 anti-Zika antibody; a (5) low dose (5.0E+11 VG/mouse/vector) or a (6) high dose (1.0E+12 VG/mouse/vector) of both AAV2/8.SerpinAP.Cas9 (SEQ ID NO: 39) and AAV2/8.hU6gRNA1.REGN4446 HC T2A mRORss LC (SEQ ID NO: 9; 1E12 VG/mouse) for liver-inserted expression of REGN4446 anti-Zika antibody; or (7) 200 μg of CHO-purified REGN4446 anti-Zika antibody. Groups (1)-(6) were injected intravenously via tail vein injection. Groups (5) and (6) were injected 21 days prior to the start of the challenge. Groups (1)-(4) were injected 14 days prior to challenge. Group (7) was injected subcutaneously 2 days prior to challenge. One day prior to challenge, all mice were bled retro-orbitally and serum was collected in order to run a human FC ELISA and determine circulating titers of human monoclonal antibody (either off-target control or REGN4446) in each mouse. Mice were weighed pre-challenge and then infected with 10⁵ ffu FSS13025 virus intraperitoneally. Mice were then weighed every 24 hours for up to 14 days post Zika virus delivery. Mice were sacrificed once weight loss reached >20% of challenge day weight. All remaining mice were sacrificed day 14.

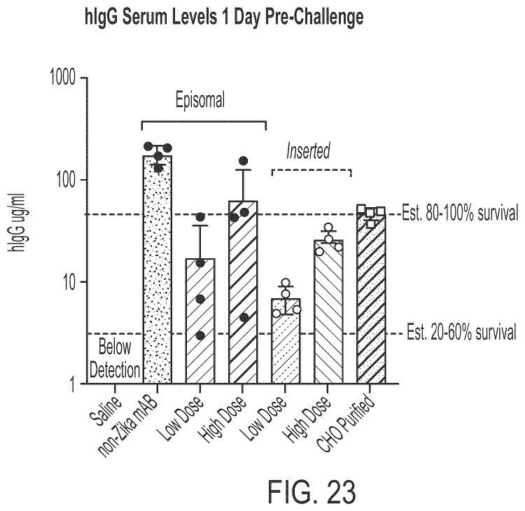

FIG. 23 shows the titer of hIgG detected by FC ELISA in each animal one-day pre-challenge. The height of each bar is the average titer per group with each point representing the titer for an individual animal within that group. The same FC ELISA protocol outlined for FIG. 3 was used with serum collected from each mouse. Estimated survival is plotted in the dotted lines based on previous challenge experiments using CHO-purified REGN4504 or REGN4446 anti-Zika antibodies. Episomal and PBS injections were performed 14 days prior to challenge, and inserted (dual AAV) were performed 21 days prior to challenge. The CHO-purified group was injected with 200 μg of REGN4446 two days prior to challenge.

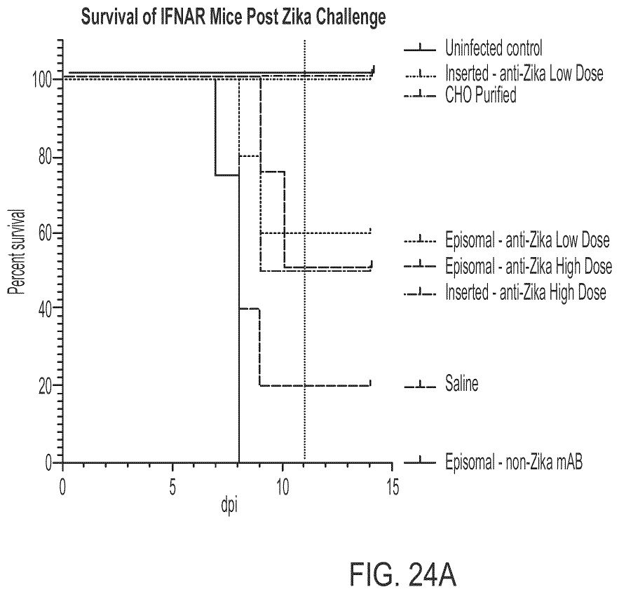

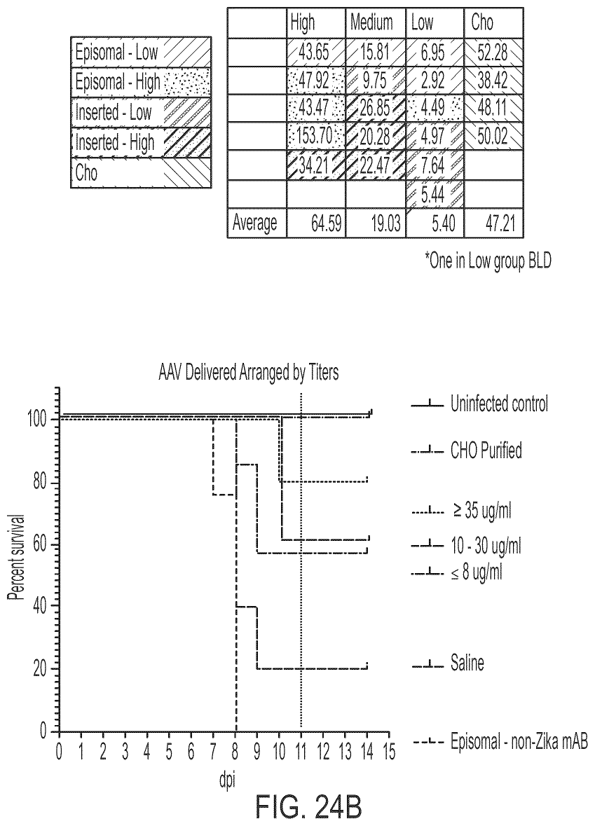

FIG. 24A shows the survival data results with animals grouped by VG/mouse delivered. As shown in FIG. 23, with each dose group there is high variability in the amount of circulating mAB measured 1 day prior to challenge, especially in the episomal groups. In addition there were four mice per group. Therefore, another way to look at the data is to group the mice by amount of circulating mAB at the time of challenge instead of by the type of AAV delivery and dose, which is shown in FIG. 24B. FIG. 24B shows the data from FIG. 24A rearranged so animals are grouped by titer of circulating AAV-delivered REGN4446 regardless of whether it was delivered by episome or dual AAV strategy at high or low dose. The values in the table in the top part of FIG. 24B are the levels of mAB measured 1 day prior to challenge in μg/mL, and the coding is the type of AAV that delivered the mAB template (either single AAV for episomal expression or dual AAV for Cas9-mediated integration and a low or high dose for either). Although the dose response is obscured if the data are plotted and grouped by type of AAV delivered as in FIG. 24A, FIG. 24B shows that we generated functional mAB that shows a dose response to the challenge.

### Example 2. Insertion of Anti-Hemagglutinin Antibody or Anti-PcrV Antibody Genes into Mouse Albumin Locus

The same strategy is used to integrate and express anti-hemagglutinin (anti-HA; influenza) antibody or and anti-PcrV (Pseudomonas aeruginosa) antibody. See, e.g., WO 2016/100807, herein incorporated by reference in its entirety for all purposes. Tests are then performed to determine if the antibodies expressed from the albumin locus prevent infection in the mice.

In a first experiment, the AAV donor sequence was the AAV2/8 AlbSA 3263 anti-HA (influenza) antibody donor sequence set forth in SEQ ID NO: 16. The donor comprised an antibody light chain and an antibody heavy chain linked by a P2A self-cleavage peptide. The sequence identifiers for the sequences are provided in Table 9 below. See also WO 2016/100807 (H1H11729P), herein incorporated by reference in its entirety for all purposes. The coding sequence for the donor construct integrated at the mouse albumin locus (including endogenous mouse albumin exon 1: mAlbss-LC-P2A-HC REGN3263) is set forth in SEQ ID NO: 120.

TABLE 9
Anti-HA Antibody Sequences (REGN3263).

| | Protein | DNA |
|---|---|---|
| Sequence | SEQ ID NO | SEQ ID NO |
| Light Chain | 18 | 17 |
| Light Chain Variable Region | 112 | 111 |
| Light Chain CDR1 | 76 | 97 |
| Light Chain CDR2 | 77 | 98 |
| Light Chain CDR3 | 78 | 99 |
| Heavy Chain | 20 | 19 |
| Heavy Chain Variable Region | 114 | 113 |
| Heavy Chain CDR1 | 79 | 100 |
| Heavy Chain CDR2 | 80 | 101 |
| Heavy Chain CDR3 | 81 | 102 |

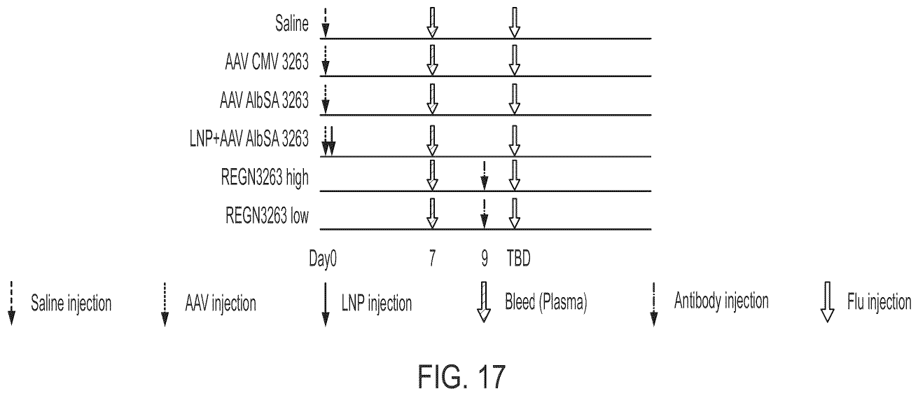

The experimental design for the first experiment (anti-HA) is set forth in FIG. 17. Five C57BL/6 mice are used per group. Lipid nanoparticles (LNPs) are injected at a concentration of 2 mg/kg, and AAV AlbSA 3263 (3E11) or AAV CMV 3263 (1E11) is injected on Day 0 without LNP or with co-injection of the LNP on Day 0. Six groups are included in the experiment: (1) LNP delivering Cas9 mRNA and gRNA 1 v1 plus AAV2/8 AlbSA 3263; (2) AAV2/8 AlbSA 3263 a1one; (3) AAV2/8 CMV 3263 a1one; (4) REGN3263 antibody injection (high dose); (5) REGN3263 antibody injection (low dose); and (6) a saline negative control. As shown in FIG. 17, the LNP and AAV2/8 injections are on Day 0, and the antibody injections (high dose and low dose positive controls) are on Day 9. Plasma bleeds are obtained at Day 7 (i.e., Week 1). Influenza virus is injected thereafter to test whether the antibodies expressed from the albumin locus prevent infection in the mice.

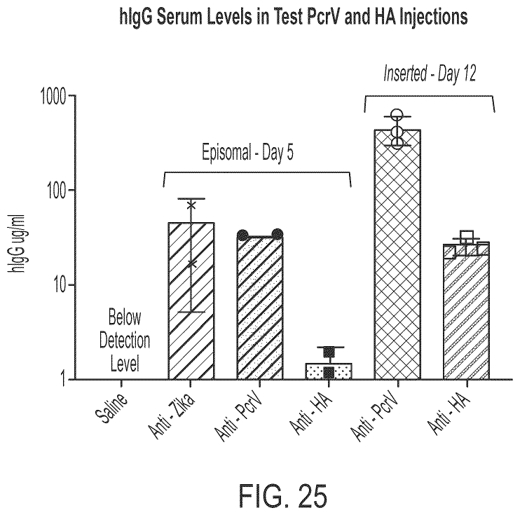

To demonstrate additional monoclonal antibodies being expressed using both the episomal and dual AAV strategies, C57BL/6 female mice (9 weeks old) were injected with one of 3 mABs in the AAV2/8 episomal format: (1) AAV2/8.CASI.REGN4446 HC T2A LC (SEQ ID NO: 10); (2) H1H29339P anti-PcrV (CAG promoter HC_T2A_RORss_LC); or (3) H1H11829N₂ anti-HA (CAG promoter LC_T2A_RORss_HC). REGN4446 is an IgG4 uber stealth format. See, e.g., U.S. Pat. No. 10,556,952, herein incorporated by reference in its entirety for all purposes. H1H29339P and H1H11829N₂ are IgG1 formats. The sequence identifiers for the H1H11829N₂ antibody sequences are provided in Table 10 below. See also WO 2016/100807, herein incorporated by reference in its entirety for all purposes. Virus was delivered at a dose of 1E12 VG/mouse via tail vein injection. Mice were bled retro-orbitally, and serum was collected for analysis at day 5, 20, and 30. Titers of circulating human IgG were measured using an FC ELISA. The same FC ELISA protocol outlined for FIG. 3 was used with serum collected from each mouse. Matching CHO-purified protein corresponding to each mAB was used to generate the standard curves for each set of serum samples independently. Only the values for the first timepoint are shown in FIG. 25.

TABLE 10
Anti-HA Antibody Sequences (H1H11829N2).

| | Protein | DNA |
|---|---|---|
| Sequence | SEQ ID NO | SEQ ID NO |
| Light Chain | 126 | 125 |
| Light Chain Variable Region | 142 | 141 |
| Light Chain CDR1 | 129 | 135 |
| Light Chain CDR2 | 130 | 136 |
| Light Chain CDR3 | 131 | 137 |
| Heavy Chain | 128 | 127 |
| Heavy Chain Variable Region | 144 | 143 |
| Heavy Chain CDR1 | 132 | 138 |
| Heavy Chain CDR2 | 133 | 139 |
| Heavy Chain CDR3 | 134 | 140 |

In addition, pRosa26@XbaI-loxP-Cas9-2A-eGFP female mice (22 weeks old) were injected with AAV2/8 carrying gRNA1 and one of two antibody expression cassettes: (1) H1H29339P anti-PcrV (HC_T2A_RORss_LC); or (2) H1H11829N₂ anti-HA (LC_T2A_RORss_HC) (SEQ ID NO: 145). Virus was delivered at a dose of 1E12 VG/mouse via tail vein injection. Mice were bled retro-orbitally, and serum was collected for analysis at day 12, 27, and 37. Titers of circulating human IgG were measured using an FC ELISA. The same FC ELISA protocol outlined for FIG. 3 was used with serum collected from each mouse. Matching CHO-purified protein corresponding to each mAB was used to generate the standard curves for each set of serum samples independently. Only the values for the first timepoint are shown in FIG. 25. The hIgG values as detected by a human FC ELISA for individual pRosa26@XbaI-loxP-Cas9-2A-eGFP female mice (22 weeks old) injected with AAV2/8 carrying gRNA1 and the H1H29339P anti-PcrV (HC_T2A_RORss_LC) expression cassette are displayed in Table 11. The data in FIG. 25 show that, like anti-Zika antibodies, anti-PcrV and anti-HA monoclonal antibodies can be expressed in vivo using AAV-mediated insertion strategies.

TABLE 11
hIgG Values.

| PcrV | Titer D12 | Titer D27 | Titer D37 |
|---|---|---|---|
| Sample | (μg/mL) | (μg/mL) | (μg/mL) |
| Inserted 1 | 412.65 | 602.74 | 1017.94 |
| Inserted 2 | 617.43 | 904.37 | 1081.30 |
| Inserted 3 | 308.00 | 408.60 | 1000.25 |

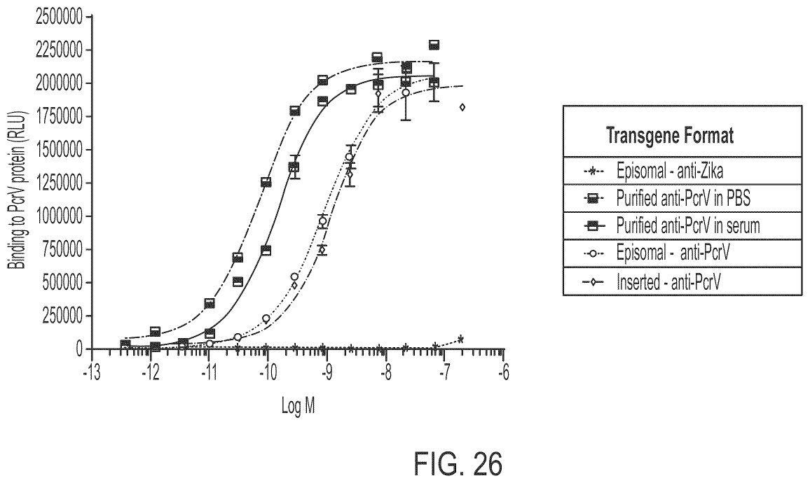

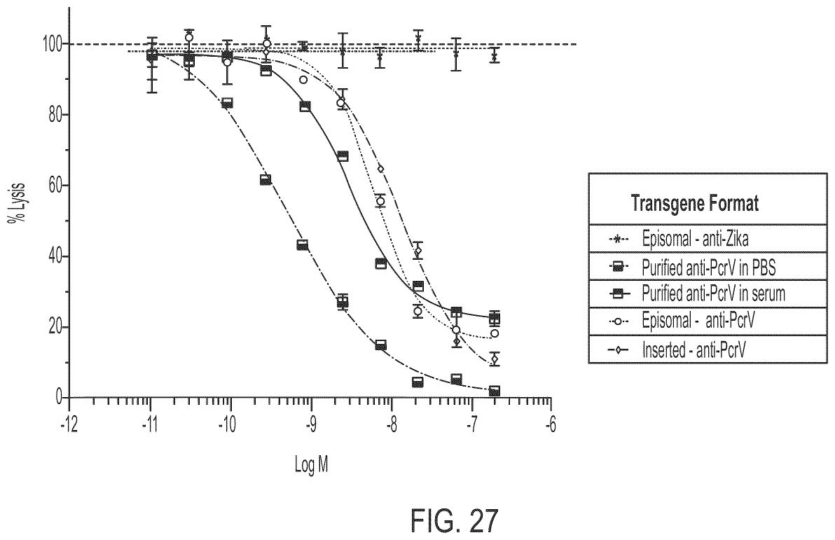

FIGS. 26 and 27, respectively, show the binding and neutralization/cytotoxicity data for serum H1H29339P anti-PcrV mAB from mice in the above described experiment. The samples included CHO-purified H1H29339P spiked into PBS, CHO-purified H1H29339P spiked into vehicle injected mouse serum, serum from a mouse injected with the episomal format of REGN4446 anti-Zika mAB AAV2/8.CASI.REGN4446 HC T2A LC (SEQ ID NO: 10), serum from a mouse injected with the episomal format of H1H29339P anti-PcrV mAB (CAG HC T2A RORss_LC), and serum from a mouse injected with the insertion format of H1H29339P anti-PcrV mAB (HC_T2A_RORss_LC). Episomal samples were from serum collected 5 days post-injection. Insertion sample was from serum collected 12 days post-injection. Episomal and liver-inserted anti-PcrV monoclonal antibodies appeared to be slightly less effective in binding and neutralization compared to CHO-produced purified antibody in vitro. FIG. 26 and Table 12 show binding of episomal and liver-inserted anti-PcrV monoclonal antibodies from mouse serum is slightly weaker than CHO-produced monoclonal antibodies. FIG. 27 and Table 12 show neutralization of episomal and liver-inserted anti-PcrV monoclonal antibodies from mouse serum is within 2-5 fold of CHO-produced monoclonal antibodies.

ELISA binding of anti-PcrV containing serum from AAV delivery to P. aeruginosa PcrV recombinant proteins (FIG. 26) was performed as follows: MicroSorp 96-well plates were coated with 0.2 μg per well of recombinant full-length P. aeruginosa PcrV (GenScript) and incubated overnight at 4° C. The following morning, plates were washed three times with wash buffer (Imidazole buffered saline with Tween-20) and blocked for 2 hours at 25° C. with 200 μL of blocking buffer (3% BSA in PBS). Plates were washed once and titrations of anti-PcrV antibody (ranging from 333 nM-0.1 μM with 1:3 serial dilutions in 0.5% BSA/0.05% Tween-20/PBS) or dilutions of serum (starting at 1:300 dilution with 1:3 serial dilutions in 0.5% BSA/0.05% Tween-20/PBS) were added to the protein-containing wells and incubated for one hour at 25° C. Wells were washed three times and then incubated with 100 ng/ml anti-human HRP secondary antibody per well for one hour at 25° C. 100 μL of SuperSignal ELISA Pico Chemiluminescent Substrate was added to each well and signal was detected (Victor X3 plate reader, Perkin Elmer). Luminescence values were analyzed by a four-parameter logistic equation over a 12-point response curve (GraphPad Prism).

The neutralization/cytotoxicity assay for FIG. 27 was performed as follows: A549 cells were seeded at a density of approximately 5×10⁵ cells per mL in Ham's F-12K (supplemented with 10% heat-inactivated FBS and L-glutamine) into 96-well clear bottom-black tissue culture treated plates and incubated overnight at 37° C. with 5% CO₂. The next day, media was removed from the cells and replaced with 100 μL assay medium (DMEM without phenol red, supplemented with 10% heat-inactivated FBS). Meanwhile, log phase culture of P. aeruginosa strains 6077 (Gerald Pier, Brigham and Women's Hospital, Harvard University) was prepared as follows: overnight P. aeruginosa culture was grown in LB, diluted 1:50 in fresh LB and grown to OD₆₀₀=~1 at 37° C. with shaking. Culture was washed once with assay media and diluted to OD₆₀₀=0.03 in PBS. Equal volume of bacteria in 50 μL was mixed with 50 μL of titrations of anti-PcrV antibody (ranging from 333 nM-17 μM with 1:3 serial dilutions) or dilutions of serum (starting at 1:100 dilution with 1:3 serial dilutions and incubated for 30-45 minutes at 25°. Media was removed from the A549 cells, replaced with 100 μL of bacteria:Ab mixes and incubated for two hours at 37° C. with 5% CO₂. Cell death was determined using the CytoTox-Glo™ Assay kit (Promega). Luminescence values were analyzed by a four-parameter logistic equation over a 10-point response curve (GraphPad Prism).

TABLE 12
Anti-PcrV mAB Binding and Neutralization.

| Transgene Format | Binding EC50 | Neutralization IC50 |
|---|---|---|
| Episomal - anti-Zika | 2.04E−07 | ~8.89E−12 |
| Purified anti-PcrV in PBS | 6.83E−11 | 5.15E−10 |
| Purified anti-PcrV in Serum | 1.40E−10 | 3.07E−09 |
| Episomal - anti-PcrV | 9.13E−10 | 6.48E−09 |
| Inserted - anti-PcrV | 1.18E−09 | 1.40E−08 |

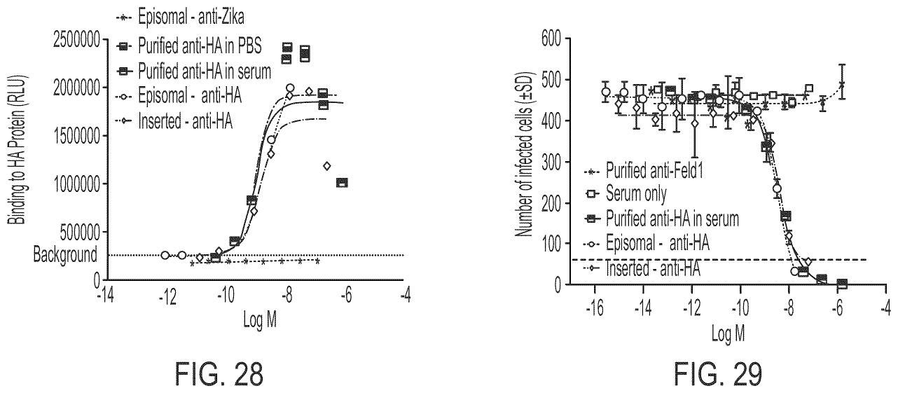

FIGS. 28 and 29, respectively, show the binding and neutralization data for serum H1H11829N₂ anti-HA mAB from mice in the above described experiment. The samples included CHO-purified H1H11829N₂ spiked into PBS, CHO-purified H1H11829N₂ spiked into vehicle injected mouse serum, serum from a mouse injected with the episomal format of REGN4446 anti-Zika mAB AAV2/8.CASI.REGN4446 HC T2A LC (SEQ ID NO: 10), serum from a mouse injected with the episomal format of H1H11829N₂ anti-HA mAB (CAG LC T2A RORss_HC), and serum from a mouse injected with the insertion format of H1H11829N₂ anti-HA mAB (LC_T2A_RORss_HC) (SEQ ID NO: 145). Episomal samples were from serum collected 5 days post-injection. Insertion sample was from serum collected 12 days post-injection. The isotype control is CHO-purified anti-FELD1. Episomal and liver-inserted anti-HA monoclonal antibodies were functionally equivalent to CHO-produced purified antibody in vitro. FIG. 28 shows comparable binding of episomal and liver-inserted anti-HA monoclonal antibodies in mouse serum, and FIG. 29 shows equivalent neutralization of episomal and liver-inserted anti-HA monoclonal antibodies in mouse serum.

MDCK London cells were seeded at 40,000 cells/well in 50 μL of infection media (DMEM containing 1% sodium pyruvate, 0.21% Low IgG BSA solution, and 0.5% Gentamicin) in a 96-well plate. The cells were incubated at 37° C. 5% CO₂ for four hours. Plates were then infected with 50 μL of H1N1 A/Puerto Rico/08/1934 at a dilution of 10\hat{ }-4, tapped gently and placed back at 37° C. 5% CO₂ for 20 hours. Subsequently, plates were washed once with PBS and fixed with 50 μL of 4% PFA in PBS and incubated for 15 minutes at room temperature. Plates were washed three times with PBS and blocked with 300 μL of StartingBlock Blocking Buffer for one hour at room temperature. CHO-purified H1H11829N₂ anti-HA antibody spiked into PBS or naïve mouse serum (starting at 100 μg/mL antibody concentration) or serum from mice injected AAV with episomal or inserted H1H11892N₂ anti-HA or episomal REGN4446 anti-Zika formats were titrated 1:4 to a final concentration of 1.2E-4 ug/mL in StartingBlock Blocking Buffer. After incubation, Blocking Buffer was removed from plates and diluted antibodies were added onto cells at 75 μL/well. Plates were incubated for one hour at room temperature. Following incubation, plates were washed three times with Wash Buffer (imidazole-buffered saline and Tween® 20 diluted to 1× in Milli-Q water) and overlaid with 75 μL/well of secondary antibody (Donkey anti-Human IgG HRP-conjugated) diluted 1:2000 in Blocking buffer. Secondary solution was incubated on plates for one hour at room temperature. Subsequently, plates were washed three times with Wash Buffer and 75 μL/well of developing substrate ELISA Pico substrate prepared 1:1 was added. Plates were read immediately for luminescence on the Molecular Devices Spectramax i3x plate reader.

MDCK London cells below passage 10 were seeded at a density of approximately 8×10³ cells per well in MDCK medium (DMEM supplemented with 10% heat-inactivated FBS HyClone, L-glutamine, and Gentamycin) into 96-well clear bottom-black tissue culture treated plates and incubated overnight at 37° C. with 5% CO₂. Serum from mice injected with either the episomal format or the insertion format of H1H11829N₂ anti-HA antibody was diluted 1:10 and then samples were serially diluted 6-fold across a 96-well V-bottom microtiter plate for a total of 11 serum concentrations. CHO-purified H1H11829N₂ anti-HA antibody was diluted into naïve mouse serum as a positive control. CHO-purified anti-FELD1 was spiked into naïve mouse serum as a negative isotype control also at 200 μg/mL. Influenza A virus H1N1 A/PR/08/34 (ATCC, cat #VR-1469, lot #58101202) was thawed on ice, diluted just before use, and combined 1:1 with prediluted serum antibodies. Medium was removed from the MDCK cells and replaced with 60 μL of antibody:virus mixture in duplicate. Cells were then incubated for 20 hours at 37° C., 5% CO₂ to allow foci formation. The following day, the antibody:virus mixture was aspirated off, cells were washed and were then fixed with 4% paraformaldehyde for 30 minutes. Plates were then washed, blocked with 200 μL blocking buffer (Life Technologies, cat #37538 and 0.1% Triton X-100) for 1 hour at room temperature. Blocking buffer was removed and 75 μL diluted primary antibody (Mouse anti-influenza A NP antibody Millipore, cat #MAB8251) was added to incubate overnight at 4° C. Plates were then washed 2× with PBS and secondary antibody (Goat α-mouse AlexaFluor 488 conjugated antibody) was applied for 1 hour at room temperature. Plates were washed 3× with PBS and immediately read using the CTL Universal Immunospot Analyzer. The plates were imaged with automatic focus and uninfected and virus-only control wells were used to set the minimum and maximum fluorescence settings. Fluorescent foci were selected as the settings to count, and the plates were read. Data were then plotted in GraphPad Prism as the number of fluorescent (infected) cells counted vs the LOG M of the antibody concentration.

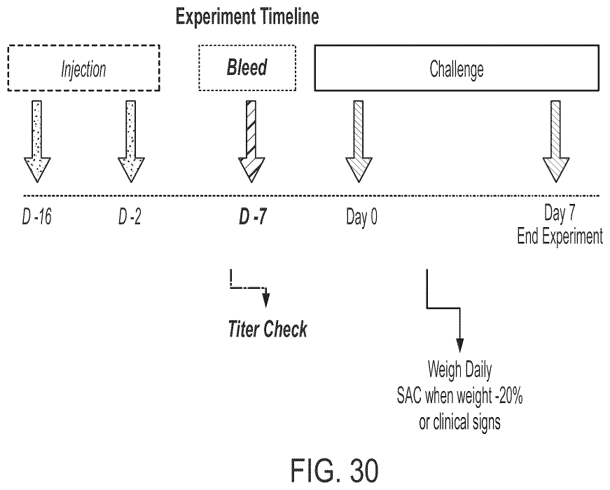

To test the functionality of the anti-PcrV monoclonal antibodies produced from either the episomal or dual AAV insertion strategies, an in vivo Pseudomonas challenge model was employed. See FIG. 30. Female C57 BL/6NCrl-Elite and female BALB/c Elite mice (5 weeks old) were divided into 10 groups of N=5 mice/group/species. The groups received either an injection of (1) PBS, (2) AAV2/8 to episomally express an isotype control antibody H1H11829N₂ anti-HA (CAG LC_T2A_RORss_HC), a (3) low dose (1.0E+10 VG/Mouse) or (4) high dose (1.0E+11 VG/mouse) of AAV2/8 to episomally express the H1H29339P anti-PcrV antibody driven by a CAG promoter (HC_T2A_RORss_LC format), a (5) low dose (1E+11 VG/mouse/vector) or (6) high dose (1E+12 VG/mouse/vector) of two AAVs, one carrying gRNA1 and the H1H29339P anti-PcrV mAb expression cassette (HC_T2A_RORss_LC) and AAV2/8.SerpinAP.Cas9 (SEQ ID NO: 39), a (7) low dose (0.2 mg/kg) or (8) high dose (1.0 mg/kg) of CHO-purified H1H29339P anti-PcrV mAB, or (9) 1.0 mg/kg of REGN684 hIgG1 isotype control. Group 10 was a group of mice that served as an uninfected control. Another group (Group 11) served as a non-protected, infected control (bacteria-only). Groups (1)-(6) were injected intravenously via tail vein injection 16 days prior to the start of the challenge. Groups (7)-(9) were injected subcutaneously 2 days prior to challenge. An additional N=5 mice were also injected subcutaneously with PBS for additional vehicle-only control mice bringing the total number of mice in group (1) to 10/species. Seven days prior to challenge, mice in groups (1)-(6) were bled retro-orbitally and serum was collected in order to run a human FC ELISA and determine circulating titers of human mAB (either iso-type control or H1H23933P) in each mouse. Mice were weighed on day of challenge and then inoculated with Pseudomonas aeruginosa strain 6077 through intranasal injection. Mice were then weighed every 24 hours for up to 7 days post-bacterial administration. Mice were sacrificed once weight loss reached >20% or mice showed other indications of clinical distress such as: lethargy; non-responsive to stimuli; ruffled fur, hunched posture, shaking; or “neurological” signs (head tilt, spinning, falling to one side). Mice that were found to be moribund, that is unable to right themselves when placed on back, were also sacrificed. All remaining mice were sacrificed on Day 7 post-bacterial-infection.

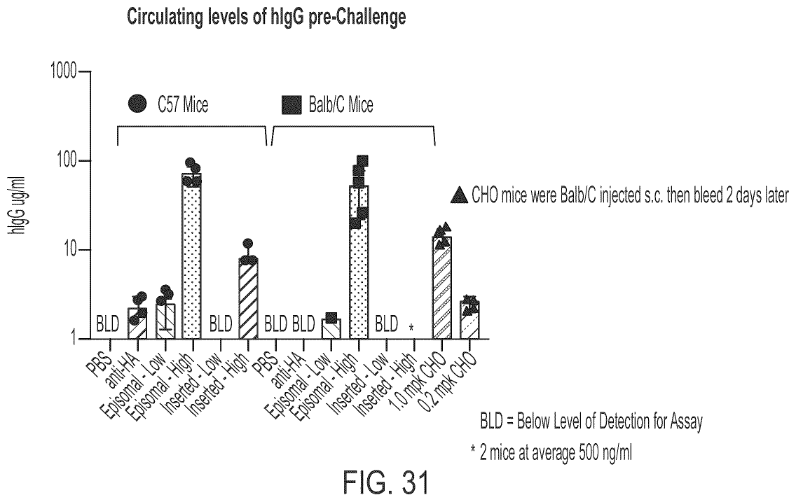

FIG. 31 shows hIgG titers of mice injected with AAV nine days prior (this is 7 days prior to challenge). A human FC ELISA was performed (as described in methods for FIG. 3) to determine the level of hIgG circulating in mouse serum 9 days after delivery of monoclonal antibody cassettes using AAV as described in the experiment above. Several values were below the detection limits (100 ng/ml) of the assay at this timepoint. In a separate experiment, age-matched BALB/c-elite mice were injected with low dose (0.2 mg/kg) or high dose (1.0 mg/kg) of CHO-purified H1H29339P anti-PcrV monoclonal antibody, and serum was collected two days later to determine expected circulating human IgG levels at time of a challenge that correspond to these doses. These values are the bars on the right side of the graph. In line with past observations, AAV8 transduces C57BL/6 mice more efficiently than BALB/c. As a result, values of secreted protein that results from successful transduction of either a single AAV (episomal) or dual AAV (inserted) strategy were lower in the BALB/c mice as expected. Since the insertion strategy requires successful transduction of two different AAVs, the reduced infectivity reduces the observed titers between strains even further than when only one AAV is needed to lead to secretion of protein.

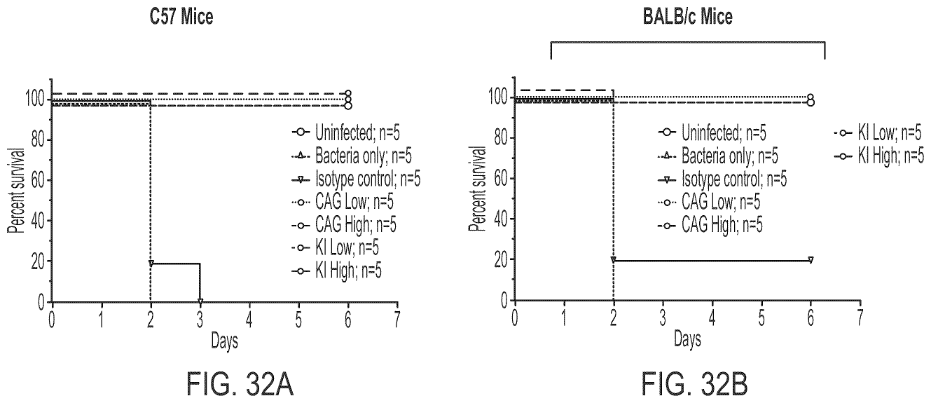

FIGS. 32A and 32B show the results of groups (2)-(6) and (10)-(11) in the Pseudomonas challenge experiment outlined above (FIG. 30). These are the groups with AAV delivery of the monoclonal antibody along with the uninfected and bacteria-only controls. In the C57BL/6NCrl-Elite mice, all AAV episomal delivered isotype control (2) and non-protected, infected mice (11) did not survive the challenge. All uninfected mice (10) and mice generating the H1H29339P anti-PcrV mAB from the liver through either episomal AAV expression or insertion into the first intron of the albumin locus using the dual AAV strategy survived irrespective of whether a low or high dose was administered (3)-(6). See FIG. 32A. In BALB/c-elite mice, 4 of 5 AAV episomal delivered isotype control (2), all non-protected, infected mice (11), and all dual AAV insertion strategy low dose mice (5) did not survive the challenge. All uninfected mice (10) and mice generating the H1H29339P anti-PcrV mAB from the liver through episomal AAV expression survived whether dosed low or high (3)-(4). All mice generating the H1H29339P anti-PcrV mAB from dual AAV strategy survived that received a high dose (6). See FIG. 32B.

In summary, we have shown successful insertion of multiple different antibody genes into the albumin locus, and we have shown that the antibody produced is functionally equivalent to CHO-produced purified antibody in vitro and provide protection in in vivo challenge models. These experiments were with antibodies of multiple IgG types. All of the Zika data is with REGN4504 which is IgG1 or REGN4446 which is an IgG4 uber stealth format, and the anti-PcrV and anti-HA antibodies are IgG1 format. We have shown the expression, functionality, and protective effects with antibodies targeting a virus (anti-Zika or anti-HA) and with antibodies targeting a bacterium (anti-PcrV). Similarly, we have tested inserted antibody genes in which the heavy chain is first (anti-PcrV and anti-Zika), and we have tested antibody genes in which the light chain is first (anti-HA and anti-Zika). Likewise, we have tested multiple different 2A proteins between the two antibody chains (anti-PcrV was T2A with heavy chain first, anti-HA was T2A with light chain first, and we tested F2A, P2A, and T2A in anti-Zika with heavy chain first).

1.-113. (canceled)

114. A method for inserting an antibody coding sequence into the first intron of an albumin locus in one or more liver cells in a subject that is a human or non-human primate, comprising introducing into the one or more liver cells in subject: (a) a nuclease agent that targets a target site in the first intron of the albumin locus or one or more nucleic acids encoding the nuclease agent; and (b) an exogenous donor nucleic acid comprising the antibody coding sequence,
wherein the nuclease agent cleaves the target site and the antibody coding sequence is inserted into the first intron of the albumin locus to produce a modified albumin locus,
wherein the antibody is not a single-chain antibody,
wherein the antibody comprises a heavy chain and a separate light chain,
wherein a heavy chain coding sequence is upstream of a light chain coding sequence in the antibody coding sequence,
wherein the heavy chain coding sequence comprises V_{H}, D_{H}, and J_{H} segments, and the light chain coding sequence comprises V_{L} and J_{L} gene segments,
wherein the heavy chain and the light chain are linked by a 2A peptide,
wherein the 2A peptide is a T2A peptide,
wherein a nucleic acid encoding a furin cleavage site is included between the heavy chain coding sequence and the light chain coding sequence, and wherein the furin cleavage site is included upstream of the 2A peptide,
wherein the heavy chain coding sequence is operably linked to an endogenous albumin secretion signal sequence in the modified albumin locus, and the antibody coding sequence comprises an exogenous secretion signal sequence upstream of the light chain coding sequence,
wherein the inserted antibody coding sequence is operably linked to an endogenous albumin promoter in the modified albumin locus, and
wherein the antibody targets a disease-associated antigen.

115. The method of claim 114, wherein expression of antibody in the subject has a prophylactic or therapeutic effect against the disease in the subject.

116. The method of claim 114, wherein the nuclease agent is a zinc finger nuclease (ZFN), a Transcription Activator-Like Effector Nuclease (TALEN), or a Clustered Regularly Interspaced Short Palindromic Repeats (CRISPR)-associated (Cas) protein and a guide RNA (gRNA).

117. The method of claim 116, wherein the nuclease agent is the Cas protein and the gRNA, wherein the Cas protein is a Cas9 protein, and wherein the gRNA comprises:
(a) a CRISPR RNA (crRNA) that targets the target site, wherein the target site is immediately flanked by a Protospacer Adjacent Motif (PAM) sequence; and
(b) a trans-activating CRISPR RNA (tracrRNA).

118. The method of claim 117, wherein the gRNA comprises 2′-O-methyl analogs and 3′ phosphorothioate internucleotide linkages at the first three 5′ and 3′ terminal RNA residues.

119. The method of claim 114, wherein:
(I) the antibody coding sequence is inserted via non-homologous end joining; and
(II) the exogenous donor nucleic acid does not comprise homology arms.

120. The method of claim 114, wherein:
(I) the antibody coding sequence is inserted via homology-directed repair;
(II) the exogenous donor nucleic acid is single-stranded;
(III) the exogenous donor nucleic acid is double-stranded; or
(IV) the antibody coding sequence in the exogenous donor nucleic acid is flanked on each side by the target site for the nuclease agent, wherein the nuclease agent cleaves the target sites flanking the antibody coding sequence, wherein the target site in the albumin locus is no longer present if the antibody coding sequence is inserted into the albumin locus in the correct orientation but it is reformed if the antibody coding sequence is inserted into the albumin locus in the opposite orientation, and wherein the exogenous donor nucleic acid is delivered by adeno-associated virus (AAV)-mediated delivery, and cleavage of the target sites flanking the antibody coding sequence removes the inverted terminal repeats of the AAV.

121. The method of claim 114, wherein:
(I) the disease-associated antigen is a cancer-associated antigen, an infectious-disease-associated antigen, a viral antigen, or a bacterial antigen; and/or
(II) the antibody is a neutralizing antibody or a broadly neutralizing antibody.

122. The method of claim 121, wherein the bacterial antigen is a Pseudomonas aeruginosa PcrV antigen.

123. The method of claim 121, wherein:
(1) the viral antigen is an influenza hemagglutinin antigen, wherein the antibody comprises a light chain comprising light chain CDRs 1-3 and a heavy chain comprising heavy chain CDRs 1-3, wherein:
(I) the light chain comprises a sequence at least 90% identical to the sequence set forth in SEQ ID NO: 18 and the heavy chain comprises a sequence at least 90% identical to the sequence set forth in SEQ ID NO: 20,
wherein the light chain CDRs 1-3 comprise sequences identical to the sequences set forth in SEQ ID NOS: 76-78, respectively, and the heavy chain CDRs 1-3 comprise sequences identical to the sequences set forth in SEQ ID NOS: 79-81, respectively; or
(II) the light chain comprises a sequence at least 90% identical to the sequence set forth in SEQ ID NO: 126 and the heavy chain comprises a sequence at least 90% identical to the sequence set forth in SEQ ID NO: 128,
wherein the light chain CDRs 1-3 comprise sequences identical to the sequences set forth in SEQ ID NOS: 129-131, respectively, and the heavy chain CDRs 1-3 comprise sequences identical to the sequences set forth in SEQ ID NOS: 132-134, respectively;
(2) the viral antigen is a Zika Envelope (Env) antigen, wherein the antibody comprises a light chain comprising light chain CDRs 1-3 and a heavy chain comprising heavy chain CDRs 1-3, wherein:
the light chain comprises a sequence at least 90% identical to the sequence set forth in SEQ ID NO: 3 and the heavy chain comprises a sequence at least 90% identical to the sequence set forth in SEQ ID NO: 5,
wherein the light chain CDRs 1-3 comprise sequences identical to the sequences set forth in SEQ ID NOS: 64-66, respectively, and the heavy chain CDRs 1-3 comprise sequences identical to the sequences set forth in SEQ ID NOS: 67-69, respectively; or
(3) the viral antigen is a Zika Envelope (Env) antigen, wherein the antibody comprises a light chain comprising light chain CDRs 1-3 and a heavy chain comprising heavy chain CDRs 1-3, wherein:
the light chain comprises a sequence at least 90% identical to the sequence set forth in SEQ ID NO: 13 and the heavy chain comprises a sequence at least 90% identical to the sequence set forth in SEQ ID NO: 15,
wherein the light chain CDRs 1-3 comprise sequences identical to the sequences set forth in SEQ ID NOS: 70-72, respectively, and the heavy chain CDRs 1-3 comprise sequences identical to the sequences set forth in SEQ ID NOS: 73-75, respectively.

124. The method of claim 114, wherein:
(I) the nuclease agent or the one or more nucleic acids encoding the nuclease agent and the exogenous donor nucleic acid are introduced in separate delivery vehicles; and/or
(II) the nuclease agent or the one or more nucleic acids encoding the nuclease agent and the exogenous donor nucleic acid are introduced sequentially.

125. The method of claim 114, wherein:
(I) the nuclease agent or the one or more nucleic acids encoding the nuclease agent and the exogenous donor nucleic acid are introduced together in the same delivery vehicle; and/or
(II) the nuclease agent or the one or more nucleic acids encoding the nuclease agent and the exogenous donor nucleic acid are introduced simultaneously.

126. The method of claim 114, wherein:
(I) the nuclease agent or the one or more nucleic acids encoding the nuclease agent and the exogenous donor nucleic acid are introduced in single doses or multiple doses;
(II) the nuclease agent or the one or more nucleic acids encoding the nuclease agent and the exogenous donor nucleic acid are delivered via intravenous injection; or
(III) the nuclease agent or the one or more nucleic acids encoding the nuclease agent and the exogenous donor nucleic acid are introduced via lipid-nanoparticle-mediated delivery or via adeno-associated virus (AAV)-mediated delivery.

127. The method of claim 126, wherein the nuclease agent or the one or more nucleic acids encoding the nuclease agent and the exogenous donor nucleic acid are both introduced by AAV-mediated delivery.

128. The method of claim 126, wherein the nuclease agent or the one or more nucleic acids encoding the nuclease agent and the exogenous donor nucleic acid are introduced by two different AAV vectors.

129. The method of claim 126, wherein:
(I) the nuclease agent or the one or more nucleic acids encoding the nuclease agent is introduced via lipid-nanoparticle-mediated delivery, wherein the nuclease agent is a Clustered Regularly Interspaced Short Palindromic Repeats (CRISPR)-associated 9 (Cas9) protein and a guide RNA (gRNA), and wherein the Cas9 in the lipid nanoparticle is in the form of mRNA, and the gRNA in the lipid nanoparticle is in the form of RNA; and/or
(II) the exogenous donor nucleic acid is introduced via AAV-mediated delivery.

130. The method of claim 129, wherein the AAVis AAV8 or AAV2/8.

131. The method of claim 114, wherein:
(I) the nuclease agent comprises a Clustered Regularly Interspaced Short Palindromic Repeats (CRISPR)-associated 9 (Cas9) and a guide RNA (gRNA), wherein the method comprises introducing the gRNA and an mRNA encoding the Cas9 via lipid-nanoparticle-mediated delivery, and the exogenous donor nucleic acid is introduced via AAV8-mediated or AAV2/8-mediated delivery; or
(II) the nuclease agent comprises a Clustered Regularly Interspaced Short Palindromic Repeats (CRISPR)-associated 9 (Cas9) and a guide RNA (gRNA), wherein the method comprises introducing a DNA encoding the Cas9 via AAV8-mediated delivery in a first AAV8 or AAV2/8-mediated delivery in a first AAV2/8, and introducing the exogenous donor nucleic acid and a DNA encoding the gRNA via AAV8-mediated delivery in a second AAV8 or AAV2/8-mediated delivery in a second AAV2/8.

132. The method of claim 114, wherein expression of the antibody in the subject results in plasma levels of at least 2.5 μg/mL, at least 5 μg/mL, at least 10 μg/mL, at least 100 μg/mL, at least 200 μg/mL, at least 300 μg/mL, at least 400 μg/mL, at least 500 μg/mL, at least 600 μg/mL, at least 700 μg/mL, at least 800 μg/mL, at least 900 μg/mL, or at least 1000 μg/mL 2 weeks, 4 weeks, 8 weeks, 12 weeks, or 16 weeks after introducing the nuclease agent or the one or more nucleic acids encoding the nuclease agent and the exogenous donor nucleic acid.

133. The method of claim 114, wherein the subject is the human.

134. The method of claim 114, wherein the nuclease agent is a Clustered Regularly Interspaced Short Palindromic Repeats (CRISPR)-associated 9 (Cas9) protein and a guide RNA (gRNA),
wherein the nuclease agent or the one or more nucleic acids encoding the nuclease agent and the exogenous donor nucleic acid are delivered via lipid-nanoparticle-mediated delivery, adeno-associated-virus 8 (AAV8)-mediated delivery, or AAV2/8-mediated delivery,
wherein the antibody targets a viral antigen or a bacterial antigen, and
wherein the antibody is a broadly neutralizing antibody.


SEQUENCE LISTING (146 sequences)

SEQ ID NO: 1 - DNA, 2943 bp, synthetic construct
  source 1..2943: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgttaggtcagtgaagagaagaacaaaaagcagcatattacagttagttgtcttcatcaatctttaaatatgttgtgtggtttttctctccctgtttccacagccgaaatagtgctgacccagtcaccagataccctgagcctgagtcctggggaacgggcaacactcagttgtagggcatcccagagtgtgtctagtaattatctggcttggtaccagcaaaaaccggggcaggctccccgactgctgatctatggcgcaagcagccgagccaccggtattccagatcgatttagtggatctggaagtggaactgacttcacgttgacaatatcaagactggaacccgaagatttcgctgtgtattattgccagcgctacggtaccagccccctgacattcggggggggaacgaaggttgaaataaaacgcaccgtcgcggcgccatctgtattcatttttcccccgtctgatgagcaactgaaatcagggaccgcgtccgtggtctgccttctgaacaatttttacccgagagaggcgaaagtccagtggaaggtggataatgcgcttcagtcaggtaactctcaggagagcgtcacagagcaagactctaaagattcaacttacagcctttcctccaccctgactctgtccaaggccgactacgagaaacataaggtctatgcctgcgaagtaactcatcaaggtcttagttcacccgtcacgaaaagttttaataggggggagtgtagaaaacggaggggatcaggggcgactaacttttcattgcttaagcaagcaggagacgtggaagagaatcccgggccccataggccgcgacgacgggggaccagaccccctcctttggccctgctggctgctttgcttctcgcggcgcgaggagcggacgctcaggtacagctcgttgagagcggaggtggggttgtgcagcctgggagatctctccgcctcagttgcgccgcctcaggttttacgttcaattattatggcatgcattgggttagacaagctccggggaaggggttggaatgggtagccgtaattagttacgacggaaccaataagtattatgctgacagtgtgaagggtcgatttacgacatcccgggataactccaagaacacattgtaccttcaaatgaattctttgcgggcggaagatactgcactctattattgtgcgagagatcgagggggcagatttgactactggggccaaggaatacaggttactgtatcatctgcttcaactaagggtccgagcgtatttccccttgctccttgcagccgatcaacaagtgaaagtacagctgctttgggttgccttgtgaaagattatttccctgagcctgtgactgtttcctggaattcaggtgctcttactagcggggttcatacatttcccgctgtactccagtcaagcgggctctatagtctcagtagcgtagtaacggtaccctcttcatcacttgggacaaagacgtacacatgcaatgtagaccataagccgtctaatacgaaagttgataaaagggtagaatccaaatatggcccgccgtgtccgccttgtccagctccgggcggtgggggccccagtgtattcctgtttccccctaaaccgaaggatacgcttatgattagtcgaacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatgactcgagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtagatctaacttgtttattgcagcttataatggttacaaataaagcaatagcatcacaaatttcacaaataaagcatttttttcactgcattctagttgtggtttgtccaaactcatcaatgtatcttatcatgtctgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 2 - DNA, 645 bp, synthetic construct
  source 1..645: mol_type = other DNA
gaaatagtgctgacccagtcaccagataccctgagcctgagtcctggggaacgggcaacactcagttgtagggcatcccagagtgtgtctagtaattatctggcttggtaccagcaaaaaccggggcaggctccccgactgctgatctatggcgcaagcagccgagccaccggtattccagatcgatttagtggatctggaagtggaactgacttcacgttgacaatatcaagactggaacccgaagatttcgctgtgtattattgccagcgctacggtaccagccccctgacattcggggggggaacgaaggttgaaataaaacgcaccgtcgcggcgccatctgtattcatttttcccccgtctgatgagcaactgaaatcagggaccgcgtccgtggtctgccttctgaacaatttttacccgagagaggcgaaagtccagtggaaggtggataatgcgcttcagtcaggtaactctcaggagagcgtcacagagcaagactctaaagattcaacttacagcctttcctccaccctgactctgtccaaggccgactacgagaaacataaggtctatgcctgcgaagtaactcatcaaggtcttagttcacccgtcacgaaaagttttaataggggggagtgt

SEQ ID NO: 3 - protein, 215 aa, synthetic construct
EIVLTQSPDTLSLSPGERATLSCRASQSVSSNYLAWYQQKPGQAPRLLIYGASSRATGIPDRFSGSGSGTDFTLTISRLEPEDFAVYYCQRYGTSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - DNA, 1329 bp, synthetic construct
  source 1..1329: mol_type = other DNA
caggtacagctcgttgagagcggaggtggggttgtgcagcctgggagatctctccgcctcagttgcgccgcctcaggttttacgttcaattattatggcatgcattgggttagacaagctccggggaaggggttggaatgggtagccgtaattagttacgacggaaccaataagtattatgctgacagtgtgaagggtcgatttacgacatcccgggataactccaagaacacattgtaccttcaaatgaattctttgcgggcggaagatactgcactctattattgtgcgagagatcgagggggcagatttgactactggggccaaggaatacaggttactgtatcatctgcttcaactaagggtccgagcgtatttccccttgctccttgcagccgatcaacaagtgaaagtacagctgctttgggttgccttgtgaaagattatttccctgagcctgtgactgtttcctggaattcaggtgctcttactagcggggttcatacatttcccgctgtactccagtcaagcgggctctatagtctcagtagcgtagtaacggtaccctcttcatcacttgggacaaagacgtacacatgcaatgtagaccataagccgtctaatacgaaagttgataaaagggtagaatccaaatatggcccgccgtgtccgccttgtccagctccgggcggtgggggccccagtgtattcctgtttccccctaaaccgaaggatacgcttatgattagtcgaacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaa

SEQ ID NO: 5 - protein, 443 aa, synthetic construct
QVQLVESGGGVVQPGRSLRLSCAASGFTFNYYGMHWVRQAPGKGLEWVAVISYDGTNKYYADSVKGRFTTSRDNSKNTLYLQMNSLRAEDTALYYCARDRGGRFDYWGQGIQVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPGGGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 6 - DNA, 3854 bp, synthetic construct
  misc_difference 468..487: note = n is a, c, g, or t
  source 1..3854: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcgctagctgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccnnnnnnnnnnnnnnnnnnnngttttagagctagaaatagcaagttaaaataaggctagtccgttatcaacttgaaaaagtggcaccgagtcggtgctttttttctagaccacctaagggttctcagatgcacccttacgcgttaggtcagtgaagagaagaacaaaaagcagcatattacagttagttgtcttcatcaatctttaaatatgttgtgtggtttttctctccctgtttccacagcccaggtgcagctggtggagtcggggggaggcgtggtccagcctgggaggtccctgagactctcctgtgcagcctctggattcaccttcaattactatggcatgcactgggtccgccaggctccaggcaaggggctggagtgggtggcagtcatatcatatgatggaactaataaatactatgcagactccgtgaagggccgattcaccacctccagagacaattccaagaacacgctgtatctgcagatgaacagcctgagagctgaggacacggctctgtattactgtgcgagagatcgcggtggccgctttgactactggggccagggaatccaggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccaggcggtggcggaccatcagtcttcctgttccccccaaaacccaaggacactctctacatcacccgggagcctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaacgtaaacgaagaggatccggggtgaagcaaaccttgaatttcgatctcctgaagttggctggcgatgtggagagtaatcccggcccaaagtgggtaacctttctcctcctcctcttcgtctccggctctgctttttccaggggtgtgtttcgccgagaaattgtgttgacgcagtctccagacaccctgtctttgtctccaggggaaagagccaccctctcctgcagggccagtcagagtgttagcagcaactacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcggtatggtacctcaccgctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaagcggccgcgtttaaactcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcagaattcctgcagctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaggtggccacctaagggttctcagatgcagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 7 - DNA, 3845 bp, synthetic construct
  misc_difference 468..487: note = n is a, c, g, or t
  source 1..3845: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcgctagctgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccnnnnnnnnnnnnnnnnnnnngttttagagctagaaatagcaagttaaaataaggctagtccgttatcaacttgaaaaagtggcaccgagtcggtgctttttttctagaccacctaagggttctcagatgcacccttacgcgttaggtcagtgaagagaagaacaaaaagcagcatattacagttagttgtcttcatcaatctttaaatatgttgtgtggtttttctctccctgtttccacagcccaggtgcagctggtggagtcggggggaggcgtggtccagcctgggaggtccctgagactctcctgtgcagcctctggattcaccttcaattactatggcatgcactgggtccgccaggctccaggcaaggggctggagtgggtggcagtcatatcatatgatggaactaataaatactatgcagactccgtgaagggccgattcaccacctccagagacaattccaagaacacgctgtatctgcagatgaacagcctgagagctgaggacacggctctgtattactgtgcgagagatcgcggtggccgctttgactactggggccagggaatccaggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccaggcggtggcggaccatcagtcttcctgttccccccaaaacccaaggacactctctacatcacccgggagcctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaacgtaaacgaagaggatccggggcgactaacttttcattgcttaagcaagcaggagacgtggaagagaatcccgggcccaagtgggtaacctttctcctcctcctcttcgtctccggctctgctttttccaggggtgtgtttcgccgagaaattgtgttgacgcagtctccagacaccctgtctttgtctccaggggaaagagccaccctctcctgcagggccagtcagagtgttagcagcaactacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcggtatggtacctcaccgctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaagcggccgcgtttaaactcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcagaattcctgcagctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaggtggccacctaagggttctcagatgcagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 8 - DNA, 3842 bp, synthetic construct
  misc_difference 468..487: note = n is a, c, g, or t
  source 1..3842: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcgctagctgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccnnnnnnnnnnnnnnnnnnnngttttagagctagaaatagcaagttaaaataaggctagtccgttatcaacttgaaaaagtggcaccgagtcggtgctttttttctagaccacctaagggttctcagatgcacccttacgcgttaggtcagtgaagagaagaacaaaaagcagcatattacagttagttgtcttcatcaatctttaaatatgttgtgtggtttttctctccctgtttccacagcccaggtgcagctggtggagtcggggggaggcgtggtccagcctgggaggtccctgagactctcctgtgcagcctctggattcaccttcaattactatggcatgcactgggtccgccaggctccaggcaaggggctggagtgggtggcagtcatatcatatgatggaactaataaatactatgcagactccgtgaagggccgattcaccacctccagagacaattccaagaacacgctgtatctgcagatgaacagcctgagagctgaggacacggctctgtattactgtgcgagagatcgcggtggccgctttgactactggggccagggaatccaggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccaggcggtggcggaccatcagtcttcctgttccccccaaaacccaaggacactctctacatcacccgggagcctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaacgtaaacgaagaggatccggggagggccggggcagcctgctgacctgcggagacgtggaggagaaccctggccccaagtgggtaacctttctcctcctcctcttcgtctccggctctgctttttccaggggtgtgtttcgccgagaaattgtgttgacgcagtctccagacaccctgtctttgtctccaggggaaagagccaccctctcctgcagggccagtcagagtgttagcagcaactacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcggtatggtacctcaccgctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaagcggccgcgtttaaactcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcagaattcctgcagctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaggtggccacctaagggttctcagatgcagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 9 - DNA, 3857 bp, synthetic construct
  misc_difference 468..487: note = n is a, c, g, or t
  source 1..3857: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcgctagctgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccnnnnnnnnnnnnnnnnnnnngttttagagctagaaatagcaagttaaaataaggctagtccgttatcaacttgaaaaagtggcaccgagtcggtgctttttttctagaccacctaagggttctcagatgcacccttacgcgttaggtcagtgaagagaagaacaaaaagcagcatattacagttagttgtcttcatcaatctttaaatatgttgtgtggtttttctctccctgtttccacagcccaggtgcagctggtggagtcggggggaggcgtggtccagcctgggaggtccctgagactctcctgtgcagcctctggattcaccttcaattactatggcatgcactgggtccgccaggctccaggcaaggggctggagtgggtggcagtcatatcatatgatggaactaataaatactatgcagactccgtgaagggccgattcaccacctccagagacaattccaagaacacgctgtatctgcagatgaacagcctgagagctgaggacacggctctgtattactgtgcgagagatcgcggtggccgctttgactactggggccagggaatccaggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccaggcggtggcggaccatcagtcttcctgttccccccaaaacccaaggacactctctacatcacccgggagcctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaacgtaaacgaagaggatccggggagggccggggcagcctgctgacctgcggagacgtggaggagaaccctggcccccacagacctagacgtcgtggaactcgtccacctccactggcactgctcgctgctctcctcctggctgcacgtggtgctgatgcagaaattgtgttgacgcagtctccagacaccctgtctttgtctccaggggaaagagccaccctctcctgcagggccagtcagagtgttagcagcaactacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcggtatggtacctcaccgctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaagcggccgcgtttaaactcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcagaattcctgcagctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaggtggccacctaagggttctcagatgcagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 10 - DNA, 4437 bp, synthetic construct
  source 1..4437: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctcgggcaaagccacgcgtaggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactaaaacaggtaagtccggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgatgcctctactaaccatgttcatgttttctttttttttctacaggtcctgggtgacgaacaggctagcatcgatgccaccatgcacagacctagacgtcgtggaactcgtccacctccactggcactgctcgctgctctcctcctggctgcacgtggtgctgatgcacaggtgcagctggtggagtcggggggaggcgtggtccagcctgggaggtccctgagactctcctgtgcagcctctggattcaccttcaattactatggcatgcactgggtccgccaggctccaggcaaggggctggagtgggtggcagtcatatcatatgatggaactaataaatactatgcagactccgtgaagggccgattcaccacctccagagacaattccaagaacacgctgtatctgcagatgaacagcctgagagctgaggacacggctctgtattactgtgcgagagatcgcggtggccgctttgactactggggccagggaatccaggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccaggcggtggcggaccatcagtcttcctgttccccccaaaacccaaggacactctctacatcacccgggagcctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaacgtaaacgaagaggatccggggagggccggggcagcctgctgacctgcggagacgtggaggagaaccctggcccccacagacctagacgtcgtggaactcgtccacctccactggcactgctcgctgctctcctcctggctgcacgtggtgctgatgcagaaattgtgttgacgcagtctccagacaccctgtctttgtctccaggggaaagagccaccctctcctgcagggccagtcagagtgttagcagcaactacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcggtatggtacctcaccgctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaagcggccgcggtttaaactcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcagaattcctgcagctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggggtaaccaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 11 - DNA, 3863 bp, synthetic construct
  source 1..3863: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtggagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgtcaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttgcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacatcgattgaattcgccaccatgcacagacctagacgtcgtggaactcgtccacctccactggcactgctcgctgctctcctcctggctgcacgtggtgctgatgcagaaattgtgttgacgcagtctccagacaccctgtctttgtctccaggggaaagagccaccctctcctgcagggccagtcagagtgttagcagcaactacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcggtatggtacctcaccgctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtcgtaaacgaagaggatccggggagggccggggcagcctgctgacctgcggagacgtggaggagaaccctggccccatgcacagacctagacgtcgtggaactcgtccacctccactggcactgctcgctgctctcctcctggctgcacgtggtgctgatgcacaggtgcagctggtggagtcggggggaggcgtggtccagcctgggaggtccctgagactctcctgtgcagcctctggattcaccttcaattactatggcatgcactgggtccgccaggctccaggcaaggggctggagtgggtggcagtcatatcatatgatggaactaataaatactatgcagactccgtgaagggccgattcaccacctccagagacaattccaagaacacgctgtatctgcagatgaacagcctgagagctgaggacacggctctgtattactgtgcgagagatcgcggtggccgctttgactactggggccagggaatccaggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccaggcggtggcggaccatcagtcttcctgttccccccaaaacccaaggacactctctacatcacccgggagcctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatgactcgagagatctaacttgtttattgcagcttataatggttacaaataaagcaatagcatcacaaatttcacaaataaagcatttttttcactgcattctagttgtggtttgtccaaactcatcaatgtatcttatcatgtctgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 12 - DNA, 645 bp, synthetic construct
  source 1..645: mol_type = other DNA
gaaattgtgttgacgcagtctccagacaccctgtctttgtctccaggggaaagagccaccctctcctgcagggccagtcagagtgttagcagcaactacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcggtatggtacctcaccgctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 13 - protein, 215 aa, synthetic construct; identical to SEQ ID NO: 3

SEQ ID NO: 14 - DNA, 1329 bp, synthetic construct
  source 1..1329: mol_type = other DNA
caggtgcagctggtggagtcggggggaggcgtggtccagcctgggaggtccctgagactctcctgtgcagcctctggattcaccttcaattactatggcatgcactgggtccgccaggctccaggcaaggggctggagtgggtggcagtcatatcatatgatggaactaataaatactatgcagactccgtgaagggccgattcaccacctccagagacaattccaagaacacgctgtatctgcagatgaacagcctgagagctgaggacacggctctgtattactgtgcgagagatcgcggtggccgctttgactactggggccagggaatccaggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccaggcggtggcggaccatcagtcttcctgttccccccaaaacccaaggacactctctacatcacccgggagcctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaa

SEQ ID NO: 15 - protein, 443 aa, synthetic construct
QVQLVESGGGVVQPGRSLRLSCAASGFTFNYYGMHWVRQAPGKGLEWVAVISYDGTNKYYADSVKGRFTTSRDNSKNTLYLQMNSLRAEDTALYYCARDRGGRFDYWGQGIQVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPGGGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - DNA, 2237 bp, synthetic construct
  source 1..2237: mol_type = other DNA
aaaagcagcatattacagttagttgtcttcatcaatctttaaatatgttgtgtggtttttctctccctgtttccacagccgacatacagatgacgcagtccccttccagcctcagcgcatcagtgggggacagagtcactatcacttgcagggcttctcagggcattagaaacaacttgggctggtaccaacagaagcctctgaaggcacctaaacggttgatttacgccgccagctctttgcaatctggggtgccttccagattcagcggctctggctcaggaaccgaatttaccctgaccattagcagcttgcaaccggaggatttcgctacctactattgcttgcagtataataactatccctggaccttcggtcaaggtaccaaggtcgagataaagcggaccgttgctgccccttctgtgttcatctttcccccctcagatgaacagcttaagagcggaacggcaagtgtagtatgccttcttaataatttctaccctagagaagccaaagttcagtggaaagtagataatgctttgcaaagcggaaactctcaagaatcagttacagaacaagactccaaagactcaacatactcactttcatcaacgctcaccctgtctaaagccgattacgagaagcacaaagtttacgcctgtgaggttacacatcagggtctcagtagtcctgtgactaagtcttttaaccggggggaatgcagaaaacggaggggatcaggggcgactaacttttcattgcttaagcaagcaggagacgtggaagagaatcccgggccccacagacctagacgtcgtggaactcgtccacctccactggcactgctcgctgctctcctcctggctgcacgtggtgctgatgcacaggtccagctcgtccaatccggggcggaagtcaaaaagagcggctcatccgtcaaggtctcctgtaaggcctcaggtgggacatttagtagttatgccatctcctgggttcgccaggctccgggacagggcttggagtggatgggtggaatcataccgatctttggtacaccctcatacgcgcagaaattccaagaccgcgtcacgatcacgactgacgaatccacgagcaccgtttacatggagttgtcttcactgagaagtgaggacactgcagtgtattattgtgcaaggcagcagccagtgtaccaatataatatggatgtctggggtcaaggcaccaccgtgaccgtgtcctccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagtccctctccctgtctccgggtaaatga

SEQ ID NO: 17 - DNA, 642 bp, synthetic construct
  source 1..642: mol_type = other DNA
gacatacagatgacgcagtccccttccagcctcagcgcatcagtgggggacagagtcactatcacttgcagggcttctcagggcattagaaacaacttgggctggtaccaacagaagcctctgaaggcacctaaacggttgatttacgccgccagctctttgcaatctggggtgccttccagattcagcggctctggctcaggaaccgaatttaccctgaccattagcagcttgcaaccggaggatttcgctacctactattgcttgcagtataataactatccctggaccttcggtcaaggtaccaaggtcgagataaagcggaccgttgctgccccttctgtgttcatctttcccccctcagatgaacagcttaagagcggaacggcaagtgtagtatgccttcttaataatttctaccctagagaagccaaagttcagtggaaagtagataatgctttgcaaagcggaaactctcaagaatcagttacagaacaagactccaaagactcaacatactcactttcatcaacgctcaccctgtctaaagccgattacgagaagcacaaagtttacgcctgtgaggttacacatcagggtctcagtagtcctgtgactaagtcttttaaccggggggaatgc

SEQ ID NO: 18 - protein, 214 aa, synthetic construct
DIQMTQSPSSLSASVGDRVTITCRASQGIRNNLGWYQQKPLKAPKRLIYAASSLQSGVPSRFSGSGSGTEFTLTISSLQPEDFATYYCLQYNNYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - DNA, 1353 bp, synthetic construct
  source 1..1353: mol_type = other DNA
caggtccagctcgtccaatccggggcggaagtcaaaaagagcggctcatccgtcaaggtctcctgtaaggcctcaggtgggacatttagtagttatgccatctcctgggttcgccaggctccgggacagggcttggagtggatgggtggaatcataccgatctttggtacaccctcatacgcgcagaaattccaagaccgcgtcacgatcacgactgacgaatccacgagcaccgtttacatggagttgtcttcactgagaagtgaggacactgcagtgtattattgtgcaaggcagcagccagtgtaccaatataatatggatgtctggggtcaaggcaccaccgtgaccgtgtcctccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagtccctctccctgtctccgggtaaatga

SEQ ID NO: 20 - protein, 450 aa, synthetic construct
QVQLVQSGAEVKKSGSSVKVSCKASGGTFSSYAISWVRQAPGQGLEWMGGIIPIFGTPSYAQKFQDRVTITTDESTSTVYMELSSLRSEDTAVYYCARQQPVYQYNMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - DNA, 100 bp, synthetic construct
  source 1..100: mol_type = other DNA
taggtcagtgaagagaagaacaaaaagcagcatattacagttagttgtcttcatcaatctttaaatatgttgtgtggtttttctctccctgtttccacag

SEQ ID NO: 22 - DNA, 12 bp, synthetic construct
  source 1..12: mol_type = other DNA
agaaaacggagg

SEQ ID NO: 23 - protein, 4 aa, synthetic construct
RKRR

SEQ ID NO: 24 - DNA, 57 bp, synthetic construct
  source 1..57: mol_type = other DNA
gcgactaacttttcattgcttaagcaagcaggagacgtggaagagaatcccgggccc

SEQ ID NO: 25 - protein, 19 aa, synthetic construct
ATNFSLLKQAGDVEENPGP

SEQ ID NO: 26 - DNA, 66 bp, synthetic construct
  source 1..66: mol_type = other DNA
gtgaagcaaaccttgaatttcgatctcctgaagttggctggcgatgtggagagtaatcccggccca

SEQ ID NO: 27 - protein, 22 aa, synthetic construct
VKQTLNFDLLKLAGDVESNPGP

SEQ ID NO: 28 - DNA, 54 bp, synthetic construct
  source 1..54: mol_type = other DNA
gagggccggggcagcctgctgacctgcggagacgtggaggagaaccctggcccc

SEQ ID NO: 29 - protein, 18 aa, synthetic construct
EGRGSLLTCGDVEENPGP

SEQ ID NO: 30 - protein, 20 aa, synthetic construct
QCTNYALLKLAGDVESNPGP

SEQ ID NO: 31 - DNA, 84 bp, synthetic construct
  source 1..84: mol_type = other DNA
cataggccgcgacgacgggggaccagaccccctcctttggccctgctggctgctttgcttctcgcggcgcgaggagcggacgct

SEQ ID NO: 32 - DNA, 84 bp, synthetic construct
  source 1..84: mol_type = other DNA
cacagacctagacgtcgtggaactcgtccacctccactggcactgctcgctgctctcctcctggctgcacgtggtgctgatgca

SEQ ID NO: 33 - protein, 28 aa, synthetic construct
HRPRRRGTRPPPLALLAALLLAARGADA

SEQ ID NO: 34 - DNA, 69 bp, synthetic construct
  source 1..69: mol_type = other DNA
aagtgggtaacctttctcctcctcctcttcgtctccggctctgctttttccaggggtgtgtttcgccga

SEQ ID NO: 35 - protein, 21 aa, synthetic construct
EIVLTQSPDTLSLSPGERATL

SEQ ID NO: 36 - DNA, 247 bp, synthetic construct
  source 1..247: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgt

SEQ ID NO: 37 - DNA, 131 bp, synthetic construct
  source 1..131: mol_type = other DNA
aacttgtttattgcagcttataatggttacaaataaagcaatagcatcacaaatttcacaaataaagcatttttttcactgcattctagttgtggtttgtccaaactcatcaatgtatcttatcatgtctg

SEQ ID NO: 38 - DNA, 72 bp, synthetic construct
  source 1..72: mol_type = other DNA
ggttccatggtgtaatggttagcactctggactctgaatccagcgatccgagttcaaatctcggtggaacct

SEQ ID NO: 39 - DNA, 4733 bp, synthetic construct
  source 1..4733: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctacgcgtgggggaggctgctggtgaatattaaccaaggtcaccccagttatcggaggagcaaacaggggctaagtccacgggcataaattggtctgcgcaccagcaccaatctagtgccaccatggacaagcccaagaaaaagcggaaagtgaagtacagcatcggcctggacatcggcaccaactctgtgggctgggccgtgatcaccgacgagtacaaggtgcccagcaagaaattcaaggtgctgggcaacaccgacaggcacagcatcaagaagaacctgatcggcgccctgctgttcgacagcggcgaaacagccgaggccaccagactgaagagaaccgccagaagaagatacaccaggcggaagaacaggatctgctatctgcaagagatcttcagcaacgagatggccaaggtggacgacagcttcttccacagactggaagagtccttcctggtggaagaggacaagaagcacgagagacaccccatcttcggcaacatcgtggacgaggtggcctaccacgagaagtaccccaccatctaccacctgagaaagaaactggtggacagcaccgacaaggccgacctgagactgatctacctggccctggcccacatgatcaagttcagaggccacttcctgatcgagggcgacctgaaccccgacaacagcgacgtggacaagctgttcatccagctggtgcagacctacaaccagctgttcgaggaaaaccccatcaacgccagcggcgtggacgccaaggctatcctgtctgccagactgagcaagagcagaaggctggaaaatctgatcgcccagctgcccggcgagaagaagaacggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgttcctggccgccaagaacctgtctgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagaaatcttcttcgaccagagcaagaacggctacgccggctacatcgatggcggcgctagccaggaagagttctacaagttcatcaagcccatcctggaaaagatggacggcaccgaggaactgctcgtgaagctgaacagagaggacctgctgagaaagcagagaaccttcgacaacggcagcatcccccaccagatccacctgggagagctgcacgctatcctgagaaggcaggaagatttttacccattcctgaaggacaaccgggaaaagatcgagaagatcctgaccttcaggatcccctactacgtgggccccctggccagaggcaacagcagattcgcctggatgaccagaaagagcgaggaaaccatcaccccctggaacttcgaggaagtggtggacaagggcgccagcgcccagagcttcatcgagagaatgacaaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaggccatcgtggacctgctgttcaagaccaacagaaaagtgaccgtgaagcagctgaaagaggactacttcaagaaaatcgagtgcttcgactccgtggaaatctccggcgtggaagatagattcaacgcctccctgggcacataccacgatctgctgaaaattatcaaggacaaggacttcctggataacgaagagaacgaggacattctggaagatatcgtgctgaccctgacactgtttgaggaccgcgagatgatcgaggaaaggctgaaaacctacgctcacctgttcgacgacaaagtgatgaagcagctgaagagaaggcggtacaccggctggggcaggctgagcagaaagctgatcaacggcatcagagacaagcagagcggcaagacaatcctggatttcctgaagtccgacggcttcgccaaccggaacttcatgcagctgatccacgacgacagcctgacattcaaagaggacatccagaaagcccaggtgtccggccagggcgactctctgcacgagcatatcgctaacctggccggcagccccgctatcaagaagggcatcctgcagacagtgaaggtggtggacgagctcgtgaaagtgatgggcagacacaagcccgagaacatcgtgatcgagatggctagagagaaccagaccacccagaagggacagaagaactcccgcgagaggatgaagagaatcgaagagggcatcaaagagctgggcagccagatcctgaaagaacaccccgtggaaaacacccagctgcagaacgagaagctgtacctgtactacctgcagaatggccgggatatgtacgtggaccaggaactggacatcaacagactgtccgactacgatgtggaccatatcgtgcctcagagctttctgaaggacgactccatcgataacaaagtgctgactcggagcgacaagaacagaggcaagagcgacaacgtgccctccgaagaggtcgtgaagaagatgaagaactactggcgacagctgctgaacgccaagctgattacccagaggaagttcgataacctgaccaaggccgagagaggcggcctgagcgagctggataaggccggcttcatcaagaggcagctggtggaaaccagacagatcacaaagcacgtggcacagatcctggactcccggatgaacactaagtacgacgaaaacgataagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgaaatcaccctggccaacggcgagatcagaaagcgccctctgatcgagacaaacggcgaaaccggggagatcgtgtgggataagggcagagacttcgccacagtgcgaaaggtgctgagcatgccccaagtgaatatcgtgaaaaagaccgaggtgcagacaggcggcttcagcaaagagtctatcctgcccaagaggaacagcgacaagctgatcgccagaaagaaggactgggaccccaagaagtacggcggcttcgacagccctaccgtggcctactctgtgctggtggtggctaaggtggaaaagggcaagtccaagaaactgaagagtgtgaaagagctgctggggatcaccatcatggaaagaagcagctttgagaagaaccctatcgactttctggaagccaagggctacaaagaagtgaaaaaggacctgatcatcaagctgcctaagtactccctgttcgagctggaaaacggcagaaagagaatgctggcctctgccggcgaactgcagaagggaaacgagctggccctgcctagcaaatatgtgaacttcctgtacctggcctcccactatgagaagctgaagggcagccctgaggacaacgaacagaaacagctgtttgtggaacagcataagcactacctggacgagatcatcgagcagatcagcgagttctccaagagagtgatcctggccgacgccaatctggacaaggtgctgtctgcctacaacaagcacagggacaagcctatcagagagcaggccgagaatatcatccacctgttcaccctgacaaacctgggcgctcctgccgccttcaagtactttgacaccaccatcgaccggaagaggtacaccagcaccaaagaggtgctggacgccaccctgatccaccagagcatcaccggcctgtacgagacaagaatcgacctgtctcagctgggaggcgacaagagacctgccgccactaagaaggccggacaggccaaaaagaagaagtgagcggccgcatgctttatttgtgaaatttgtgatgctattgctttatttgtaaccattataagctgcaataaacaagttaacaacaacaattgcattcattttatgtttcaggttcagggggaggtgtgggaggttttttaaaagatctggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 40 - DNA, 247 bp, synthetic construct
  source 1..247: mol_type = other DNA
tcgagtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgctagcgc

SEQ ID NO: 41 - DNA, 209 bp, synthetic construct
  source 1..209: mol_type = other DNA
gcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagctt

SEQ ID NO: 42 - DNA, 179 bp, synthetic construct
  source 1..179: mol_type = other DNA
cgcccaccaggtcttgcccaaggtcttacataagaggactcttggactctcagcgatgtcaacgaccgaccttgaggcatacttcaaagactgtttgtttaaggactgggaggagttgggggaggagattaggttaaaggtctttgtagggcataaattggtctgcgcaccagcaccaa

SEQ ID NO: 43 - DNA, 103 bp, synthetic construct
  source 1..103: mol_type = other DNA
gggggaggctgctggtgaatattaaccaaggtcaccccagttatcggaggagcaaacaggggctaagtccacgggcataaattggtctgcgcaccagcaccaa

SEQ ID NO: 44 - DNA, 150 bp, synthetic construct
  source 1..150: mol_type = other DNA
cgcccaccaggtcttgcccaaggtcttacataagaggactcttggactctcagcgatgtcaacgaccgaccttgaggcatacttcaaagactgtttgtttaaggactgggaggagttgggggaggagattaggttaaaggtctttgtagg

SEQ ID NO: 45 - DNA, 74 bp, synthetic construct
  source 1..74: mol_type = other DNA
gggggaggctgctggtgaatattaaccaaggtcaccccagttatcggaggagcaaacaggggctaagtccacgg

SEQ ID NO: 46 - DNA, 29 bp, synthetic construct
  source 1..29: mol_type = other DNA
gcataaattggtctgcgcaccagcaccaa

SEQ ID NO: 47 - DNA, 5016 bp, synthetic construct
  misc_difference 220..239: note = n is a, c, g, or t
  source 1..5016: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctacgcgtggttccatggtgtaatggttagcactctggactctgaatccagcgatccgagttcaaatctcggtggaacctnnnnnnnnnnnnnnnnnnnngttttagagctagaaatagcaagttaaaataaggctagtccgttatcaacttgaaaaagtggcaccgagtcggtgctttttttctcgagtcgagtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgctagcgcactagtgccaccatggacaagaagtacagcatcggcctggacatcggcaccaactctgtgggctgggccgtgatcaccgacgagtacaaggtgcccagcaagaaattcaaggtgctgggcaacaccgacaggcacagcatcaagaagaacctgatcggcgccctgctgttcgacagcggcgaaacagccgaggccaccagactgaagagaaccgccagaagaagatacaccaggcggaagaacaggatctgctatctgcaagagatcttcagcaacgagatggccaaggtggacgacagcttcttccacagactggaagagtccttcctggtggaagaggacaagaagcacgagagacaccccatcttcggcaacatcgtggacgaggtggcctaccacgagaagtaccccaccatctaccacctgagaaagaaactggtggacagcaccgacaaggccgacctgagactgatctacctggccctggcccacatgatcaagttcagaggccacttcctgatcgagggcgacctgaaccccgacaacagcgacgtggacaagctgttcatccagctggtgcagacctacaaccagctgttcgaggaaaaccccatcaacgccagcggcgtggacgccaaggctatcctgtctgccagactgagcaagagcagaaggctggaaaatctgatcgcccagctgcccggcgagaagaagaacggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgttcctggccgccaagaacctgtctgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagaaatcttcttcgaccagagcaagaacggctacgccggctacatcgatggcggcgctagccaggaagagttctacaagttcatcaagcccatcctggaaaagatggacggcaccgaggaactgctcgtgaagctgaacagagaggacctgctgagaaagcagagaaccttcgacaacggcagcatcccccaccagatccacctgggagagctgcacgctatcctgagaaggcaggaagatttttacccattcctgaaggacaaccgggaaaagatcgagaagatcctgaccttcaggatcccctactacgtgggccccctggccagaggcaacagcagattcgcctggatgaccagaaagagcgaggaaaccatcaccccctggaacttcgaggaagtggtggacaagggcgccagcgcccagagcttcatcgagagaatgacaaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaggccatcgtggacctgctgttcaagaccaacagaaaagtgaccgtgaagcagctgaaagaggactacttcaagaaaatcgagtgcttcgactccgtggaaatctccggcgtggaagatagattcaacgcctccctgggcacataccacgatctgctgaaaattatcaaggacaaggacttcctggataacgaagagaacgaggacattctggaagatatcgtgctgaccctgacactgtttgaggaccgcgagatgatcgaggaaaggctgaaaacctacgctcacctgttcgacgacaaagtgatgaagcagctgaagagaaggcggtacaccggctggggcaggctgagcagaaagctgatcaacggcatcagagacaagcagagcggcaagacaatcctggatttcctgaagtccgacggcttcgccaaccggaacttcatgcagctgatccacgacgacagcctgacattcaaagaggacatccagaaagcccaggtgtccggccagggcgactctctgcacgagcatatcgctaacctggccggcagccccgctatcaagaagggcatcctgcagacagtgaaggtggtggacgagctcgtgaaagtgatgggcagacacaagcccgagaacatcgtgatcgagatggctagagagaaccagaccacccagaagggacagaagaactcccgcgagaggatgaagagaatcgaagagggcatcaaagagctgggcagccagatcctgaaagaacaccccgtggaaaacacccagctgcagaacgagaagctgtacctgtactacctgcagaatggccgggatatgtacgtggaccaggaactggacatcaacagactgtccgactacgatgtggaccatatcgtgcctcagagctttctgaaggacgactccatcgataacaaagtgctgactcggagcgacaagaacagaggcaagagcgacaacgtgccctccgaagaggtcgtgaagaagatgaagaactactggcgacagctgctgaacgccaagctgattacccagaggaagttcgataacctgaccaaggccgagagaggcggcctgagcgagctggataaggccggcttcatcaagaggcagctggtggaaaccagacagatcacaaagcacgtggcacagatcctggactcccggatgaacactaagtacgacgaaaacgataagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgaaatcaccctggccaacggcgagatcagaaagcgccctctgatcgagacaaacggcgaaaccggggagatcgtgtgggataagggcagagacttcgccacagtgcgaaaggtgctgagcatgccccaagtgaatatcgtgaaaaagaccgaggtgcagacaggcggcttcagcaaagagtctatcctgcccaagaggaacagcgacaagctgatcgccagaaagaaggactgggaccccaagaagtacggcggcttcgacagccctaccgtggcctactctgtgctggtggtggctaaggtggaaaagggcaagtccaagaaactgaagagtgtgaaagagctgctggggatcaccatcatggaaagaagcagctttgagaagaaccctatcgactttctggaagccaagggctacaaagaagtgaaaaaggacctgatcatcaagctgcctaagtactccctgttcgagctggaaaacggcagaaagagaatgctggcctctgccggcgaactgcagaagggaaacgagctggccctgcctagcaaatatgtgaacttcctgtacctggcctcccactatgagaagctgaagggcagccctgaggacaacgaacagaaacagctgtttgtggaacagcataagcactacctggacgagatcatcgagcagatcagcgagttctccaagagagtgatcctggccgacgccaatctggacaaggtgctgtctgcctacaacaagcacagggacaagcctatcagagagcaggccgagaatatcatccacctgttcaccctgacaaacctgggcgctcctgccgccttcaagtactttgacaccaccatcgaccggaagaggtacaccagcaccaaagaggtgctggacgccaccctgatccaccagagcatcaccggcctgtacgagacaagaatcgacctgtctcagctgggaggcgacggaggcggctcacccaaaaagaaaaggaaagtctaatctagaatgctttatttgtgaaatttgtgatgctattgctttatttgtaaccattataagctgcaataaacaagttaacaacaacaattgcattcattttatgtttcaggttcagggggaggtgtgggaggttttttaaagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 48 - DNA, 4978 bp, synthetic construct
  misc_difference 220..239: note = n is a, c, g, or t
  source 1..4978: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctacgcgtggttccatggtgtaatggttagcactctggactctgaatccagcgatccgagttcaaatctcggtggaacctnnnnnnnnnnnnnnnnnnnngttttagagctagaaatagcaagttaaaataaggctagtccgttatcaacttgaaaaagtggcaccgagtcggtgctttttttctcgaggcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttactagtgccaccatggacaagaagtacagcatcggcctggacatcggcaccaactctgtgggctgggccgtgatcaccgacgagtacaaggtgcccagcaagaaattcaaggtgctgggcaacaccgacaggcacagcatcaagaagaacctgatcggcgccctgctgttcgacagcggcgaaacagccgaggccaccagactgaagagaaccgccagaagaagatacaccaggcggaagaacaggatctgctatctgcaagagatcttcagcaacgagatggccaaggtggacgacagcttcttccacagactggaagagtccttcctggtggaagaggacaagaagcacgagagacaccccatcttcggcaacatcgtggacgaggtggcctaccacgagaagtaccccaccatctaccacctgagaaagaaactggtggacagcaccgacaaggccgacctgagactgatctacctggccctggcccacatgatcaagttcagaggccacttcctgatcgagggcgacctgaaccccgacaacagcgacgtggacaagctgttcatccagctggtgcagacctacaaccagctgttcgaggaaaaccccatcaacgccagcggcgtggacgccaaggctatcctgtctgccagactgagcaagagcagaaggctggaaaatctgatcgcccagctgcccggcgagaagaagaacggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgttcctggccgccaagaacctgtctgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagaaatcttcttcgaccagagcaagaacggctacgccggctacatcgatggcggcgctagccaggaagagttctacaagttcatcaagcccatcctggaaaagatggacggcaccgaggaactgctcgtgaagctgaacagagaggacctgctgagaaagcagagaaccttcgacaacggcagcatcccccaccagatccacctgggagagctgcacgctatcctgagaaggcaggaagatttttacccattcctgaaggacaaccgggaaaagatcgagaagatcctgaccttcaggatcccctactacgtgggccccctggccagaggcaacagcagattcgcctggatgaccagaaagagcgaggaaaccatcaccccctggaacttcgaggaagtggtggacaagggcgccagcgcccagagcttcatcgagagaatgacaaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaggccatcgtggacctgctgttcaagaccaacagaaaagtgaccgtgaagcagctgaaagaggactacttcaagaaaatcgagtgcttcgactccgtggaaatctccggcgtggaagatagattcaacgcctccctgggcacataccacgatctgctgaaaattatcaaggacaaggacttcctggataacgaagagaacgaggacattctggaagatatcgtgctgaccctgacactgtttgaggaccgcgagatgatcgaggaaaggctgaaaacctacgctcacctgttcgacgacaaagtgatgaagcagctgaagagaaggcggtacaccggctggggcaggctgagcagaaagctgatcaacggcatcagagacaagcagagcggcaagacaatcctggatttcctgaagtccgacggcttcgccaaccggaacttcatgcagctgatccacgacgacagcctgacattcaaagaggacatccagaaagcccaggtgtccggccagggcgactctctgcacgagcatatcgctaacctggccggcagccccgctatcaagaagggcatcctgcagacagtgaaggtggtggacgagctcgtgaaagtgatgggcagacacaagcccgagaacatcgtgatcgagatggctagagagaaccagaccacccagaagggacagaagaactcccgcgagaggatgaagagaatcgaagagggcatcaaagagctgggcagccagatcctgaaagaacaccccgtggaaaacacccagctgcagaacgagaagctgtacctgtactacctgcagaatggccgggatatgtacgtggaccaggaactggacatcaacagactgtccgactacgatgtggaccatatcgtgcctcagagctttctgaaggacgactccatcgataacaaagtgctgactcggagcgacaagaacagaggcaagagcgacaacgtgccctccgaagaggtcgtgaagaagatgaagaactactggcgacagctgctgaacgccaagctgattacccagaggaagttcgataacctgaccaaggccgagagaggcggcctgagcgagctggataaggccggcttcatcaagaggcagctggtggaaaccagacagatcacaaagcacgtggcacagatcctggactcccggatgaacactaagtacgacgaaaacgataagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgaaatcaccctggccaacggcgagatcagaaagcgccctctgatcgagacaaacggcgaaaccggggagatcgtgtgggataagggcagagacttcgccacagtgcgaaaggtgctgagcatgccccaagtgaatatcgtgaaaaagaccgaggtgcagacaggcggcttcagcaaagagtctatcctgcccaagaggaacagcgacaagctgatcgccagaaagaaggactgggaccccaagaagtacggcggcttcgacagccctaccgtggcctactctgtgctggtggtggctaaggtggaaaagggcaagtccaagaaactgaagagtgtgaaagagctgctggggatcaccatcatggaaagaagcagctttgagaagaaccctatcgactttctggaagccaagggctacaaagaagtgaaaaaggacctgatcatcaagctgcctaagtactccctgttcgagctggaaaacggcagaaagagaatgctggcctctgccggcgaactgcagaagggaaacgagctggccctgcctagcaaatatgtgaacttcctgtacctggcctcccactatgagaagctgaagggcagccctgaggacaacgaacagaaacagctgtttgtggaacagcataagcactacctggacgagatcatcgagcagatcagcgagttctccaagagagtgatcctggccgacgccaatctggacaaggtgctgtctgcctacaacaagcacagggacaagcctatcagagagcaggccgagaatatcatccacctgttcaccctgacaaacctgggcgctcctgccgccttcaagtactttgacaccaccatcgaccggaagaggtacaccagcaccaaagaggtgctggacgccaccctgatccaccagagcatcaccggcctgtacgagacaagaatcgacctgtctcagctgggaggcgacggaggcggctcacccaaaaagaaaaggaaagtctaatctagaatgctttatttgtgaaatttgtgatgctattgctttatttgtaaccattataagctgcaataaacaagttaacaacaacaattgcattcattttatgtttcaggttcagggggaggtgtgggaggttttttaaagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 49 - DNA, 4948 bp, synthetic construct
  misc_difference 220..239: note = n is a, c, g, or t
  source 1..4948: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctacgcgtggttccatggtgtaatggttagcactctggactctgaatccagcgatccgagttcaaatctcggtggaacctnnnnnnnnnnnnnnnnnnnngttttagagctagaaatagcaagttaaaataaggctagtccgttatcaacttgaaaaagtggcaccgagtcggtgctttttttctcgagcgcccaccaggtcttgcccaaggtcttacataagaggactcttggactctcagcgatgtcaacgaccgaccttgaggcatacttcaaagactgtttgtttaaggactgggaggagttgggggaggagattaggttaaaggtctttgtagggcataaattggtctgcgcaccagcaccaaactagtgccaccatggacaagaagtacagcatcggcctggacatcggcaccaactctgtgggctgggccgtgatcaccgacgagtacaaggtgcccagcaagaaattcaaggtgctgggcaacaccgacaggcacagcatcaagaagaacctgatcggcgccctgctgttcgacagcggcgaaacagccgaggccaccagactgaagagaaccgccagaagaagatacaccaggcggaagaacaggatctgctatctgcaagagatcttcagcaacgagatggccaaggtggacgacagcttcttccacagactggaagagtccttcctggtggaagaggacaagaagcacgagagacaccccatcttcggcaacatcgtggacgaggtggcctaccacgagaagtaccccaccatctaccacctgagaaagaaactggtggacagcaccgacaaggccgacctgagactgatctacctggccctggcccacatgatcaagttcagaggccacttcctgatcgagggcgacctgaaccccgacaacagcgacgtggacaagctgttcatccagctggtgcagacctacaaccagctgttcgaggaaaaccccatcaacgccagcggcgtggacgccaaggctatcctgtctgccagactgagcaagagcagaaggctggaaaatctgatcgcccagctgcccggcgagaagaagaacggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgttcctggccgccaagaacctgtctgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagaaatcttcttcgaccagagcaagaacggctacgccggctacatcgatggcggcgctagccaggaagagttctacaagttcatcaagcccatcctggaaaagatggacggcaccgaggaactgctcgtgaagctgaacagagaggacctgctgagaaagcagagaaccttcgacaacggcagcatcccccaccagatccacctgggagagctgcacgctatcctgagaaggcaggaagatttttacccattcctgaaggacaaccgggaaaagatcgagaagatcctgaccttcaggatcccctactacgtgggccccctggccagaggcaacagcagattcgcctggatgaccagaaagagcgaggaaaccatcaccccctggaacttcgaggaagtggtggacaagggcgccagcgcccagagcttcatcgagagaatgacaaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaggccatcgtggacctgctgttcaagaccaacagaaaagtgaccgtgaagcagctgaaagaggactacttcaagaaaatcgagtgcttcgactccgtggaaatctccggcgtggaagatagattcaacgcctccctgggcacataccacgatctgctgaaaattatcaaggacaaggacttcctggataacgaagagaacgaggacattctggaagatatcgtgctgaccctgacactgtttgaggaccgcgagatgatcgaggaaaggctgaaaacctacgctcacctgttcgacgacaaagtgatgaagcagctgaagagaaggcggtacaccggctggggcaggctgagcagaaagctgatcaacggcatcagagacaagcagagcggcaagacaatcctggatttcctgaagtccgacggcttcgccaaccggaacttcatgcagctgatccacgacgacagcctgacattcaaagaggacatccagaaagcccaggtgtccggccagggcgactctctgcacgagcatatcgctaacctggccggcagccccgctatcaagaagggcatcctgcagacagtgaaggtggtggacgagctcgtgaaagtgatgggcagacacaagcccgagaacatcgtgatcgagatggctagagagaaccagaccacccagaagggacagaagaactcccgcgagaggatgaagagaatcgaagagggcatcaaagagctgggcagccagatcctgaaagaacaccccgtggaaaacacccagctgcagaacgagaagctgtacctgtactacctgcagaatggccgggatatgtacgtggaccaggaactggacatcaacagactgtccgactacgatgtggaccatatcgtgcctcagagctttctgaaggacgactccatcgataacaaagtgctgactcggagcgacaagaacagaggcaagagcgacaacgtgccctccgaagaggtcgtgaagaagatgaagaactactggcgacagctgctgaacgccaagctgattacccagaggaagttcgataacctgaccaaggccgagagaggcggcctgagcgagctggataaggccggcttcatcaagaggcagctggtggaaaccagacagatcacaaagcacgtggcacagatcctggactcccggatgaacactaagtacgacgaaaacgataagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgaaatcaccctggccaacggcgagatcagaaagcgccctctgatcgagacaaacggcgaaaccggggagatcgtgtgggataagggcagagacttcgccacagtgcgaaaggtgctgagcatgccccaagtgaatatcgtgaaaaagaccgaggtgcagacaggcggcttcagcaaagagtctatcctgcccaagaggaacagcgacaagctgatcgccagaaagaaggactgggaccccaagaagtacggcggcttcgacagccctaccgtggcctactctgtgctggtggtggctaaggtggaaaagggcaagtccaagaaactgaagagtgtgaaagagctgctggggatcaccatcatggaaagaagcagctttgagaagaaccctatcgactttctggaagccaagggctacaaagaagtgaaaaaggacctgatcatcaagctgcctaagtactccctgttcgagctggaaaacggcagaaagagaatgctggcctctgccggcgaactgcagaagggaaacgagctggccctgcctagcaaatatgtgaacttcctgtacctggcctcccactatgagaagctgaagggcagccctgaggacaacgaacagaaacagctgtttgtggaacagcataagcactacctggacgagatcatcgagcagatcagcgagttctccaagagagtgatcctggccgacgccaatctggacaaggtgctgtctgcctacaacaagcacagggacaagcctatcagagagcaggccgagaatatcatccacctgttcaccctgacaaacctgggcgctcctgccgccttcaagtactttgacaccaccatcgaccggaagaggtacaccagcaccaaagaggtgctggacgccaccctgatccaccagagcatcaccggcctgtacgagacaagaatcgacctgtctcagctgggaggcgacggaggcggctcacccaaaaagaaaaggaaagtctaatctagaatgctttatttgtgaaatttgtgatgctattgctttatttgtaaccattataagctgcaataaacaagttaacaacaacaattgcattcattttatgtttcaggttcagggggaggtgtgggaggttttttaaagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 50 - DNA, 4872 bp, synthetic construct
  misc_difference 220..239: note = n is a, c, g, or t
  source 1..4872: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctacgcgtggttccatggtgtaatggttagcactctggactctgaatccagcgatccgagttcaaatctcggtggaacctnnnnnnnnnnnnnnnnnnnngttttagagctagaaatagcaagttaaaataaggctagtccgttatcaacttgaaaaagtggcaccgagtcggtgctttttttctcgaggggggaggctgctggtgaatattaaccaaggtcaccccagttatcggaggagcaaacaggggctaagtccacgggcataaattggtctgcgcaccagcaccaaactagtgccaccatggacaagaagtacagcatcggcctggacatcggcaccaactctgtgggctgggccgtgatcaccgacgagtacaaggtgcccagcaagaaattcaaggtgctgggcaacaccgacaggcacagcatcaagaagaacctgatcggcgccctgctgttcgacagcggcgaaacagccgaggccaccagactgaagagaaccgccagaagaagatacaccaggcggaagaacaggatctgctatctgcaagagatcttcagcaacgagatggccaaggtggacgacagcttcttccacagactggaagagtccttcctggtggaagaggacaagaagcacgagagacaccccatcttcggcaacatcgtggacgaggtggcctaccacgagaagtaccccaccatctaccacctgagaaagaaactggtggacagcaccgacaaggccgacctgagactgatctacctggccctggcccacatgatcaagttcagaggccacttcctgatcgagggcgacctgaaccccgacaacagcgacgtggacaagctgttcatccagctggtgcagacctacaaccagctgttcgaggaaaaccccatcaacgccagcggcgtggacgccaaggctatcctgtctgccagactgagcaagagcagaaggctggaaaatctgatcgcccagctgcccggcgagaagaagaacggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgttcctggccgccaagaacctgtctgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagaaatcttcttcgaccagagcaagaacggctacgccggctacatcgatggcggcgctagccaggaagagttctacaagttcatcaagcccatcctggaaaagatggacggcaccgaggaactgctcgtgaagctgaacagagaggacctgctgagaaagcagagaaccttcgacaacggcagcatcccccaccagatccacctgggagagctgcacgctatcctgagaaggcaggaagatttttacccattcctgaaggacaaccgggaaaagatcgagaagatcctgaccttcaggatcccctactacgtgggccccctggccagaggcaacagcagattcgcctggatgaccagaaagagcgaggaaaccatcaccccctggaacttcgaggaagtggtggacaagggcgccagcgcccagagcttcatcgagagaatgacaaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaggccatcgtggacctgctgttcaagaccaacagaaaagtgaccgtgaagcagctgaaagaggactacttcaagaaaatcgagtgcttcgactccgtggaaatctccggcgtggaagatagattcaacgcctccctgggcacataccacgatctgctgaaaattatcaaggacaaggacttcctggataacgaagagaacgaggacattctggaagatatcgtgctgaccctgacactgtttgaggaccgcgagatgatcgaggaaaggctgaaaacctacgctcacctgttcgacgacaaagtgatgaagcagctgaagagaaggcggtacaccggctggggcaggctgagcagaaagctgatcaacggcatcagagacaagcagagcggcaagacaatcctggatttcctgaagtccgacggcttcgccaaccggaacttcatgcagctgatccacgacgacagcctgacattcaaagaggacatccagaaagcccaggtgtccggccagggcgactctctgcacgagcatatcgctaacctggccggcagccccgctatcaagaagggcatcctgcagacagtgaaggtggtggacgagctcgtgaaagtgatgggcagacacaagcccgagaacatcgtgatcgagatggctagagagaaccagaccacccagaagggacagaagaactcccgcgagaggatgaagagaatcgaagagggcatcaaagagctgggcagccagatcctgaaagaacaccccgtggaaaacacccagctgcagaacgagaagctgtacctgtactacctgcagaatggccgggatatgtacgtggaccaggaactggacatcaacagactgtccgactacgatgtggaccatatcgtgcctcagagctttctgaaggacgactccatcgataacaaagtgctgactcggagcgacaagaacagaggcaagagcgacaacgtgccctccgaagaggtcgtgaagaagatgaagaactactggcgacagctgctgaacgccaagctgattacccagaggaagttcgataacctgaccaaggccgagagaggcggcctgagcgagctggataaggccggcttcatcaagaggcagctggtggaaaccagacagatcacaaagcacgtggcacagatcctggactcccggatgaacactaagtacgacgaaaacgataagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgaaatcaccctggccaacggcgagatcagaaagcgccctctgatcgagacaaacggcgaaaccggggagatcgtgtgggataagggcagagacttcgccacagtgcgaaaggtgctgagcatgccccaagtgaatatcgtgaaaaagaccgaggtgcagacaggcggcttcagcaaagagtctatcctgcccaagaggaacagcgacaagctgatcgccagaaagaaggactgggaccccaagaagtacggcggcttcgacagccctaccgtggcctactctgtgctggtggtggctaaggtggaaaagggcaagtccaagaaactgaagagtgtgaaagagctgctggggatcaccatcatggaaagaagcagctttgagaagaaccctatcgactttctggaagccaagggctacaaagaagtgaaaaaggacctgatcatcaagctgcctaagtactccctgttcgagctggaaaacggcagaaagagaatgctggcctctgccggcgaactgcagaagggaaacgagctggccctgcctagcaaatatgtgaacttcctgtacctggcctcccactatgagaagctgaagggcagccctgaggacaacgaacagaaacagctgtttgtggaacagcataagcactacctggacgagatcatcgagcagatcagcgagttctccaagagagtgatcctggccgacgccaatctggacaaggtgctgtctgcctacaacaagcacagggacaagcctatcagagagcaggccgagaatatcatccacctgttcaccctgacaaacctgggcgctcctgccgccttcaagtactttgacaccaccatcgaccggaagaggtacaccagcaccaaagaggtgctggacgccaccctgatccaccagagcatcaccggcctgtacgagacaagaatcgacctgtctcagctgggaggcgacggaggcggctcacccaaaaagaaaaggaaagtctaatctagaatgctttatttgtgaaatttgtgatgctattgctttatttgtaaccattataagctgcaataaacaagttaacaacaacaattgcattcattttatgtttcaggttcagggggaggtgtgggaggttttttaaagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 51 - RNA, 16 nt, synthetic construct
  source 1..16: mol_type = other RNA
gttttagagctatgct

SEQ ID NO: 52 - RNA, 67 nt, synthetic construct
  source 1..67: mol_type = other RNA
agcatagcaagttaaaataaggctagtccgttatcaacttgaaaaagtggcaccgagtcggtgcttt

SEQ ID NO: 53 - RNA, 77 nt, synthetic construct
  source 1..77: mol_type = other RNA
gttttagagctagaaatagcaagttaaaataaggctagtccgttatcaacttgaaaaagtggcaccgagtcggtgct

SEQ ID NO: 54 - RNA, 82 nt, synthetic construct
  source 1..82: mol_type = other RNA
gttggaaccattcaaaacagcatagcaagttaaaataaggctagtccgttatcaacttgaaaaagtggcaccgagtcggtgc

SEQ ID NO: 55 - RNA, 76 nt, synthetic construct
  source 1..76: mol_type = other RNA
gttttagagctagaaatagcaagttaaaataaggctagtccgttatcaacttgaaaaagtggcaccgagtcggtgc

SEQ ID NO: 56 - RNA, 86 nt, synthetic construct
  source 1..86: mol_type = other RNA
gtttaagagctatgctggaaacagcatagcaagtttaaataaggctagtccgttatcaacttgaaaaagtggcaccgagtcggtgc

SEQ ID NO: 57 - RNA, 83 nt, synthetic construct
  source 1..83: mol_type = other RNA
gttttagagctagaaatagcaagttaaaataaggctagtccgttatcaacttgaaaaagtggcaccgagtcggtgcttttttt

SEQ ID NO: 58-60 - skipped (3 entries)

SEQ ID NO: 61 - DNA, 4176 bp, synthetic construct
  source 1..4176: mol_type = other DNA
atggacaagcccaagaaaaagcggaaagtgaagtacagcatcggcctggacatcggcaccaactctgtgggctgggccgtgatcaccgacgagtacaaggtgcccagcaagaaattcaaggtgctgggcaacaccgacaggcacagcatcaagaagaacctgatcggcgccctgctgttcgacagcggcgaaacagccgaggccaccagactgaagagaaccgccagaagaagatacaccaggcggaagaacaggatctgctatctgcaagagatcttcagcaacgagatggccaaggtggacgacagcttcttccacagactggaagagtccttcctggtggaagaggacaagaagcacgagagacaccccatcttcggcaacatcgtggacgaggtggcctaccacgagaagtaccccaccatctaccacctgagaaagaaactggtggacagcaccgacaaggccgacctgagactgatctacctggccctggcccacatgatcaagttcagaggccacttcctgatcgagggcgacctgaaccccgacaacagcgacgtggacaagctgttcatccagctggtgcagacctacaaccagctgttcgaggaaaaccccatcaacgccagcggcgtggacgccaaggctatcctgtctgccagactgagcaagagcagaaggctggaaaatctgatcgcccagctgcccggcgagaagaagaacggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgttcctggccgccaagaacctgtctgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagaaatcttcttcgaccagagcaagaacggctacgccggctacatcgatggcggcgctagccaggaagagttctacaagttcatcaagcccatcctggaaaagatggacggcaccgaggaactgctcgtgaagctgaacagagaggacctgctgagaaagcagagaaccttcgacaacggcagcatcccccaccagatccacctgggagagctgcacgctatcctgagaaggcaggaagatttttacccattcctgaaggacaaccgggaaaagatcgagaagatcctgaccttcaggatcccctactacgtgggccccctggccagaggcaacagcagattcgcctggatgaccagaaagagcgaggaaaccatcaccccctggaacttcgaggaagtggtggacaagggcgccagcgcccagagcttcatcgagagaatgacaaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaggccatcgtggacctgctgttcaagaccaacagaaaagtgaccgtgaagcagctgaaagaggactacttcaagaaaatcgagtgcttcgactccgtggaaatctccggcgtggaagatagattcaacgcctccctgggcacataccacgatctgctgaaaattatcaaggacaaggacttcctggataacgaagagaacgaggacattctggaagatatcgtgctgaccctgacactgtttgaggaccgcgagatgatcgaggaaaggctgaaaacctacgctcacctgttcgacgacaaagtgatgaagcagctgaagagaaggcggtacaccggctggggcaggctgagcagaaagctgatcaacggcatcagagacaagcagagcggcaagacaatcctggatttcctgaagtccgacggcttcgccaaccggaacttcatgcagctgatccacgacgacagcctgacattcaaagaggacatccagaaagcccaggtgtccggccagggcgactctctgcacgagcatatcgctaacctggccggcagccccgctatcaagaagggcatcctgcagacagtgaaggtggtggacgagctcgtgaaagtgatgggcagacacaagcccgagaacatcgtgatcgagatggctagagagaaccagaccacccagaagggacagaagaactcccgcgagaggatgaagagaatcgaagagggcatcaaagagctgggcagccagatcctgaaagaacaccccgtggaaaacacccagctgcagaacgagaagctgtacctgtactacctgcagaatggccgggatatgtacgtggaccaggaactggacatcaacagactgtccgactacgatgtggaccatatcgtgcctcagagctttctgaaggacgactccatcgataacaaagtgctgactcggagcgacaagaacagaggcaagagcgacaacgtgccctccgaagaggtcgtgaagaagatgaagaactactggcgacagctgctgaacgccaagctgattacccagaggaagttcgataacctgaccaaggccgagagaggcggcctgagcgagctggataaggccggcttcatcaagaggcagctggtggaaaccagacagatcacaaagcacgtggcacagatcctggactcccggatgaacactaagtacgacgaaaacgataagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgaaatcaccctggccaacggcgagatcagaaagcgccctctgatcgagacaaacggcgaaaccggggagatcgtgtgggataagggcagagacttcgccacagtgcgaaaggtgctgagcatgccccaagtgaatatcgtgaaaaagaccgaggtgcagacaggcggcttcagcaaagagtctatcctgcccaagaggaacagcgacaagctgatcgccagaaagaaggactgggaccccaagaagtacggcggcttcgacagccctaccgtggcctactctgtgctggtggtggctaaggtggaaaagggcaagtccaagaaactgaagagtgtgaaagagctgctggggatcaccatcatggaaagaagcagctttgagaagaaccctatcgactttctggaagccaagggctacaaagaagtgaaaaaggacctgatcatcaagctgcctaagtactccctgttcgagctggaaaacggcagaaagagaatgctggcctctgccggcgaactgcagaagggaaacgagctggccctgcctagcaaatatgtgaacttcctgtacctggcctcccactatgagaagctgaagggcagccctgaggacaacgaacagaaacagctgtttgtggaacagcataagcactacctggacgagatcatcgagcagatcagcgagttctccaagagagtgatcctggccgacgccaatctggacaaggtgctgtctgcctacaacaagcacagggacaagcctatcagagagcaggccgagaatatcatccacctgttcaccctgacaaacctgggcgctcctgccgccttcaagtactttgacaccaccatcgaccggaagaggtacaccagcaccaaagaggtgctggacgccaccctgatccaccagagcatcaccggcctgtacgagacaagaatcgacctgtctcagctgggaggcgacaagagacctgccgccactaagaaggccggacaggccaaaaagaagaagtga

SEQ ID NO: 62 - protein, 1391 aa, synthetic construct
MDKPKKKRKVKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKK

SEQ ID NO: 63 - DNA, 4218 bp, synthetic construct
  source 1..4218: mol_type = other DNA
atggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggacaagaagcacgagagacaccccatcttcggcaacatcgtggacgaggtggcctaccacgagaagtaccccaccatctaccacctgagaaagaaactggtggacagcaccgacaaggccgacctgagactgatctacctggccctggcccacatgatcaagttcagaggccacttcctgatcgagggcgacctgaaccccgacaacagcgacgtggacaagctgttcatccagctggtgcagacctacaaccagctgttcgaggaaaaccccatcaacgccagcggcgtggacgccaaggctatcctgtctgccagactgagcaagagcagaaggctggaaaatctgatcgcccagctgcccggcgagaagaagaacggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgttcctggccgccaagaacctgtctgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagaaatcttcttcgaccagagcaagaacggctacgccggctacatcgatggcggcgctagccaggaagagttctacaagttcatcaagcccatcctggaaaagatggacggcaccgaggaactgctcgtgaagctgaacagagaggacctgctgagaaagcagagaaccttcgacaacggcagcatcccccaccagatccacctgggagagctgcacgctatcctgagaaggcaggaagatttttacccattcctgaaggacaaccgggaaaagatcgagaagatcctgaccttcaggatcccctactacgtgggccccctggccagaggcaacagcagattcgcctggatgaccagaaagagcgaggaaaccatcaccccctggaacttcgaggaagtggtggacaagggcgccagcgcccagagcttcatcgagagaatgacaaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaggccatcgtggacctgctgttcaagaccaacagaaaagtgaccgtgaagcagctgaaagaggactacttcaagaaaatcgagtgcttcgactccgtggaaatctccggcgtggaagatagattcaacgcctccctgggcacataccacgatctgctgaaaattatcaaggacaaggacttcctggataacgaagagaacgaggacattctggaagatatcgtgctgaccctgacactgtttgaggaccgcgagatgatcgaggaaaggctgaaaacctacgctcacctgttcgacgacaaagtgatgaagcagctgaagagaaggcggtacaccggctggggcaggctgagcagaaagctgatcaacggcatcagagacaagcagagcggcaagacaatcctggatttcctgaagtccgacggcttcgccaaccggaacttcatgcagctgatccacgacgacagcctgacattcaaagaggacatccagaaagcccaggtgtccggccagggcgactctctgcacgagcatatcgctaacctggccggcagccccgctatcaagaagggcatcctgcagacagtgaaggtggtggacgagctcgtgaaagtgatgggcagacacaagcccgagaacatcgtgatcgagatggctagagagaaccagaccacccagaagggacagaagaactcccgcgagaggatgaagagaatcgaagagggcatcaaagagctgggcagccagatcctgaaagaacaccccgtggaaaacacccagctgcagaacgagaagctgtacctgtactacctgcagaatggccgggatatgtacgtggaccaggaactggacatcaacagactgtccgactacgatgtggaccatatcgtgcctcagagctttctgaaggacgactccatcgataacaaagtgctgactcggagcgacaagaacagaggcaagagcgacaacgtgccctccgaagaggtcgtgaagaagatgaagaactactggcgacagctgctgaacgccaagctgattacccagaggaagttcgataacctgaccaaggccgagagaggcggcctgagcgagctggataaggccggcttcatcaagaggcagctggtggaaaccagacagatcacaaagcacgtggcacagatcctggactcccggatgaacactaagtacgacgaaaacgataagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgaaatcaccctggccaacggcgagatcagaaagcgccctctgatcgagacaaacggcgaaaccggggagatcgtgtgggataagggcagagacttcgccacagtgcgaaaggtgctgagcatgccccaagtgaatatcgtgaaaaagaccgaggtgcagacaggcggcttcagcaaagagtctatcctgcccaagaggaacagcgacaagctgatcgccagaaagaaggactgggaccccaagaagtacggcggcttcgacagccctaccgtggcctactctgtgctggtggtggctaaggtggaaaagggcaagtccaagaaactgaagagtgtgaaagagctgctggggatcaccatcatggaaagaagcagctttgagaagaaccctatcgactttctggaagccaagggctacaaagaagtgaaaaaggacctgatcatcaagctgcctaagtactccctgttcgagctggaaaacggcagaaagagaatgctggcctctgccggcgaactgcagaagggaaacgagctggccctgcctagcaaatatgtgaacttcctgtacctggcctcccactatgagaagctgaagggcagccctgaggacaacgaacagaaacagctgtttgtggaacagcataagcactacctggacgagatcatcgagcagatcagcgagttctccaagagagtgatcctggccgacgccaatctggacaaggtgctgtctgcctacaacaagcacagggacaagcctatcagagagcaggccgagaatatcatccacctgttcaccctgacaaacctgggcgctcctgccgccttcaagtactttgacaccaccatcgaccggaagaggtacaccagcaccaaagaggtgctggacgccaccctgatccaccagagcatcaccggcctgtacgagacaagaatcgacctgtctcagctgggaggcgacaagagacctgccgccactaagaaggccggacaggccaaaaagaagaagtgagcggccgcttaatta

SEQ ID NO: 64 - protein, 7 aa, synthetic construct
QSVSSNY

SEQ ID NO: 65 - skipped

SEQ ID NO: 66 - protein, 9 aa, synthetic construct
QRYGTSPLT

SEQ ID NO: 67 - protein, 8 aa, synthetic construct
GFTFNYYG

SEQ ID NO: 68 - protein, 8 aa, synthetic construct
ISYDGTNK

SEQ ID NO: 69 - protein, 10 aa, synthetic construct
ARDRGGRFDY

SEQ ID NO: 70 - protein, 7 aa, synthetic construct
QSVSSNY

SEQ ID NO: 71 - skipped

SEQ ID NO: 72 - protein, 9 aa, synthetic construct
QRYGTSPLT

SEQ ID NO: 73 - protein, 8 aa, synthetic construct
GFTFNYYG

SEQ ID NO: 74 - protein, 8 aa, synthetic construct
ISYDGTNK

SEQ ID NO: 75 - protein, 10 aa, synthetic construct
ARDRGGRFDY

SEQ ID NO: 76 - protein, 6 aa, synthetic construct
QGIRNN

SEQ ID NO: 77 - skipped

SEQ ID NO: 78 - protein, 9 aa, synthetic construct
LQYNNYPWT

SEQ ID NO: 79 - protein, 8 aa, synthetic construct
GGTFSSYA

SEQ ID NO: 80 - protein, 8 aa, synthetic construct
IIPIFGTP

SEQ ID NO: 81 - protein, 13 aa, synthetic construct
ARQQPVYQYNMDV

SEQ ID NO: 82 - DNA, 21 bp, synthetic construct
  source 1..21: mol_type = other DNA
ggaacccctagtgatggagtt

SEQ ID NO: 83 - DNA, 16 bp, synthetic construct
  source 1..16: mol_type = other DNA
cggcctcagtgagcga

SEQ ID NO: 84 - DNA, 21 bp, synthetic construct
  source 1..21: mol_type = other DNA
cactccctctctgcgcgctcg

SEQ ID NO: 85 - DNA, 21 bp, synthetic construct
  source 1..21: mol_type = other DNA
cagagtgtgtctagtaattat

SEQ ID NO: 86 - skipped

SEQ ID NO: 87 - DNA, 27 bp, synthetic construct
  source 1..27: mol_type = other DNA
cagcgctacggtaccagccccctgaca

SEQ ID NO: 88 - DNA, 24 bp, synthetic construct
  source 1..24: mol_type = other DNA
ggttttacgttcaattattatggc

SEQ ID NO: 89 - DNA, 24 bp, synthetic construct
  source 1..24: mol_type = other DNA
attagttacgacggaaccaataag

SEQ ID NO: 90 - DNA, 30 bp, synthetic construct
  source 1..30: mol_type = other DNA
gcgagagatcgagggggcagatttgactac

SEQ ID NO: 91 - DNA, 21 bp, synthetic construct
  source 1..21: mol_type = other DNA
cagagtgttagcagcaactac

SEQ ID NO: 92 - skipped

SEQ ID NO: 93 - DNA, 27 bp, synthetic construct
  source 1..27: mol_type = other DNA
cagcggtatggtacctcaccgctcact

SEQ ID NO: 94 - DNA, 24 bp, synthetic construct
  source 1..24: mol_type = other DNA
ggattcaccttcaattactatggc

SEQ ID NO: 95 - DNA, 24 bp, synthetic construct
  source 1..24: mol_type = other DNA
atatcatatgatggaactaataaa

SEQ ID NO: 96 - DNA, 30 bp, synthetic construct
  source 1..30: mol_type = other DNA
gcgagagatcgcggtggccgctttgactac

SEQ ID NO: 97 - DNA, 18 bp, synthetic construct
  source 1..18: mol_type = other DNA
cagggcattagaaacaac

SEQ ID NO: 98 - skipped

SEQ ID NO: 99 - DNA, 27 bp, synthetic construct
  source 1..27: mol_type = other DNA
ttgcagtataataactatccctggacc

SEQ ID NO: 100 - DNA, 24 bp, synthetic construct
  source 1..24: mol_type = other DNA
ggtgggacatttagtagttatgcc

SEQ ID NO: 101 - DNA, 24 bp, synthetic construct
  source 1..24: mol_type = other DNA
atcataccgatctttggtacaccc

SEQ ID NO: 102 - DNA, 39 bp, synthetic construct
  source 1..39: mol_type = other DNA
gcaaggcagcagccagtgtaccaatataatatggatgtc

SEQ ID NO: 103 - DNA, 324 bp, synthetic construct
  source 1..324: mol_type = other DNA
gaaatagtgctgacccagtcaccagataccctgagcctgagtcctggggaacgggcaacactcagttgtagggcatcccagagtgtgtctagtaattatctggcttggtaccagcaaaaaccggggcaggctccccgactgctgatctatggcgcaagcagccgagccaccggtattccagatcgatttagtggatctggaagtggaactgacttcacgttgacaatatcaagactggaacccgaagatttcgctgtgtattattgccagcgctacggtaccagccccctgacattcggggggggaacgaaggttgaaataaaa

SEQ ID NO: 104 - protein, 108 aa, synthetic construct
EIVLTQSPDTLSLSPGERATLSCRASQSVSSNYLAWYQQKPGQAPRLLIYGASSRATGIPDRFSGSGSGTDFTLTISRLEPEDFAVYYCQRYGTSPLTFGGGTKVEIK

SEQ ID NO: 105 - DNA, 351 bp, synthetic construct
  source 1..351: mol_type = other DNA
caggtacagctcgttgagagcggaggtggggttgtgcagcctgggagatctctccgcctcagttgcgccgcctcaggttttacgttcaattattatggcatgcattgggttagacaagctccggggaaggggttggaatgggtagccgtaattagttacgacggaaccaataagtattatgctgacagtgtgaagggtcgatttacgacatcccgggataactccaagaacacattgtaccttcaaatgaattctttgcgggcggaagatactgcactctattattgtgcgagagatcgagggggcagatttgactactggggccaaggaatacaggttactgtatcatct

SEQ ID NO: 106 - protein, 117 aa, synthetic construct
QVQLVESGGGVVQPGRSLRLSCAASGFTFNYYGMHWVRQAPGKGLEWVAVISYDGTNKYYADSVKGRFTTSRDNSKNTLYLQMNSLRAEDTALYYCARDRGGRFDYWGQGIQVTVSS

SEQ ID NO: 107 - DNA, 324 bp, synthetic construct
  source 1..324: mol_type = other DNA
gaaattgtgttgacgcagtctccagacaccctgtctttgtctccaggggaaagagccaccctctcctgcagggccagtcagagtgttagcagcaactacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcggtatggtacctcaccgctcactttcggcggagggaccaaggtggagatcaaa

SEQ ID NO: 108 - protein, 108 aa, synthetic construct; identical to SEQ ID NO: 104

SEQ ID NO: 109 - DNA, 351 bp, synthetic construct
  source 1..351: mol_type = other DNA
caggtgcagctggtggagtcggggggaggcgtggtccagcctgggaggtccctgagactctcctgtgcagcctctggattcaccttcaattactatggcatgcactgggtccgccaggctccaggcaaggggctggagtgggtggcagtcatatcatatgatggaactaataaatactatgcagactccgtgaagggccgattcaccacctccagagacaattccaagaacacgctgtatctgcagatgaacagcctgagagctgaggacacggctctgtattactgtgcgagagatcgcggtggccgctttgactactggggccagggaatccaggtcaccgtctcctca

SEQ ID NO: 110 - protein, 117 aa, synthetic construct; identical to SEQ ID NO: 106

SEQ ID NO: 111 - DNA, 321 bp, synthetic construct
  source 1..321: mol_type = other DNA
gacatacagatgacgcagtccccttccagcctcagcgcatcagtgggggacagagtcactatcacttgcagggcttctcagggcattagaaacaacttgggctggtaccaacagaagcctctgaaggcacctaaacggttgatttacgccgccagctctttgcaatctggggtgccttccagattcagcggctctggctcaggaaccgaatttaccctgaccattagcagcttgcaaccggaggatttcgctacctactattgcttgcagtataataactatccctggaccttcggtcaaggtaccaaggtcgagataaag

SEQ ID NO: 112 - protein, 107 aa, synthetic construct
DIQMTQSPSSLSASVGDRVTITCRASQGIRNNLGWYQQKPLKAPKRLIYAASSLQSGVPSRFSGSGSGTEFTLTISSLQPEDFATYYCLQYNNYPWTFGQGTKVEIK

SEQ ID NO: 113 - DNA, 360 bp, synthetic construct
  source 1..360: mol_type = other DNA
caggtccagctcgtccaatccggggcggaagtcaaaaagagcggctcatccgtcaaggtctcctgtaaggcctcaggtgggacatttagtagttatgccatctcctgggttcgccaggctccgggacagggcttggagtggatgggtggaatcataccgatctttggtacaccctcatacgcgcagaaattccaagaccgcgtcacgatcacgactgacgaatccacgagcaccgtttacatggagttgtcttcactgagaagtgaggacactgcagtgtattattgtgcaaggcagcagccagtgtaccaatataatatggatgtctggggtcaaggcaccaccgtgaccgtgtcctcc

SEQ ID NO: 114 - protein, 120 aa, synthetic construct
QVQLVQSGAEVKKSGSSVKVSCKASGGTFSSYAISWVRQAPGQGLEWMGGIIPIFGTPSYAQKFQDRVTITTDESTSTVYMELSSLRSEDTAVYYCARQQPVYQYNMDVWGQGTTVTVSS

SEQ ID NO: 115 - DNA, 2220 bp, synthetic construct
  source 1..2220: mol_type = other DNA
atgaagtgggtaacctttctcctcctcctcttcgtctccggctctgctttttccaggggtgtgtttcgccgagaagcacccgaaatagtgctgacccagtcaccagataccctgagcctgagtcctggggaacgggcaacactcagttgtagggcatcccagagtgtgtctagtaattatctggcttggtaccagcaaaaaccggggcaggctccccgactgctgatctatggcgcaagcagccgagccaccggtattccagatcgatttagtggatctggaagtggaactgacttcacgttgacaatatcaagactggaacccgaagatttcgctgtgtattattgccagcgctacggtaccagccccctgacattcggggggggaacgaaggttgaaataaaacgcaccgtcgcggcgccatctgtattcatttttcccccgtctgatgagcaactgaaatcagggaccgcgtccgtggtctgccttctgaacaatttttacccgagagaggcgaaagtccagtggaaggtggataatgcgcttcagtcaggtaactctcaggagagcgtcacagagcaagactctaaagattcaacttacagcctttcctccaccctgactctgtccaaggccgactacgagaaacataaggtctatgcctgcgaagtaactcatcaaggtcttagttcacccgtcacgaaaagttttaataggggggagtgtagaaaacggaggggatcaggggcgactaacttttcattgcttaagcaagcaggagacgtggaagagaatcccgggccccataggccgcgacgacgggggaccagaccccctcctttggccctgctggctgctttgcttctcgcggcgcgaggagcggacgctcaggtacagctcgttgagagcggaggtggggttgtgcagcctgggagatctctccgcctcagttgcgccgcctcaggttttacgttcaattattatggcatgcattgggttagacaagctccggggaaggggttggaatgggtagccgtaattagttacgacggaaccaataagtattatgctgacagtgtgaagggtcgatttacgacatcccgggataactccaagaacacattgtaccttcaaatgaattctttgcgggcggaagatactgcactctattattgtgcgagagatcgagggggcagatttgactactggggccaaggaatacaggttactgtatcatctgcttcaactaagggtccgagcgtatttccccttgctccttgcagccgatcaacaagtgaaagtacagctgctttgggttgccttgtgaaagattatttccctgagcctgtgactgtttcctggaattcaggtgctcttactagcggggttcatacatttcccgctgtactccagtcaagcgggctctatagtctcagtagcgtagtaacggtaccctcttcatcacttgggacaaagacgtacacatgcaatgtagaccataagccgtctaatacgaaagttgataaaagggtagaatccaaatatggcccgccgtgtccgccttgtccagctccgggcggtgggggccccagtgtattcctgtttccccctaaaccgaaggatacgcttatgattagtcgaacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 116 - DNA, 2214 bp, synthetic construct
  source 1..2214: mol_type = other DNA
atgaagtgggtaacctttctcctcctcctcttcgtctccggctctgctttttccaggggtgtgtttcgccgagaagcaccccaggtgcagctggtggagtcggggggaggcgtggtccagcctgggaggtccctgagactctcctgtgcagcctctggattcaccttcaattactatggcatgcactgggtccgccaggctccaggcaaggggctggagtgggtggcagtcatatcatatgatggaactaataaatactatgcagactccgtgaagggccgattcaccacctccagagacaattccaagaacacgctgtatctgcagatgaacagcctgagagctgaggacacggctctgtattactgtgcgagagatcgcggtggccgctttgactactggggccagggaatccaggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccaggcggtggcggaccatcagtcttcctgttccccccaaaacccaaggacactctctacatcacccgggagcctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaacgtaaacgaagaggatccggggtgaagcaaaccttgaatttcgatctcctgaagttggctggcgatgtggagagtaatcccggcccaaagtgggtaacctttctcctcctcctcttcgtctccggctctgctttttccaggggtgtgtttcgccgagaaattgtgttgacgcagtctccagacaccctgtctttgtctccaggggaaagagccaccctctcctgcagggccagtcagagtgttagcagcaactacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcggtatggtacctcaccgctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaa

SEQ ID NO: 117 - DNA, 2205 bp, synthetic construct
  source 1..2205: mol_type = other DNA
atgaagtgggtaacctttctcctcctcctcttcgtctccggctctgctttttccaggggtgtgtttcgccgagaagcaccccaggtgcagctggtggagtcggggggaggcgtggtccagcctgggaggtccctgagactctcctgtgcagcctctggattcaccttcaattactatggcatgcactgggtccgccaggctccaggcaaggggctggagtgggtggcagtcatatcatatgatggaactaataaatactatgcagactccgtgaagggccgattcaccacctccagagacaattccaagaacacgctgtatctgcagatgaacagcctgagagctgaggacacggctctgtattactgtgcgagagatcgcggtggccgctttgactactggggccagggaatccaggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccaggcggtggcggaccatcagtcttcctgttccccccaaaacccaaggacactctctacatcacccgggagcctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaacgtaaacgaagaggatccggggcgactaacttttcattgcttaagcaagcaggagacgtggaagagaatcccgggcccaagtgggtaacctttctcctcctcctcttcgtctccggctctgctttttccaggggtgtgtttcgccgagaaattgtgttgacgcagtctccagacaccctgtctttgtctccaggggaaagagccaccctctcctgcagggccagtcagagtgttagcagcaactacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcggtatggtacctcaccgctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaa

SEQ ID NO: 118 - DNA, 2202 bp, synthetic construct
  source 1..2202: mol_type = other DNA
atgaagtgggtaacctttctcctcctcctcttcgtctccggctctgctttttccaggggtgtgtttcgccgagaagcaccccaggtgcagctggtggagtcggggggaggcgtggtccagcctgggaggtccctgagactctcctgtgcagcctctggattcaccttcaattactatggcatgcactgggtccgccaggctccaggcaaggggctggagtgggtggcagtcatatcatatgatggaactaataaatactatgcagactccgtgaagggccgattcaccacctccagagacaattccaagaacacgctgtatctgcagatgaacagcctgagagctgaggacacggctctgtattactgtgcgagagatcgcggtggccgctttgactactggggccagggaatccaggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccaggcggtggcggaccatcagtcttcctgttccccccaaaacccaaggacactctctacatcacccgggagcctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaacgtaaacgaagaggatccggggagggccggggcagcctgctgacctgcggagacgtggaggagaaccctggccccaagtgggtaacctttctcctcctcctcttcgtctccggctctgctttttccaggggtgtgtttcgccgagaaattgtgttgacgcagtctccagacaccctgtctttgtctccaggggaaagagccaccctctcctgcagggccagtcagagtgttagcagcaactacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcggtatggtacctcaccgctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaa

SEQ ID NO: 119 - DNA, 2217 bp, synthetic construct
  source 1..2217: mol_type = other DNA
atgaagtgggtaacctttctcctcctcctcttcgtctccggctctgctttttccaggggtgtgtttcgccgagaagcaccccaggtgcagctggtggagtcggggggaggcgtggtccagcctgggaggtccctgagactctcctgtgcagcctctggattcaccttcaattactatggcatgcactgggtccgccaggctccaggcaaggggctggagtgggtggcagtcatatcatatgatggaactaataaatactatgcagactccgtgaagggccgattcaccacctccagagacaattccaagaacacgctgtatctgcagatgaacagcctgagagctgaggacacggctctgtattactgtgcgagagatcgcggtggccgctttgactactggggccagggaatccaggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccaggcggtggcggaccatcagtcttcctgttccccccaaaacccaaggacactctctacatcacccgggagcctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaacgtaaacgaagaggatccggggagggccggggcagcctgctgacctgcggagacgtggaggagaaccctggcccccacagacctagacgtcgtggaactcgtccacctccactggcactgctcgctgctctcctcctggctgcacgtggtgctgatgcagaaattgtgttgacgcagtctccagacaccctgtctttgtctccaggggaaagagccaccctctcctgcagggccagtcagagtgttagcagcaactacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcggtatggtacctcaccgctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaa

SEQ ID NO: 120 - DNA, 2238 bp, synthetic construct
  source 1..2238: mol_type = other DNA
atgaagtgggtaacctttctcctcctcctcttcgtctccggctctgctttttccaggggtgtgtttcgccgagaagcacccgacatacagatgacgcagtccccttccagcctcagcgcatcagtgggggacagagtcactatcacttgcagggcttctcagggcattagaaacaacttgggctggtaccaacagaagcctctgaaggcacctaaacggttgatttacgccgccagctctttgcaatctggggtgccttccagattcagcggctctggctcaggaaccgaatttaccctgaccattagcagcttgcaaccggaggatttcgctacctactattgcttgcagtataataactatccctggaccttcggtcaaggtaccaaggtcgagataaagcggaccgttgctgccccttctgtgttcatctttcccccctcagatgaacagcttaagagcggaacggcaagtgtagtatgccttcttaataatttctaccctagagaagccaaagttcagtggaaagtagataatgctttgcaaagcggaaactctcaagaatcagttacagaacaagactccaaagactcaacatactcactttcatcaacgctcaccctgtctaaagccgattacgagaagcacaaagtttacgcctgtgaggttacacatcagggtctcagtagtcctgtgactaagtcttttaaccggggggaatgcagaaaacggaggggatcaggggcgactaacttttcattgcttaagcaagcaggagacgtggaagagaatcccgggccccacagacctagacgtcgtggaactcgtccacctccactggcactgctcgctgctctcctcctggctgcacgtggtgctgatgcacaggtccagctcgtccaatccggggcggaagtcaaaaagagcggctcatccgtcaaggtctcctgtaaggcctcaggtgggacatttagtagttatgccatctcctgggttcgccaggctccgggacagggcttggagtggatgggtggaatcataccgatctttggtacaccctcatacgcgcagaaattccaagaccgcgtcacgatcacgactgacgaatccacgagcaccgtttacatggagttgtcttcactgagaagtgaggacactgcagtgtattattgtgcaaggcagcagccagtgtaccaatataatatggatgtctggggtcaaggcaccaccgtgaccgtgtcctccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagtccctctccctgtctccgggtaaatga

SEQ ID NO: 121 - RNA, 72 nt, synthetic construct
  source 1..72: mol_type = other RNA
aaacagcatagcaagttaaaataaggctagtccgttatcaacttgaaaaagtggcaccgagtcggtgctttt

SEQ ID NO: 122 - RNA, 82 nt, synthetic construct; identical to SEQ ID NO: 54
  source 1..82: mol_type = other RNA

SEQ ID NO: 123 - RNA, 80 nt, synthetic construct
  source 1..80: mol_type = other RNA
gttttagagctagaaatagcaagttaaaataaggctagtccgttatcaacttgaaaaagtggcaccgagtcggtgctttt

SEQ ID NO: 124 - RNA, 92 nt, synthetic construct
  source 1..92: mol_type = other RNA
gtttaagagctatgctggaaacagcatagcaagtttaaataaggctagtccgttatcaacttgaaaaagtggcaccgagtcggtgctttttt

SEQ ID NO: 125 - DNA, 645 bp, synthetic construct
  source 1..645: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 126 - protein, 215 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 127 - DNA, 1350 bp, synthetic construct
  source 1..1350: mol_type = other DNA
caggtccacctggtgcagtctgggccagaggtgaagaagcctgggtcctcggtgaaggtctcctgcaaggcttctggagtcaccttcatcagtcatgctatcagctgggtgcgacaggcccctggacaagggcttgaatgggtgggaggaatcatcgctatctttggtacaacaaactacgcacagaagttccagggcagagtcacggttacaacggacaaatccacgaacacagtctacatggaattgagcagactgagatctgaggacacggccatttattactgtgcgcgaggtgagacctactacgagggaaactttgacttctggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagtccctctccctgtctccgggtaaa

SEQ ID NO: 128 - protein, 450 aa, synthetic construct
QVHLVQSGPEVKKPGSSVKVSCKASGVTFISHAISWVRQAPGQGLEWVGGIIAIFGTTNYAQKFQGRVTVTTDKSTNTVYMELSRLRSEDTAIYYCARGETYYEGNFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 129 - protein, 6 aa, synthetic construct
QSISSY

SEQ ID NO: 130 - skipped

SEQ ID NO: 131 - protein, 10 aa, synthetic construct
QQSYSTPPIT

SEQ ID NO: 132 - protein, 8 aa, synthetic construct
GVTFISHA

SEQ ID NO: 133 - protein, 8 aa, synthetic construct
IIAIFGTT

SEQ ID NO: 134 - protein, 13 aa, synthetic construct
ARGETYYEGNFDF

SEQ ID NO: 135 - DNA, 18 bp, synthetic construct
  source 1..18: mol_type = other DNA
cagagcattagcagctat

SEQ ID NO: 136 - skipped

SEQ ID NO: 137 - DNA, 30 bp, synthetic construct
  source 1..30: mol_type = other DNA
caacagagttacagtacccctccgatcacc

SEQ ID NO: 138 - DNA, 24 bp, synthetic construct
  source 1..24: mol_type = other DNA
ggagtcaccttcatcagtcatgct

SEQ ID NO: 139 - DNA, 24 bp, synthetic construct
  source 1..24: mol_type = other DNA
atcatcgctatctttggtacaaca

SEQ ID NO: 140 - DNA, 39 bp, synthetic construct
  source 1..39: mol_type = other DNA
gcgcgaggtgagacctactacgagggaaactttgacttc

SEQ ID NO: 141 - DNA, 324 bp, synthetic construct
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 142 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 143 - DNA, 360 bp, synthetic construct
  source 1..360: mol_type = other DNA
caggtccacctggtgcagtctgggccagaggtgaagaagcctgggtcctcggtgaaggtctcctgcaaggcttctggagtcaccttcatcagtcatgctatcagctgggtgcgacaggcccctggacaagggcttgaatgggtgggaggaatcatcgctatctttggtacaacaaactacgcacagaagttccagggcagagtcacggttacaacggacaaatccacgaacacagtctacatggaattgagcagactgagatctgaggacacggccatttattactgtgcgcgaggtgagacctactacgagggaaactttgacttctggggccagggaaccctggtcaccgtctcctca

SEQ ID NO: 144 - protein, 120 aa, synthetic construct
QVHLVQSGPEVKKPGSSVKVSCKASGVTFISHAISWVRQAPGQGLEWVGGIIAIFGTTNYAQKFQGRVTVTTDKSTNTVYMELSRLRSEDTAIYYCARGETYYEGNFDFWGQGTLVTVSS

SEQ ID NO: 145 - DNA, 3873 bp, synthetic construct
  misc_feature 1..141: note = ITR
  misc_feature 204..467: note = hU6
  misc_feature 468..570: note = gRNA1
  misc_feature 610..709: note = SA
  misc_feature 712..1356: note = H1H11829N2 LC
  misc_feature 1357..1368: note = Furin
  misc_feature 1369..1377: note = Linker
  misc_feature 1378..1431: note = T2A
  misc_feature 1432..1518: note = mROR with ATG
  misc_feature 1519..2868: note = H1H11829N2 HC
  misc_feature 2880..3467: note = WPRE
  misc_feature 3480..3695: note = bGH PA
  misc_feature 3733..3873: note = ITR
  source 1..3873: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcgctagctgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacctgcatctgagaacccttagggttttagagctagaaatagcaagttaaaataaggctagtccgttatcaacttgaaaaagtggcaccgagtcggtgctttttttctagaccacctaagggttctcagatgcacccttacgcgttaggtcagtgaagagaagaacaaaaagcagcatattacagttagttgtcttcatcaatctttaaatatgttgtgtggtttttctctccctgtttccacagccgacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtcgtaaacgaagaggatccggggagggccggggcagcctgctgacctgcggagacgtggaggagaaccctggccccatgcacagacctagacgtcgtggaactcgtccacctccactggcactgctcgctgctctcctcctggctgcacgtggtgctgatgcacaggtccacctggtgcagtctgggccagaggtgaagaagcctgggtcctcggtgaaggtctcctgcaaggcttctggagtcaccttcatcagtcatgctatcagctgggtgcgacaggcccctggacaagggcttgaatgggtgggaggaatcatcgctatctttggtacaacaaactacgcacagaagttccagggcagagtcacggttacaacggacaaatccacgaacacagtctacatggaattgagcagactgagatctgaggacacggccatttattactgtgcgcgaggtgagacctactacgagggaaactttgacttctggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagtccctctccctgtctccgggtaaataggtttaaactcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcagaattcctgcagctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaggtggccacctaagggttctcagatgcagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 146 - DNA, 2157 bp, synthetic construct
  source 1..2157: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtcgtaaacgaagaggatccggggagggccggggcagcctgctgacctgcggagacgtggaggagaaccctggccccatgcacagacctagacgtcgtggaactcgtccacctccactggcactgctcgctgctctcctcctggctgcacgtggtgctgatgcacaggtccacctggtgcagtctgggccagaggtgaagaagcctgggtcctcggtgaaggtctcctgcaaggcttctggagtcaccttcatcagtcatgctatcagctgggtgcgacaggcccctggacaagggcttgaatgggtgggaggaatcatcgctatctttggtacaacaaactacgcacagaagttccagggcagagtcacggttacaacggacaaatccacgaacacagtctacatggaattgagcagactgagatctgaggacacggccatttattactgtgcgcgaggtgagacctactacgagggaaactttgacttctggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagtccctctccctgtctccgggtaaa